(19) United States
(12) Patent Application Publication
(10) Pub. No.: US 2025/0257367 A1
(43) Pub. Date: Aug. 14, 2025

(54) GENE THERAPY FOR GAUCHER DISEASE

(71) Applicant: AstraZeneca Ireland Limited, Dublin (IE)

(72) Inventors: Robert D. Bell, Framingham, MA (US); Ting-Wen Cheng, Cambridge, MA (US); Jatin Narula, Cambridge, MA (US); Clark Qun Pan, Cambridge, MA (US); Suryanarayan Somanathan, Cambridge, MA (US); David Wayne Souza, Millinocket, ME (US); Ricardos Tabet, Cambridge, MA (US)

(73) Assignee: AstraZeneca Ireland Limited, Dublin (IE)

(21) Appl. No.: 18/729,960

(22) PCT Filed: Jan. 18, 2023
(86) PCT No.: PCT/IB2023/050426

§ 371 Date: Jul. 18, 2024

Related U.S. Application Data
(60) Provisional application No. 63/267,046, filed on Jan. 21, 2022.
(60) Provisional application No. 63/382,502, filed on Nov. 5, 2022.

(51) Int. Cl. C12N 15/86; A61K 48/00; C12N 9/24

(52) U.S. Cl. CPC C12N 15/86; C12N 9/2405; C12Y 302/01045; A61K 48/00; C12N 2750/14143; C12N 2750/14152

(57) ABSTRACT

The disclosure describes improved vectors, such as adeno-associated virus (AAV) vectors, for expressing beta-gluco-cerebrosidase (GCase) in transduced cells, and use of such vectors to increase the amount of GCase in subjects experiencing a GCase deficiency, such as subjects with Gaucher disease Type 1.

### BACKGROUND OF THE INVENTION

Gaucher disease (GD) is an autosomal recessive lysosomal storage disorder resulting from mutations in the gene (GBA) encoding β-glucocerebrosidase. Insufficient activity of this enzyme in lysosomes results in the intracellular accumulation of the glycolipid glucocerebroside throughout the body, but particularly in bone marrow, spleen and liver. While symptoms, age of onset, and severity vary greatly among individuals, common symptoms include hepatosplenomegaly, anemia, thrombocytopenia, and skeletal abnormalities.

Gaucher disease presents as three major clinical subtypes. Type 1, which is most common, does not involve the nervous system, whereas type 2 (acute neuronopathic) and type 3 (subacute neuronopathic) result in glucocerebroside accumulation in the brain, and are therefore associated with neurological complications. Two less common forms of the disorder are known as well, which include perinatal lethal GD and cardiovascular GD.

Two approaches to treating GD have been developed to date. Enzyme replacement therapy (ERT) for GD seeks to compensate for the deficient β-glucocerebrosidase activity by intraveneously infusing recombinant versions of the enzyme. Examples of approved ERT drugs for GD type 1 include imiglucerase and velaglucerase alfa. Although ERT is effective for reducing at least some symptoms of GD, infusions must be given every two weeks, on average, and therapy is lifelong. In addition, it is not uncommon for GD patients undergoing ERT to develop neutralizing antibodies against the exogenously supplied GCase, which reduces efficacy, or may require cessation of ERT. The second, and more recently developed approach, substrate reduction therapy (SRT), involves oral administration of small molecule drugs that inhibit biosynthesis of GlcCer, the GCase substrate. Examples of approved SRT drugs for GD type 1 include eliglustat and miglustat. SRT, however, is only approved in adults, not pediatric GD patients, is associated with a number of side effects, and also requires lifelong treatment to manage the disease process and symptoms associated with GD.

In view of the disadvantages associated with the current standard of care, there exists a need in the art for improved ways of treating Gaucher disease type 1, and in particular therapies that require less frequent administration.

### SUMMARY OF THE INVENTION

To address the need in the art, the present disclosure provides improved adeno-associated virus (AAV) vectors for expressing beta-glucocerebrosidase (GCase) protein, methods of producing such AAV vectors, and methods of using such AAV vectors to prevent or treat diseases or disorders in subjects characterized by a deficiency in the amount of GCase protein and/or enzymatic activity of GCase protein, including but not limited to Gaucher disease type 1.

Certain enumerated non-limiting embodiments (E) of the inventions of the disclosure are set forth below. These and related embodiments are described in further detail in the Detailed Description, including the Examples and Drawings. Those skilled in the art will recognize or will be able to ascertain, using no more than routine experimentation, equivalents to the specific embodiments described herein.

E1. A recombinant adeno-associated virus (AAV) vector genome comprising a nucleotide sequence encoding beta-glucocerebrosidase (GCase) protein.

E2. The AAV vector genome of E1, wherein said GCase protein comprises a secretion signal peptide sequence and a mature polypeptide sequence.

E3. The AAV vector genome of E1 to E2, wherein the mature polypeptide sequence of said GCase protein is from a wild-type human GCase protein.

E4. The AAV vector genome of E1 to E3, wherein the mature polypeptide sequence of said GCase protein comprises, consists essentially of, or consists of the amino acid sequence of SEQ ID NO: 40.

E5. The AAV vector genome of E1 to E4, wherein said secretion signal peptide sequence is from a wild-type human GCase protein.

E6. The AAV vector genome of E5, wherein said secretion signal peptide sequence comprises the amino acid sequence of SEQ ID NO:39.

E7. The AAV vector genome of E1 to E4, wherein said secretion signal peptide sequence is from a protein other than GCase.

E8. The AAV vector genome of E7, wherein said secretion signal peptide sequence is from an immunoglobulin protein.

E9. The AAV vector genome of E8, wherein said secretion signal peptide sequence comprises the amino acid sequence of SEQ ID NO:38.

E10. The AAV vector genome of E1 to E4, wherein the amino acid sequence of said GCase protein is provided by the amino acid sequence of SEQ ID NO:16.

E11. The AAV vector genome of E1 to E10, wherein said nucleotide sequence encoding the mature polypeptide sequence of said GCase protein is a wild-type nucleotide sequence.

E12. The AAV vector genome of E1 to E10, wherein said nucleotide sequence encoding the mature polypeptide sequence of said GCase protein is a codon-optimized nucleotide sequence.

E13. The AAV vector genome of E12, wherein the codon-optimized nucleotide sequence has a reduced number of CpG di-nucleotides compared to a wild-type nucleotide sequence encoding the mature polypeptide sequence of said GCase protein.

E14. The AAV vector genome of E13, wherein the nucleotide sequence encoding GCase protein has 1-50, 1-45, 1-40, 1-35, 1-30, 1-25, 1-20, 1-15, 1-10, or 1-5 fewer CpG di-nucleotides compared to a wild type nucleotide sequence encoding the mature polypeptide sequence of said GCase protein.

E15. The AAV vector genome of E13 to E14, wherein said wild-type nucleotide sequence encoding the mature polypeptide sequence of said GCase protein is comprised by the nucleotide sequence of SEQ ID NO:28, SEQ ID NO:29, SEQ ID NO:30, SEQ ID NO:31, or SEQ ID NO:32.

E16. The AAV vector genome of E13, wherein the nucleotide sequence encoding GCase protein is devoid of any CpG di-nucleotides.

E17. The AAV vector genome of E1 to E13, wherein the nucleotide sequence encoding the mature polypeptide sequence of said GCase protein is at least 50%, 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to the nucleotide sequence of SEQ ID NO:13.

E18. The AAV vector genome of E1 to E13, wherein the nucleotide sequence encoding the mature polypeptide sequence of said GCase protein is identical to the nucleotide sequence of SEQ ID NO:13.

E19. The AAV vector genome of E1 to E13, wherein the nucleotide sequence encoding said GCase protein is identical to the nucleotide sequence of SEQ ID NO:41.

E20. The AAV vector genome of E1 to E19, wherein said genome comprises at least one AAV inverted terminal repeat (ITR).

E21. The AAV vector genome of E20, wherein the nucleotide sequence of said ITR is wild-type.

E22. The AAV vector genome of E20, wherein the nucleotide sequence of said ITR is modified.

E23. The AAV vector genome of E22, wherein the nucleotide sequence of said ITR is modified to reduce or eliminate the ability of the ITR to undergo terminal resolution.

E24. The AAV vector genome of E22, wherein the nucleotide sequence of said ITR is modified to inactivate the terminal resolution site.

E25. The AAV vector genome of E22, wherein the nucleotide sequence of said ITR is modified to reduce or eliminate the ability of the ITR to support packaging into a capsid.

E26. The AAV vector genome of E22, wherein the nucleotide sequence of said ITR is modified to inactivate the D region.

E27. The AAV vector genome of E20 to E21, wherein said ITR is an AAV2 ITR.

E28. The AAV vector genome of E27, wherein said AAV2 ITR is truncated.

E29. The AAV vector genome of E20, wherein said ITR comprises, consists essentially of, or consists of the nucleotide sequence of SEQ ID NO:7, SEQ ID NO: 15, SEQ ID NO: 18, or SEQ ID NO:19, or the complement or reverse complement of each of said sequences.

E30. The AAV vector genome of E20 to E21, wherein said ITR is other than an AAV2 ITR.

E31. The AAV vector genome of E1 to E30, wherein said vector genome comprises a first AAV ITR positioned at it 5′ terminus and a second AAV ITR positioned at its 3′ terminus.

E32. The AAV vector genome of E31, wherein said vector genome further comprises a third AAV ITR.

E33. The AAV vector genome of E32, wherein said third ITR is modified to inactivate the terminal resolution site.

E34. The AAV vector genome of E1 to E33, wherein said vector genome further comprises a transcription control region operably linked with said nucleotide sequence encoding GCase protein.

E35. The AAV vector genome of E34, wherein said transcription control region is constitutive.

E36. The AAV vector genome of E34, wherein said transcription control region is inducible.

E37. The AAV vector genome of E34, wherein said transcription control region is tissue specific.

E38. The AAV vector genome of E37, wherein said transcription control region is liver tissue specific.

E39. The AAV vector genome of E34 to E38, wherein said transcription control region comprises a promoter sequence.

E40. The AAV vector genome of E39, wherein said transcription control region further comprises an enhancer sequence.

E41. The AAV vector genome of E40, wherein said enhancer sequence is positioned 5′ of the promoter.

E42. The AAV vector genome of E40, wherein said enhancer sequence is positioned 3′ of the promoter.

E43. The AAV vector genome of E39, wherein said promoter sequence is liver tissue specific.

E44. The AAV vector genome of E40, wherein said enhancer sequence is liver tissue specific.

E45. The AAV vector genome of E40, wherein each of said promoter sequence and enhancer sequence is liver tissue specific.

E46. The AAV vector genome of E39, wherein said promoter sequence is derived from the human albumin (ALB) gene.

E47. The AAV vector genome of E40, wherein said enhancer sequence is derived from the human albumin (ALB) gene or the alpha-1-microglobulin/bikunin precursor (AMBP) gene.

E48. The AAV vector genome of E46, wherein said promoter sequence comprises, consists essentially of, or consists of the nucleotide sequence of SEQ ID NO:10, or a promoter functional subsequence, modification or variant thereof.

E49. The AAV vector genome of E47, wherein said enhancer sequence comprises, consists essentially of, or consists of the nucleotide sequence of SEQ ID NO:9 or SEQ ID NO:8, respectively, or an enhancer functional subsequence, modification or variant thereof.

E50. The AAV vector genome of E1 to E49, wherein said vector genome further comprises a transcription termination signal sequence.

E51. The AAV vector genome of E50, wherein said transcription termination signal sequence is a polyadenylation (poly(A)) signal sequence.

E52. The AAV vector genome of E51, wherein said transcription termination signal sequence is derived from the bovine growth hormone (bGH) gene.

E53. The AAV vector genome of E52, wherein said transcription termination signal sequence comprises, consists essentially of, or consists of the nucleotide sequence of SEQ ID NO:14, or a transcription termination signal functional subsequence, modification or variant thereof.

E54. The AAV vector genome of E1 to E53, wherein said vector genome further comprises an intron sequence.

E55. The AAV vector genome of E54, wherein said intron sequence is positioned within and interrupts the nucleotide sequence encoding said GCase protein.

E56. The AAV vector genome of E54, wherein said intron sequence does not interrupt the nucleotide sequence encoding said GCase protein.

E57. The AAV vector genome of E56, wherein said intron sequence is positioned 5′ of the nucleotide sequence encoding said GCase protein.

E58. The AAV vector genome of E57, wherein said intron sequence is positioned 3′ of the promoter and 5′ of the nucleotide sequence encoding said GCase protein.

E59. The AAV vector genome of E54 to E58, wherein said intron sequence is derived from the human beta globin (HBB) gene.

E60. The AAV vector genome of E59, wherein said intron sequence comprises, consists essentially of, or consists of the nucleotide sequence of SEQ ID NO:11.

E61. The AAV vector genome of E1 to E60, wherein said vector genome further comprises a post-transcriptional regulatory element (PRE) sequence.

E62. The AAV vector genome of E61, wherein said PRE sequence is positioned 3′ of the nucleotide sequence encoding said GCase protein and 5′ of the transcription termination signal sequence.

E63. The AAV vector genome of E61 to E62, wherein said PRE sequence is a WPRE or a HPRE sequence.

E64. The AAV vector genome of E63, wherein said PRE sequence comprises, consists essentially of, or consists of the nucleotide sequence of SEQ ID NO:27.

E65. The AAV vector genome of E1 to E64, wherein said vector genome further comprises a binding site for a microRNA (miRNA).

E66. The AAV vector genome of E65, wherein said miRNA binding site is positioned 3′ of the nucleotide sequence encoding said GCase protein and 5′ of the transcription termination signal sequence.

E67. The AAV vector genome of E1 to E64, wherein said vector genome further comprises a stuffer or filler nucleotide sequence of sufficient length such that the entire length of said AAV vector genome is approximately 4.5 to 5.0 kilobases.

E68. The AAV vector genome of E1 to E64, wherein said vector genome comprises a first AAV ITR, a transcription control region in operable linkage with said nucleotide sequence encoding GCase protein, a transcription termination signal sequence, and a second AAV ITR.

E69. The AAV vector genome of E68, wherein said vector genome comprises in 5′ to 3′ order said first AAV ITR, said transcription control region in operable linkage with said nucleotide sequence encoding GCase protein, said transcription termination signal sequence, and said second AAV ITR.

E70. The AAV vector genome of E69, wherein said transcription control region comprises a promoter positioned 5′ of said nucleotide sequence encoding GCase protein and an enhancer positioned 5′ of said promoter.

E71. The AAV vector genome of E68 to E70, wherein said vector genome further comprises an intron positioned between said promoter and said nucleotide sequence encoding GCase protein.

E72. The AAV vector genome of E68 to E70, wherein said vector genome further comprises an intron positioned within and interrupting said nucleotide sequence encoding GCase protein.

E73. The AAV vector genome of E68 to E72, wherein said vector genome further comprises a PRE positioned between said nucleotide sequence encoding GCase protein and said poly(A) signal sequence.

E74. The AAV vector genome of E68 to E73, wherein said first AAV ITR is positioned at the 5′ terminus of said vector genome and said second AAV ITR is positioned at the 3′ terminus of said vector genome.

E75. The AAV vector genome of E74, wherein said vector genome further comprises a third AAV ITR positioned between said first and second AAV ITRs.

E76. The AAV vector genome of E75, wherein the terminal resolution site of said third AAV ITR is inactivated.

E77. The AAV vector genome of E68 to E76, wherein said transcription control region is liver tissue specific.

E78. The AAV vector genome of E77, wherein said transcription control region comprises a first copy of an AMBP gene enhancer sequence, a second copy of an of an AMBP gene enhancer sequence, an ALB gene enhancer sequence, and an ALB gene promoter sequence.

E79. The AAV vector genome of E78, wherein each of said first and second copies of an AMBP gene enhancer sequence comprise, consist essentially of, or consist of the nucleotide sequence of SEQ ID NO:8, said ALB gene enhancer sequence comprises, consists essentially of, or consists of the nucleotide sequence of SEQ ID NO:9, and said ALB gene promoter sequence comprises, consists essentially of, or consists of the nucleotide sequence of SEQ ID NO:10.

E80. The AAV vector genome of E71 to E79, wherein said intron sequence is derived from the human beta globin (HBB) gene.

E81. The AAV vector genome of E80, wherein said intron sequence comprises, consists essentially of, or consists of the nucleotide sequence of SEQ ID NO:11.

E82. The AAV vector genome of E68 to E81, wherein said transcription termination signal sequence is derived from the bovine growth hormone (bGH) gene.

E83. The AAV vector genome of E82, wherein said transcription termination signal sequence comprises, consists essentially of, or consists of the nucleotide sequence of SEQ ID NO:14, or a transcription termination signal functional subsequence, modification or variant thereof.

E84. The AAV vector genome of E68 to E83, further comprising a modified TBP intron 2 sequence positioned 3′ of said transcription termination signal sequence and 5′ of said second AAV ITR.

E85. The AAV vector genome of E1 to E4, wherein said vector genome comprises in 5′ to 3′ order:
(a) a first AAV2 ITR,
(b) a first copy of an AMBP gene enhancer sequence
(c) a second copy of an AMBP gene enhancer sequence
(d) an ALB gene enhancer sequence
(e) an ALB gene promoter sequence
(f) a human beta globin (HBB) gene intron sequence
(g) a nucleotide sequence encoding a human GCase protein operably linked with said first and second copies of an AMBP enhancer, said ALB enhancer and said ALB promoter, wherein said GCase protein comprises a secretion signal peptide sequence and a mature polypeptide sequence,
(h) a bovine growth hormone (bGH) gene transcription termination signal sequence
(i) a modified TBP intron 2 sequence; and
(j) a second AAV2 ITR.

E86. The AAV vector genome of E85, wherein each of said first and second copies of an AMBP gene enhancer sequence comprise, consist essentially of, or consist of the nucleotide sequence of SEQ ID NO:8; said ALB gene enhancer sequence comprises, consists essentially of, or consists of the nucleotide sequence of SEQ ID NO:9; said ALB gene promoter sequence comprises, consists essentially of, or consists of the nucleotide sequence of SEQ ID NO:10; human beta globin (HBB) gene intron sequence comprises, consists essentially of, or consists of the nucleotide sequence of SEQ ID NO:11; nucleotide sequence encoding a human GCase protein comprises, consists essentially of, or consists of the nucleotide sequence of SEQ ID NO:41; and said bovine growth hormone (bGH) gene transcription termination signal sequence comprises, consists essentially of, or consists of the nucleotide sequence of SEQ ID NO:14.

E87. The AAV vector genome of E85 to E86, wherein each of said first and second AAV2 ITRs comprises, consists essentially of, or consists of the nucleotide sequence of SEQ ID NO:7, SEQ ID NO: 15, SEQ ID NO:18, or SEQ ID NO:19, or the complement or reverse complement of each of said sequences.

E87. The AAV vector genome of E85 to E87, wherein the nucleotide sequence of said vector genome comprises, consists essentially of, or consists of the nucleotide sequence of SEQ ID NO: 17, or the reverse complement thereof.

E88. The AAV vector genome of E1 to E87, wherein said vector genome is equal to or less than 5 kilobases in length.

E89. An AAV vector comprising an AAV capsid and the AAV vector genome of E1 to E88, wherein said vector genome is encapsidated by said capsid.

E90. The AAV vector of E89, wherein said AAV capsid is hepatotropic.

E91. The AAV vector of E89 to E90, wherein said AAV capsid is selected from the group of consisting of: AAV2, AAV3B, AAV5, AAV6, AAV7, AAV8, AAV9, AAV13, AAVrh.74, AAVrh.10, AAV-DJ, AAV-LK03, AAV-KP1, AAV-hu.Lvr01, AAV-hu.Lvr02, AAV-hu.Lvr03, AAV-hu.Lvr04, AAV-hu.Lvr05, AAV-hu.Lvr06, AAV-hu.Lvr07, AAV-Anc80, AAV-NP40, AAV-NP59, AAV-NP84, AAV-hu.37, AAV-rh.8, AAV-rh.64R1, RHM4-1, RHM15-1, RHM15-2, RHM15-3/RHM15-5, RHM15-4, and RHM15-6, AAVHSC7, AAVHSC15, and AAVHSC17.

E92. The AAV vector of E89 to E90, wherein said AAV capsid comprises a VP1 protein comprising or consisting of an amino acid sequence selected from the group of consisting of: SEQ ID NO: 1 and SEQ ID NO:4.

E93. The AAV vector of E89 to E92, wherein said vector genome is a single-stranded DNA genome.

E94. The AAV vector of E89 to E92, wherein said vector genome is a self-complementary DNA genome.

E95. The AAV vector of E93 to E94, wherein said vector genome is in the plus polarity.

E96. The AAV vector of E93 to E94, wherein said vector genome is in the minus polarity.

E97. An AAV vector comprising an AAV3B capsid and an AAV vector genome, wherein the nucleotide sequence of said vector genome comprises or consists of the nucleotide sequence of SEQ ID NO:17, or the reverse complement thereof, and wherein said vector genome is encapsidated by said capsid.

E98. A pharmaceutical composition comprising the AAV vector of E89 to E97 and a pharmaceutically acceptable excipient.

E99. A method of increasing the amount of active GCase protein in peripheral blood mononuclear cells (PBMC) of a human subject diagnosed with a deficiency of GCase enzymatic activity, or reducing the amount of glucosylsphingosine in serum of said subject, comprising administering to said subject an amount of the AAV vector or composition of E1 to E98 effective to increase amounts of active PBMC GCase protein, or reduce amounts of serum glucosylsphingosine.

E100. The method of E99, wherein said method is effective to increase the amount of active GCase protein in PBMCs of said subject to at least or about 5%, 10%, 15%, 20%, 25%, 30%, 35%, 40%, 45%, 50%, 60%, 70%, 80%, 90%, or 100% of the amount of active GCase protein in the PBMCs of healthy humans.

E101. The method of E100, wherein the amount of active GCase protein in PBMCs of healthy humans ranges from about 20 to 130 picograms per million (pg/1E6) cells or averages about 70 pg/1E6 cells.

E102. The method of E99, wherein said method is effective to increase the amount of active GCase protein in PBMCs of said subject by at least 25%, 50%, 75%, 100%, 125%, 150%, 175%, 200%, 225%, 250%, 300%, 400%, or 500% compared to the average amount of active GCase protein in PBMCs in said subject prior to administration of said AAV vector or composition.

E103. The method of E99, wherein said method is effective to increase the amount of active GCase protein in PBMCs of said subject to at least or about 10, 15, 20, 25, 30, 35, 40, 45, 50, 55, 60, 65, 70, 75, 80, 85, 90, 95, 100, 105, 110, 115, 120, 125, 130, 140, or 150 pg/1E6 cells.

E104. The method of E99, wherein said method is effective to reduce the amount of glucosylceramide or glucosylsphingosine in serum of said subject by at least 5%, 10%, 15%, 20%, 25%, 30%, 35%, 40%, 45%, 50%, 60%, 70%, 80%, or 90% compared to the average amount of serum glucosylceramide or glucosylsphingosine in said subject prior to administration of said AAV vector or composition.

E105. The method of E99, wherein said method is effective to reduce the amount of glucosylceramide or glucosylsphingosine in serum of said subject to not more than about 200, 175, 150, 125, 100, 75, 50, 25, or 10 nanograms per mL serum.

E106. The method of E99 to E105, wherein said increased amount of active GCase protein in PBMCs or reduced amount of glucosylceramide or glucosylsphingosine in serum of said subject occurs within 10, 15, 20, 25, 30, 35, 40, 45, 50, 55, or 60 days of administration of said AAV vector or composition.

E107. A method of reducing the severity of at least one symptom or sign in a human subject caused by deficiency of GCase enzymatic activity comprising administering to said subject an amount of the AAV vector or composition of E1 to E98 effective to reduce the severity of such symptom or sign.

E108. The method of E107, wherein the symptom or sign is selected from the group consisting of: hepatosplenomegaly, hepatomegaly, splenomegaly, anemia, leukoneutropenia, pancytopenia, thrombocytopenia, monoclonal hypergammaglobulinemia, polyclonal hypergammaglobulinemia, anorexia, chronic fatigue, avascular bone necrosis, bone pain, osteolysis, osteonecrosis, and osteopenia.

E109. The method of E99 to E108, wherein the deficiency of GCase enzymatic activity in said subject is caused by a homozygous or heterozygous mutation in the gene encoding GCase protein that reduces the amount or enzymatic activity of the GCase protein that is expressed from the gene relative to the average amount or activity of GCase proteins in healthy humans with a non-mutated GBA gene.

E110. The method of E99 to E109, wherein said subject is diagnosed with Gaucher disease Type 1.

E111. A method of preventing or treating Gaucher disease Type 1 in a human subject comprising administering to said subject a prophylactically or therapeutically effective amount of the AAV vector or composition of E1 to E98 effective to prevent or treat Gaucher disease Type 1 in said subject.

E112. The method of E99 to E111, wherein the effective amount of said AAV vector is a dose ranging from 1×10¹⁰ to 1×10¹⁵ vector genomes per kilogram (vg/kg) of subject body weight.

E113. The method of E99 to E112, wherein said AAV vector or composition is administered to said subject intravenously.

E114. Use of the AAV vector of E1 to E97 in the manufacture of a medicament for treating or preventing Gaucher disease Type 1 in a human subject.

E115. A DNA plasmid comprising the nucleotide sequence of the AAV vector genome of E1 to E88.

E116. A host cell for AAV vector production comprising the DNA plasmid of E115.

E117. The host cell of E116, wherein said host cell is a HEK293 cell.

E118. The host cell of E116 to E117, wherein said host cell further comprises a gene encoding an AAV Rep protein, such as contained in a DNA plasmid.

E119. The host cell of E116 to E118, wherein said host cell further comprises a gene encoding an AAV VP1 capsid protein, such as contained in a DNA plasmid.

E120. The host cell of E116 to E119, wherein said host cell further comprises a gene coding for a viral helper factor, such as contained in a DNA plasmid.

E121. A method of making a AAV vector, comprising: incubating the host cell of E120 under conditions sufficient to allow the production of AAV vectors, and purifying the AAV vectors produced thereby.

E122. An AAV vector produced by the method of E121.

### BRIEF DESCRIPTION OF THE DRAWINGS

FIG. 1. GCase enzymatic activity produced in Huh7 cells after transduction with different doses of three AAV vectors for expressing human GCase protein.

FIG. 2. Transduction of liver and spleen by two AAV GCase vectors administered to D409V mice quantified as the number of vector genomes normalized to the amount of cellular genomic DNA in tissue samples from treated and control mice.

FIG. 3. Transduction of liver and spleen by two AAV GCase vectors administered to D409V mice quantified as the number of vector genomes normalized to the number of haploid cellular genomes in tissue samples from treated and control mice.

FIG. 4. Transduction of liver and spleen by two AAV GCase vectors administered to D409V mice quantified as the amount of expressed human GCase mRNA normalized to expression of a cellular housekeeping gene in tissue samples from treated and control mice.

FIG. 5A. Amount of human GCase enzyme present in liver samples from D409V mice administered two AAV GCase vectors as determined using an LC-MS/MS assay.

FIG. 5B. Amount of human GCase enzyme present in spleen samples from D409V mice administered two AAV GCase vectors as determined using an LC-MS/MS assay.

FIG. 5C. Amount of human GCase enzyme present in serum samples from D409V mice administered two AAV GCase vectors as determined using an LC-MS/MS assay.

FIG. 6. Amount of human GCase enzyme present in liver samples from D409V mice administered two AAV GCase vectors visualized by immunohistochemistry as compared to control mice. Numbers indicate different test animals.

FIG. 7A. Amount of human GCase enzymatic activity present in liver samples from D409V mice administered two AAV GCase vectors as determined using a fluorogenic substrate assay. Dotted line indicates GCase enzymatic activity in vehicle treated D409V mice.

FIG. 7B. Amount of human GCase enzymatic activity present in spleen samples from D409V mice administered two AAV GCase vectors as determined using a fluorogenic substrate assay. Dotted line indicates GCase enzymatic activity in vehicle treated D409V mice.

FIG. 7C. Amount of human GCase enzymatic activity present in serum samples from D409V mice administered two AAV GCase vectors as determined using a fluorogenic substrate assay.

FIG. 8A. Amount of glucosylsphingosine present in liver samples from D409V mice administered two AAV GCase vectors as determined using an LC-MS/MS assay. Dotted line indicates elevated glucosylsphingosine level in untreated D409V mice.

FIG. 8B. Amount of glucosylsphingosine present in spleen samples from D409V mice administered two AAV GCase vectors as determined using an LC-MS/MS assay. Dotted line indicates elevated glucosylsphingosine level in untreated D409V mice.

FIG. 8C. Amount of glucosylsphingosine present in serum samples from D409V mice administered two AAV GCase vectors as determined using an LC-MS/MS assay. Dotted line indicates elevated glucosylsphingosine level in untreated D409V mice.

FIG. 9. Transduction of liver, spleen, and dorsal root ganglia by two AAV GCase vectors administered to healthy cynomolgus monkeys quantified as the number of vector genomes normalized to the amount of cellular genomic DNA in tissue samples from treated and control monkeys.

FIG. 10. Transduction of liver, spleen, and dorsal root ganglia by two AAV GCase vectors administered to healthy cynomolgus monkeys quantified as the number of vector genomes normalized to the number of haploid cellular genomes in tissue samples from treated and control monkeys.

FIG. 11. Transduction of liver, spleen, and dorsal root ganglia by two AAV GCase vectors administered to healthy cynomolgus monkeys quantified as the amount of expressed human GCase mRNA normalized to expression of a cellular housekeeping gene in tissue samples from treated and control monkeys.

FIG. 12A. Amount of human GCase enzyme present in liver samples from healthy cynomolgus monkeys administered four ascending doses of an AAV GCase vector as determined using an LC-MS/MS assay. Dotted line indicates the mean value of GCase protein level in 10 to 15 healthy human samples. Gray area represents the standard deviation of healthy human GCase concentration.

FIG. 12B. Amount of human GCase enzyme present in spleen samples from healthy cynomolgus monkeys administered four ascending doses of an AAV GCase vector as determined using an LC-MS/MS assay. Dotted line indicates the mean value of GCase protein level in 10 to 15 healthy human samples. Gray area represents the standard deviation of healthy human GCase concentration.

FIG. 13A. Amount of human GCase enzyme present in liver samples from healthy cynomolgus monkeys administered two ascending doses (3E12 vg/kg and 1E13 vg/kg) of an AAV GCase vector visualized by immunohistochemistry as compared to control monkeys. Numbers indicate different test animals.

FIG. 13B. Amount of human GCase enzyme present in liver samples from healthy cynomolgus monkeys administered two ascending doses (3E13 vg/kg and 5E13 vg/kg) of an AAV GCase vector visualized by immunohistochemistry as compared to control monkeys. Numbers indicate different test animals.

### DETAILED DESCRIPTION OF THE INVENTION

The following discussion is directed to various embodiments of the invention. The term “invention” is not intended to refer to any particular embodiment or otherwise limit the scope of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be an example of that embodiment, and not intended to intimate that the scope of the disclosure, including the claims, is limited to that embodiment.

### Certain Definitions

As used herein, “adeno-associated virus vector” means an adeno-associated virus (AAV) comprising a naturally occurring or non-naturally occurring AAV capsid encapsidating a vector genome. Adeno-associated virus vector may be abbreviated “AAV vector,” and depending on context, may be referred to by synonymous terms, such as “recombinant AAV vector,” “rAAV vector,” “rAAV,” or just “vector.”

As used herein, “vector genome” means an AAV genome modified both to include a heterologous nucleotide sequence and to render any AAV vector containing the vector genome replication incompetent, such as by inactivating or deleting an endogenous AAV rep and/or cap gene.

As used herein, “heterologous nucleotide sequence” means a nucleotide sequence that is introduced into an organism (including a virus) from a different organism (including an organism). The sequence of a heterologous nucleotide sequence may be the same as one that occurs in nature, or may be a modified version thereof, or even partially or entirely synthetic.

As used herein, “expression cassette” means a nucleotide sequence comprising a transgene operably linked with regulatory regions or elements for controlling the initiation and termination of transcription of the transgene from DNA into RNA.

As used herein, “transgene” means a nucleotide sequence that encodes at least one polypeptide, and/or the nucleotide sequence coding for at least one functional RNA molecule. Transgene may be referred to by the synonymous term “gene of interest.”

As used herein, “host cell” means a cell in which AAV vectors are produced. Producer cells and packaging cells are examples of host cells. Host cells can be mammalian or insect, or from other organisms, whether single or multi-cellular.

As used herein, the term “purify,” and the related terms “purified” and “purification,” when used in connection with an AAV vector, or sample or preparation thereof, indicates a relative increase or improvement in purity compared with a starting material containing the vector, and/or a prior intermediate purification step in some scheme of sequential purification steps intended to purify the biological product, and does not require a particular qualitative or quantitative degree of purity, unless otherwise specified.

As used herein, “transduction” means the introduction into a target cell of the genome of an AAV vector. Transduction is distinguished from infection, the latter term being used to refer to the introduction into a cell of the genome of a replication competent adeno-associated virus.

As used herein, “target cell” means a cell that an AAV vector is designed or intended to transduce, or is experimentally observed to be transduced by an AAV vector, whether in vitro, or in vivo in a subject.

As used herein, “subject” means an organism to which an AAV vector is administered for purposes of preventing or treating a disease, disorder, or condition.

### Gaucher Disease

Glycosphingolipids (GSLs) are bioactive glycolipids located in the outer leaflet of the plasma membrane, as well as extracellularly, having a variety of functions, including a role in cell signaling. Biosynthesis of many GSLs includes the step of glucosylation of ceremide, occurring on the cytoplasmic face of the ER and early Golgi apparatus, forming glucosylceramide (also called glucocerebroside; abbreviated GlcCer or GL1), after which glycosyltransferases in the Golgi lumen attach additional sugar molecules, resulting in more complex glycan structures. Aerts, J M F G, et al., Curr Op Chem Biol, 53:204 (2019); Schnaar, R L and Kinoshita, T, Glycosphingolipids (Chapter 11), in Essentials of Glycobiology [Internet], 3^{rd}. Ed., CSH Laboratory Press, Varki, A, et al., eds. (2017). During catabolism, GSLs are endocytosed and degraded in lysosomes, where they are acted upon by glycosidases that sequentially remove terminal sugar moieties until ceremide is left, which is cleaved by lysosomal acid ceramidase into its constituents, a free fatty acid and sphingosine.

Among the lysosomal glycosidases responsible for degrading GSLs to their lipid core is beta-glucocerebrosidase (also known as glucosylceramidase, acid beta-glucosidase, or beta-D-glucosyl-N-acylsphingosine glucohydrolase; EC 3.2.1.45; abbreviated GCase), a lysosomal membrane-associated glycoprotein, encoded by the GBA gene, that catalyzes hydrolysis of glucocerebroside to D-glucose and ceramide. A variety of so-called lysosomal storage diseases are caused by deficiency of activity of the lysosomal glycosidases involved in GSL metabolism, and deficient activity of β-glucocerebrosidase results in a pathologic intracellular accumulation of GlcCer, which is the underlying cause of Gaucher disease (GD). The major non-CNS source of GlcCer is the breakdown of senescent blood cells and tissue debris, and the incompletely metabolized GlcCer substrate is stored in cells of monocyte-macrophage lineage of the reticuloendothelial system.

Although GCase deficiency is the root cause of GD, the disorder's clinical manifestations are highly variable, ranging from neonatal lethality to mild symptoms. The level of GCase activity measured in white blood cells does not with disease severity, and even genotype is not a reliable predictor of phenotype. It is yet to be completely understood the extent to which other variables, such as interaction with other gene products, epigenetic mechanisms, and environmental factors determine the disease course in particular individuals.

Clinically, GD presents in five forms. The most common form in Caucasians is type 1 (about 90% of GD cases), the non-neuropathic type, whereas types 2 and 3 present with primary neurological complications. Two rarer forms have been described as well.

Gaucher disease Type 1 (abbreviated “GD1”), which can be diagnosed at any age, but particularly young adulthood, is associated with bone disease (osteopenia, focal lytic or sclerotic lesions, osteonecrosis (which may not be reversible once it occurs, even with therapy), and fractures), hepatomegaly, splenomegaly, cytopenia (anemia, leukopenia, and/or thrombocytopenia), lung disease (interstitial lung disease, alveolar/lobar consolidation, and/or pulmonary arterial hypertension), and the absence of primary central nervous system disease. Splenomegaly is very common (at least 90% of patients), hepatomegaly common (60-80% of patients), as is bone disease (at least 70% of patients), and cytopenia is nearly universal in untreated GD. In addition to the more common clinical signs, GD type 1 can present coagulation, immunologic and metabolic abnormalities, as well as certain malignancies (e.g., multiple myeloma). Painful bone crises can also occur.

Gaucher disease types 2 and 3 usually manifest at younger ages than type 1, and while associated with many of the same visceral symptoms, are distinct by including primary CNS involvement. Drastic reduction of lysosomal sphingolipid breakdown associated with certain GBA mutations leads to the accumulation of toxic byproducts originating from the turnover of membrane gangliosides in neurons. With GD type 2 (acute neuronopathic form; less than 5% of GD cases), disease onset occurs before two years of age and progresses rapidly to death by age two to four years. Neurological symptoms include bulbar signs, pyramidal signs, and cognitive impairment. Bone disease is absent. With GD type 3 (subacute/chronic neuronopathic; about 5% of GD cases), onset frequently occurs in childhood, but the disease progression is much slower and patients can survive into their third or fourth decade. Neurological symptoms include oculomotor apraxia, seizures, and progressive myoclonic epilepsy.

The perinatal-lethal form (less than 1% of GD cases), due to complete lack of GCase activity causing severe impairment of the skin's barrier function, is associated with ichthyosiform or collodion skin abnormalities, nonimmune hydrops fetalis, and pyramidal signs, whereas the cardiovascular form is characterized by calcification of the aortic and mitral valves, mild splenomegaly, corneal opacities, and supranuclear ophthalmoplegia. The cardiovascular GD phenotype is the only one described as being highly correlated with a particular genotype, i.e., homozygosity for the p.Asp448His variant.

Interestingly, pathogenic variants of GCase are associated with increased risk of developing Parkinson's disease (PD) and Lewy body dementia, in individuals with GD and those who are merely heterozygous, suggesting a role for aberrant glucosylceramide metabolism in the etiology of PD.

A defining feature of GD is accumulation of GlcCer in macrophages (often called Gaucher cells or storage cells), present in spleen, liver, bone marrow, lymph nodes and lung, and it is believed that these cells contribute to many of GD's symptoms. Gaucher cells are reported to be phenotypically similar to so-called alternatively activated macrophages, and produce and secrete a variety of proteins and byproducts that can modulate inflammation, coagulation, complement activation, and function of tissues and organs throughout the body in ways believed to contribute to pathogenesis and symptom manifestation. Boven L A, et al., Am J Clin Pathol, 122 (3): 359-69 (2004).

Among the products made by Gaucher cells are secreted proteins that find their way into the circulation and can serve as plasma biomarkers for the presence of these cells and therefore GD. These include the enzyme chitotriosidase, the chemokine CCL18/PARC, and another protein called glycoprotein nonmetastatic melanoma protein B (gpNMB). Gaucher cells have also been demonstrated to adapt to GCase deficiency by increasing production of a number of products derived from the excess GlcCer as a substrate or cofactor, which are detectable in the plasma. These include increased production of the ganglioside GM3, glycosyl-β-cholesterol (GlcChol), and glucosylsphingosine (GlcSph), which results from deacylation of GlcCer by lysosomal acid ceramidase to its sphingoid base. The latter product in particular may be involved in GD pathogenesis and can be monitored to track response to ERT or SRT. Murugesan V, et al., Am J Hematol, 91:1082-9 (2016).

Gaucher disease is caused by a variety of mutations associated with the human GBA gene (also known as GBA1, GCB, and GLUC), which encodes GCase protein. Located at chromosomal region 1q22, mutations in the GBA gene cause GD to be predominantly inherited as an autosomal recessive disease. The GBA gene contains 12 exons, which give rise to 5 known transcriptional variants encoding 3 protein isoforms. Transcriptional variant 1 (2291 nt long; RefSeq NM_000157.4), variant 2 (2344 nt long; RefSeq NM_001005741.3), and variant 3 (2325 nt long; NM_001005742.3) each encode the GCase isoform 1 precursor, a 536 amino acid long protein (RefSeq NP_000148.2) including a 39 amino acid long signal peptide sequence, and a 497 amino acid long mature polypeptide sequence containing 5 potential N-linked glycosylation sites. Transcript variant 4 (RefSeq 2161 nt long; NM_001171811.2) encodes GCase isoform 2, a 449 amino acid long protein, and variant 5 (RefSeq 2144 nt long; NM_001171812.2) encodes GCase isoform 3, a 487 amino acid long protein.

At least 200 deleterious GBA gene mutations associated with GD1 have been identified, including missense and nonsense mutations, splice junction variants, deletions and insertions of one or more nucleotides, as well as recombination with a pseudogene (GBAP) positioned downstream of GBA. Pathogenic mutations have been associated with mRNA instability, premature translation termination and loss of protein, as well as mutations affecting enzymatic activity of the protein.

GD1 prevalence varies in different populations. For example, while more common in individuals of Ashkenazi Jewish ancestry (about 1-2:1000), GD1 is much less frequent in other Caucasian populations (about 1-2:100,000). Confirmed diagnosis of GD1 is made phenotypically by detecting low GCase enzymatic activity (0-15% of normal) in blood leukocytes or mononuclear cells, and molecular genetically by detecting biallelic pathogenic mutations in the GBA gene, such as by sequence analysis of GBA, followed by gene-targeted deletion-duplication analysis to test for involvement of the nearby pseudogene if no pathogenic variants are found. Although many mutations are pathogenic for GD1, the four most common, accounting for about 90% of pathogenic variants in Ashkenazi Jews and about 50-60% in non-Jewish populations are c.84dupG (also called 84GG), c.115+1G>A (also called IVS2+1), p.Asn409Ser (also called or p.N370S or c.1226A>G), and p.Leu483Pro (also called p.L444P or c.1448T>C).

Three recombinant GCase preparations are approved for treating GD1 by enzyme replacement therapy. Velaglucerase alfa (trade name VPRIV®) has the same amino acid sequence as mature isoform 1 GCase; imiglucerase (trade name Cerezyme®) has the same amino acid sequence as mature isoform 1 GCase, except for an Arg495His substitution; and taliglucerase alfa (trade name Elelyso®) has the same amino acid sequence as mature isoform 1 GCase, except for an 2 additional amino acids at its N-terminus (Glu-Phe) and 7 additional amino acids at its C-terminus (Asp-Leu-Leu-Val-Asp-Thr-Met).

### Adeno-Associated Virus (AAV)

The disclosure provides vectors created from recombinantly modified adeno-associated virus (AAV). AAV vectors are capable of delivering genes, which may be under the control of transcriptional and other regulatory elements, into targeted cells via transduction. By supplying a functional copy of a gene to a target cell in which the endogenous version is missing or mutated, AAV vectors are useful in gene therapy for a variety of diseases and disorders.

AAV is a small non-enveloped, apparently non-pathogenic parvovirus that depends on certain other viruses to supply gene products, known as helper factors, essential to its own replication, a quirk of biology that has made AAV well-suited to serve as a recombinant vector. For example, adenovirus (AdV) can serve as a helper virus by providing certain adenoviral factors, such as the E1A, E1B55K, E2A, and E4ORF6 proteins, and the VA RNA, in cells co-infected by adenovirus and AAV. Other helper viruses, such as herpes simplex virus, have been identified as well. The dependence of AAV replication on accessory factors supplied by other viruses led AAV to be characterized as a type of dependovirus. AAV virions have two major structural features, called the capsid and genome, respectively. The capsid is an icosahedral protein shell that encloses and protects (encapsidates) the viral genome, which contains genes and other sequences required for viral replication in infected cells.

The AAV genome is a single strand of DNA containing two genes called rep and cap. In AAV2, a naturally occurring AAV that infects humans and is particularly well characterized biologically, the genome is about 4.7 kilobases long. By virtue of alternative splicing of the transcripts from two promoters, the rep gene is capable of producing four related multifunctional proteins called Rep (called Rep 78, Rep 68, Rep 52 and Rep 40 in AAV2, named according to their apparent molecular weights) which are involved in viral gene expression, and replication and packaging of genomes. Alternative splicing of the transcript from the single promoter controlling the single cap gene produces three related structural proteins, VP1, VP2, and VP3, a total of 60 of which self-assemble to form the virus's icosahedral capsid in a ratio of approximately 1:1:10, respectively. VP1 is longest of the three VP proteins, and contains amino acids in its amino terminal region that are absent from VP2, which in turn is longer than VP3 and contains amino acids in its amino terminal region that are absent from VP3. In addition to containing the genome, capsid proteins mediate specific binding interactions with receptors on the surface of target cells, based on which AAV can be restricted in their ability to infect certain animal species, and even tissues within the same type of animal, a phenomenon called tropism. For example, one type of AAV may preferentially infect liver cells (e.g., hepatocytes) as compared to muscle or neuronal cells.

In addition to the rep and cap genes, intact AAV genomes have a relatively short (145 nucleotides in AAV2) sequence element positioned at each of their 5′ and 3′ ends called an inverted terminal repeat (ITR). ITRs contain nested palindromic sequences that can self-anneal through Watson-Crick base pairing to form a T-shaped, or hairpin, secondary structure. In AAV2, ITRs have been demonstrated to have important functions required for the viral life cycle, including converting the single stranded DNA genome into double stranded form required for gene expression, as well as packaging by Rep proteins of single stranded AAV genomes into capsid assemblies.

Numerous naturally occurring types of AAV have been discovered in different species. At one time, only six types of primate AAV had been isolated from biological samples (AAV1, AAV2, AAV3, AAV4, AAV5, and AAV6), the first five of which were sufficiently distinct structurally to be classified as different serotypes based on antibody cross reactivity experiments. Later, two novel AAVs, called AAV7 and AAV8 were discovered by PCR amplification of DNA from rhesus monkeys using primers targeting highly conserved regions in the cap genes of the previously discovered AAVs. Gao, G, et al., Novel adeno-associated viruses from rhesus monkeys as vectors for human gene therapy, PNAS (USA) 99 (18): 11854-11859 (2002). Subsequently, a similar approach was used to clone numerous novel AAVs from human and non-human primate tissues, vastly expanding the scope of known AAV capsid protein sequences. Gao, G, et al., Clades of Adeno-Associated Viruses Are Widely Disseminated in Human Tissues, J Virol. 78 (12): 6381-6388 (2004). Many AAV capsid protein sequences are highly similar to each other, or previously identified AAVs, and while often referred to as distinct AAV “serotypes,” not all such capsids would necessarily be expected to be immunologically distinguishable if tested by antibody cross reactivity. AAVs, or AAV capsids, which are not serologically distinguishable from a defined serotype but contain capsid proteins with a different amino acid sequence are better termed variants of the known serotype. Numerous capsids made from naturally and non-naturally occuring capsid proteins have found utility in creating AAV gene therapy vectors.

As established studying AAV2, after binding its cognate receptor on a cell surface, the AAV viral particle enters the cell via endocytosis. Upon reaching the low pH of lysosomes, capsid proteins undergo a conformational change which allows the capsid to escape into the cytosol and then be transported into the nucleus. Once there, the capsid disassembles, releasing the genome which is acted on by cellular DNA polymerases to synthesize the second DNA strand starting at the ITR at the 3′ end, which functions as a primer after self-annealing. Expression of the rep and cap genes can then commence, followed by formation and release from the cell of new viral particles.

### AAV Vectors

The relative simplicity of AAV structure and life cycle, and the fact that it is not known to be pathogenic in humans, inspired researchers to engineer AAV and investigate if it could be converted from a virus to a recombinant vector for gene therapy. Briefly, this was done by cloning the entire genome of AAV2, including both ITRs, into a plasmid, removing the rep and cap genes into a separate plasmid, and replacing them with a heterologous gene expression cassette comprising a promoter controlling a protein encoding transgene. Thus, the only viral genomic sequences retained in the vector genome were the ITRs, due to their critical function in packaging and gene expression, and without which AAV vectors could not be produced or function to express the transgene after transduction of target cells. Finally, to avoid the need for co-infection with a helper virus, necessary for replication of AAV virions, genes for the so-called helper factors (such as, in the case of AdV, the E1A, E1B55K, E2A, E4ORF6, and VA RNA helper factors) were cloned into a third plasmid.

When the three plasmids (which are sometimes called the transgene, rep/cap, and helper plasmids) were transfected together into mammalian host cells, Rep and capsid proteins, and the helper virus factors were expressed from their respective plasmids. These gene products then functioned in the host cells to replicate the vector genome into single stranded DNA from the plasmid on which its sequence resided, assemble capsids, and package the single stranded genomes into the capsids, forming vectors. The vectors could then be purified from the host cells. Because the rep and cap genes existed in trans on a different plasmid, outside their usual context flanked by ITRs, they were not packaged into the vectors. Consequently, while AAV vectors produced this way were able to bind to and convey the expression cassette within their genomes into target cells, they are unable to replicate and create new vector particles.

If vectors function as intended, after transduction, the expression cassette will be transcriptionally active and produce the gene product encoded by the transgene in the target cell. AAV vectors are highly versatile because vector genomes comprising a variety of transgenes under the control of different functional sequences and regulatory elements in various configurations can be designed and paired with a variety of naturally occurring and engineered capsids, with different tropisms and other properties. Many types of gene products can therefore be produced, with a degree of control over the types of cells that are transduced and amount of gene product that is made.

AAV vectors comprise a vector genome encapsidated by an AAV capsid. In some embodiments, the AAV vector genome comprises at least one AAV inverted terminal repeat (ITR) and a heterologous nucleotide sequence with a desired function when present or expressed in a transduced target cell. In some embodiments, the heterologous nucleotide sequence originates from a different type of virus, or an entirely different type of organism, such as an animal, plant, protist, fungus, bacteria, archaea, or other type of organism. In some embodiments, the heterologous nucleotide sequence replaces some or all of the native AAV rep and/or cap genes so that the vector is incapable of expressing functional Rep or VP proteins in transduced target cells. In some embodiments, the entire sequence of the vector genome consists of heterologous nucleotide sequences except for AAV inverted terminal repeat sequences positioned at the ends of the genome.

### Expression Cassettes

In some embodiments, the heterologous nucleotide sequence comprises or consists of an expression cassette comprising a transgene operably linked with a promoter and optionally one or more enhancers, serving to control transcription initiation of the transgene from DNA into RNA, as well as a transcription termination element, such as a polyadenylation signal sequence, serving to terminate transcription of the transgene into RNA. AAV vectors can comprise more than one transgene, either as part of one transcriptional unit, or each being part of its own transcriptional unit. As described in later sections, expression cassettes can further comprise additional sequence elements designed to influence transcription, transcript stability, translation, or other functions.

As AAV vectors are typically designed, the structure of the expression cassette, and the genome overall, is limited by the packaging capacity of the capsid, so that the length of the transgene when combined with all other elements in the genome required for vector function, such as the transcriptional control elements and ITRs, does not exceed approximately 5 kilobases in the case of AAV2, although other types of capsids may have greater or smaller packaging limits. Within the size constraints, however, there is great flexibility in choice of transgenes, ITRs, and the other elements required for the vector to function for its intended purpose.

For purposes of gene therapy, the transgene can be any gene, the product of which would be understood to prevent or treat, although not necessarily cure, any disease, disorder or condition of a subject in need of prevention or treatment. In some embodiments, gene therapy is intended to prevent or treat a disease, disorder or condition characterized by an abnormally low amount or even absence of a product produced by a naturally occurring gene in a subject, such as might occur due to a loss of function mutation. Relating to such embodiments, the transgene can be one intended to compensate for the subject's defective gene by providing to at least some of the subject's cells the same or similar gene product when expressed. A non-limiting example would be a vector designed to express a functional version of clotting factor IX for use in gene therapy of hemophilia B, which is caused by a loss of function mutation in the native factor IX gene. In other embodiments, however, the transgene could be one intended to counteract the effects of a deleterious gain of function mutation in targeted cells. In some embodiments, the transgene can encode a transcriptional activator to increase the activity of an endogenous gene which produces a desirable gene product, or conversely a transcriptional repressor to decrease the activity of an endogenous gene which produces a deleterious gene product.

In some embodiments, the transgene can encode for a polypeptide, or code for an RNA molecule with a function distinct from encoding protein, such as a regulatory non-coding RNA molecule (e.g., micro RNA, small interfering RNA, piwi-acting RNA, enhancer RNA, long non-coding RNA, etc.). Protein encoding sequences in a transgene can be codon-optimized, and translation start sites (e.g., Kozak sequence) can be modified to increase or decrease their tendency to initiate translation. In some embodiments, a transgene encoding amino acid sequence can contain one or more open reading frames, and/or contain one or more splice donor and acceptor site pairs to permit alternative splicing of different messages and polypeptide sequences from such messages. Transgenes encoding proteins further comprise one or more stop codons to end translation of the polypeptide chain.

In some embodiments, a vector genome can be designed for purposes of editing or otherwise modifying the genome of a target cell. For example, a vector genome can include an expression cassette or transgene flanked by homology arms intended to promote homologous recombination between the vector genome and the target cell genome. In another example, a vector genome can be designed to carry out CRISPR gene editing by expressing a guide RNA (gRNA) and/or an endonuclease, such as Cas9 or related endonucleases, such as SaCas9, capable of binding the gRNA and cleaving a DNA sequence targeted by the gRNA.

### GCase Transgenes

In some embodiments, AAV vectors of the disclosure comprise a vector genome comprising an expression cassette comprising a coding sequence for a GCase protein. Coding sequence for any GCase protein with enzymatic activity capable of cleaving the beta-glucosidic linkage of glycosylceramide (also called glucosylceramide, glucosylcerebroside, GlcCer) can be used, including, without limitation, human lysosomal GCase proteins isoform numbers 1, 2, and 3 (RefSeq NP_000148.2, RefSeq NP_001165282.1, and RefSeq NP_001165283.1, respectively), as well as GCase proteins from non-human species. In other embodiments, the GCase protein can include any naturally occurring variants of human GCase protein that do not contain pathogenic mutations, such as premature translation termination codons, or amino acid substitutions, insertions or deletions that substantially reduce GCase enzymatic activity, and/or protein stability. In yet other embodiments, the GCase protein can include engineered variants of human GCase protein that retain GCase enzymatic activity, such as the variants of GCase isoform 1 used in the recombinant replacement therapies imiglucerase and taliglucerase alfa, as well as other engineered variants, including chimeric variants and variants with amino acid substitutions, insertions or deletions designed to increase GCase specific activity, add or remove glycosylation sites, or sites for other post-translational modifications, or alter other aspects of GCase structure or function. In some embodiments, the native secretion signal peptide sequence is modified or replaced entirely with a secretion signal peptide sequence from a similar or entirely different secreted protein.

For use in AAV vectors of the disclosure, the nucleotide sequence encoding the GCase protein can be any nucleotide sequence capable of encoding the desired GCase protein in the type of cell desired to be transduced by the vector. In some embodiments, the nucleotide sequence encoding GCase protein (transgene) is the same as exists in a naturally occurring gene encoding a GCase, such as a human GCase, such as any of the naturally occurring human nucleotide sequences encoding human GCase isoform number 1, such as RefSeq NM_000157.4 (or nucleotides 138-1748 inclusive), RefSeq NM_001005741.3 (or nucleotides 191-1801 inclusive), and RefSeq NM_001005742.3 (or nucleotides 172-1782 inclusive); or encoding human GCase isoform number 2, such as RefSeq NM_001171811.2 (or nucleotides 269-1618 inclusive); or encoding human GCase isoform number 3, such as RefSeq NM_001171812.2 (or nucleotides 138-1601 inclusive).

In other embodiments, the nucleotide sequence encoding GCase protein can differ at one or more nucleotide positions compared to a naturally occurring nucleotide sequence and, by virtue of the redundancy in the genetic code, still encode the identical GCase protein as the naturally occurring gene sequence. In some embodiments, the nucleotide sequence encoding GCase protein can be intentionally modified to affect its function in transduced cells, such as to eliminate sequence motifs capable of stimulating an innate immune response, to eliminate cryptic splice junctions, to eliminate alternative start codons, to increase the stability of the corresponding mRNA, and/or to increase the rate of translation of mRNA into protein. In other embodiments, the nucleotide sequence encoding GCase protein can be intronless, or can include one or more introns interupting the coding sequence, but which are removed by the splicing apparatus in transduced cells so as to allow translation of the desired GCase protein.

In some embodiments, AAV vectors of the disclosure comprise a transgene encoding a GCase protein comprising a mature GCase polypeptide, a non-limiting example of which is the amino acid sequence of SEQ ID NO:40. In some other embodiments, AAV vectors of the disclosure comprise a transgene encoding a GCase precursor protein comprising or consisting of a secretion signal peptide and a mature GCase polypeptide, where non-limiting examples of the secretion signal peptide include the amino acid sequences of SEQ ID NO:38 or SEQ ID NO:39, and a non-limiting example of the mature GCase polypeptide includes the amino acid sequence of SEQ ID NO:40. In some embodiments, AAV vectors of the disclosure comprise a transgene encoding a mature GCase polypeptide, a non-limiting example of which is the nucleotide sequence of SEQ ID NO:13. In some other embodiments, AAV vectors of the disclosure comprise a transgene encoding a GCase precursor protein, non-limiting examples of which include the nucleotide sequences of SEQ ID NO:28, SEQ ID NO: 29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, or SEQ ID NO:41.

In some other embodiments, AAV vectors of the disclosure comprise a transgene encoding a mature GCase polypeptide or a GCase precursor protein where the protein sequence encoded by the transgene is highly similar, or identical to the protein sequence of a reference sequence, but where the nucleotide sequences of the transgene and reference sequence share a certain percent identity, the differences corresponding to positions within codons that do not change the corresponding amino acid. For example, in some embodiments, the transgene encodes the same GCase protein as SEQ ID NO:28 and has a nucleotide sequence at least 50%, 55%, 60%, 65%, 70%, 75%, 80%, 85%, 86%, 87%, 88%, 89%, 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, or 99.9% identical to SEQ ID NO:28; the transgene encodes the same GCase protein as SEQ ID NO:29 and has a nucleotide sequence at least 50%, 55%, 60%, 65%, 70%, 75%, 80%, 85%, 86%, 87%, 88%, 89%, 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, or 99.9% identical to SEQ ID NO: 29; the transgene encodes the same GCase protein as SEQ ID NO:30 and has a nucleotide sequence at least 50%, 55%, 60%, 65%, 70%, 75%, 80%, 85%, 86%, 87%, 88%, 89%, 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, or 99.9% identical to SEQ ID NO:30; the transgene encodes the same GCase protein as SEQ ID NO:31 and has a nucleotide sequence at least 50%, 55%, 60%, 65%, 70%, 75%, 80%, 85%, 86%, 87%, 88%, 89%, 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, or 99.9% identical to SEQ ID NO:31; the transgene encodes the same GCase protein as SEQ ID NO: 32 and has a nucleotide sequence at least 50%, 55%, 60%, 65%, 70%, 75%, 80%, 85%, 86%, 87%, 88%, 89%, 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, or 99.9% identical to SEQ ID NO: 32; the transgene encodes the same GCase protein as SEQ ID NO:33 and has a nucleotide sequence at least 50%, 55%, 60%, 65%, 70%, 75%, 80%, 85%, 86%, 87%, 88%, 89%, 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, or 99.9% identical to SEQ ID NO:33; the transgene encodes the same GCase protein as SEQ ID NO:34 and has a nucleotide sequence at least 50%, 55%, 60%, 65%, 70%, 75%, 80%, 85%, 86%, 87%, 88%, 89%, 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, or 99.9% identical to SEQ ID NO:34; the transgene encodes the same GCase protein as SEQ ID NO: 35 and has a nucleotide sequence at least 50%, 55%, 60%, 65%, 70%, 75%, 80%, 85%, 86%, 87%, 88%, 89%, 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, or 99.9% identical to SEQ ID NO: 35; the transgene encodes the same GCase protein as SEQ ID NO:36 and has a nucleotide sequence at least 50%, 55%, 60%, 65%, 70%, 75%, 80%, 85%, 86%, 87%, 88%, 89%, 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, or 99.9% identical to SEQ ID NO:36; the transgene encodes the same GCase protein as SEQ ID NO:37 and has a nucleotide sequence at least 50%, 55%, 60%, 65%, 70%, 75%, 80%, 85%, 86%, 87%, 88%, 89%, 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, or 99.9% identical to SEQ ID NO:37; or the transgene encodes the same GCase protein as SEQ ID NO: 41 and has a nucleotide sequence at least 50%, 55%, 60%, 65%, 70%, 75%, 80%, 85%, 86%, 87%, 88%, 89%, 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, or 99.9% identical to SEQ ID NO: 41.

The percentage of nucleotide sequence identity between a reference sequence and a transgene can be determined by any method known in the art. For example, in some embodiments, the nucleotide sequences of the reference sequence and the transgene (or the amino acid sequences encoded by them) can be aligned and compared over their entire lengths and a percent nucleotide sequence identity calculated using a computer algorithm. An exemplary algorithm for globally aligning and comparing nucleotide sequences is the Needleman-Wunsch algorithm. In other embodiments, however, a local alignment algorithm, such as the BLAST algorithm can be used. Needleman, S B, and Wunsch, C D, J Mol Biol, 48 (3): 443-53 (1970); States D J, et al., Methods: A companion to Methods in Enzymology 3:66-70 (1991); Pearson, W R, Curr Protoc Bioinformatics, 43:3.5.1-3.5.9 (2013). In some embodiments, where one or the other of the reference and transgene sequences contains non-coding sequence, such as an intron or a stop codon, then the non-coding sequence(s) are ignored and only the protein coding sequence within the reference and transgene sequences are aligned and compared. Once the optimal global alignment between a reference sequence and a transgene is established, the percent of identical nucleotides between the aligned sequences can be calculated

As is known in the art, sequence comparison algorithms can allow users to define substitution scores and gap penalties, parameters used to calculate alignment scores for the numerous possible alignments that can be made. The alignment with the highest score is then considered optimal. The substitution score involves assigning a numerical reward for matches and penalty for mismatches. Exemplary sets of respective match and mismatch scores include 1,−1; 1,−2; 1,−3; 1,−4; 2,−3; 4,−5, although others are possible. The gap cost involves assigning a numerical penalty for existence of a gap (insertion or deletion of a nucleotide) as well as as penalty for extending the width of the gap once formed. Increasing the gap costs will result in alignments which decrease the number of gaps introduced. Exemplary sets of respective costs for gap existence and extension include 0,−4; −2,−2; −2,−4; −3,−3; −4,−2; −4,−4; −5,−2; −6,−2, although others are possible. In some embodiments, the alignment and comparison of the reference and transgene sequences is carried out using the default substitution scores and gap penalties, and any other default settings, provided with computer software or algorithm for performing the analysis.

### Secretion Signal Peptide Sequences

According to certain embodiments, AAV vectors of the disclosure can be designed and constructed to transduce cells in which GCase protein deficiency contributes directly to the etiology of GD1, for example, cells of the monocyte and macrophage lineage which form Gaucher cells as a result of accumulation of incompletely metabolized glucosylceramide substrate. In such embodiments, the GCase produced after vector transduction desirably remains in the cells where its presence can compensate for the loss of function of the native GCase genes.

In other embodiments, however, AAV vectors of the disclosure can be designed and constructed to transduce cells in organs, such as liver hepatocytes, from which GCase produced after vector transduction is secreted and travels throughout the body, such as in the blood, where it can be taken and up and utilized by other cells, such as monocytes and macrophages. In such embodiments, it is often useful to design the vector so that the GCase protein includes a secretion signal peptide sequence to ensure that the GCase protein can be secreted from transduced cells into the circulation. Otherwise, insufficient amounts of GCase may be available to monocytes and macrophages, or other cells, to correct for the loss of function of the native GCase genes.

Any secretion signal peptide sequence known in the art to be effective to cause a protein to be secreted from a cell in which it is synthesized may be used in connection with the AAV vectors of the disclosure. Desirably, but not necessarily, such signal peptides are removed from the protein by the cell in the process of secretion. In some embodiments, the naturally occurring secretion signal peptide in the human GCase protein precursor isoform 1 can be used, the amino acid sequence of which is provided by SEQ ID NO:39. In other embodiments, however, the native GCase signal peptide can be replaced with a signal peptide from a different secreted protein from humans or another species, for example, to improve the rate at which GCase made in transduced cells is secreted, or for some other reason, such as reduced immunogenicity.

Numerous heterologous secretion signal peptides are known in the art and can be used to facilitate secretion of GCase from cells, such as liver cells, transduced with AAV vectors of the disclosure. In some embodiments, the signal peptide sequence originates from any of a variety of proteins made by and secreted from hepatocytes or cells in liver. Non-limiting examples include signal peptide sequences from human proteins such as Alpha-1-antitrypsin, Alpha-1-antichymotrypsin, Alpha-1-acid glycoprotein 1, Serum albumin, Phosphatidylethanolamine-binding protein 1, Haptoglobin, Plasminogen activator inhibitor 1, Beta-2-microglobulin, Peptidyl-prolyl cis-trans isomerase B, Retinol-binding protein 4, Fetuin-A, Complement C3, Apolipoprotein A-I, Endoplasmic reticulum chaperone BiP, Complement factor B, Protein AMBP, Apolipoprotein E, Clusterin, Cathepsin D, Serotransferrin, Alpha-1-acid glycoprotein 2, and Complement C4-B, with many others being possible. The amino acid sequences and encoding nucleotide sequences for these and other secreted proteins are available in public sequence databases, such as Genbank. In other embodiments, secretion signal peptide sequences for use with vectors of the disclosure can originate with proteins, human or otherwise, that are high secreted from non-liver cells, such as human or murine IgG heavy chain or kappa or lambda light chains. For example, in one non-limiting embodiment, the signal peptide from the murine IgG heavy chain can be used, the amino acid sequence of which is provided as SEQ ID NO:38. The amino acid sequence of naturally occurring secretion signal peptides can be modified to desirably alter their function, as can the nucleotide sequence encoding such wild-type or modified signal peptides in order to achieve a desired type of sequence optimization, such as removal of CpG motifs. In yet other embodiments, entirely synthetic secretion signal peptide sequences can be used.

### Transcription Control Regions

AAV vectors of the disclosure intended to express GCase protein in and/or from transduced cells can further comprise, as part of the vector genome, one or more transcription control regions in operable linkage with the transgene encoding the GCase polypeptide sequence. As discussed further below, different types of transcription control regions are known in the art which can be used to control initiation of transcription of the transgene into RNA. As used herein, the term “operable linkage,” and variations such as “operative linkage,” “operably linked,” and “operatively linked,” refers to a functional relationship between the transcription control region and transgene, so that the control region can affect transcription of the transgene (whether positively or negatively), without specifying any particular spatial or structural relationship between them. Thus, for example, a transcription control region could be operably linked with a transgene even though it is positioned 5′ or 3′ of the transgene, and/or positioned immediately adjacent to or distal from the transgene. Transcription control regions can be constituitively active, active in specific cells or tissues, inducibly active in response to some environmental stimulus, be derived from a naturally occuring gene (of any suitable species), and can be modified to improve or change its function, or even be entirely synthetic.

In some embodiments, a transcription control region comprises a promoter region, which comprises the minimal DNA sequence required to initiate transcription by the transcription apparatus in transduced cells (e.g., a TATA box or initiator sequence), often as well as one or more additional proximal elements that act singly or cooperatively to increase the rate of transcription from the basal promoter. Depending on its sequence, a promoter can initiate transcription by RNA polymerase I, II, or III, but promoters from protein encoding genes, which are usually transcribed by RNA pol II, are often used in AAV vectors intended to express a polypeptide, such as GCase, in transduced cells.

In other embodiments, a transcription control region comprises or further comprises at least one enhancer region, which functions to further increase the rate of gene transcription beyond what the basal promoter alone can sustain. In their natural context, enhancers are often positioned distal from the promoter of the gene on which they act, sometimes tens to hundreds or thousands of basepairs upstream (i.e., 5′), but enhancers can also occur elsewhere, such as in introns or downstream (i.e., 3′) of the gene on which they act. While promoter regions may contain proximal enhancer elements (subsequences which, if removed, would reduce transcription from the basal promoter), enhancers do not usually contain sequences that can function as a basal promoter. Although in nature enhancer regions are often positioned distally to the promoter of the gene on which they act, enhancer regions, or enhancer elements from within larger enhancer regions (such elements often corresponding to DNA binding sites for transcription factors), can sometimes retain at least some of their transcription enhancing function when removed from their natural context and repositioned much closer to a promoter, whether from the same or even a different gene.

Enhancer and promoter regions of genes described in the scientific literature may be too large to be accommodated by the packaging capacity of AAV capsids when combined with a transgene and other genomic elements required for vector function. Accordingly, in some embodiments, functional subsequences within longer enhancer or promoter regions can be identified using methods familiar to those of ordinary skill, and the shorter functional subsequences incorporated into transcription control regions for use in the vectors of the disclosure. In this manner, the size of transcription control regions can be reduced while maintaining their desired function. Using this approach functional elements from naturally occurring enhancers or promoters can be combined in novel ways, such as by modifying their number, spacing and/or arrangement, to create hybrid or synthetic enhancers and or promoters with improved properties. In some embodiments, the enhancer and promoter can each be derived from the same, naturally occurring gene, whereas in other embodiments, the enhancer and promoter can originate from entirely different genes, including genes of different species.

In some embodiments, from the perspective of a coding strand (i.e., plus strand) single stranded DNA AAV vector genome, a promoter sequence is positioned 5′ of a downstream sequence to be transcribed into RNA, such as a transgene encoding a protein, such as GCase. In some embodiments, an enhancer element or region, if present, can be positioned 5′ of the promoter sequence, or instead be positioned elsewhere in the genome, such as in a 5′ or 3′ untranslated region (UTR) adjacent the transgene, in an intron, 3′ of a transcription termination signal sequence, or elsewhere. In some embodiments, a vector genome can comprise more than one enhancer region (of same or different types), which can be positioned adjacent to each other, or spaced apart, and/or separated by other functional elements within the genome. In some embodiments, the same enhancer element or region is provided in a tandemly arranged array of repeating units, such as 2, 3, 4, or more.

In some embodiments, transcription control regions for use in the AAV vectors of the disclosure are non-tissue specific, meaning that they are constitutively active in many different cell types, although not necessarily all. According to some embodiments, non-tissue specific transcription control regions include promoters (which may include enhancer elements proximal to a basal promoter) derived from certain viruses, such as the human cytomegalovirus major immediate early gene (CMV-IE) (Boshart, M, et al., Cell, 41:521-30 (1985); Yew, N L, et al., Hum Gene Ther, 8:575-84 (1997)); simian virus 40 (SV40); as well as the retroviral long terminal repeat (LTR) promoters from Rous sarcoma virus (RSV) and Moloney murine leukemia virus (MoMLV). In other embodiments, non-tissue specific transcription control regions include promoters (which may include proximal enhancer elements) can be derived from genes active in many different cell types, including from different types of animals, such as the human polypeptide chain elongation factor (EF1α) gene; the phosphoglycerate kinase (PGK) gene; the ubiquitin C (UbiC) gene; the chicken beta-actin (CBA) gene; the U1a1 or U1b2 small nuclear RNA promoters (Bartlett, J S, et al., Proc Natl Acad Sci USA 93:8852-7 (1996); Wu, Z, et al., Mol Ther, 16 (2): 280-9 (2008)); the histone H2 or histone H3 promoters (Hurt, M M, et al., Mol Cell Biol 11:2929-36 (1991); Wu, Z, et al., Mol Ther, 16 (2): 280-9 (2008)).

Likewise, enhancer regions can be derived from viruses and genes active in different cell types from different types of animals. As noted, in some embodiments, a promoter and enhancer derived from the same gene can be combined to create a transcription control region for use in the vectors of the disclosure, but enhancers and promoters from different genes can be combined to create hybrid transcription control regions. A commonly used example is the 1.6 kb hybrid enh/pro region called CAG (or CAGGS) comprising the CMV immediate-early enhancer, the chicken beta actin (CBA) gene promoter and the CBA intron/exon 1 (Niwa, H, et al., Gene, 108:193-9 (1991); Ikawa, M, et al., Dev Growth Differ, 37:455-9 (1995)), and later modifications that reduced its size, including one in which the CBA intron was replaced with a smaller simian virus 40 (SV40) intron (Wang, Z, et al., Gene Ther, 10:2105-11 (2003)), and another called CBA hybrid intron (CBh) which replaced the SV40 intron with a hybrid intron composed of a 5′ donor splice site from the CBA 5′ UTR and a 3′ acceptor splice site from the MVM intron (Gray, S J, et al., Hum Gene Ther, 22:1143-53 (2011)).

In some embodiments, transcription control regions for use in the AAV vectors of the disclosure can be liver tissue specific, meaning that they are more or most active in directing expression of a transgene in liver cells, such as hepatocytes, compared to cells of other tissues or organs. Without wishing to be bound by theory, one mechanism by which liver tissue specificity may occur is the presence in an enhancer and/or promoter of one or more specific binding sites for DNA binding transcriptional activator proteins preferentially expressed in liver cells. Use of a liver tissue specific transcription control region can be advantageous, in some embodiments, by reducing or even preventing transgene expression in non-liver cells that may be transduced by a vector, which can reduce the risk of off-target effects.

Liver-specific transcription control regions can be derived from genes that are naturally expressed at high levels in liver, examples of which include the genes for albumin, transthyretin (prealbumin), alpha 1-antitrypsin, prothrombin, and many others. Certain liver-specific genes expressed at high level have both enhancers and promoters, which may be included in transcription control regions. In some embodiments an enhancer and a promoter derived from the same gene may be combined in a liver-specific transcription control region, whereas in other embodiments, an enhancer from one gene and a promoter from a different gene may be combined in a hybrid liver-specific transcription control region. When from the same gene, a transcription control region sequence comprising one or more enhancers and promoter may be copied as it exists in the native gene from which it is derived, or engineered to reduce its length, such as by deleting non-transcriptionally active sequences separating the one or more enhancers and the promoter. In some embodiments, enhancers and promoters for use in liver-specific transcription control regions may derive from genes of different species. In yet other embodiments, the sequence of an enhancer and/or a promoter in a transcription control region can be modified relative to its original sequence by changing, adding or removing nucleotides to improve its function, such as increasing transcription activator binding, reducing transcription repressor binding, or reducing the size of the transcription control region. In some embodiments, it is the enhancer that provides for liver-specific expression and the promoter is not itself liver-specific, whereas in other embodiments, it is the promoter that provides for liver-specific expression and the enhancer, if present, is not itself liver-specific but is capable of increasing the rate of transcription from the liver-specific promoter. For example, a strong viral enhancer, such as the human CMV major immediate early gene enhancer, could be paired with a liver-specific promoter, or a strong liver-specific enhancer, such as from the albumin gene, could be paired with a strong viral promoter, such as the SV40 early promoter. In other embodiments, both the enhancer and promoter each are liver-specific. In some embodiments, different enhancer regions can be combined to form chimeric enhancer regions that are used in transcription control regions in AAV vectors of the disclosure.

Non-limiting examples of enhancers and promoters that may be used in and comprised by liver-specific transcription control regions of AAV vectors of the disclosure include enhancers (at least three of which have been characterized) and promoter from the albumin gene (Minghetti, P P, et al., J Biol Chem, 261 (15): 6747-57 (1986); Frain, M, et al., Mol Cell Biol, 10 (3): 991-9 (1990); Hayashi, Y, et al., J Biol Chem, 267 (21): 14580-5 (1992)); enhancer(s) and promoter from the mouse or human alpha-1-antitrypsin (A1AT) genes (Costa, R H, et al., Mol Cell Biol, 9 (4): 1415-25 (1989); De Simone, V, et al., EMBO J, 6 (9): 2759-66 (1987); Monaci, P, et al., EMBO J, 7 (7): 2075-87 (1988); Hafenrichter, D G, et al., Blood, 84:3394-3404 (1994); Le, M, et al., Blood, 89:1254-59 (1997)); enhancer(s) and promoter from the transthyretin (TTR) gene (Tsuszuki, T, et al., J Biol Chem, 260 (22): 12224-7 (1985); Costa, R H, et al., Mol Cell Biol, 6 (12): 4697-708 (1986)); enhancer(s) and the promoter from the alpha fetoprotein (AFP) gene; enhancer(s) and the promoter from the prothrombin gene (Bancroft, J D, et al., Biochem, 31:12469-76 (1992)); and the HCR1 and HCR2 enhancer regions from the human apolipoprotein E/C-I gene cluster (Allan, C M, et al., J Biol Chem, 272 (46): 29113-9 (1997); Dang, Q, et al., J Biol Chem, 270 (38): 22577-85 (1995); Simone, W S, et al., J Biol Chem, 268 (11): 8221-9 (1993)). Any of the foregoing enhancers can be combined with any of the foregoing promoters from the same or different genes to construct liver-specific transcription control regions for use in the AAV vectors of the disclosure.

A variety of liver tissue specific transcription control regions representing hybrid combinations of enhancers and promoters from different genes (and from the same or different species, such as mouse, rat or human), or transcription control regions from liver-specific genes engineered to reduce their size or improve their performance have been described, any of which can be used in the AAV vectors of the disclosure to express GCase protein in transduced liver cells. Non-limiting examples include the HCR1 enhancer region from the human ApoE gene combined with the promoter from the human alpha 1-antitrypsin (hAAT) gene, of which various versions have been created seeking to reduce the size of the combined elements (Miao, C H, et al., Mol Ther, 1:522-532 (2000); Dang Q, et al., J Biol Chem 270:22577-22585 (1995); Davidoff A M, et al., Mol Ther 11:875-888 (2005); Hafenrichter D G, et al., Blood 84:3394-3404 (1994); Mcintosh J, et al., Blood 121:3335-3344 (2013); Nathwani A C, et al., Blood 107:2653-2661 (2006)); the TTR gene minimal enhancer and promoter (Yan, C, et al., EMBO J, 9:869-78 (1990); Samadani, U and Costa, R H, Mol Cell Biol, 16:6273-84 (1996)); the human factor IX gene promoter combined with multimerized liver transcription factor binding sites (site 5 elements) (GTV) (Hoag, H, et al., Gene Ther, 6:1584-89 (1999)); the human factor VIII gene promoter combined with multimerized HNF-1 binding sites from the same gene (PF8+5HNF-1) (Notley, C, et al., Hum Gene Ther, 13:1583-93 (2002)); a basic mouse albumin promoter combined with a short intron derived from the SV40 intron (Sarkar, R, et al., J Thromb Haemost, 1:220-6 (2003)); two copies of the α1-microglobulin/bikunin enhancer combined with the thyroxine hormone-binding globulin gene promoter (Rouet P, et al., J Biol Chem 267:20765-20773 (1992); Hayashi Y, et al., Mol Endocrinol 7:1049-1060 (1993); III, C R, et al., Blood Coagul Fibrinolysis, 8 (Suppl 2): S23-S30 (1997); Wang, L, et al., Proc Natl Acad Sci USA, 96:3906-10 (1999)); Wang L, et al., Blood 105:3079-3086 (2005)); the albumin promoter combined with the α-fetoprotein MERII enhancer (Wooddell, C I, et al., J Gene Med, 10:551-63 (2008)); and randomly assembled hepatocyte-specific transcription factor binding sites linked to the murine transthyretin promoter (Vigna E, et al., Mol Ther 11:763-775 (2005)).

Additional references relating to liver tissue specific transcription control regions include Schiedner G, et al., Nat Genet 18:180-183 (1998); Xiao, W, et al., J Virol 72:10222-10226 (1998); Kuriyama S, et al., Cell Struct Funct 16:503-510 (1991); Chuah, M K, et al., Mol. Ther., 22:1605-1613 (2014); and Nair, N., et al., Blood, 123:3195-3199 (2014).

In some embodiments, an HCR enhancer sequence can comprise or consist of base pairs 134 to 442 of GenBank U32510.1, and a hAAT promoter can comprise or consist of base pairs 1747 to 2001 of GenBank K02212.1.

In some embodiments, a transcription control region of AAV vectors of the disclosure can comprise or consist of a human albumin (hALB) gene promoter which, in some embodiments, comprises or consists of the nucleotide sequence of SEQ ID NO:10. In some embodiments, a transcription control region can comprise or consist of, or further comprise, a human albumin gene proximal enhancer which, in some embodiments, comprises or consists of the nucleotide sequence of SEQ ID NO:9. In some embodiments, a transcription control region can comprise or consist of, or further comprise, an alpha-1-microglobulin/bikunin precursor (AMBP) enhancer which, in some embodiments, comprises or consists of the nucleotide sequence of SEQ ID NO:8. In some embodiments, a transcription control region can further comprise more than one copy of said AMBP enhancer sequence, such as 2, 3, 4, or more such sequences, which can be arranged in tandem. In some embodiments, a transcription control region can comprise or consist of, in 5′ to 3′ order, two repeats of the AMBP enhancer sequence, a hALB proximal enhancer sequence, and a hALB promoter sequence. In some embodiments, such arrangement of elements can comprise or consist of the nucleotide sequence of SEQ ID NO:42.

### Transcription Termination Signal Sequences

In some embodiments, AAV vectors of the disclosure comprise a vector genome comprising a transcription terminator sequence positioned, from the perspective of a coding (plus) strand single stranded DNA vector genome, 3′ of the transgene. In some embodiments, another sequence, such as 3′ untranslated region (UTR) sequence, can be positioned between the transgene sequence and the transcription terminator sequence. General information about transcription termination can be found, e.g., in Proudfoot, N J, Genes Devel, 25:1770-82 (2011); Kuehner, J N, et al., Nat Rev Mol Cell Biol, 12:283-94 (2011); Porrua, O and Libri, D, Nat Rev Mol Cell Biol, 16:190-202 (2015).

In some embodiments, particulary when the transgene contains protein encoding-sequence (as opposed to the sequence of an RNA with some function other than encoding protein), the transcription terminator sequence can be a polyadenylation signal sequence (abbreviated variously as “polyA,” “pA,” “poly(A)” or “p (A)”). In some embodiments, pA signal sequences can be derived from naturally occurring genes and used in vectors, whereas in other embodiments, pA signals can be modified, such as by shortening them compared to their natural counterparts, or altering their sequence to make them more efficient at transcription termination. In other embodiments, pA signals can be hybrid sequences, combining pA sequences from different genes, or synthetic.

Non-limiting examples of pA signals that may be used in the vectors of the disclosure include the pA signal from the bovine growth hormone gene (bGH pA); human, mouse or rabbit beta-globin gene; SV40 late gene; sNRP1; spA; herpes simplex virus thymidine kinase gene (HSV TK); or adenovirus type 5 L3 polyadenylation site, with may others being possible. In other embodiments, transcription terminators for use in vectors of the disclosure include those that terminate RNA transcripts without directing polyadenylation, such as the histone H4 gene mRNA 3′ end processing signal (Whitelaw, E, et al., Nucleic Acids Res, 14:7059-70 (1986)).

In some embodiments, AAV vectors of the disclosure comprise vector genomes comprising a transgene, transcription of which is terminated by inclusion of a poly(A) site derived from the bovine growth hormone gene (bGH) which, in some embodiments, comprises or consists of the nucleotide sequence of SEQ ID NO:14; of a poly(A) site from SV40 virus which, in some embodiments, comprises or consists of the nucleotide sequence of SEQ ID NO:24 or SEQ ID NO: 25; or of a poly(A) site from the rabbit beta-globin gene which, in some embodiments, comprises or consists of the nucleotide sequence of SEQ ID NO:26.

### Other Vector Genomic Elements

In addition to transcription control regions and transcription termination signal, other sequences, including cis-regulatory elements, can be included in genomes of AAV vectors of the disclosure to improve, control or modulate transgene expression and/or translation in transduced cells, or confer other functions to the vectors. Such elements include, without limitation, untranslated regions from the 5′ and/or 3′ ends of genes, non-coding exons, introns, splice donor and acceptor sites, lox sites, internal ribosome entry sites (IRES), sequence encoding 2A peptides, elements that stabilize RNA transcripts, binding sites for regulatory miRNAs, micro RNA (miRNA) sequences, elements that enhance nuclear export of mRNAs, including viral post-transcriptional regulatory elements, such as the woodchuck hepatitis virus post-transcriptional regulatory element (WPRE), as well as any other element demonstrated empirically to improve transgene expression, even if the mechanism may be uncertain. In other embodiments, vector genomes can include so-called stuffer or filler sequences, which are intended only to increase the overall length of the vector genome to a desired size, for example, to achieve a length close to but still under the packaging capacity of a particular capsid, and thereby reduce the likelihood of adventitiously packaging truncated vector genomes or non-vector DNA into capsids.

Introns can, in some embodiments, be included in vector genomes to increase transgene expression and/or transcript stability. In some embodiments, a protein encoding transgene is provided in which the sequence of exons and intron(s) is the same as in the naturally ocurring gene. In a gene possessing multiple exons and introns, however, one or more of the introns can be removed so as to minimize the overall length to facilitate the inclusion of other elements while not exceeding the capsid packaging capacity. In other embodiments, however, an intron can be provided from an entirely different gene than the gene providing the coding sequence for the vector transgene. Whether the intron is from the same or a different gene as the transgene, the intron can be modified from its original sequence, for example by changing certain nucleotides, or removing internal sequences to reduce its overall length while maintaining the splice donor and acceptor sequence motifs required for efficient splicing to occur, or other intronic cis elements important for function (for example, enhancers that may reside in the original unmodified intron sequence). Introns can also be hybrid, where the splice donor portion of the intron from one gene is paired with the splice acceptor portion of the intron from a different gene, or synthetic, with sequence that does not correspond to any known gene's intron. Introns, in some embodiments, can be positioned within, and therefore interupt, the coding sequence of a transgene (and be provided with the donor and acceptor sites necessary for efficient splicing to occur), whereas in other embodiments an intron is present, but does not interupt the protein coding sequence, and is instead positioned either 5′ or 3′ of the coding sequence. Where an intron does not interupt coding sequence, it may be provided with some exonic sequence carried over from its original genetic context, so long as the exonic sequence does not contain a cryptic translation start signal. In some embodiments, an intron can be positioned 3′ of a promoter (from the perspective of plus strand ssDNA vector genome) and 5′ of the coding sequence. In other embodiments, an intron can be positioned distally from coding sequence in a vector genome, either upstream or downstream.

Non-limiting examples of introns that may be used in the AAV vectors of the disclosure include the small intron from the minute virus of mice (MVM) (Haut, D D and Pintel, D J, J Virol, 72 (3): 1834-43 (1998); Haut, D D and Pintel, D J, Virology, 258:84-94 (1999)); internally deleted intron 1 from human clotting factor IX (FIXm1 and FIXm2) (Kurachi, S, et al., J Biol Chem, 270 (10): 5276-81 (1995)); chimeric beta globin splice donor and immunoglobulin heavy chain splice acceptor intron (GenBank U47120.2 nucleotides 890-1022); intron 1 from the mouse alpha globin gene; and the SV40 small t antigen intron which can comprise or consist of base pairs 4644 to 4552 of GenBank record J02400.1, and which can be modified at positions 4582 (g to c), 4580 (g to c), 4578 (a to c), and 4561 (a to t), as reported in Nathwani, A C, et al., Blood, 107 (7): 2653-61 (2006).

In some embodiments, post-transcriptional regulatory elments (PRE) can be included in vector genomes to increase transgene expression. Examples of PRE include the woodchuck hepatitis virus post-transcriptional regulatory element (WPRE), the hepatitis B virus post-transcriptional regulatory element (HPRE), and modifications thereof. Donello, J E, et al., J Virol, 72 (6): 5085-92 (1998); Loeb, J E, et al., Hum Gene Ther, 10:2295-2305 (1999); Zanta-Boussif, M A, et al., Gene Ther, 16:605-19 (2009); Patricio, M I, et al., Mol Ther Nucleic Acids, 6:198-208 (2017); US Pub. Pat. Appl. 2018-0353620 A1. In some embodiments, from the perspective of a coding (plus) strand single stranded DNA vector genome, at least one WPRE sequence can be positioned downstream of a transgene (thus, 3′ of the stop codon for a transgene encoding a polypeptide) and upstream of the poly(A) signal sequence. In some embodiments, a plurality of PRE can be included, such as 2, 3, or more PRE, of the same or different type, which can be arranged in tandem. In some embodiments,

In some embodiments, AAV vectors of the disclosure comprise vector genomes comprising a WPRE element which, in some embodiments, comprises or consists of the nucleotide sequence of SEQ ID NO:27.

### AAV Inverted Terminal Repeats (ITRs)

Positioned at the termini of the adeno-associated virus genome are unique nucleotide sequences called inverted terminal repeats (abbreviated, “ITR”) which function as origins of viral DNA replication and as priming sites to support the conversion, in infected cells, of the single-stranded (ssDNA) genome into a double-stranded form (dsDNA) competent to support transcription of the rep and cap protein-encoding genes, also contained in the virus genome. The ITRs also function in packaging of replicated ssDNA genomes into AAV capsids. AAV ITRs contain multipalindromic sequences that can fold back on themselves via intrastrand complementary base pairing to form dsDNA T-shaped hairpin secondary structures.

As explained further below, vector genomes of AAV vectors of the disclosure can include one or more AAV ITRs, which function similarly as they do in unmodified virus. Unless otherwise, use of the term “inverted terminal repeat” or “ITR” herein includes intact full-length ITRs, as well as ITRs with modified sequences (such as truncations, internal deletions (such as of a trs or D sequence), additions, and substitutions of one or more nucleotides) that retain one or more of the functions attributable to ITRs (even if less efficiently compared to an intact ITR of the same type), including but not limited to rescue of vector genome from recombinant DNA (such as a plasmid), vector genome replication, and/or packaging of vector genome into assembled capsids.

As they exist in packaged viral and vector genomes, the ITR positioned at the 3′ end of the ssDNA genome will have a free 3′ hydroxyl group, whereas the ITR positioned at the opposite 5′ end of the ssDNA genome will have a free 5′ end. The 5′ ITR can also referred to as the “left” ITR, and the 3′ ITR can also referred to as the “right” ITR. In the context of a plasmid, however, such as might be used in vector production, the vector genome sequence will exist in double-stranded form, such that there will be two sets each of 5′ ITRs and 3′ ITRs. To avoid ambiguity therefore, it should be specified which strand an ITR sits on to distinguish among them. In the absence of such specification, reference to the ITRs of a vector genome in double-stranded form, such as in a plasmid, is with respect to the plus or sense strand, i.e., the DNA strand on which the sequence of the transgene is the same as the coding sequence for a polypeptide product of the transgene, or of a functional RNA, where the transgene is not protein encoding

In wild-type AAV2, the ITRs are 145 bases long, of which the terminal 125 bases comprises the palindromic subsequences. When annealed, the AAV2 ITR contains two double-stranded palindromes, B-B′ and C-C′, forming the arms of the hairpin, which are joined to a larger double-stranded palindrome, A-A′, forming the hairpin's stem. The ITR further contains a D sequence toward the non-terminal end of the ITR, which does not have a complementary sequence within the ITR and therefore remains single-stranded, but does have a complementary sequence in the D region of the ITR at the opposite end of the genome (thus, D and D′). The A-A′ stem structure includes a Rep-binding element (RBE) containing tetranucleotide repeat motifs to which the large AAV Rep proteins bind for purposes of introducing a sequence-specific and strand-specific nick at the terminal resolution site (trs) in the ITR sequence (between nucleotides 124 and 125 in the AAV2 ITR, counting from the 3′ end), a step required for DNA replication of the viral genome to occur.

When they renature, ITRs can fold into two configurations, called flip and flop, in which the sequence between the A and A′ inverted repeats is present as the reverse complement with respect to the other configuration. With respect to the 5′ ITR (left ITR), the order of terminal palindromic sequences for the flip configuration is 5′-ABB′CC′A′D-3′, and the order for the flop configuration is 5′-ACC′BB′A′D-3′. With respect to the 3′ ITR (right ITR), the order of terminal palindromic sequences for the flip configuration is 3′-A′B′BC′CAD′-5′, and the order for the flop configuration is 3′-A′C′CB′BAD′-5′. Consequently, the flip configuration has the B′B palindrome closest to the free 3′ end, whereas the flop configuration has the C′C palindrome closest to the free 3′ end (Lusby, E, et al., J Virol, 34:402-9 (1980); Srivastava, A, et al., J Virol, 45 (2): 555-64 (1983); Samulski, R J, et al., Cell, 33:135-143 (1983)).

It is hypothesized that ITR secondary structure supports viral DNA replication by a self-priming single-strand displacement elongation mechanism initiated by endogenous cellular DNA polymerase at the ITR with the free 3′ hydroxyl group. Strand elongation leads to the formation of a monomeric dsDNA genome replicative intermediate with one covalently closed end. The duplex ITR at the open end refolds (isomerizes) into a double hairpin structure, forming a new 3′ ITR which is elongated while the complementary strand is displaced. The large AAV Rep proteins bind to the ITR at the closed end (downstream) and nicks the DNA at its terminal resolution site, initiating a second DNA replication complex that copies the downstream ITR before the DNA replication complex which initiated at the open end reaches it. The original replication complex displaces the opposite strand (whose ITR was just newly synthesized), and completes replication to what had been the closed end of the genome, now open with duplex ITRs available to isomerize into a double hairpin. Thus, the monomeric dsDNA genome replicative intermediate is recreated to start the cycle of replication over again, while the displaced ssDNA genome (whose 3′ ITR had been newly created) can be packaged into a virus particle.

The ssDNA genomes that are replicated will include both positive (plus, or sense) and negative (minus, or anti-sense) strand polarities, and evidence suggests that they are individually packaged into capsids with equal efficiency. Consequently, preparations of AAV vector particles, like the viruses from which they are adapted, can in some embodiments contain sense or antisense ssDNA genomes in about equal proportion. ITRs can also be modified, however, by selective removal of the D sequence from one of the two ITRs used to generate AAV vectors, which restricts packaging to either the negative or the positive strand of the vector genome (Wang, X-S, et al., J Virol, 70 (3): 1668-77 (1996)). Thus, in some other embodiments, a preparation of AAV vectors can contain vector particles in which most or substantially all the vector genomes are either postively standed or negatively stranded.

After infection, AAV virions are transported to the nucleus, where the ssDNA genomes are released from the capsid. Before the viral rep and cap genes can be expressed, it is hypothesized that the ssDNA genome must first be converted to dsDNA through complementary strand synthesis by cellular DNA polymerase initiating strand elongation at the 3′ ITR, a process that is believed to be slow and inefficient. It is also hypothesized that a faster mechanism may exist to form intracellular dsDNA genomes, in which complementary positive and negative ssDNA genomes originating from different virions infecting the same cell encounter each other in the nucleus and hybridize via intermolecular base pairing. Such duplex genomes could then support transcription without first requiring elongation by cellular DNA polymerase.

In designing AAV vectors, the only AAV viral DNA sequences retained in the vector genome are the ITRs because of their critical roles in DNA replication and packaging during production, and conversion of ssDNA genomes to dsDNA after transduction. The sequences encoding Rep and Cap proteins, and viral helper functions, which are also needed to produce vectors, can be provided in trans in a variety of ways known in the art. When the vector genome sequence, such as might be included in a plasmid used for AAV vector production, includes two intact ITRs, and is of a length that does not exceed the AAV capsid packaging capacity of ˜5 kb, ssDNA genomes can be packaged, as noted above, but the requirement for dsDNA conversion can result in lower than desired transduction efficiency due to the inefficiency of that step before gene expression can occur. A strategy to overcome the requirement for dsDNA conversion by endogenous cellular DNA polymerase, and potentially improve transduction efficiency and expression of heterologous sequence, such as a therapeutic transgene, relies on replicating and packaging “self-complementary” AAV vector genomes (scAAV) which, because they contain positive and negative strand sequences in the same DNA molecule, can quickly renature to form a dsDNA transcriptional template through intramolecular base pairing (i.e., intramolecular hybridization) after capsid uncoating in the target cell nucleus.

Production of self-complementary genomes can be faciliated in at least two ways, both of which rely on failure of Rep to nick the ITR at the terminal resolution site during replication of the DNA molecule that is to become the genome. In the natural replication cycle of AAV, dimeric dsDNA genome replicative intermediate molecules can occur when Rep fails to nick the terminal resolution site of the downstream ITR of the monomeric genome replicative intermediate possessing the double hairpins at the open end and the single downstream ITR at the closed end. When this occurs, replication by the initial DNA replication complex (starting at the 3′ ITR at the open end) continues through the downstream (closed end) ITR as well as the displaced strand to form a dimeric dsDNA genome. This molecule is similar to the monomeric dsDNA genome replicative intermediate described above, but contains two genomes. Because it has an open end with duplex ITRs, it can isomerize to start a cycle of DNA replication as with the monomeric form. Alternatively, the closed end hairpin can undergo terminal resolution forming duplex ITRs which can isomerize so that DNA synthesis initiates from the resolved end. In either case, replication of the dimeric template generates a new dimeric dsDNA genome replicative intermediate, as well as displacing a ssDNA dimeric inverted repeat genome containing a 5′ ITR, viral genome sequence of one polarity, a central ITR, viral genome sequence of opposite polarity, and a 3′ ITR.

Ordinarily, the ssDNA dimeric inverted repeat viral genome will not be packaged because it exceeds the normal capsid packaging capacity. However, by designing vector genomes of reduced length so that ssDNA dimeric inverted repeat genomes, when formed, would not exceed the AAV packaging capacity, it is possible to produce vector particles containing self-complementary genomes. In practice, vector preparations produced this way can contain a mixture of particles in which are packaged one scDNA genome, or one or two monomeric ssDNA genomes, the proportions of all of which can vary between preparations.

After packaging, it is likely that scDNA genomes reside within capsids in single-stranded form (similar to ssDNA non-self complementary genomes), but then rapidly anneal after capsid uncoating to form a dsDNA molecule with a covalently closed ITR at one end and two open ITRs at the other end, resembling the structure of a conventional viral genome after dsDNA conversion through self-priming. Thus, while not wishing to be bound by theory, a principal difference between so-called ssDNA and scDNA genomes is not the topology while encapsidated, but rather the topology each type of genome likely acquires after capsid uncoating and genome release within transduced cells.

A further modification, however, allows greater control over the production of vector particles containing scDNA genomes. Specifically, by mutating or deleting the terminal resolution site from one ITR, such as in the plasmid containing the vector genome sequence used for vector production, it is possible to inhibit or eliminate single-strand nicking at that ITR during the vector genome replication cycle. As a consequence, the replication complex initiated at the unmutated ITR progresses through the mutated hairpin and back to the initiating end, resulting in a dimeric dsDNA genome replicative intermediate, as in the case where terminal resolution of a wild type ITR does not occur by chance. This intermediate, however, would contain a closed wild type ITR at one end, mutated duplex ITR in the middle of molecule, and duplex open ITRs at the opposite end that are capable of isomerization. This molecule can then undergo normal rounds of replication and strand-displacement from the wild type ITRs at each end, to produce displaced daughter genome copies containing a 5′ wild type ITR, vector genome sequence of one polarity, mutated ITR in the middle, vector genome sequence of the opposite polarity, and a wild type ITR at the 3′ end. If the heterologous sequence contains a protein-encoding transgene, the scDNA genome would contain both the coding sequence and its complement in the same DNA molecule. Although such genomes are believed to be packaged in single-stranded form inside capsids, they are also believed to be able to rapidly self-anneal into double-stranded form inside the nuclei of transduced cells due to the presence of significant amounts of self-complementary sequence outside the ITRs, in which form they can support transcription of the heterologous sequence. Production of scAAV from constructs containing mutated ITRs can yield more than 90% scDNA genomes.

In AAV2 capsids, which have a packaging capacity of approximately 4.7 kb, then excluding the ITR sequences, ssDNA genomes can accommodate approximately 4.4 kb of heterologous sequence, whereas scDNA vector genomes can accommodate about 2.2 kb. According to a particular non-limiting embodiment, the genome construct size (such as might be contained within a plasmid for AAV vector production) for producing scAAV vectors is about ˜2,500 nucleotides long, comprising a ˜2,200 nucleotide long heterologous sequence plus two ITRs (one wild type and one mutated). This would result produce an scAAV genome ˜4,700 nucleotides long, which is below the typical AAV capsid packaging capacity.

ITR terminal resolution sites can be disrupted in various ways to facilitate production of scAAV vectors. For example, an exogneous sequence can be inserted into the terminal resolution site (trs) sequence itself, or into an adjacent sequence of the ITR, such as between the Rep binding element and the trs. Alternatively, the trs sequence could be deleted partially or in its entirety. In other embodiments, the adjacent D region can be deleted partially or in its entirety. In yet other embodiments, nucleotides within the trs can be substituted with different nucleotides that reduce frequency of trs nicking by Rep. Other ways of rendering ITRs non-resolvable are within the ordinary skill in the art.

Further information about AAV viral and vector genome replication, as well as design of scAAV, can be found in Mccarty, D M, et al., Gene Therapy, 10:2112-18 (2003); McCarty, D M, et al., Gene Therapy, 8:1248-54 (2001); McCarty, D M, Molecular Therapy, 16 (10): 1648-56 (2008); U.S. Pat. No. 7,790,154.

In some embodiments, AAV vector genomes of AAV vectors of the disclosure can contain one or more AAV ITRs originating from different AAV serotypes and variants, have different sequences and lengths, and be positioned at different locations within the genome. The sequence of AAV ITRs for use in the vectors of the disclosure can be wild-type or modified. In other embodiments, AAV ITR sequences can also be included as part of vector genome sequences in plasmids and other types of vectors, such as baculoviruses, used to introduce the vector genome sequence into host cells for purposes of vector production.

ITRs from AAV2 are frequently used in producing AAV vectors, but alternative embodiments can include using ITRs from any AAV serotype or variant including, for example, ITRs from AAV1, AAV3, AAV3B, AAV4, AAV5, AAV6, AAV7, AAV8, or AAV9, or any other AAV serotype or variant known or yet to be discovered, so long as such ITRs are functional for vector genome replication and packaging, and transgene expression. ITRs could also include modifications to the sequence of a wild-type ITR sequence or be fully synthetic. A modified ITR can be at least 90%, 91%, 92%, 93%, 94%, 95%, 96%, 97%, 98%, or 99% identical to a wild type ITR sequence, such as that from AAV2. In some embodiments, an ITR is modified by adding, deleting and/or changing nucleotides to disrupt a terminal resolution site (trs), and/or a D sequence of an ITR, or to change some other subsequence with an ITR. A vector genome, in some embodiments, can comprise ITRs from different AAV serotypes or variants.

In some embodiments, the ITRs chosen for use in vector production will be from the same serotype or variant as the capsid. For example, AAV2 ITRs can be used in conjunction with AAV2 capsids. In other embodiments, however, vectors can be pseudotyped or hybrid, meaning that a vector genome with ITRs from one serotype or variant can be packaged into a capsid from a different serotype or variant. For example, a vector genome with ITRs from AAV2 could be packaged into a capsid from AAV8 (or any other serotype or variant capsid). This pseudotype is often abbreviated AAV2/8, where the number before the slash indicates the origin of the ITRs, and the number after the slash indicates the origin of the capsid.

According to some embodiments, AAV vectors of the disclosure can contain vector genomes with different numbers of ITRs. For example, intact AAV viral genomes usually possess two ITRs, one each positioned at the 5′ and 3′ termini respectively, and AAV vectors produced using intact ITRs and packaged with ssDNA genomes can also have two ITRs similarly positioned. As discussed, above, however vectors can be designed in such a way that their genomes include three ITRs, two at the ends of the genome as in virus or convential vectors, but one additional at or near the middle (intact or mutated), as in self-complementary vectors. It has also been observed, however that with some vectors, particularly if the genome length exceeds the average packaging capacity of a capsid, 5′ ITRs may be truncated or missing entirely due, it is hypothsized, to premature or variable packaging termination. Although defective interfering particles can result, it is also hypothesized that such particles could nevertheless support transgene expression as a result of complementation of otherwise incomplete genomes (Kapranov, P, et al., Hum Gene Ther, 23:46-55 (2012)). Accordingly, in some embodiments AAV vector particles of the disclosure can contain genomes comprising a single functional ITR, such as at the 3′ end or the 5′, and where at the opposite end is a non-functional truncated ITR, or no ITR sequence. Thus, in some embodiments of AAV vector genomes of AAV vectors of the disclosure, an AAV ITR can be positioned at the 5′ end of the genome, or at the 3′ end of the genome, or at both the 5′ and 3′ ends of the genome, as well as in other positions.

Another source of heterogeneity that can occur in AAV vectors can arise from the differential presence of the flip and flop ITR configurations in any particular genome. Thus, for example, in some embodiments, any particular vector particle in a sample might contain a genome with an ITR at both ends of the genome in the flip configuration, or with an ITR at both ends of the genome in the flop configuration, or with a flip ITR at the 5′ end and a flop ITR at the 3′ end, or a flop ITR at the 5′ end and a flip ITR at the 3′. When combined with the observation that single-stranded DNA genomes can occur as postively or negatively stranded, a sample of AAV vectors could comprise eight possible configurations, in similar or potentially different proportions. Unless otherwise specified, genomes of AAV vectors of the disclosure are not limited to any of these configurations; any or all could be packaged by such vectors.

In some embodiments, a AAV vector genome includes an intact full length ITR at each of its 5′ and 3′ ends. Thus, for example, if both ITRs are from AAV2, such full-length ITRs would be 145 nucleotides long. In other embodiments, however, one or more of the ITRs may be truncated, missing one or more terminal nucleotides relative to the canonical full-length sequence for that type of ITR. Thus, for example, an ITR may lack at least or about 1, 2, 3, 4, 5, 6, 7, 8, 9, 10, 11, 12, 13, 14, 15, 16, 17, 18, 19, 20 nucleotides, or more, from its terminal end relative to the canonical full-length sequence for that type of ITR. Such truncated ITRs can occur in genomes packaged by AAV vectors of the disclosure, but also in vector genome sequences used in the production of AAV vectors, such as in a plasmid. For example, it has been observed that truncated ITRs can still function to produce AAV due to capacity for self-repair if sequences missing from one ITR are retained in the other (Wang, X-S, et al., J Mol Biol, 250:573-80 (1995); Samulski, R J, et al., Cell, 33:135-43 (1983)).

In some embodiments, AAV vector genomes of AAV vectors of the disclosure can contain one or more AAV ITRs comprising, consisting essentially of, or consisting of the nucleotide sequence of any one or more of SEQ ID NO:7, SEQ ID NO:15, SEQ ID NO:18, or SEQ ID NO:19, or the complement or reverse complement of any of such sequences.

### Sequence Optimization

In some embodiments, one or more sequences within an AAV vector genome can be optimized to improve its functional characteristics relative to a starting reference sequence. For example, and without limitation, any protein coding sequence in a vector genome can be codon-optimized relative to the wild type sequence, based on the degeneracy of the genetic code and codon usage biases known to exist between different species and between proteins expressed at high or low levels in the same species. Such codon biases can be identified using a codon adaptation index for a particular species, for example. Codon adaptation index (CAI) is explained in more detail in Sharp, P M and Li, W H, Nucleic Acids Res, 15:1281-95 (1987). In some embodiments, coding sequences are human codon-optimized, meaning the coding sequences are optimized based on human codon biases. Codon-optimization can be facilitated using various algorithms known in the art. As is known in the art, different CAI can be constructed based upon which highly expressed genes, such as human genes, are analyzed. An exemplary human CAI is reported in Haas, J, et al., Current Biology, 6 (3): 315-24 (1996). If desired, protein coding sequences can be codon-optimized for species other than human as well.

With the goal of increasing protein expression levels, different codon-optimization strategies have been proposed and implemented. For example, the most frequently used synonymous codon (i.e., one coding for the same amino acid) can be substituted at each position where it does not occur. Alternatively, codon usage can be adjusted over the entire coding sequence so that it is proportional to the natural codon bias distribution of the host organism. In some embodiments, codon replacement is limited to ones that occur relatively rarely in highly expressed proteins in a species, for example, with a frequency of 10% of less, as reflected in a CAI.

In some embodiments, a protein coding sequence to be expressed by a AAV vector of the disclosure is codon-optimized by substituting at least one rare codon with a more common synonymous codon. In some embodiments, at least 5%, 10%, 20%, 30%, 40%, 50%, 60%, 70%, 80%, 90%, or 95%, and in some embodiments 100% of rare codons in the protein coding sequence are replaced with a more frequently used synonymous codon as reflected in a CAI, such as a human CAI. In some embodiments, a rare codon is one that occurs at a frequency of less than or equal to 10%, 9%, 8%, 7%, 6%, or 5%, as reflected in a CAI, such as a human CAI.

In some embodiments, a protein coding sequence to be expressed by an AAV vector of the disclosure is codon-optimized by replacing one or more codons with a more frequently used synonymous codon as reflected in a CAI, such as a human CAI, so that the CAI value calculated for the overall coding sequence is increased relative to the starting non-codon optimized sequence, which in some embodiments is the wild type coding sequence of a protein. Thus, in some embodiments, the CAI value of a starting reference sequence is calculated with reference to a particular CAI reference table and one or more codons are replaced with more frequent synonymous codons so that the overall CAI value of the now codon-optimized coding sequence is increased by at least or about 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.55, 0.60, or 0.70.

As is known in the art, the presence of hypomethylated CpG dinucleotides in nucleic acid can stimulate immune responses which eliminate transduced cells. Depleting CpG dinucleotides in vector genomes can therefore increase the likelihood that vector transduction the will result in long-term gene expression. Wright, J F, Mol Ther, 28 (3): 701-3 (2020). In view of the potentially detrimental effects of CpG dinucleotides, in some embodiments, any sequence within the genome, including for example, enhancers, promoters, introns, open reading frames that encode protein or functional RNA, transcriptional terminators, 5′ and/or 3′ untranslated region (UTR) sequences, ITRs, or any other sequence can be modified to remove one or more CpG dinucleotides, as long as doing so does not unacceptably interfere with or disrupt some desirable function of the modified element. Because the function of certain elements within vector genomes, such as ITRs, promoters, and enhancers, can be highly dependent on the identity of particular nucleotides in certain positions, there may be more limited opportunities to significantly deplete such elements of CpG dinucleotides. Because AAV vector genomes of both polarities (e.g., sense and antisense, with respect to the coding sequence of a transgene within the genome) are packaged into capsids in about equal proportions, a strategy of CpG depletion can, in some embodiments, be directed to reducing or eliminating CpG motifs from the nucleotide sequence of vector genomes of both polarities, not just vector genomes that contain protein coding sequence in the sense orientation.

In some embodiments, at least 5%, 10%, 20%, 30%, 40%, 50%, 60%, 70%, 80%, 90%, 95%, 96%, 97%, 98%, or 99% of CpG dinucleotides in a coding sequence or the overall vector genome sequence (with respect to the sense and/or antisense strand) are deleted, or replaced, relative to a reference starting sequence. In other embodiments at least 1, 5, 10, 15, 20, 25, 30, 35, 40, 45, 50, 55, 60, 65, 70, 75, 80, 85, 90, 95, 100, or more CpG dinucleotides, or a range between any of the foregoing values, are deleted, or replaced, relative to a starting reference sequence. In other embodiments, between 1-5, 5-10, 10-15, 15-20, 20-25, 25-30, 30-35, 35-40, 40-45, 45-50, 50-55, 55-60, 60-65, 65-70, 70-75, 75-80, 80-85, 85-90, 90-95, or 95-100, CpG dinucleotides are deleted, or replaced, relative to a reference starting sequence.

In other embodiments, sequence optimization can increase or decrease the overall GC content relative to a starting reference sequence. Thus, in some embodiments, the overall percentage of G or C nucleotides in a transgene or the genome overall, can be increased, relative to a starting reference sequence, such as a wild type protein encoding sequence, by at least or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, or 40 percent, or more. In other embodiments, the overall percentage of G or C nucleotides in a transgene or the genome overall, can be decreased, relative to a starting reference sequence, such as a wild type protein encoding sequence, by at least or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, or 40 percent, or more.

As will be appreciated by those of ordinary skill, when optimizing coding sequence, the goal of substituting with more prevalent codons for any particular amino acid in a species (such as human) may be incompatible with other optimization strategies because introduction of more frequently used codons could introduce CpG motifs, or elimination of CpG might require use of rarely occurring codons, or codon optimization might increase or decrease GC content in undesired ways. In these instances, it may be necessary to design and test different optimized coding sequences (encoding the same polypeptide) to identify versions that strike an acceptable balance between the different optimization strategies to achieve improved protein expression.

Transgene and vector genome sequence can be optimized by changing features in addition to codon bias and CpG content. For example, any of the following features that sometimes occur in sequences and negatively impact transgene expression can be identified (either conceptually, such as by using algorithms, or empirically) and altered or eliminated so as to reduce their effect: cryptic splice sites; premature transcriptional termination signal sequences (e.g., polyA sequences); translational start sites (e.g., IRES) other than for the intended initiator methionine; sequence regions with high GC content; mRNA 5′ end sequences that can form hairpins; and AU rich elements (ARE) in mRNA 3′ untranslated regions which can be bound by destablilizing RNA binding proteins. Other sequence features that can appear in transgenes and vector genomes which, when altered, can enhance transgene expression will be familiar to those of ordinary skill in the art.

In other embodiments, transgene or vector sequence can be modified to enhance functionality. For example, the first intended start codon in a protein coding sequences may only weakly support translation initiation from that site, in which case, the surrounding sequence can be altered to match the so-called Kozak consensus sequence for translation initiation in eukaryotes. Kozak, M., Gene, 234 (2): 187-208 (1999).

In some embodiments, CpG depletion (partial or complete), or other types of sequence optimization of the transgene coding sequence, such as codon-optimization, can improve protein expression from the transgene compared to the same vector including a non-optimized reference starting sequence, such as a wild type coding sequence from which the optimized sequence is derived. Thus, for example, the optimized coding sequence of a transgene may express at least 25%, 50%, 75%, 100%, 150%, 200%, 250%, 300%, 350%, 400%, 450%, 500%, or more efficiently compared to a non-optimized reference starting sequence, such as the wild type version of the coding sequence.

### AAV Capsids

AAV vectors of the disclosure can utilize capsids made from any AAV capsid protein, whether naturally occurring or modified, including those presently known or yet to be discovered or developed, which are suitable for transducing cells in a subject to express GCase protein from a vector transgene.

Choice of which capsid protein to use in creating an AAV vector (and the corresponding cap gene sequence to be used in its production) can be guided by many considerations and factors. As noted above, by virtue of interacting specifically with certain cell surface receptors, different AAV capsids can have different cell or tissue tropisms, which can be an advantage when it is desired to preferentially transduce certain tissues versus others. For example, to express a transgene product preferentially in muscle, one might produce a vector using a capsid with tropism for muscle over liver. Conversely, to express a transgene product in liver, one might produce a vector using a capsid with tropism for liver over muscle or other tissues.

Other factors may be important as well. Research has established, for example, that some humans have high neutralizing antibody titers to certain capsids as a result of exposure to naturally occurring AAVs, which can interfere with the ability of AAV vectors with the same or similar capsids to transduce target cells. Thus, in designing a vector for gene therapy, choice of capsid may in some cases be guided by the immunogenicity of the capsid, and/or the seroprevalence of the patients to be treated. Other considerations that may influence capsid choice include manufacturability and stability during storage, with others being known in the art.

AAV vectors of the disclosure can use capsids made from capsid proteins from naturally occurring AAVs, as well as modified or engineered capsid proteins. For example, naturally occurring capsid proteins can be modified by inserting or deleting amino acids or peptides, or by introducing amino acid substitutions, in the VP1, VP2, and/or VP3 protein sequence intended to improve capsid function in some way, such as tissue tropism, immunogenicity, stability, or manufacturability. Other examples include novel capsids with improved properties created by swapping amino acids or domains from one known capsid to another (mosaic or chimeric capsids), or using DNA shuffling and directed evolution methods.

In some embodiments, AAV vectors of the disclosure can comprise a capsid from known AAV serotypes and variants, as well as non-naturally occurring capsids, including, without limitation AAV1, AAV2, AAV3A, AAV3B, AAV4, AAV5, AAV6, AAV7, AAV8, AAV9, AAVrh10, AAVrh74, AAV-DJ, AAV-PHP.B, Anc80, AAV2.5, and AAV2i8, with many others being possible. In some embodiments, capsids of AAV vectors of the disclosure include a VP1, a VP2, and/or a VP3 AAV capsid protein which is a variant or derivative of a known VP1, VP2, or VP3 AAV capsid protein. In some embodiments, the amino acid sequence of such variant or derivative AAV capsid protein can be at least or about 70%, 75%, 80%, 85%, 86%, 87%, 88%, 89%, 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, or 99.9% identical to the amino acid sequence of any known AAV capsid VP1, VP2, or VP3 protein sequence, including, without limitation, the AAV capsid VP1, VP2, or VP3 proteins of AAV1, AAV2, AAV3A, AAV3B, AAV4, AAV5, AAV6, AAV7, AAV8, AAV9, AAVrh10, AAVrh74, AAV-DJ, AAV-PHP.B, Anc80, AAV2.5, and AAV2i8, or any other suitable AAV capsid, including, for example, the hepatotropic capsids discussed below. In some other embodiments, the amino acid sequence of such variant or derivative AAV capsid protein differs (whether due to deletion, insertion, or substitution of amino acids) by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, or more amino acids from a known AAV capsid VP1, VP2, or VP3 protein amino acid sequence including, without limitation, the AAV capsid VP1, VP2, or VP3 proteins of AAV1, AAV2, AAV3A, AAV3B, AAV4, AAV5, AAV6, AAV7, AAV8, AAV9, AAVrh10, AAVrh74, AAV-DJ, AAV-PHP.B, Anc80, AAV2.5, and AAV2i8, or any other suitable AAV capsid, including, for example, the hepatotropic capsids discussed below.

### Hepatotropic AAV Capsids

In some embodiments, AAV vectors of the disclosure comprise a hepatotropic capsid. A hepatotropic capsid is an AAV capsid with tropism for liver cells, such as hepatocytes. According to some embodiments, a capsid for use in the AAV vectors of the disclosure is hepatotropic for human liver cells, such as human hepatocytes. As will be appreciated by those of ordinary skill, hepatotropism does not mean that a capsid is capable of transducing only liver cells. Rather, a hepatotropic capsid will exhibit some degree of greater propensity to transduce liver cells as compared to other types of cells or tissues, even if that propensity is not absolute, and even if the hepatotropic capsid exhibits even greater propensity for transduction of some other type of cell or tissue than liver cells. In some non-limiting embodiments, a hepatotropic AAV capsid is one that binds to heparan sulfate proteoglycan (HSPG), which has been implicated as a cellular receptor mediating AAV infection of human hepatocytes.

Examples of hepatotropic capsids include, but are not limited to: AAV2; AAV3B; AAV5; AAV6; AAV7; AAV8; AAV9; AAV13; AAV-DJ (Grimm, D, et al., J Virol, 82:5887-5911 (2008)); AAV-LK03 (Lisowski, L, et al., Nature 506:382-6 (2014)); AAV-KP1; AAV-hu.Lvr01 (Genbank Accession No. QPP19816); AAV-hu.Lvr02 (Genbank Accession No. QPP19818); AAV-hu.Lvr03 (Genbank Accession No. QPP19819); AAV-hu.Lvr04 (Genbank Accession No. QPP19821); AAV-hu.Lvr05 (Genbank Accession No. QPP19824); AAV-hu.Lvr06 (Genbank Accession No. QPP19825); AAV-hu.Lvr07 (Genbank Accession No. QPP19828); AAV-rh.10; AAV-rh.74; AAV-Anc80 (Zinn, E, et al., Cell Rep, 12:1056-68 (2015)); AAV-NP40, AAV-NP59, and AAV-NP84 (Paulk, N K, et al., Molecular Therapy, 26 (1): 289-303 (2018)); AAV-hu.37; AAV-rh.8; AAV-rh.64R1; capsids comprising a VP1 protein comprising or consisting of SEQ ID NO:5, SEQ ID NO:6, SEQ ID NO:7, SEQ ID NO:8, SEQ ID NO: 9, and SEQ ID NO:10 from WO 2015/013313 (called RHM4-1, RHM15-1, RHM15-2, RHM15-3/RHM15-5, RHM15-4, and RHM15-6, respectively); AAVHSC7, AAVHSC15, and AAVHSC17 (Smith, L J, et al., Mol Ther, 22 (9): 1625-34 (2014); Ellsworth, J L, et al., PLOS One, 14 (11): e0225582 (2019)).

### AAV Vectors for Expressing GCase Protein

According to certain embodiments, AAV vectors of the disclosure comprise an AAV vector genome comprising a transgene encoding a beta-glucocerebrosidase (GCase) protein, such as human GCase protein isoform 1. In some embodiments, the transgene comprises coding sequence for a secretion signal peptide sequence, as well as coding sequence for the mature form of the GCase protein. In some embodiments, the secretion signal peptide sequence is the same that exists in the naturally occurring GCase protein, but signal peptides from heterologous proteins can be used as well, such as the signal peptide from the IgG heavy chain polypeptide, such as a murine IgG heavy chain polypeptide. In some embodiments, human GCase mature polypeptide comprises the amino acid sequence of SEQ ID NO:40, the amino acid sequence of the wild-type human GCase secretion signal peptide comprises SEQ ID NO:39, and the amino acid sequence of a secretion signal peptide from a murine IgG heavy chain polypeptide comprises SEQ ID NO: 38. In some embodiments, the amino acid sequence of a chimeric GCase precursor protein including a murine IgG heavy chain polypeptide and human GCase mature polypeptide comprises SEQ ID NO:16.

In some embodiments, the GCase protein coding nucleotide sequence of the transgene is the same nucleotide sequence as that of a wild-type human GCase coding sequence, non-limiting examples of which include SEQ ID NO:28, SEQ ID NO:29, SEQ ID NO:30, SEQ ID NO:31, or SEQ ID NO:32. In other embodiments, however, the coding sequence can be optimized, such as by deletion of some or all CpG dinucleotides, while still encoding the same GCase protein, a non-limiting example of which includes SEQ ID NO:13, which encodes the mature polypeptide, or SEQ ID NO: 41, which encodes chimeric GCase protein comprising a murine IgG H-chain secretion signal peptide sequence and the human mature polypeptide. In some embodiments, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, or more CpG dinucleotides are removed from the GCase protein transgene coding sequence, or any range between an including any of the foregoing specifically enumerated values. In some embodiments, the nucleotide sequence encoding GCase protein is devoid of CpG dinucleotides. In some embodiments, a nucleotide sequence encoding GCase protein comprises 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, or 0 CpG dinucleotides, or any range between an including any of the foregoing specifically enumerated values, wherein the nucleotide sequence from which CpG dinucleotides are removed relative to a reference sequence, such as the wild-type sequence, is either the sense strand containing the coding sequence, or the antisense strand, i.e., the reverse complement of the sense strand.

In some embodiments, the nucleotide sequence of the transgene encoding the human GCase mature polypeptide is the same as that provided in SEQ NO: 13, whereas in related embodiments, the transgene can be at least 50%, 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to the nucleotide sequence of SEQ ID NO:13 while encoding the identical amino acid sequence as that encoded by SEQ ID NO:13. In other embodiments, the nucleotide sequence of the transgene encoding the chimeric GCase precursor protein is the same as that provided in SEQ NO: 41, whereas in related embodiments, the transgene can be at least 50%, 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to the nucleotide sequence of SEQ ID NO:41 while encoding the identical amino acid sequence as that encoded by SEQ ID NO:41. In other embodiments, the nucleotide sequence of the transgene encoding the human GCase mature polypeptide is the same as that provided in SEQ NO: 28, SEQ NO: 29, or SEQ NO: 30, whereas in related embodiments, the transgene can be at least 50%, 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to the nucleotide sequence of SEQ NO: 28, SEQ NO: 29, or SEQ NO: 30, respectively, while encoding the identical amino acid sequences as those encoded by SEQ NO: 28, SEQ NO: 29, or SEQ NO: 30, respectively.

In some embodiments, vector genomes of the AAV vectors of the disclosure further comprise at least one AAV inverted terminal repeat (ITR), positioned at the 5′ and/or the 3′ end of the genome. In some embodiments, vector genomes can further comprise at least a second AAV ITR positioned at the opposite end of the genome from the first AAV ITR. In some embodiments, vector genomes comprise an AAV ITR positioned at its 5′ terminus. In some embodiments, vector genomes comprise an AAV ITR positioned at its 3′ terminus. In some embodiments, vector genomes comprise a first AAV ITR positioned at its 5′ terminus and a second AAV ITR positioned at its 3′ terminus. In some embodiments, vector genomes comprise a first AAV ITR positioned at its 5′ terminus and a second AAV ITR positioned at its 3′ terminus and a third AAV ITR positioned between said first and second AAV ITRs.

AAV ITRs for use in the vectors of the disclosure can be of any type, such as an AAV2 ITR or a non-AAV2 ITR, and can be full length or truncated, and can have the same sequence as any known AAV viral ITR as it exists in nature (wild-type sequence), or can be modified. Exemplary non-limiting types of modifications include reducing the number of CpG dinucleotides occurring in the ITR sequence, reducing or eliminating the ability of the ITR sequence to undergo terminal resolution by AAV Rep proteins, such as by mutating, deleting or otherwise inactivating the terminal resolution site (trs), as well as reducing or eliminating the ability of the ITR to support packaging into a capsid, such as by mutating, deleting or otherwise inactivating the D sequence of the ITR sequence. In some embodiments, the vector genome can further comprise at least a third AAV ITR, such as one positioned between the ends of the genome, such as near or at the center of the vector genome sequence. In some of these embodiments, the third ITR can be modified, such as by inactivating its terminal resolution site such that the vector genome, including the transgene, is self-complementary. In some embodiments, vector genomes comprise an AAV ITR comprising, consisting essentially of, or consisting of the nucleotide sequence of SEQ ID NO:7, SEQ ID NO:15, SEQ ID NO: 18, or SEQ ID NO:19, or the complement or reverse complement of each of such sequences.

In some embodiments, vector genomes of the AAV vectors of the disclosure further comprise a transcription control region operably linked with the transgene encoding the GCase protein. In some embodiments, the transcription control region can be inducible, constitutively active, or cell-type or tissue-type specific, such as being active mostly or exclusively in hepatocytes, or liver (thus, hepatocyte specific or liver tissue specific). In some embodiments, the transcription control region comprises or consists of a promoter, and can further comprise at least one enhancer region or element. Any region or element in the transcription control region can be derived from a human gene, or a non-human gene, such as a rat, mouse, bovine, non-human primate, chicken, or viral gene, or other species or type of organism. Regions or elements of the transcription control region can be contiguous with each other, or be separated by other functional sequences of the vector genome. Thus, for example, a promoter region could be proximal and 5′ (upstream) of the transgene, whereas an enhancer region or element could be anywhere else in the vector genome, such as distally upstream, or elsewhere, such as distally 3′ (downstream) of the transgene. Any region or element of a transcription control region can be cell type or tissue specific, such as liver tissue specific. Thus, a promoter can be liver cell or tissue specific, an enhancer region or element can be liver cell or tissue specific, or both the promoter and enhancer(s), acting alone or in concert, can be liver cell or tissue specific.

In some embodiments a transcription control region for use in the vectors of the disclosure can contain a promoter sequence derived from the human albumin (ALB) gene, where such promoter is the entire human ALB gene promoter, or a promoter functional subsequence of such human ALB gene's promoter. Thus, for example, the promoter can comprise, consist essentially of, or consist of the nucleotide sequence of SEQ ID NO:10, or a promoter functional subsequence, modification or variant thereof.

In some embodiments a transcription control region for use in the vectors of the disclosure can contain an enhancer sequence derived from the human albumin (ALB) gene where such enhancer sequence is the entire human ALB gene enhancer region, or an enhancer functional subsequence of such human ALB gene's enhancer region. Thus, for example, the enhancer sequence can comprise, consist essentially of, or consist of the nucleotide sequence of SEQ ID NO:9, or an enhancer functional subsequence, modification or variant thereof. In some embodiments a transcription control region for use in the vectors of the disclosure can contain an enhancer sequence derived from the alpha-1-microglobulin/bikunin precursor (AMBP) gene where such enhancer sequence is the entire AMBP gene enhancer region, or an enhancer functional subsequence of such AMBP gene's enhancer region. Thus, for example, the enhancer sequence can comprise, consist essentially of, or consist of the nucleotide sequence of SEQ ID NO: 8, or an enhancer functional subsequence, modification or variant thereof.

In some embodiments, vector genomes of the AAV vectors of the disclosure further comprise a transcription termination signal sequence, such as a a polyadenylation (poly(A)) signal sequence, such as a pA signal sequence derived from the bovine growth hormone (bGH) gene, or a transcription termination functional subsequence of such bGH gene's pA signal sequence. Thus, for example, the transcription termination signal sequence can comprise, consist essentially of, or consist of the nucleotide sequence of SEQ ID NO: 14, or transcription termination functional subsequence, modification or variant thereof.

In some embodiments, vector genomes of the AAV vectors of the disclosure further comprise an intron sequence, which can be positioned within and interupt the transgene encoding the GCase protein, or can be positioned elsewherein in the vector genome, and not interupt the coding sequence, such as being positioned 5′ of the transgene, or being positioned 3′ of the transgene, or elsewhere, such as being positioned 3′ of a promoter and 5′ of the transgene. In some embodiments, an intron sequence can be derived from the human beta globin (HBB) gene which, in some embodiments, can comprise, consist essentially of, or consist of the nucleotide sequence of SEQ ID NO:11.

In some embodiments, vector genomes of the AAV vectors of the disclosure further comprise additional functional sequences, such as a viral post-transcriptional regulatory element (PRE) sequence, such as a woodchuck hepatitis virus post-transcriptional regulatory element (WPRE), or a Hepatitis B virus posttranscriptional regulatory element (HPRE), any of which can be positioned 3′ of the transgene and 5′ of the transcription termination signal sequence, or elsewhere in the genome. In some embodiments, the PRE can be a WPRE comprising, consisting essentially of, or consisting of the nucleotide sequence of SEQ ID NO:27, or a functional subsequence, modification or variant thereof. Other sequences that may find utility in vector genomes of the disclosure include, without limitation, a binding site for a microRNA (miRNA), which can be positioned 3′ of the transgene and 5′ of the transcription termination signal sequence, or elsewhere in the genome, and stuffer or filler nucleotide sequences which, while not necessarily intended to directly affect expression of the transgene (although such property could be present) are included so that the overall length of the vector genome is of a particular size, for example sufficiently long so as to approximate the packaging capacity of a particular AAV capsid, so as to reduce the amount of contaminating non-full length vector genomic DNA that is packaged in capsids.

In some embodiments, vector genomes of the AAV vectors of the disclosure comprise a first AAV ITR, a transcription control region in operable linkage with a transgene encoding GCase protein, a transcription termination signal sequence, and a second AAV ITR. In related embodiments, these elements can be arranged sequentially in 5′ to 3′ order in a nucleic acid molecule which corresponds to a single-stranded vector genome in the sense orientation, or in the sense strand of a double-stranded DNA molecule comprising the genome sequence, such as might occur in a plasmid used to producing vectors in host cells. Conversely, these elements can be arranged sequentially in 3′ to 5′ order in a complementary nucleic acid molecule which corresponds to a single-stranded vector genome in the antisense orientation, or in the antisense strand of a double-stranded DNA molecule comprising the genome sequence. Thus, for example (and not by way of limitation), if a vector genome in the sense orientation comprised an enhancer, a promoter, a transgene encoding a protein, and a poly(A) signal sequence in 5′ to 3′ order, the complementary antisense vector genome sequence would comprise those same elements, starting from its 5′ end, in the order of poly(A), transgene, promoter, enhancer, except that the nucleotide sequence of each of the elements (and any connected sequence between them) in the 5′ to 3′ direction would be the reverse complement of that of the same element in the sense stranded vector genome sequence. In the case of a self-complementary vector genome (scAAV), the arrangement of elements would occur in both 5′ to 3′ order over about half of its sequence, and then in 3′ to 5′ order in the complementary half.

In some embodiments, vector genomes of the AAV vectors of the disclosure comprise in 5′ to 3′ order a first AAV ITR from AAV2 at the 5′ terminus of the genome, a transcription control region, an intron, a transgene encoding GCase protein in operable linkage with the transcription control region, a transcription termination signal sequence, and a second AAV ITR from AAV2 at the 3′ terminus of the genome. In some embodiments, the transcription control region comprises an enhancer region and a promoter, either or both of which can be liver tissue specific, such as (in 5′ to 3′ order) an enhancer region comprising two tandem copies of an AMBP gene enhancer sequence, each comprising, consisting essentially of, or consisting of the nucleotide sequence of SEQ ID NO:8, an ALB gene enhancer sequence, comprising, consisting essentially of, or consisting of the nucleotide sequence of SEQ ID NO:9, and an ALB gene promoter sequence, comprising, consisting essentially of, or consisting of the nucleotide sequence of SEQ ID NO:10. In some embodiments, the intron is derived from the human beta globin (HBB) gene and comprises, consists essentially of, or consists of the nucleotide sequence of SEQ ID NO:11. In some embodiments, the transcription termination signal sequence is derived from the bovine growth hormone (bGH) gene and and comprises, consists essentially of, or consists of the nucleotide sequence of SEQ ID NO:14. In some embodiments, the AAV vector genome further comprises a modified TBP intron 2 sequence positioned 3′ of the transcription termination signal sequence and 5′ of said second AAV2 ITR. In any of the foregoing embodiments, the transgene encoding human GCase protein can comprise, consist essentially of, or consist of the nucleotide sequence of SEQ ID NO:41, or encode a GCase protein comprising a mature polypeptide identical in sequence to amino acids 20-516 of SEQ ID NO:16 and comprise a nucleotide sequence at least 50%, 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to the nucleotide sequence of SEQ ID NO:13. In any of the foregoing embodiments, either or both of the first and second AAV2 ITRs can be full length or truncated, and can be in the flip or flop configuration. In any of the foregoing embodiments, either or both of the first and second AAV2 ITRs can comprise, consist essentially of, or consist of the nucleotide sequence of SEQ ID NO:7, SEQ ID NO:15, SEQ ID NO: 18, or SEQ ID NO:19, or the reverse complement of each of such sequences. In any of the foregoing embodiments, the vector genome can comprise, consist essentially of, or consist of the nucleotide sequence of SEQ ID NO:17, or the reverse complement thereof. In any of the foregoing embodiments, the vector genome can be single-stranded, meaning that it is not self-complementary (outside of the ITRs), and can have length ranging from about 4000 to 5000 nucleotides. In any of the foregoing embodiments, the vector genome can be in the sense orientation, or in the antisense orientation. In any of the foregoing embodiments, the vector genome can be encapsidated by an AAV capsid, such as a hepatotropic AAV capsid, non-limiting examples of which include capsids from AAV2, AAV3B, AAV5, AAV6, AAV7, AAV8, AAV9, AAV13, AAVrh.74, AAVrh.10, AAV-DJ, AAV-LK03, AAV-KP1, AAV-hu.Lvr01, AAV-hu.Lvr02, AAV-hu.Lvr03, AAV-hu.Lvr04, AAV-hu.Lvr05, AAV-hu.Lvr06, AAV-hu.Lvr07, AAV-Anc80, AAV-NP40, AAV-NP59, AAV-NP84, AAV-hu.37, AAV-rh.8, AAV-rh.64R1, RHM4-1, RHM15-1, RHM15-2, RHM15-3/RHM15-5, RHM15-4, and RHM15-6, AAVHSC7, AAVHSC15, and AAVHSC17, or an AAV capsid comprising a VP1 protein comprising or consisting of an amino acid sequence selected from the group of consisting of: SEQ ID NO:1 and SEQ ID NO:4. In any of the foregoing embodiments, the AAV vector can comprise an AAV vector genome encapsidated in a capsid from AAV3B, where the nucleotide sequence of the genome comprises, consists essentially of, or consists of the nucleotide sequence of SEQ ID NO:17, or the reverse complement thereof.

In some embodiments, vector genomes of the AAV vectors of the disclosure further comprise a third AAV ITR positioned between the first and second AAV ITRs, such as in the middle of the vector genome (even if not exactly in the middle) which, in some embodiments, can have a mutated or altered terminal resolution site that does not undergo terminal resolution. In these embodiments, the vector genome can be self-complementary, and can have ranging from about 4000 to 5000 nucleotides when packaged in a capsid, or length ranging from about 2000 to 2500 nucleotides when its sequence is contained in a plasmid suitable for use in producing scAAV vectors in host cells.

### AAV Vector Production

As known in the art, AAV vectors can be produced, including at large scale, in a variety of ways. AAV vectors, for example, can be made in mammalian or insect cells and then purified. The traditional approach, which does not rely on coinfection with a helper virus, involves use of three plasmids as discussed above. One plasmid contains genes for helper virus factors, a second contains the AAV genome sequence in double stranded form, and the third contains AAV rep and cap genes. The rep/cap plasmid often contains a rep gene from AAV2, although this is not a requirement, and the cap gene sequence is chosen based on which AAV capsid protein is desired to constitute the capsid. In practice, the three plasmids are often separately replicated in bacteria, purified, mixed in solution together in predetermined proportions, and then mixed with a transfection agent. The transfection mixture is then used to transfect suitable mammalian host cells (in adherent or suspension cell culture) which are incubated for sufficient time (e.g., 48 to 72 hours, etc.) and under conditions sufficient for the host cells to express the helper factors and the rep and cap genes, and for AAV vector genome to be replicated from its plasmid template and packaged into capsids. In some embodiments, the host cells are HEK293 cells, which constitutively express AdV helper factors E1A and E1B, such that the helper plasmid only need contain the AdV E2A, E4ORF6, and VA RNA genes. Use of other mammalian host cells that do not produce AdV or other viral helper factor on their own would necessitate use of a helper plasmid containing whichever helper factors are missing or are otherwise required. Although the so-called triple transfection method described above is commonly employed, there is no requirement that the genes for the helper factors, and rep and cap genes, be provided on separate plasmids. In principle all these genes could be housed in one plasmid, for example, in which case two plasmids can be used in the transfection.

Seeking more efficient methods of producing AAV vector at large scale, stable cell lines have been created that contain some but not all the components that would otherwise need to be introduced into cells by transient transfection. Packaging cell lines contain stably integrated AAV rep and cap genes. Production of AAV in packaging cells requires them to be transiently transfected with a plasmid containing an AAV vector genome and infected with a helper virus. It is also possible to produce AAV vectors in packaging cells without transfection by first infecting them with an AdV (either wild type or in which the E2b gene is deleted) which supplies AdV E1 gene products, which induce rep and cap expression in the cells, as well as helper factors required for AAV replication, followed by infection with a replication deficient hybrid AdV in which an AAV vector genome replaces the E1 gene in the genome of the hybrid virus.

In another option, producer cell lines contain stably integrated AAV rep and cap genes, and also an AAV vector genome. Production of AAV in producer cells requires them to be infected with a helper virus. Packaging and producer cells are described further in, e.g., Martin, J, et al., Generation and characterization of adeno-associated virus producer cell lines for research and preclinical vector production, Hum. Gene Methods, 24:253-269 (2013); Gao, G P, et al., High-titer adeno-associated viral vectors from a Rep/Cap cell line and hybrid shuttle virus, Hum. Gene Ther., 9:2353-62 (1998); Martin, J, et al., Generation and Characterization of Adeno-Associated Virus Producer Cell Lines for Research and Preclinical Vector Production, Hum. Gene Ther. Meth., 24:253-69 (2013); Clement, N and J C Grieger, Manufacturing of recombinant adeno-associated viral vectors for clinical trials, Mol. Ther. Meth. & Clin. Dev. (2016) 3, 16002 (doi: 10.1038/mtm.2016.2). Other cellular systems for producing AAV vectors in mammalian cells, including at commercial scale, are possible.

The baculovirus system has also been employed to produce AAV vectors, in which Sf9 insect cells are infected with recombinant baculovirus vectors that variously contain the AAV rep and cap genes and the AAV genome. The exogenous genes are expressed, followed by genome packaging into vector particles within the cells. In early versions of the system, each component, rep, cap, and genome, were carried by three separate baculoviruses. Later, modifications were made, such as combining rep and cap into a single baculovirus, so that only two types of baculovirus were required, as well as producing Sf9 cell lines containing stably integrated AAV rep and cap genes, which only require infection with a single type of recombinant baculovirus containing an AAV vector genome. Use of the baculovirus system to produce AAV vector is described further in, e.g., Urabe, M, et al., Insect Cells as a Factory to Produce Adeno-Associated Virus Type 2 Vectors, Hum. Gene Ther., 13:1935-43 (2002); Virag, T, et al., Producing recombinant adeno-associated virus in foster cells: Overcoming production limitations using a baculovirus-insect cell expression strategy, Hum. Gene Ther., 20:807-17 (2009); Smith, R H, et al., A simplified baculovirus-AAV expression vector system coupled with one-step affinity purification yields high-titer rAAV stocks from insect cells, Mol. Ther., 17:1888-96 (2009); Mietzsch, M, et al., OneBac: platform for scalable and high-titer production of adeno-associated virus serotype 1-12 vectors for gene therapy, Hum. Gene. Ther. 25 (3): 212-22 (2014). Other cellular systems for producing AAV vectors in insect cells, including at commercial scale, are possible.

### Host Cells

As used herein, “host cells” means cells suitable for or adapted to in vitro production of AAV vectors. Host cells are often clonal cell lines capable of dividing for multiple generations before senescence stops growth, or may even be immortal. To produce vectors, host cells can be modified, transiently or non-transiently, through the introduction of exogenous genetic information designed to direct biosynthesis in host cells of the various components required for AAV vector asembly, notably the AAV capsid proteins, Rep proteins, helper virus factors, and vector genomes. For example, host cells can be transfected with exogenously supplied nucleic acid, such as in the form of one or more DNA plasmids, containing nucleotide sequences coding for the required vector components.

Various ways are known in the art for transfecting host cells with nucleic acid. These include, without limitation, mixing nucleic acid with certain compounds that can complex with nucleic acids and then be taken up into the cells, including calcium phosphate or cationic organic compounds, such as DEAE-dextran, polyethylenimine (PEI), polylysine, polyornithine, polybrene, cyclodextrin, cationic lipids, and others known in the art. Transfection can also be performed non-chemically via electroporation and more exotic technologies, such as biolistic particle delivery. As known in the art, transfection can be transient or stable. With transient transfection, the transfected nucleic acid exists in the cell for a limited period of time and, in the case of DNA, does not integrate into the genome. With stable transfection, DNA introduced into the cell can persist for long periods either as an episomal plasmid, or integrated into a chromosome. Usually, to produce stably transfected cells, a plasmid containing a selection marker gene, as well as nucleotide sequence coding for one or more of the required vector components, is transfected into the cells which are then grown and maintained under selective pressure, i.e., conditions that kill non-transfected cells or transfected cells from which the exogenous DNA, including its selection marker, are lost for some reason. For example, plasmids can contain an antibiotic resistance gene and transfected cells can be selected for by adding the antibiotic to the media in which the cells are grown. In some embodiments, the nucleotide sequence coding for one or more of the required vector components introduced into stably transfected host cells is under the control of an inducible promoter and is not expressed, or only at a low level, unless an environmental factor, such as a drug, metal ion, or temperature increase, which induces the promoter, is introduced as the cells are grown.

In other embodiments, host cell genomes can be modified in a non-transient and targeted fashion using genetic engineering methods, such as knock-in, or gene editing methods, to direct host cells to produce one or more of the required vector components. In other embodiments, nucleotide sequence coding for one or more of the required vector components can be introduced into host cells for purposes of directing production of AAV vectors via transduction, in which host cells are infected with modified viruses containing such nucleotide sequences. Examples of viral vectors useful for such purposes include adenovirus, retroviruses (including lentiviruses), baculoviruses, vaccinia virus, and herpes simplex virus, with others being possible.

Host cells can be any type of cell known in the art to be useful for the purpose of producing AAV vectors. Host cells are often animal cells, with different types or species being possible, such as insect cells or mammalian cells, including rat, mouse, or human cells, with others being possible. In some embodiments, host cells useful for producing AAV vectors of the disclosure are mammalian host cells, examples of which include Hela cells, Cos cells, HEK293 cells (and variants of HEK293 cells, such as HEK293E, HEK293F, HEK293H, HEK293T or HEK293FT cells), A549 cells, BHK cells, Vero cells, NIH 3T3 cells, HT-1080 cells, Sp2/0 cells, NS0 cells, C127 cells, AGE1.HN cells, CAP cells, HKB-11 cells, WI-38 cells, MRC-5 cells, or PER.C6 cells, with many others being possible. In some embodiments, host cells useful for producing AAV vectors of the disclosure are insect host cells, examples of which include Sf9 cells, ExpiSf9, Sf21 cells, S2 cells, D.Mel2 cells, Tn-368 cells, or BTI-Tn-5B1-4 cells, with many others being possible. In some embodiments, host cells, including without limitation HEK293 cells, and its variants, can be adapted to growth in suspension culture.

For purposes of producing AAV vectors, host cells are often grown or maintained in culture under controlled conditions conducive to their growth and vector biosynthesis. For example, host cells can be grown in liquid media of defined chemical composition that provides all the nutrients necessary for cell growth and biosynthesis. Exemplary media includes DMEM, DMEM/F12, MEM, RPMI 1640, for mammalian host cells, and Express Five SFM, Sf-900 II SFM, Sf-900 III, or ExpiSf CD, for certain insect cells. Such media may be supplemented with antibiotics, growth factors or cytokines (produced recombinantly or present in animal serum, such as FBS) known to stimulate growth of the particular type of cells in use, as well as other ingredients that may be required for optimal biosynthesis of AAV vectors, but that would otherwise be in limiting supply. Exemplary supplements include essential amino acids, glutamine, vitamin K, insulin, BSA, or transferrin. In addition to the growth media, other culture conditions may be controlled to optimize growth and/or productivity of the cells, such as pH, temperature and CO₂ and oxygen concentration.

Host cells in culture can be grown or maintained in many containers known in the art, such as stirred tank bioreactors, wave bags, spinner flasks, hollow fiber bioreactors, or roller bottle, some of which can be designed and configured for single use or multiple use. Depending on the characteristics of the host cells in question, host cells can be grown in adherent cell culture, where the cells attach to and grow while in contact with a physical substrate, or in suspension cell culture, either where single cells float free in the media that sustains them, or while attached to bead microcarriers, which are suspended in the media. As known in the art, various technologies have been developed and can be used to grow host cells to high cell density, such as perfusion culture, which can increase the overall amount of AAV vector generated per production run.

As known in the art, samples of host cells are often maintained in frozen cell banks, such as master cell banks and working cell banks, which facilitate production of biological products in many batches over time, while ensuring consistent performance by the host cells. Before a campaign to produce an AAV vector, a frozen sample of host cells from a cell bank would typically be thawed, seeded into a small culture volume, and grown to ever higher densities or numbers in cultures of increasing volume. When host cells have reached a desired cell density and/or volume in culture, exogenous genetic material can be introduced, such as by transfection with plasmid DNA or infection or transduction with viral vectors, to cause them to begin producing the AAV vector. Alternatively, if using non-transiently modified host cells in which the nucleotide sequence coding for one or more of the required vector components is under inducible control, the environmental factor necessary to induce expression can be introduced. Host cells can then be grown or maintained in culture for time and under conditions sufficient for them to produce the AAV vectors.

### AAV Vector Purification

After biosynthesis in host cells, AAV vectors can be purified in a variety of ways known in the art. For example, in some embodiments, host cells can be lysed mechanically or chemically, such with detergent, after which host cell DNA and other components are removed, followed by steps such as density gradient centrifugation, or use of one or more chromatographic separation methods, to achieve a highly purified preparation of AAV vectors for use in research or methods of treatment.

Chromatography methods useful in the purification of AAV vectors include, without limitation, size exclusion chromatography (SEC); affinity chromatography, using any affinity ligand attached to the chromatography resin or matrix capable of specific binding to a capsid, such as an antibody, lectin, or glycan; immobilized metal chelate chromatography (IMAC); thiophilic adsorption chromatography; hydrophobic interaction chromatography (HIC); multimodal chromatography (MMC); pseudo-affinity chromatography; and ion exchange chromatography (IEX or IEC), such as anion exchange chromatography (AEX) or cation exchange chromatography (CEX).

In some embodiments, AAV vectors can be purified using antibody-based affinity chromatography in which an antibody, or antibody fragment thereof, is attached to a stationary phase (matrix or resin) loaded into a chromatography column through which a host cell lysate is pumped, followed by washing and eluting of vector which had bound to the antibodies. The antibody bound to the solid phase can be an IgG, or fragment thereof, or a single-chain camelid antibody (such as a heavy chain variable region camelid antibody), other types of antibodies being possible. Non-limiting examples of ligand affinity resins include Sepharose AVB, POROS CaptureSelect AAVX, POROS CaptureSelect AAV8, and POROS CaptureSelect AAV9. See, e.g., Terova, O, et al., Affinity Chromatography Accelerates Viral Vector Purification for Gene Therapies, BioPharm Intl. eBook pp. 27-35 (2017); Mietzsch, M, et al., Characterization of AAV-Specific Affinity Ligands: Consequences for Vector Purification and Development Strategies, Mol. Ther. Meth. & Clin. Dev., 19:362-73 (2020); Rieser, R, et al., Comparison of Different Liquid Chromatography-Based Purification Strategies for Adeno-Associated Virus Vectors, Pharmaceutics 13, 748 (2021) (doi.org/10.3390/pharmaceutics13050748).

In other embodiments, AAV vectors can be purified using ligand chromatography in which the stationary phase has attached to it the same type of ligand that certain AAVs are known to use when binding to cells, such as a glycan, sialic acid (e.g., an O-linked or N-linked sialic acid), galactose, heparin, heparan sulfate, or a proteoglycan, such as a heparan or heparin sulfate proteoglycan (HSPG). For example, an affinity matrix containing sialic acid residues can be used to purify AAV vectors with capsids that specifically bind to sialic acid (e.g., AAV1, AAV4, AAV5, or AAV6); an affinity matrix containing galactose can be used to purify AAV vectors with capsids that specifically bind to galactose (e.g., AAV9); and an affinity matrix containing heparin, heparan, or HSPG can be used to purify AAV vectors with capsids that specifically bind to HSPG (e.g., AAV2, AAV3A, AAV3B, AAV6, or AAV13).

Depending on the physicochemical characteristics of the vector, such as the charge on the capsid, AAV vectors can be further purified by performing anion exchange, cation exchange, or hydrophobic interaction chromatography. Other downstream process steps useful for purifying AAV vectors may be used as well, such as, without limitation, desalting and buffer exchange, ultrafiltration, nanofiltration, diafiltration, and tangential flow filtration (TFF). Use of more than one downstream processing step is possible, and a plurality of downsteam processing steps can be performed in any order according to the knowledge of those ordinarily skilled in the art.

### Methods of Treatment

Among other embodiments, the disclosure provides methods for treating Gaucher disease, including Type 1 Gaucher disease (GD1), or other type of GCase deficiency, by administering to a subject, such as a human subject, in need of treatment for Gaucher disease (or GCase deficiency) a therapeutically effective amount of an AAV vector of the disclosure (including, without limitation, the vector described herein as AAV3B-GBA clone 128), or a pharmaceutical composition containing such AAV vectors. Also provided is use of an AAV vector of the disclosure in the manufacture of a medicament for use in the methods of treatment disclosed herein. In addition, there is provided an AAV vector of the disclosure, or pharmaceutical composition containing such AAV vectors, for use in the methods of treatment disclosed herein.

Treatment of subjects with Gaucher disease need not result in a cure to be considered effective, where “cure” is defined as either halting disease progression, or partially or completely restoring the subject's health as it was before the onset or worsening of symptoms, or relative to healthy humans without GD1. Rather, a therapeutically effective amount of an AAV vector of the disclosure (including, without limitation, the vector described herein as AAV3B-GBA clone 128), or a pharmaceutical composition containing such AAV vectors, can be one that serves to at least partially reverse, reduce or ameloriate the extent or severity in a subject of at least one symptom or sign associated with Gaucher disease or GCase deficiency; or at least partially reverse, reduce or ameloriate the extent or severity in a subject of at least one disorder or dysfunction of the body, organ, tissue, or cell, caused by Gaucher disease or GCase deficiency; or slow the progression of Gaucher disease or other deleterious effects of GCase deficiency in a subject; or improve the quality of life of subjects with Gaucher disease or experiencing a deleterious effect of GCase deficiency. Examples of symptoms, signs, disorders or dysfunctions associated with Gaucher disease or GCase deficiency include, without limitation, hepatosplenomegaly, hepatomegaly, splenomegaly, anemia, leukoneutropenia, leukopenia, pancytopenia, thrombocytopenia, monoclonal hypergammaglobulinemia, polyclonal hypergammaglobulinemia, anorexia, chronic fatigue, bone crisis, avascular bone necrosis, bone pain, osteolysis, osteonecrosis, osteopenia, reduced quality of life (QoL), and other symptoms, signs, disorders or dysfunctions associated with Gaucher disease or GCase deficiency which are known in the art.

In some embodiments, the methods for treating GD1, or other type of GCase deficiency, disclosed herein can be used to treat GD1 or GCase deficiency in a human subject with any type of homozygous or heterozygous deleterious mutation in or affecting the GBA gene. Non-limiting examples of deleterious mutations include deletions, insertions, recombinations, splice site variants, missense, or nonsense mutations in either or both alleles of the GBA gene, mutations affecting transcriptional control regions (e.g., enhancers or promoters) of either or both alleles of the GBA gene, and/or mutations which reduce stability of mRNA expressed from either or both alleles of the GBA gene, or the amount of protein translated from such mRNA transcripts, so long as the mutation(s) results in a reduction in the amount or loss of GCase protein that is produced, and/or a reduction in the amount or loss of enzymatic activity of GCase protein that is produced. For example, and without limitation, some of the more common deleterious mutations in the human GBA gene associated with GB1 include c.1226A>G (N370S), c.1448T>C (L444P), c.84dup, c.115+1G>A (IVS2+1G>A), and the RecNcil mutation resulting from recombination between GBA and GBAP pseudogene, as well as many others which are known in the art. Methods for genotyping a subject as having a deleterious mutation in either or both alleles of the GBA gene, such as by RFLP analysis or gene or genomic sequencing, are familiar to those of ordinary skill in the art, as are methods for detecting and quantifying the amount of GCase protein and/or GCase enzymatic activity which is present in a sample from a subject.

In some embodiments, subjects are human subjects diagnosed with GD1 who were never before treated with enzyme replacement therapy (ERT) or substrate reduction therapy (SRT) for GD1 (i.e., standard of care treatment naïve), whereas in other embodiments, subjects are human subjects diagnosed with GD1 who had been undergoing ERT or SRT before treatment with the methods of the disclosure. In some embodiments, with respect to a subject with GD1 whom is receiving standard of care treatment (i.e., not treatment naïve), a therapeutically effective amount of an AAV vector of the disclosure, or a pharmaceutical composition containing such AAV vectors, permits such subjects to cease ERT or SRT, or reduce the dose and/or frequency of such ERT or SRT, and not experience worsening symptoms, signs, disorders or dysfunctions characteristic of Gaucher disease (or GCase deficiency), at least for a period of time (that is, the therapeutic effect need not be lifelong).

Therapeutic efficacy of the methods of treatment disclosed herein can be assessed in individual subjects with GD1 or other type of GCase deficiency by observing or measuring and comparing the severity or magnitude of any symptom, sign, disorder, dysfunction, or laboratory value characteristic of GD1 (or GCase deficiency) before (baseline) and after treatment. Such comparison can be performed at one or more times after treatment, such as at 0, 1, 2, 3, 6, 9, 12, 15, 18, 21, 24, 27, 30, 33, 36, 39, 42, 45, or 48 months, or some other time after treatment. The data used for comparison from individual subjects can be single data points, or the mean of a plurality of data points if available.

In some other embodiments, therapeutic efficacy can be assessed in a population (i.e., two or more) of subjects with GD1 or other type of GCase deficiency serving as their own controls by observing or measuring the severity or magnitude of any symptom, sign, disorder, dysfunction, or laboratory value characteristic of GD1 (or GCase deficiency) among the individuals within the population before (baseline) and after treatment, and comparing the averaged pre-treatment data with the averaged post-treatment data. Such comparison can be performed at one or more times after treatment, such as at 0, 1, 2, 3, 6, 9, 12, 15, 18, 21, 24, 27, 30, 33, 36, 39, 42, 45, or 48 months, or some other time after treatment. The population under study can be receiving standard of care ERT or SRT therapy for GD1 before treatment with the AAV vectors of the disclosure, and will typically cease ERT or SRT therapy (as the case may be) after vector treatment, at least during the duration of monitoring for therapeutic effect. In other embodiments, the population under study can be naïve for standard of care therapy before treatment with the AAV vectors of the disclosure. In some embodiments, studies intended to establish and quantify therapeutic efficacy can be designed to compare treatment effects in a population of subjects treated with AAV vectors of the disclosure (treatment arm) to treatment effects in a population of subjects receiving placebo (control arm). Typically, although not necessarily, subjects within treament and control arms in the study are matched as to relevant subject characteristics, such as age, sex, disease severity at time of intervention, and whether a subject has ever received or is receiving standard of care ERT or SRT therapy, or is instead treatment naïve. In other embodiments, the control population is not treated with placebo, but is instead drawn from a natural history study in which GD1 patients are observed to describe and quantify the progression of relevant disease parameters in the absence of gene therapy.

In some embodiments, the methods for treating GD1, or other type of GCase deficiency, disclosed herein are effective for treating subjects with GD1 or GCase deficiency of any age including, without limitation, subjects less than 1 year of age, such as subjects that are about 1, 2, 3, 4, 5, 6, 7, 8, 9, 10, or 11 months of age, or subjects that are at least or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70 years of age or older, or an age including and between any of the foregoing specifically enumerated ages.

In some embodiments, the methods for treating GD1, or other type of GCase deficiency, disclosed herein are effective for treating subjects with GD1 of any level or extent of severity at the time AAV vectors of the disclosure are first administered, such as mild, moderate, or severe GD1, or extent of severity of GD1 as reflected in a subject's severity score using a suitable severity scoring index, such as the GD1-DS3 or GauSSI-I, which are described further below. For example and without limitation, at the time of first treatment with AAV vectors of the disclosure, a subject or subjects may have a severity score of 19, 18, 17, 16, 15, 14, 13, 12, 11, 10, 9, 8, 7, 6, 5, 4, 3, 2, or 1 on the GD1-DS3 severity score index, or a severity score of 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 on the GauSSI-I. In some embodiments, at the time of first treatment with AAV vectors of the disclosure, a subject has not demonstrated any overt signs or symptoms of GD1, but has been diagnosed as likely to develop such signs or symtoms based on genetic testing demonstrating existence of at least one deleterious mutation in either or both alleles of the subject's GBA gene.

In some embodiments, the methods for treating GCase deficiency, including Gaucher disease Type 1 (GD1), with the AAV vectors of the disclosure (including, without limitation, the vector described herein as AAV3B-GBA clone 128) are effective to treat GD1, or any other form of GCase deficiency, for a period of time after vector administration during which the patient can forego standard of care treatment for Gaucher disease Type 1, for example, ERT or SRT therapy, without experiencing any symptoms or signs of Gaucher disease, or without experiencing any worsening of symptoms or signs of Gaucher disease that may have been present at the time of gene therapy, or at most experiencing minimal worsening of symptoms or signs of Gaucher disease that may have been present at the time of gene therapy such that the patient's overall health, function, quality of life, and/or longevity is not substantially or materially impacted. In some embodiments, this period, referred to herein as the period of therapeutic durability can be any suitable or desired period of time, including for example and without limitation, at least or about 3, 6, 9, 12, 15, 18, 21, 24, or more months, or at least or about 1, 2, 3, 4, 5, 6, 7, 8, 9, 10, 11, 12, 13, 14, 15, or more years, or at least or about 1, 2, 3, 4, 5, 6, 7, or more decades, or any span of time between any of the specifically enumerated values, or even in some embodiments, the remainder of the patient's lifespan after receiving gene therapy as described herein.

### Hematologic Manifestations

In some embodiments, the methods for treating GCase deficiency, including Gaucher disease Type 1 (GD1), with the AAV vectors of the disclosure (including, without limitation, the vector described herein as AAV3B-GBA clone 128) are effective to reduce the incidence, frequency or severity of hematological manifestations of the disease process, including anemia, thrombocytopenia and excess bleeding, among others. In some embodiments, treatment is effective to reduce in GD1 patients the frequency or extent of hematologic manifestations of the disease process within a period of time after the administration of AAV vectors of the disclosure of at most, or at least, or about 10, 15, 20, 25, 30, 35, 40, 45, 50, 55, 60, 70, 80, 90, 100, 110, 120, 130, 140, 150, 160, 170, or 180 days, or 1, 2, 3, 4, 5, 6, 7, 8, 9, 10, 11, 12, 13, 14, 15, 16, 17, or 18 months, or 1.0, 1.5, 2.0, 2.5, 3.0, 3.5, 4.0, 4.5, 5.0, 5.5, or 6.0 years, or a range of time between and encompassing any of the foregoing enumerated periods of time.

### Anemia

In some embodiments, the methods for treating GCase deficiency, including Gaucher disease Type 1 (GD1), with the AAV vectors of the disclosure (including, without limitation, the vector described herein as AAV3B-GBA clone 128) are effective to reduce the incidence, frequency or severity of anemia, a lower than normal number of red blood cells, which is often quantified by measuring the amount of hemoglobin in the blood or the percent of blood volume which is made up of red blood cells (hematocrit), and associated risks including fatigue, weakness, shortness of breath, dizziness, and others. In some embodiments, methods of the disclosure for treating GD1 can reduce the number of blood transfusions otherwise required by GD1 patients to prevent symptoms of anemia, such as fatigue, or other symptoms. In some embodiments, treatment is effective to reduce in GD1 patients the frequency or extent of anemia within a period of time after the administration of AAV vectors of the disclosure of at most, or at least, or about 10, 15, 20, 25, 30, 35, 40, 45, 50, 55, 60, 70, 80, 90, 100, 110, 120, 130, 140, 150, 160, 170, or 180 days, or 1, 2, 3, 4, 5, 6, 7, 8, 9, 10, 11, 12, 13, 14, 15, 16, 17, or 18 months, or 1.0, 1.5, 2.0, 2.5, 3.0, 3.5, 4.0, 4.5, 5.0, 5.5, or 6.0 years, or a range of time between and encompassing any of the foregoing enumerated periods of time.

In some embodiments, methods of the disclosure for treating GD1 (or other GCase deficiency) are effective to increase the amount of hemoglobin (Hb) in the blood, either in individual GD1 patients who have been treated, or as an average in a population of treated GD1 patients, for example, from less than 8 grams per deciliter (g/dL) to a greater number, such as 8.0 to 9.9 g/dL, or 10 to 12 g/dL, or a hemoglobin concentration in blood of greater than 12 g/dL in adult males, or greater than 11.5 g/dL in adult females. In some of these embodiments the GD1 patients were standard of care treatment naïve before gene therapy as described herein, whereas in other embodiments the GD1 patients were receiving standard of care ERT or SRT therapy before gene therapy.

In some embodiments, the methods of treatment are effective to increase the Hb concentration in the blood, either in individual GD1 patients who have been treated, or as an average in a population of treated GD1 patients, to at least or about 5%, 10%, 15%, 20%, 25%, 30%, 35%, 40%, 45%, 50%, 60%, 70%, 80%, 90%, 100%, or 110% of the average Hb concentration in the blood of healthy humans. In some of these embodiments the GD1 patients were standard of care treatment naïve before gene therapy as described herein, whereas in other embodiments the GD1 patients were receiving standard of care ERT or SRT therapy before gene therapy. In some embodiments, depending on the reference range used, the Hb concentration in the blood of healthy humans ranges in adult men from about 13.2 to 16.6 grams per deciliter (g/dL), or 13.8 to 17.2 g/dL, or 14.0 to 17.5 g/dL, or 14 to 18 g/dL, ranges in adult women from about 11.6 to 15.0 g/dL, or 12.1 to 15.1 g/dL, or 12.3 to 15.3 dL, or 12 to 16 g/dL, ranges in children from 1 to 6 years old 9.5 to 14.0 g/dL, and ranges in children and youths 6 to 18 years old 10.0 to 15.5 g/dL. In some embodiments, the normal reference is another value, such as about 13, 14, 15, 16, 17, or 18 g/dL for men, and 11, 12, 13, 14, 15, or 16 g/dL for women, or a range encompassing, or an integer value between, any of the foregoing specifically enumerated values.

In some embodiments, the methods of treatment are effective to increase the Hb concentration in the blood of GD1 patients, either in individual GD1 patients who have been treated, or as an average in a population of treated GD1 patients, by at least or about 1%, 2%, 3%, 4%, 5%, 6%, 7%, 8%, 9%, 10%, 11%, 12%, 13%, 14%, 15%, 16%, 17%, 18%, 19%, 20%, 21%, 23%, 24%, 25%, 26%, 27%, 28%, 29%, 30%, 31%, 32%, 33%, 34%, 35%, 36%, 37%, 38%, 39%, 40%, 50%, 60%, 70%, 80%, 90%, 100%, 110%, or more above the baseline Hb concentration prior to gene therapy, or compared to the average Hb concentration in the blood from comparable (e.g., age and sex matched) control patients with GD1 or other form of GCase deficiency. In some embodiments, the latter control groups are standard of care treatment naïve or are receiving standard of care ERT or SRT therapy. In some non-limiting embodiments, the baseline Hb concentration in the blood of standard of care treatment naïve GD1 patients or GD1 patients who are receiving standard of care ERT or SRT therapy is not more than, or is less than, or is about 170, 160, 150, 140, 130, 120, 110, 110, 90, 80, 70, 60, or 50 g/dL blood, or less, or a range encompassing, or an integer value between, any of the foregoing specifically enumerated values. In some of these embodiments the GD1 patients were standard of care treatment naïve before gene therapy as described herein, whereas in other embodiments the GD1 patients were receiving standard of care ERT or SRT therapy before gene therapy.

In some embodiments, the methods of treatment are effective to increase the Hb concentration in the blood of GD1 patients, either in individual GD1 patients who have been treated, or as an average in a population of treated GD1 patients, by at least or about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0 g/dL, or more, above the baseline Hb concentration prior to gene therapy, or compared to the average Hb concentration in the blood from comparable (e.g., age and sex matched) control patients with GD1 or other form of GCase deficiency. In some embodiments, the latter control groups are standard of care treatment naïve or are receiving standard of care ERT or SRT therapy. In some non-limiting embodiments, the baseline Hb concentration in the blood of standard of care treatment naïve GD1 patients or GD1 patients who are receiving standard of care ERT or SRT therapy is not more than, or is less than, or is about 170, 160, 150, 140, 130, 120, 110, 110, 90, 80, 70, 60, or 50 g/dL blood, or less, or a range encompassing, or an integer value between, any of the foregoing specifically enumerated values. In some of these embodiments the GD1 patients were standard of care treatment naïve before gene therapy as described herein, whereas in other embodiments the GD1 patients were receiving standard of care ERT or SRT therapy before gene therapy. In some of these embodiments, the GD1 patients were receiving standard of care ERT or SRT therapy before gene therapy as described herein, and such methods of treatment are effective to maintain, within a certain margin, the blood Hb concentration resulting from standard of care therapy. In some embodiments, the margin is less than or about 30%, 25%, 20%, 15%, 10%, 9%, 8%, 7%, 6%, 5%, 4%, 3%, 2%, 1%, or 0%.

In some embodiments, the methods of treatment are effective to increase hematocrit, which is the percentage by volume of red blood cells in blood, of GD1 patients, either in individual GD1 patients who have been treated, or as an average in a population of treated GD1 patients, to at least or about 5%, 10%, 15%, 20%, 25%, 30%, 35%, 40%, 45%, 50%, 60%, 70%, 80%, 90%, 100%, 110%, or more, of the average hematocrit of healthy humans. In some of these embodiments the GD1 patients were standard of care treatment naïve before gene therapy as described herein, whereas in other embodiments the GD1 patients were receiving standard of care ERT or SRT therapy before gene therapy. In some embodiments, depending on the reference range used, the hematocrit of healthy humans ranges in adult men from about 40% to 54% or 41% to 50%, and ranges in adult women from about 36% to 44% or 36% to 48%. In some embodiments, the normal reference is an integer value, such as 35%, 36%, 37%, 38%, 39%, 40%, 41%, 42%, 43%, 44%, 45%, 46%, 47%, 48%, 49%, 50%, 51%, 52%, 53%, 54%, or 55%.

In some embodiments, the methods of treatment are effective to increase hematocrit of GD1 patients, either in individual GD1 patients who have been treated, or as an average in a population of treated GD1 patients, by at least or about 1%, 2%, 3%, 4%, 5%, 6%, 7%, 8%, 9%, 10%, 11%, 12%, 13%, 14%, 15%, 16%, 17%, 18%, 19%, 20%, 21%, 23%, 24%, 25%, 26%, 27%, 28%, 29%, 30%, 31%, 32%, 33%, 34%, 35%, 36%, 37%, 38%, 39%, 40%, 50%, 60%, 70%, 80%, 90%, 100%, 110%, or more, above the baseline Hb concentration prior to gene therapy, or compared to the average hematocrit from comparable (e.g., age and sex matched) control patients with GD1 or other form of GCase deficiency. In some embodiments, the latter control groups are standard of care treatment naïve or are receiving standard of care ERT or SRT therapy. In some of these embodiments the GD1 patients were standard of care treatment naïve before gene therapy as described herein, whereas in other embodiments the GD1 patients were receiving standard of care ERT or SRT therapy before gene therapy.

### Thrombocytopenia

In some embodiments, methods for treating GCase deficiency, including Gaucher disease Type 1 (GD1), with the AAV vectors of the disclosure (including, without limitation, the vector described herein as AAV3B-GBA clone 128) are effective to reduce the incidence, frequency or severity of thrombocytopenia, a lower than normal number of platelets, often defined as fewer than 150,000 platelets per microliter (μL) blood, and associated risks including bruising, longer than normal clotting times, and excess bleeding. In some embodiments, treatment is effective to reduce in GD1 patients the frequency or extent of thrombocytopenia within a period of time after the administration of AAV vectors of the disclosure of at most, or at least, or about 10, 15, 20, 25, 30, 35, 40, 45, 50, 55, 60, 70, 80, 90, 100, 110, 120, 130, 140, 150, 160, 170, or 180 days, or 1, 2, 3, 4, 5, 6, 7, 8, 9, 10, 11, 12, 13, 14, 15, 16, 17, or 18 months, or 1.0, 1.5, 2.0, 2.5, 3.0, 3.5, 4.0, 4.5, 5.0, 5.5, or 6.0 years, or a range of time between and encompassing any of the foregoing enumerated periods of time.

In some embodiments, methods of the disclosure for treating GD1 can increase the platelet count, either in individual GD1 patients who have been treated, or as an average in a population of treated GD1 patients, for example, from less than 60,000/μL blood (often defined as moderate to severe thrombocytopenia) to a greater number, such as 60,000/μL to 100,000/μL or 60,000/μL to 120,000/μL, or 100,000/μL to 150,000/μL or 120,000/μL to 150,000/μL, or a platelet count greater than 150,000/μL blood. In some of these embodiments the GD1 patients were standard of care treatment naïve before gene therapy as described herein, whereas in other embodiments the GD1 patients were receiving standard of care ERT or SRT therapy before gene therapy. In some embodiments, methods of the disclosure for treating GD1 can reduce the number of platelet infusions otherwise required by GD1 patients to prevent excessive bleeding, such as before a dental procedure or surgery, or after trauma.

In some embodiments, the methods of treatment are effective to increase the platelet count, either in individual GD1 patients who have been treated, or as an average in a population of treated GD1 patients, to at least or about 5%, 10%, 15%, 20%, 25%, 30%, 35%, 40%, 45%, 50%, 60%, 70%, 80%, 90%, 100%, 110%, or more, of the average platelet count of healthy humans which, in some embodiments, can range from 150,000/μL to 450,000/μL blood. In some embodiments, the normal reference is another value, such as about 150,000/μL, 200,000/L, 250,000/μL, 300,000/μL, 350,000/μL, 400,000/μL, or 450,000/μL blood, or a range encompassing, or an integer value between, any of the foregoing specifically enumerated values. In some of these embodiments the GD1 patients were standard of care treatment naïve before gene therapy as described herein, whereas in other embodiments the GD1 patients were receiving standard of care ERT or SRT therapy before gene therapy.

In some embodiments, the methods of treatment are effective to increase the platelet count per microliter of blood of GD1 patients, either in individual GD1 patients who have been treated, or as an average in a population of treated GD1 patients, by at least or about 1%, 2%, 3%, 4%, 5%, 6%, 7%, 8%, 9%, 10%, 11%, 12%, 13%, 14%, 15%, 16%, 17%, 18%, 19%, 20%, 21%, 23%, 24%, 25%, 26%, 27%, 28%, 29%, 30%, 31%, 32%, 33%, 34%, 35%, 36%, 37%, 38%, 39%, 40%, 50%, 60%, 70%, 80%, 90%, 100%, 110%, or more, above the baseline platelet count prior to gene therapy, or compared to the average platelet count from comparable (e.g., age and sex matched) control patients with GD1 or other form of GCase deficiency. In some embodiments, the latter control groups are standard of care treatment naïve or are receiving standard of care ERT or SRT therapy. In some non-limiting embodiments, the baseline platelet count of standard of care treatment naïve GD1 patients or GD1 patients who are receiving standard of care ERT or SRT therapy is not more than, or is less than, or is about 350,000, 300,000, 250,000, 200,000, 150,000, 100,000, 90,000, 80,000, 70,000, 60,000, 50,000, 40,000, or 30,000 per μL blood, or less, or a range encompassing, or an integer value between, any of the foregoing specifically enumerated values. In some of these embodiments the GD1 patients were standard of care treatment naïve before gene therapy as described herein, whereas in other embodiments the GD1 patients were receiving standard of care ERT or SRT therapy before gene therapy.

In some embodiments, the methods of treatment are effective to increase the platelet count of GD1 patients, either in individual GD1 patients who have been treated, or as an average in a population of treated GD1 patients, by at least or about 5,000, 6,000, 7,000, 8,000, 9,000, 10,000, 15,000, 20,000, 25,000, 30,000, 35,000, 40,000, 45,000, 50,000, 55,000, 60,000, 65,000, 70,000, 75,000, 80,000, 85,000, 90,000, 95,000, 100,000, 105,000, 110,000, 115,000, 120,000, 125,000, 130,000, 135,000, 140,000, 145,000, 150,000, 160,000, 170,000, 180,000, 190,000, 200,000, 250,000, 300,000, or more platelets per microliter of blood, or a range encompassing, or an integer value between, any of the foregoing specifically enumerated values, above the baseline platelet count prior to gene therapy, or compared to the average platelet count from comparable (e.g., age and sex matched) control patients with GD1 or other form of GCase deficiency. In some embodiments, the latter control groups are standard of care treatment naïve or are receiving standard of care ERT or SRT therapy. In some non-limiting embodiments, the baseline platelet count of standard of care treatment naïve GD1 patients or GD1 patients who are receiving standard of care ERT or SRT therapy is not more than, or is less than, or is about 350,000, 300,000, 250,000, 200,000, 150,000, 100,000, 90,000, 80,000, 70,000, 60,000, 50,000, 40,000, or 30,000 per μL blood, or less, or a range encompassing, or an integer value between, any of the foregoing specifically enumerated values. In some of these embodiments the GD1 patients were standard of care treatment naïve before gene therapy as described herein, whereas in other embodiments the GD1 patients were receiving standard of care ERT or SRT therapy before gene therapy

One consequence of having a reduced platelet count is prolonged bleeding time before the blood clots, which can result in patients with GD1 or other form of GCase deficiency in excessive bleeding, such as mucocutaneous bleeding, for example, epistaxis, gingival bleeding, or menorrhagia, or postoperative hemorrhage, bleeding during birth, and spontaneous hematomas. Accordingly, in some embodiments, methods of the disclosure for treating GD1, or other forms of GCase deficiency, are effective to reduce bleeding time. Bleeding time can be measured using any method known in the art, such as the so-called Ivy method, in which normal bleeding time is less than 8 minutes, or the Duke method, in which normal bleeding time is less than 3 minutes. Thus, in some embodiments, the methods of treatment are effective to increase bleeding time from greater than 8 minutes as determined using the Ivy method to a time less than 8 minutes, or are effective to increase bleeding time from greater than 3 minutes as determined using the Duke method to a time less than 3 minutes. In some of these embodiments the GD1 patients were standard of care treatment naïve before gene therapy as described herein, whereas in other embodiments the GD1 patients were receiving standard of care ERT or SRT therapy before gene therapy.

### Leukopenia

In some embodiments, methods for treating GCase deficiency, including Gaucher disease Type 1 (GD1), with the AAV vectors of the disclosure (including, without limitation, the vector described herein as AAV3B-GBA clone 128) are effective to reduce the incidence, frequency or severity of leukopenia, a lower than normal number of white blood cells, often defined as fewer than 4,000 white blood cells per microliter (μL) blood, and associated risks including increased susceptibility to infection. In some embodiments, treatment is effective to reduce in GD1 patients the frequency or extent of leukopenia within a period of time after the administration of AAV vectors of the disclosure of at most, or at least, or about 10, 15, 20, 25, 30, 35, 40, 45, 50, 55, 60, 70, 80, 90, 100, 110, 120, 130, 140, 150, 160, 170, or 180 days, or 1, 2, 3, 4, 5, 6, 7, 8, 9, 10, 11, 12, 13, 14, 15, 16, 17, or 18 months, or 1.0, 1.5, 2.0, 2.5, 3.0, 3.5, 4.0, 4.5, 5.0, 5.5, or 6.0 years, or a range of time between and encompassing any of the foregoing enumerated periods of time.

In some embodiments, methods of the disclosure for treating GD1 increase the white blood cell count, either in individual GD1 patients who have been treated, or as an average in a population of treated GD1 patients, for example, from less than 1,900/μL blood to a greater number, such as 1,900/μL to 2,500/μL, or 2,500/μL to 4,000/μL, or a white blood cell count greater than 4,000/μL blood. In some of these embodiments the GD1 patients were standard of care treatment naïve before gene therapy as described herein, whereas in other embodiments the GD1 patients were receiving standard of care ERT or SRT therapy before gene therapy.

In some embodiments, the methods of treatment are effective to increase the white blood cell count per microliter of blood, either in individual GD1 patients who have been treated, or as an average in a population of treated GD1 patients, to at least or about 5%, 10%, 15%, 20%, 25%, 30%, 35%, 40%, 45%, 50%, 60%, 70%, 80%, 90%, 100%, 110%, or more, of the average white blood cell count of healthy humans which, in some embodiments, ranges from 4,000/μL to 11,000/μL blood. In some embodiments, the normal reference is another value, such as about 4,000/μL, 4,500/μL, 5,000/μL, 5,500/μL, 6,000/μL, 6,500/μL, 7,000/μL, 7,500/μL, 8,000/μL, 8,500/μL, 9,000/μL, 9,500/μL, 10,000/μL, 10,500/μL, or 11,000/μL blood, or a range encompassing, or an integer value between, any of the foregoing specifically enumerated values. In some of these embodiments the GD1 patients were standard of care treatment naïve before gene therapy as described herein, whereas in other embodiments the GD1 patients were receiving standard of care ERT or SRT therapy before gene therapy.

In some embodiments, the methods of treatment are effective to increase the white blood cell count per microliter of blood of GD1 patients, either in individual GD1 patients who have been treated, or as an average in a population of treated GD1 patients, by at least or about 1%, 2%, 3%, 4%, 5%, 6%, 7%, 8%, 9%, 10%, 11%, 12%, 13%, 14%, 15%, 16%, 17%, 18%, 19%, 20%, 21%, 23%, 24%, 25%, 26%, 27%, 28%, 29%, 30%, 31%, 32%, 33%, 34%, 35%, 36%, 37%, 38%, 39%, 40%, 50%, 60%, 70%, 80%, 90%, 100%, 110%, or more, above the baseline white blood cell count prior to gene therapy, or compared to the average white blood cell count from comparable (e.g., age and sex matched) control patients with GD1 or other form of GCase deficiency. In some embodiments, the latter control groups are standard of care treatment naïve or are receiving standard of care ERT or SRT therapy. In some of these embodiments the GD1 patients were standard of care treatment naïve before gene therapy as described herein, whereas in other embodiments the GD1 patients were receiving standard of care ERT or SRT therapy before gene therapy.

### Cellular GCase Levels

In some embodiments, methods for treating GCase deficiency, including Gaucher disease Type 1 (GD1), with the AAV vectors of the disclosure (including, without limitation, the vector described herein as AAV3B-GBA clone 128) are effective to increase the amount of GCase protein and/or enzymatic activity in peripheral blood mononuclear cells (PBMC), monocytes, total leukocytes, cultured fibroblasts, or other types of cell from of a patient with GD1 or other form of GCase deficiency. In some embodiments, treatment is effective to increase the amount of GCase protein and/or enzymatic activity in peripheral blood mononuclear cells (PBMC), total leukocytes, cultured fibroblasts, or other types of cell from of a patient with GD1 or other form of GCase deficiency within a period of time after the administration of an AAV vector of the disclosure of at most, or at least, or about 10, 15, 20, 25, 30, 35, 40, 45, 50, 55, 60, 70, 80, 90, 100, 110, 120, 130, 140, 150, 160, 170, or 180 days, or 1, 2, 3, 4, 5, 6, 7, 8, 9, 10, 11, 12, 13, 14, 15, 16, 17, or 18 months, or 1.0, 1.5, 2.0, 2.5, 3.0, 3.5, 4.0, 4.5, 5.0, 5.5, or 6.0 years, or a range of time between and encompassing any of the foregoing enumerated periods of time. Methods for quantifying the amount of GCase protein or enzymatic activity in PBMCs of a subject, before or after treatment, or of a healthy human serving as a control, are known in the art. See, e.g., Ysselstein, D, et al., Movement Disorders 36 (12): 2719-2730 (2021) (DOI: 10.1002/mds.28815). GCase activity, for example, can be detected and quantified using fluorogenic substrates, as described further, e.g., in DOE: 10.1021/ja5106738 and DOI: 10.1021/ja5106738.

In some embodiments, the methods of treatment are effective to increase the amount of GCase protein or enzymatic activity in PMBCs, monocytes, or leukocytes obtained from GD1 patients, either individual GD1 patients who have been treated, or as an average from a population of treated GD1 patients, to at least or about 5%, 10%, 15%, 20%, 25%, 30%, 35%, 40%, 45%, 50%, 60%, 70%, 80%, 90%, 100%, 110%, or more, of the average amount of GCase in PMBCs, monocytes, or leukocytes obtained from healthy humans. In some embodiments, the average amount of GCase in PMBCs, monocytes, or leukocytes of healthy humans ranges from about 20 to 130 picograms per million (pg/1E6) PBMCs, or averages about 70 pg/1E6 cells, or another value, such as about 30, 40, 50, 60, 80, 90, 100, 110, or 120 pg/1E6 cells, or a range encompassing, or an integer value between, any of the foregoing specifically enumerated values. See, e.g., Berger, J, et al., Intra-monocyte Pharmacokinetics of Imiglucerase Supports a Possible Personalized Management of Gaucher Disease Type 1, Clin Pharmacokinet 58:469-482 (2019) (doi.org/10.1007/s40262-018-0708-8). In some of these embodiments the GD1 patients were standard of care treatment naïve before gene therapy as described herein, whereas in other embodiments the GD1 patients were receiving standard of care ERT or SRT therapy before gene therapy.

In some embodiments, the methods of treatment are effective to increase the amount of GCase protein or enzymatic activity in PBMCs, monocytes, or leukocytes obtained from GD1 patients, either individual GD1 patients who have been treated, or as an average from a population of treated GD1 patients, by at least or about 25%, 50%, 75%, 100%, 125%, 150%, 175%, 200%, 225%, 250%, 300%, 400%, 500%, 700%, 800%, 900%, 1000%, or more, above the baseline amount of GCase in PBMCs, monocytes, or leukocytes obtained from such patient or patients before gene therapy, or compared to the average amount of GCase in PBMCs, monocytes, or leukocytes from comparable (e.g., age and sex matched) control patients with GD1 or other form of GCase deficiency. In some embodiments, the latter control groups are standard of care treatment naïve or are receiving standard of care ERT or SRT therapy. In some of these embodiments the GD1 patients were standard of care treatment naïve before gene therapy as described herein, whereas in other embodiments the GD1 patients were receiving standard of care ERT or SRT therapy before gene therapy.

In some embodiments, the methods are effective to increase the amount of GCase protein in PBMCs, monocytes, or leukocytes obtained from GD1 patients, either individual GD1 patients who have been treated, or as an average from a population of treated GD1 patients, by at least or about 10, 15, 20, 25, 30, 35, 40, 45, 50, 55, 60, 65, 70, 75, 80, 85, 90, 95, 100, 105, 110, 115, 120, 125, 130, 140, 150 pg/1E6, or more, or a range encompassing, or an integer value between, any of the foregoing specifically enumerated values, above the baseline amount of GCase in PBMCs, monocytes, or leukocytes obtained from such patient or patients before gene therapy, or compared to the average amount of GCase in PBMCs from comparable (e.g., age and sex matched) control patients with GD1 or other form of GCase deficiency. In some embodiments, the latter control groups are standard of care treatment naïve or are receiving standard of care ERT or SRT therapy. In some of these embodiments the GD1 patients were standard of care treatment naïve before gene therapy as described herein, whereas in other embodiments the GD1 patients were receiving standard of care ERT or SRT therapy before gene therapy.

### Hepatosplenomegaly

In some embodiments, methods for treating GCase deficiency, including Gaucher disease Type 1 (GD1), with the AAV vectors of the disclosure (including, without limitation, the vector described herein as AAV3B-GBA clone 128) are effective to reduce the incidence, frequency or severity of increased organ size or volume, particularly of liver and spleen. The volume of liver or spleen can be quantified using imaging techniques such as magnetic resonance imaging (MRI), x-ray computed tomography (CT), or ultrasound. In some embodiments, normal liver size can be defined as 2.5% of total body mass, and hepatomegaly can be defined as any increase in liver volume or mass (as inferred from volume) greater than 1.25 times normal (N). In some embodiments, normal spleen size can be defined as 0.2% of total body mass, and splenomegaly can be defined as any increase in spleen volume or mass (as inferred from volume) greater than 1 times normal (N). In some embodiments, treatment is effective to reduce in GD1 patients the frequency or extent of enlarged liver or spleen within a period of time after the administration of AAV vectors of the disclosure of at most, or at least, or about 10, 15, 20, 25, 30, 35, 40, 45, 50, 55, 60, 70, 80, 90, 100, 110, 120, 130, 140, 150, 160, 170, or 180 days, or 1, 2, 3, 4, 5, 6, 7, 8, 9, 10, 11, 12, 13, 14, 15, 16, 17, or 18 months, or 1.0, 1.5, 2.0, 2.5, 3.0, 3.5, 4.0, 4.5, 5.0, 5.5, or 6.0 years, or a range of time between and encompassing any of the foregoing enumerated periods of time.

In some embodiments, methods of the disclosure for treating GD1 can reduce the liver volume, either in individual GD1 patients who have been treated, or as an average in a population of treated GD1 patients, for example, from a volume greater than 2.5 times (or multiple of) normal (>2.5 MN) to a volume between 1.25 MN to 2.5 MN, or to a volume that is less than 1.25 MN (considered non-hepatomegaly). In some embodiments, the methods of treatment are effective to reduce the liver volume, either in individual GD1 patients who have been treated, or as an average in a population of treated GD1 patients, to a value of not more than, less than, or about 4.0, 3.9, 3.8, 3.7, 3.6, 3.5, 3.4, 3.3, 3.2, 3.1, 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7 MN, or smaller volume, or a range encompassing, or an integer value between, any of the foregoing specifically enumerated values. In some of these embodiments the GD1 patients were standard of care treatment naïve before gene therapy as described herein, whereas in other embodiments the GD1 patients were receiving standard of care ERT or SRT therapy before gene therapy.

In some embodiments, the methods of treatment are effective to reduce the liver volume of GD1 patients, either in individual GD1 patients who have been treated, or as an average in a population of treated GD1 patients, by at least or about 1%, 2%, 3%, 4%, 5%, 6%, 7%, 8%, 9%, 10%, 11%, 12%, 13%, 14%, 15%, 16%, 17%, 18%, 19%, 20%, 21%, 23%, 24%, 25%, 26%, 27%, 28%, 29%, 30%, 31%, 32%, 33%, 34%, 35%, 36%, 37%, 38%, 39%, 40%, 50%, or more, compared to the liver volume prior to gene therapy, or compared to the average liver volume from comparable (e.g., age and sex matched) control patients with GD1 or other form of GCase deficiency. In some embodiments, the latter control groups are standard of care treatment naïve or are receiving standard of care ERT or SRT therapy. In some non-limiting embodiments, the liver size of standard of care treatment naïve GD1 patients or GD1 patients receiving standard of care ERT or SRT therapy ranges from about 0.8 to 6 MN, or 1 to 5 MN, or 1.5 to 5 MN, or another value, such as 1.0, 1.5, 2.0, 2.5, 3.0, 3.5, 4.0, 4.5, or 5.0 MN, or a range encompassing, or an integer value between, any of the foregoing specifically enumerated values. In some of these embodiments the GD1 patients were standard of care treatment naïve before gene therapy as described herein, whereas in other embodiments the GD1 patients were receiving standard of care ERT or SRT therapy before gene therapy. In other embodiments, methods of the disclosure for treating GD1 can reduce the frequency or extent of liver pathology, including that of hepatic fibrosis or portal hypertension.

In some embodiments, methods of the disclosure for treating GD1 can reduce the spleen volume, either in individual GD1 patients who have been treated, or as an average in a population of treated GD1 patients, for example, from a volume greater than 15 times (or multiple of) normal (>15 MN) to a volume between 10 MN to 15 MN, or 5 MN to 9 MN, or to a volume that is less than 5 MN. In some embodiments, the methods of treatment are effective to reduce the spleen volume, either in individual GD1 patients who have been treated, or as an average in a population of treated GD1 patients, to a value of not more than, less than, or about 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18.5, 18.0, 17.5, 17.0, 16.5, 16.0, 15.5, 15.0, 14.5, 14.0, 13.5, 13.0, 12.5, 12.0, 11.5, 11.0, 10.5, 10.0, 9.5, 9.0, 8.5, 8.0, 7.5, 7.0, 6.5, 6.0, 5.5, 5.0, 4.5, 4.0, 4.0, 3.9, 3.8, 3.7, 3.6, 3.5, 3.4, 3.3, 3.2, 3.1, 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7 MN, or smaller volume, or a range encompassing, or an integer value between, any of the foregoing specifically enumerated values. In some of these embodiments the GD1 patients were standard of care treatment naïve before gene therapy as described herein, whereas in other embodiments the GD1 patients were receiving standard of care ERT or SRT therapy before gene therapy.

In some embodiments, the methods of treatment are effective to reduce the spleen volume of GD1 patients, either in individual GD1 patients who have been treated, or as an average in a population of treated GD1 patients, by at least or about 1%, 2%, 3%, 4%, 5%, 6%, 7%, 8%, 9%, 10%, 11%, 12%, 13%, 14%, 15%, 16%, 17%, 18%, 19%, 20%, 21%, 23%, 24%, 25%, 26%, 27%, 28%, 29%, 30%, 31%, 32%, 33%, 34%, 35%, 36%, 37%, 38%, 39%, 40%, 45%, 50%, 55%, 60%, 65%, 70%, 75%, 80%, or more, compared to the spleen volume prior to gene therapy, or compared to the average spleen volume of comparable (e.g., age and sex matched) control patients with GD1 or other form of GCase deficiency. In some embodiments, the latter control groups are standard of care treatment naïve or are receiving standard of care ERT or SRT therapy. In some non-limiting embodiments, the spleen size of standard of care treatment naïve GD1 patients or GD1 patients receiving standard of care ERT or SRT therapy ranges from about 0.8 to 60 MN, or 1 to 50 MN, or 5 to 50 MN, or another value, such as 5, 6, 7, 8, 9, 10, 11, 12, 13, 14, 15, 16, 17, 18, 19, 20, 25, 30, 35, 40, 45, or 50 MN, or a range encompassing, or an integer value between, any of the foregoing specifically enumerated values. In some of these embodiments the GD1 patients were standard of care treatment naïve before gene therapy as described herein, whereas in other embodiments the GD1 patients were receiving standard of care ERT or SRT therapy before gene therapy. In other embodiments, methods of the disclosure for treating GD1 can reduce the frequency or extent of lesions in the spleen, detectable using imaging such as MRI or ultrasound, or reduce the frequency of splenectomy in the population of treated GD1 patients compared to treatment naïve GD1 patients.

### Bone Manifestations

In some embodiments, methods for treating GCase deficiency, including Gaucher disease Type 1 (GD1), with the AAV vectors of the disclosure (including, without limitation, the vector described herein as AAV3B-GBA clone 128) are effective to reduce the incidence, frequency or severity of bone manifestations of the disease process, including bone crisis, bone pain, bone marrow infiltration by Gaucher cells, reduced bone mineral density, and the occurrence of lytic lesons, fractures and avascular necrosis. In some embodiments, treatment is effective to reduce in GD1 patients the frequency or extent of bone manifestations of the disease process within a period of time after the administration of AAV vectors of the disclosure of at most, or at least, or about 10, 15, 20, 25, 30, 35, 40, 45, 50, 55, 60, 70, 80, 90, 100, 110, 120, 130, 140, 150, 160, 170, or 180 days, or 1, 2, 3, 4, 5, 6, 7, 8, 9, 10, 11, 12, 13, 14, 15, 16, 17, or 18 months, or 1.0, 1.5, 2.0, 2.5, 3.0, 3.5, 4.0, 4.5, 5.0, 5.5, or 6.0 years, or a range of time between and encompassing any of the foregoing enumerated periods of time.

Bone crisis in GD1 often presents with the sudden occurrence of excruciating pain associated with swelling and erythema, fever and leukocytosis, after which the bone is often severely damaged so that fracture may occur followed by secondary degenerative osteoarthritis. In some embodiments, methods of the disclosure for treating GD1 reduce the frequency of bone crises in a 12 month period, either in individual GD1 patients who have been treated, or as an average in a population of treated GD1 patients, for example, from 2 or more bone crisis events to 1 or 0 events in a 12 month period. In some of these embodiments the GD1 patients were standard of care treatment naïve before gene therapy as described herein, whereas in other embodiments the GD1 patients were receiving standard of care ERT or SRT therapy before gene therapy.

Even in the absence of bone crisis, GD1 patients often experience bone pain, which can vary from mild to extreme. In some embodiments, methods of the disclosure for treating GD1 reduce the maximum degree of pain experienced in a 30 day period, either in individual GD1 patients who have been treated, or as an average in a population of treated GD1 patients, from extreme to severe, moderate, mild, very mild or no pain, or from severe to moderate, mild, very mild or no pain, or from moderate to mild, very mild or no pain, or from mild to very mild or no pain, or from very mild to no pain. In some of these embodiments the GD1 patients were standard of care treatment naïve before gene therapy as described herein, whereas in other embodiments the GD1 patients were receiving standard of care ERT or SRT therapy before gene therapy. The maximum pain experienced by a GD1 patient in a 30 day period can be assessed using a standard pain scale.

One tissue space into which Gaucher cells infiltrate is bone marrow, the burden of which can be quantified using art standard methods such magnetic resonance imaging or scintigraphy. In some embodiments, methods of the disclosure for treating GD1 reduce the frequency or extent of bone marrow infiltration by Gaucher cells as determined using one or more standard scoring systems, either in individual GD1 patients who have been treated, or as an average in a population of treated GD1 patients. Thus, for example, treating GD1 patients using AAV vectors of the disclosure can, in some embodiments, reduce bone marrow infiltration on the Rosenthal scale from a score exceeding 7, such as 8 to 11, to a lower value, such as 4 to 7, or from 1 to 3, or a value of 0. In other embodiments, treatment reduces bone marrow infiltration on the Dusseldorf scale from a score exceeding 6, such as 7 or 8, to a lower value, such as 4 to 6, or 1 to 3, or a value of 0. In other embodiments, treatment reduces bone marrow infiltration on the Terk scale from a score exceeding 2a/b, such as 3a/b, to a lower value, such as 2a/b, or to 1a/b, or to a value of 0. In other embodiments, treatment reduces bone marrow infiltration on the vertebral disk ratio (VDR) scale from a score less than 1.0 to a higher value, such as 1.0 to 1.5, or to 1.5 to 2.0, or a value of greater than 2.0. In other embodiments, treatment reduces bone marrow infiltration on the bone marrow burden (BMB) from a score exceeding 12, such as 13 to 16, to a lower value, such as 8 to 12, or 3 to 7, or 0 to 2. In other embodiments, treatment reduces bone marrow infiltration on the quantitative chemical shift index (QCSI) fat fraction scale from a score less than 0.20 to a higher value, such as 0.20 to 0.25, or 0.25 to 0.30, or a value greter than 0.30. In other embodiments, treatment reduces bone marrow infiltration on the Spanish MRI scale from a score exceeding 17, such as 18 to 24, to a lower value, such as 11 to 17, or 5 to 10, or 0 to 4. In other embodiments, treatment reduces bone marrow infiltration on the ^{99m}Tc-sestamibi scale from a score exceeding 6, such as 7 to 8, to a lower value, such as 5 to 6, or 3 to 4, or 0 to 2. In other embodiments, treatment reduces bone marrow infiltration on the ^{99m}Tc-radiocolloid scale, in comparison to the normal scintigraphic pattern, from severe to moderate, mild or normal, or from moderate to mild or normal, or from mild to normal. In some of these embodiments the GD1 patients were standard of care treatment naïve before gene therapy as described herein, whereas in other embodiments the GD1 patients were receiving standard of care ERT or SRT therapy before gene therapy. More information about bone marrow infiltration scoring scales can be found, e.g., in Rosenthal D I, et al., J Bone Joint Surg Am 68:802-8 (1986); Poll L W, et al., Skeletal Radiol 9:496-503 (2001); Terk M R, et al., Skeletal Radiol 29:563-71 (2000); Vlieger E J, et al., J Comput Assist Tomogr 26:843-8 (2002); Maas M, et al., Radiology 229:554-61 (2003); Poll L W, et al., Blood Cells Mol Dis 28:209-20 (2002).

Reduced bone mineral density (BMD) is another bone manifestation of Gaucher disease which can be reversed, at least partially, by treatment with the AAV vectors of the disclosure, as quantified using techniques such as dual-energy X-ray absorptiometry (DEXA) and expressed as a T or Z score (reflecting the standard deviation of BMD compared to the yound adult mean or an age and sex matched control, respectively). Thus, for example, in some embodiments, the methods of treating GD1 of the disclosure can increase, either in individual GD1 patients who have been treated, or as an average in a population of treated GD1 patients, the Z score from a value less than −1.5 to a value greater than −1.5, such as −1.5 to −1.0, or −1.0 to 0.0, or 0.0 to +1.0. Alternatively, in some other embodiments, the methods of treating GD1 of the disclosure can increase, either in individual GD1 patients who have been treated, or as an average in a population of treated GD1 patients, the T score from a value less than −2.5 to a value greater than −2.5, such as −2.5 to −1.0, or −1.0 to 0.0, or to a value greater than 0.0. In some of these embodiments the GD1 patients were standard of care treatment naïve before gene therapy as described herein, whereas in other embodiments the GD1 patients were receiving standard of care ERT or SRT therapy before gene therapy.

In other embodiments, treatment of GD1 patients with AAV vectors of the disclosure is effective to reduce the frequency or extent, either in individual GD1 patients who have been treated, or as an average in a population of treated GD1 patients, of other bone manifestations of the disease process, including osteonecrosis, osteolysis, avascular necrosis, medullary infarction, permanent deformities resulting from vertebral crush fractures, secondary arthropathy, presentation of the Erlenmeyer flask deformity, reduced consumption of opioid and non-opioid analgesics, need for joint replacement and, in children, delays in growth (e.g., period of time during which growth remains below the 5th percentile). In some of these embodiments the GD1 patients were standard of care treatment naïve before gene therapy as described herein, whereas in other embodiments the GD1 patients were receiving standard of care ERT or SRT therapy before gene therapy.

### Tissue and Plasma Biomarkers

The metabolic derangements caused by Gaucher disease, or GCase deficiency more broadly, can result in marked elevation of certain proteins and glycolipids in cells and tissues or the blood stream. Some of these products are overexpressed by Gaucher cells, and some are byproducts of incomplete degradation of GlcCer in lyosomes due to the deficiency of GCase activity. After treating GD1 patients, either using standard of care of gene therapy, the levels of these products and byproducts may be observed to decline, and can serve as biomarkers indicative of therapeutic efficacy. Accordingly, in some embodiments, methods for treating GCase deficiency, including Gaucher disease Type 1 (GD1), with the AAV vectors of the disclosure (including, without limitation, the vector described herein as AAV3B-GBA clone 128) are effective to reduce the levels of biomarkers which are elevated in subjects with GD1 or GCase deficiency more broadly. In some embodiments, quantifying changes in biomarker levels in subjects after treatment with AAV vectors of the disclosure is indicative of the degree of therapeutic efficacy.

### Metabolic Byproducts, Including Glucosylsphingosine

As a result of GCase deficiency, as in GD1, cellular GlcCer levels increase and are available as a substrate for other enzymes, producing toxic byproducts, the levels of which may be reduced following treatment according to the methods disclosed herein. For example, increased anabolism of GlcCer by glycosyltransferases can produce complex gangliosides, including GM3, which can be detected in plasma and spleen in GD1 patients. See, e.g., DOI: 10.1016/j.cca.2007.12.001. The enzyme β-glucosidase (GBA2) can also act on GlcCer to increase levels of Cer, a proapoptotic molecule, as well as transfer glucose from GlcCer to cholesterol, generating glycosyl-β-cholesterol (GlcChol). See, e.g., DOI: 10.1194/jlr.M064923; DOI: 10.1016/j.cbpa.2019.10.006. Additionally, GlcCer accumulating in lyosomes is converted by lysosomal acid ceramidase to its sphingoid base, glucosylsphingosine (GlcSph; also known as lyso-GL1, lyso-GB1, glucosphingosine, sphingosyl β-glucoside, glucopsychosine), which reportedly can result in an average 200-fold increase in plasma levels of the byproduct in symptomatic untreated GD1 patients. See, e.g., DOI: 10.1002/1873-3468.12104; DOI: 10.1182/blood-2011-05-352971. Methods for quantifying levels of GM3, Cer, GlcChol, and GlcSph in the plasma, serum, or tissue sample from a subject, before or after treatment, or of a healthy human serving as a control, are known in the art. Methods for detecting and quantifying GlcSph, for example, such as by using a LC/MS-MS assay, are described in Rolfs, A, et al., PLOS One 8 (11): e79732 (2013); Murugesan, V, et al., Am. J. Hematol. 91:1082-1089 (2016) (DOI: 10.1002/ajh.24491); Beasley, J, et al., Clinica Chimica Acta 511:132-137 (2020) (doi.org/10.1016/j.cca.2020.10.007); Stiles, A R, et al., Mol Genetics Metabol Rep 27:100729 (2021) (doi.org/10.1016/j.ymgmr.2021.100729). In some embodiments, glucosylsphingosine concentration can be expressed in units of nanograms per milliliter (ng/ml) of plasma, serum, or homogenized tissue sample.

In some embodiments, methods for treating GCase deficiency, including GD1, with the AAV vectors of the disclosure (including, without limitation, the vector described herein as AAV3B-GBA clone 128) are effective to reduce the amount of a byproduct produced from GlcCer, including GM3, Cer, GlcChol, or glucosylsphingosine in serum, plasma, or homogenized tissue sample obtained from a patient with GD1 or other form of GCase deficiency. In some embodiments, treatment is effective to reduce the amount of GM3, Cer, GlcChol, or glucosylsphingosine in serum, plasma, or homogenized tissue sample obtained from treated patients within a period of time after the administration of AAV vectors of the disclosure of at most, or at least, or about 10, 15, 20, 25, 30, 35, 40, 45, 50, 55, 60, 70, 80, 90, 100, 110, 120, 130, 140, 150, 160, 170, or 180 days, or 1, 2, 3, 4, 5, 6, 7, 8, 9, 10, 11, 12, 13, 14, 15, 16, 17, or 18 months, or 1.0, 1.5, 2.0, 2.5, 3.0, 3.5, 4.0, 4.5, 5.0, 5.5, or 6.0 years, or a range of time between and encompassing any of the foregoing enumerated periods of time.

In some embodiments, the methods of treatment are effective to reduce the amount of GM3, Cer, or GlcChol in serum, plasma, or homogenized tissue sample obtained from GD1 patients, either in individual GD1 patients who have been treated, or as an average in a population of treated GD1 patients, by at least or about 5%, 10%, 15%, 20%, 25%, 30%, 35%, 40%, 45%, 50%, 60%, 70%, 80%, 90%, 91%, 92%, 93%, 94%, 95%, 96%, 97%, 98%, or 99% compared to the average amounts of GM3, Cer, or GlcChol in serum, plasma, or homogenized tissue sample obtained from such patient or patients before gene therapy, or compared to the average amount of GM3, Cer, or GlcChol in serum, plasma, or homogenized tissue sample from comparable (e.g., age and sex matched) control patients with GD1 or other form of GCase deficiency. In some embodiments, the latter control groups are standard of care treatment naïve or are receiving standard of care ERT or SRT therapy. In some of these embodiments the GD1 patients were standard of care treatment naïve before gene therapy as described herein, whereas in other embodiments the GD1 patients were receiving standard of care ERT or SRT therapy before gene therapy.

In some embodiments, the methods of treatment are effective to reduce the amount of glucosylsphingosine in serum, plasma, or homogenized tissue sample obtained from GD1 patients, either in individual GD1 patients who have been treated, or as an average in a population of treated GD1 patients, by at least or about 5%, 10%, 15%, 20%, 25%, 30%, 35%, 40%, 45%, 50%, 60%, 70%, 80%, 90%, 91%, 92%, 93%, 94%, 95%, 96%, 97%, 98%, or 99% compared to the average amounts of glucosylsphingosine in serum, plasma, or homogenized tissue sample obtained from such patient or patients before gene therapy, or compared to the average amount of glucosylsphingosine in serum, plasma, or homogenized tissue sample from comparable (e.g., age and sex matched) control patients with GD1 or other form of GCase deficiency. In some embodiments, the latter control groups are standard of care treatment naïve or are receiving standard of care ERT or SRT therapy. In some embodiments, the amount of glucosylsphingosine present in serum, plasma, or homogenized tissue sample from standard of care treatment naïve GD1 patients or GD1 patients receiving standard of care ERT or SRT therapy ranges from about 3 ng/ml to 1000 ng/ml, 4 ng/ml to 600 ng/ml, 10 ng/mL to 500 ng/ml, 20 ng/ml to 400 ng/ml, 50 ng/ml to 300 ng/ml, 100 ng/mL to 300 ng/ml, or 140 ng/ml to 220 ng/ml, or averages about 180 ng/mL, or another value, such as about 50, 60 70, 80, 90, 100, 110, 120, 130, 140, 145, 150, 155, 160, 165, 170, 175, 180, 185, 190, 195, 200, 210, 220, 230, 240, 250, 260, 270, 280, 290, 300, 320, 325, 340, 350, 360, 375, 380, 400, 425, 440, 450, 460, 475, 480, 500, 525, 540, 550, 560, 575, 580, 600, 650, 700, 750, 800, 850, 900, 950, 1,000, 1,500, or 2,000 ng/ml, or a range encompassing, or an integer value between, any of the foregoing specifically enumerated values. In some of these embodiments the GD1 patients were standard of care treatment naïve before gene therapy as described herein, whereas in other embodiments the GD1 patients were receiving standard of care ERT or SRT therapy before gene therapy.

In some embodiments, the methods of treatment are effective to reduce the amount of glucosylsphingosine in serum, plasma, or homogenized tissue sample obtained from GD1 patients, either in individual GD1 patients who have been treated, or as an average in a population of treated GD1 patients, to not more than, to less than, or to about 1,500, 1,000, 900, 800, 750, 700, 650, 600, 550, 500, 450, 400, 350, 300, 250, 240, 230, 225, 220, 215, 210, 205, 200, 195, 190, 185, 180, 175, 170, 165, 160, 155, 150, 145, 140, 135, 130, 125, 120, 115, 110, 105, 100, 95, 90, 85, 80, 75, 70, 65, 60, 55, 50, 25, 20, 15, 10, 9, 8, 7, 6, 5, 4, 3, 2, 1.5, or 1 ng/ml serum, plasma, or homogenized tissue sample, or a range encompassing, or an integer value between, any of the foregoing specifically enumerated values. In some of these embodiments the GD1 patients were standard of care treatment naïve before gene therapy as described herein, whereas in other embodiments the GD1 patients were receiving standard of care ERT or SRT therapy before gene therapy.

### Chitotriosidase

Among proteins overexpressed and secreted into the blood circulation by Gaucher cells in GD1 is the enzyme chitotriosidase, elevated levels of which serves as a biomarker for Gaucher disease. Reportedly, chitotriosidase activity can be elevated on average by 1000-fold in plasma of symptomatic untreated GD1 patients. Methods for quantifying levels of chitotriosidase activity in the plasma, serum, or tissue sample from a subject, before or after treatment, or of a healthy human serving as a control, are known in the art, such as by measuring the amount fluorescent light produced by enzymatic cleavage of specific artificial fluorogenic substrates, such as 4-methylumbelliferyl-chitotrioside, 4′-deoxy-chitobiose-4-methylumbelliferone, 4-methylumbelliferyl-β-D-triacetylchitotriosidase, or others known in the art. For additional information about this biomarker see, e.g., DOI: 10.1172/JCI117084; DOI: 10.1016/S0074-7696 (06) 52001-7; DOI: 10.1074/jbc.M301804200; DOI: 10.1016/j.cbpa.2019.10.006; DOI: 10.1007/s10545-014-9711-x. In some embodiments, chitotriosidase activity can be expressed in terms of the mass per volume of fluorogenic substrate which is cleaved per hour, or in units of nanomoles per milliliter per hour (nmol/mL/hour).

In some embodiments, methods for treating GCase deficiency, including Gaucher disease Type 1 (GD1), with the AAV vectors of the disclosure (including, without limitation, the vector described herein as AAV3B-GBA clone 128) are effective to reduce the amount of chitotriosidase protein and/or enzymatic activity in serum or plasma obtained from a patient with GD1 or other form of GCase deficiency. In some embodiments, treatment is effective to reduce the amount of chitotriosidase protein and/or enzymatic activity in serum or plasma obtained from treated patients within a period of time after the administration of AAV vectors of the disclosure of at most, or at least, or about 10, 15, 20, 25, 30, 35, 40, 45, 50, 55, 60, 70, 80, 90, 100, 110, 120, 130, 140, 150, 160, 170, or 180 days, or 1, 2, 3, 4, 5, 6, 7, 8, 9, 10, 11, 12, 13, 14, 15, 16, 17, or 18 months, or 1.0, 1.5, 2.0, 2.5, 3.0, 3.5, 4.0, 4.5, 5.0, 5.5, or 6.0 years, or a range of time between and encompassing any of the foregoing enumerated periods of time.

In some embodiments, methods of the disclosure for treating GD1 can reduce the amount of chitotriosidase enzymatic activity in serum or plasma, either in individual GD1 patients who have been treated, or as an average in a population of treated GD1 patients, for example, from more than 15,000 nmol/mL/hr to a lower number, such as 4,000 to 15,000 nmol/mL/hr, or 600 to 4,000 nmol/mL/hr, or to a value less than 600 nmol/mL/hr. In some of these embodiments the GD1 patients were standard of care treatment naïve before gene therapy as described herein, whereas in other embodiments the GD1 patients were receiving standard of care ERT or SRT therapy before gene therapy.

In some embodiments, the methods of treatment are effective to reduce the amount of chitotriosidase enzymatic activity in serum or plasma obtained from GD1 patients, either in individual GD1 patients who have been treated, or as an average in a population of treated GD1 patients, by at least or about 5%, 10%, 15%, 20%, 25%, 30%, 35%, 40%, 45%, 50%, 60%, 70%, 80%, 90%, 91%, 92%, 93%, 94%, 95%, 96%, 97%, 98%, or 99% compared to the average amounts of chitotriosidase enzymatic activity in serum or plasma obtained from such patient or patients before gene therapy, or compared to the average amount of chitotriosidase enzymatic activity in serum or plasma from comparable (e.g., age and sex matched) control patients with GD1 or other form of GCase deficiency. In some embodiments, the latter control groups are standard of care treatment naïve or are receiving standard of care ERT or SRT therapy. In some embodiments, the amount of chitotriosidase enzymatic activity in serum or plasma from standard of care treatment naïve GD1 patients or GD1 patients receiving standard of care ERT or SRT therapy ranges from about 200 to 100,000 nmol/mL/hr, 400 to 80,000 nmol/mL/hr, 1,000 to 50,000 nmol/mL/hr, 2,000 to 40,000 nmol/mL/hr, 4,000 to 20,000 nmol/mL/hr, or 5,000 to 15,000 nmol/mL/hr, or is another value, such as about 300, 400, 500, 600, 700, 800, 900, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 25,000, 30,000, 40,000, 50,000, 60,000, 70,000, or 80,000 nmol/mL/hr, or a range encompassing, or an integer value between, any of the foregoing specifically enumerated values. In some of these embodiments the GD1 patients were standard of care treatment naïve before gene therapy as described herein, whereas in other embodiments the GD1 patients were receiving standard of care ERT or SRT therapy before gene therapy.

In some embodiments, the methods of treatment are effective to reduce the amount of chitotriosidase enzymatic activity in serum or plasma obtained from GD1 patients, either in individual GD1 patients who have been treated, or as an average in a population of treated GD1 patients, to not more than, to less than, or to about 25,000, 20,000, 19,000, 18,000, 17,000, 16,000, 15,000, 14,000, 13,000, 12,000, 11,000, 10,000, 9,000, 8,000, 7,000, 6,000, 5,000, 4,000, 3,000, 2,000, 1,000, 900, 800, 700, 600, 500, 400, 300, 200, 100, nmol/mL/hr, or less, or a range encompassing, or an integer value between, any of the foregoing specifically enumerated values. In some of these embodiments the GD1 patients were standard of care treatment naïve before gene therapy as described herein, whereas in other embodiments the GD1 patients were receiving standard of care ERT or SRT therapy before gene therapy.

### CCL18-PARC

Another biomarker protein produced and secreted by Gaucher cells is the chemokine CCL18/PARC (chemokine (C-C motif) ligand 18; pulmonary and activation-regulated chemokine), which can be elevated 20-fold to 50-fold in plasma of symptomatic untreated GD1 patients. Methods for quantifying levels of CCL18/PARC in the plasma, serum, or tissue sample from a subject, before or after treatment, or of a healthy human serving as a control, are known in the art, such as by ELISA using specific antibodies against the protein. For additional information about this biomarker see, e.g., DOI: 10.1016/j.bbalip.2013.11.004; DOI: 10.1182/blood-2003-05-1612.

In some embodiments, methods for treating GCase deficiency, including Gaucher disease Type 1 (GD1), with the AAV vectors of the disclosure (including, without limitation, the vector described herein as AAV3B-GBA clone 128) are effective to reduce the amount of CCL18 in serum or plasma obtained from a patient with GD1 or other form of GCase deficiency. In some embodiments, treatment is effective to reduce the amount of CCL18 in serum or plasma obtained from treated patients within a period of time after the administration of AAV vectors of the disclosure of at most, or at least, or about 10, 15, 20, 25, 30, 35, 40, 45, 50, 55, 60, 70, 80, 90, 100, 110, 120, 130, 140, 150, 160, 170, or 180 days, or 1, 2, 3, 4, 5, 6, 7, 8, 9, 10, 11, 12, 13, 14, 15, 16, 17, or 18 months, or 1.0, 1.5, 2.0, 2.5, 3.0, 3.5, 4.0, 4.5, 5.0, 5.5, or 6.0 years, or a range of time between and encompassing any of the foregoing enumerated periods of time.

In some embodiments, methods of the disclosure for treating GD1 can reduce the amount of CCL18 in serum or plasma, either in individual GD1 patients who have been treated, or as an average in a population of treated GD1 patients, for example, from more than 1,000 ng/mL to a lower number, such as 237 to 1,000 ng/ml, or 72 to 237 ng/ml, or to a value less than 72 ng/ml serum or plasma. In some of these embodiments the GD1 patients were standard of care treatment naïve before gene therapy as described herein, whereas in other embodiments the GD1 patients were receiving standard of care ERT or SRT therapy before gene therapy.

In some embodiments, the methods of treatment are effective to reduce the amount of CCL18 in serum or plasma obtained from GD1 patients, either in individual GD1 patients who have been treated, or as an average in a population of treated GD1 patients, by at least or about 5%, 10%, 15%, 20%, 25%, 30%, 35%, 40%, 45%, 50%, 60%, 70%, 80%, 90%, 91%, 92%, 93%, 94%, 95%, 96%, 97%, 98%, or 99% compared to the average amounts of CCL18 in serum or plasma obtained from such patient or patients before gene therapy, or compared to the average amount of CCL18 in serum or plasma from comparable (e.g., age and sex matched) control patients with GD1 or other form of GCase deficiency. In some embodiments, the latter control groups are standard of care treatment naïve or are receiving standard of care ERT or SRT therapy. In some embodiments, the amount of CCL18 in serum or plasma from standard of care treatment naïve GD1 patients or GD1 patients receiving standard of care ERT or SRT therapy ranges from about 40 to 1,500 ng/ml, 100 to 1,200 ng/ml, 200 to 1,000 ng/mL, or 400 to 800 ng/ml, or is another value, such as about 40, 50, 60 70, 80, 90, 100, 110, 120, 130, 140, 145, 150, 155, 160, 165, 170, 175, 180, 185, 190, 195, 200, 210, 220, 230, 240, 250, 260, 270, 280, 290, 300, 320, 325, 340, 350, 360, 375, 380, 400, 425, 440, 450, 460, 475, 480, 500, 525, 540, 550, 560, 575, 580, 600, 650, 700, 750, 800, 850, 900, 950, 1,000, 1,100, 1,200, 1,300, 1,400, or 1,500 ng/ml, or a range encompassing, or an integer value between, any of the foregoing specifically enumerated values. In some of these embodiments the GD1 patients were standard of care treatment naïve before gene therapy as described herein, whereas in other embodiments the GD1 patients were receiving standard of care ERT or SRT therapy before gene therapy.

In some embodiments, the methods of treatment are effective to reduce the amount of CCL18 in serum or plasma obtained from GD1 patients, either in individual GD1 patients who have been treated, or as an average in a population of treated GD1 patients, to not more than, to less than, or to about

1,500, 1,400, 1,300, 1,200, 1,100, 1,000, 950, 900, 850, 800, 750, 700, 650, 600, 550, 500, 450, 400, 350, 325, 300, 275, 250, 240, 230, 225, 220, 215, 210, 205, 200, 195, 190, 185, 180, 175, 170, 165, 160, 155, 150, 145, 140, 135, 130, 125, 120, 115, 110, 105, 100, 95, 90, 85, 80, 75, 70, 65, 60, 55, 50, 45, 40, 35, 30, 25, 20, 15, 10, 5 ng/mL serum or plasma, or less, or a range encompassing, or an integer value between, any of the foregoing specifically enumerated values. In some of these embodiments the GD1 patients were standard of care treatment naïve before gene therapy as described herein, whereas in other embodiments the GD1 patients were receiving standard of care ERT or SRT therapy before gene therapy.

### Gaucher Severity Score

In some embodiments, methods for treating Gaucher disease Type 1 (GD1) with the AAV vectors of the disclosure (including, without limitation, the vector described herein as AAV3B-GBA clone 128) are effective to reduce the severity of Gaucher disease as measured using a Gaucher disease severity score index, several of which are known in the art, either in individual GD1 patients who have been treated, or as an average in a population of treated GD1 patients. For example, in some embodiments, the methods for treating Gaucher disease disclosed herein are effective to reduce severity of Gaucher disease as determined using the Zimran Severity Score Index (SSI), (as described in DOI: 10.1016/s0140-6736 (89) 90536-9 and Zimran A, et al., Gaucher disease. Clinical, laboratory, radiologic, and genetic features of 53 patients. Medicine 71:337-353 (1992)); as determined using Di Rocco et al.'s Gaucher Severity Score Index for type I patients (GauSS-I) (as described in DOI: 10.3324/haematol.12379); or as determined using Weinreb et al.'s Gaucher Disease type I severity scoring system (GD-DS3) (as described in Weinreb, N J, et al., Genetics Med 12 (1): 44-51 (2010)), use of other severity scoring indexes being possible. In some embodiments, treatment is effective to reduce in GD1 patients the severity of Gaucher disease as measured using a Gaucher disease severity score index within a period of time after the administration of AAV vectors of the disclosure of at most, or at least, or about 10, 15, 20, 25, 30, 35, 40, 45, 50, 55, 60, 70, 80, 90, 100, 110, 120, 130, 140, 150, 160, 170, or 180 days, or 1, 2, 3, 4, 5, 6, 7, 8, 9, 10, 11, 12, 13, 14, 15, 16, 17, or 18 months, or 1.0, 1.5, 2.0, 2.5, 3.0, 3.5, 4.0, 4.5, 5.0, 5.5, or 6.0 years, or a range of time between and encompassing any of the foregoing enumerated periods of time.

In some embodiments, the methods for treating Gaucher disease disclosed herein are effective to reduce severity of Gaucher disease from severe to moderate or mild, or from moderate to mild, as determined using the Zimran score index or, using the same index, reducing the severity score from an integer value in the range of 26 to 51 points to a lower value, such as 25, 24, 23, 22, 21, 20, 19, 18, 17, 16, 15, 14, 13, 12, 11, 10, 9, 8, 7, 6, 5, 4, 3, 2, 1, or 0, or from an integer value in the range of 11 to 25 points to a lower value, such as 10, 9, 8, 7, 6, 5, 4, 3, 2, 1, or 0 points. In some embodiments, the methods for treating Gaucher disease disclosed herein are effective to reduce severity of Gaucher disease as determined using the Di Rocco GauSS-I score index from an integer value in the range of 1 to 42 points to a lower value, such 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, or 0 points. In some embodiments, the methods for treating Gaucher disease disclosed herein are effective to reduce severity of Gaucher disease from severe to marked, moderate, mild, or borderline, or from marked to moderate, mild, or borderline, or from moderate to mild, or borderline, or from mild to borderline, as determined using the Weinreb GD-DS3 score index or, using the same index, reducing the severity score from an integer value in the range of 10 to 19 points to a lower value, such as 9, 8, 7, 6, 5, 4, 3, 2, 1, or 0, or from an integer value in the range of 6 to 9 points to a lower value, such as 8, 7, 6, 5, 4, 3, 2, 1, or 0, or from an integer value in the range of 3 to 6 points to a lower value, such as 5, 4, 3, 2, 1, or 0, or from a value of 3 points to 2, 1, or 0, or from a value of 2 points to 1 or 0, or from a value of 1 point to 0.

### Other Beneficial Treatment Effects

In other embodiments, treatment of GD1 patients with AAV vectors of the disclosure (including, without limitation, the vector described herein as AAV3B-GBA clone 128) is effective, either in individual GD1 patients who have been treated, or as an average in a population of treated GD1 patients, to reduce the frequency or extent of other manifestations of the Gaucher disease process, including endocrine and metabolic disorders, neurological symptoms, and other symptoms. Thus, in some embodiments, the methods of treating GD1 disclosed herein are effective to reduce the incidence of low appetite, underweight, elevation of basal hepatic glucose, Type 2 diabetes, low levels of high-density lipoprotein cholesterol, peripheral neuropathy, elevated risk of developing Parkinson's disease, elevated risk of multiple myeloma and non-myeloma, lung infiltration by Gaucher cells and associated pulmonary fibrosis, restrictive lung disease secondary to spinal deformation, pulmonary arterial hypertension, respiratory failure, kidney glomeruli infiltration by Gaucher cells and associated proteinuria and haematuria, yellow-brown hyperpigmentation resulting from skin involvement, ocular manifestations, and myocardial or heart valvular involvement.

### Methods of Prevention

Among other embodiments, the disclosure provides methods for preventing Gaucher disease, including Type 1 Gaucher disease (GD1), or other type of GCase deficiency, by administering to a subject, such as a human subject, in need of prevention for Gaucher disease (or GCase deficiency) a prophylactically effective amount of an AAV vector of the disclosure (including, without limitation, the vector described herein as AAV3B-GBA clone 128), or a pharmaceutical composition containing such AAV vectors. Also provided is use of an AAV vector of the disclosure in the manufacture of a medicament for use in the methods of prophylaxis disclosed herein. In addition, there is provided an AAV vector of the disclosure, or a pharmaceutical composition containing such AAV vectors, for use in the methods of prophylaxis disclosed herein.

In some embodiments, administering a prophylactically effective amount an AAV vector of the disclosure (including, without limitation, the vector described herein as AAV3B-GBA clone 128), or pharmaceutical composition containing such AAV vectors, to a subject is effective to prevent initiation or onset in the subject of Gaucher disease; is effective to prevent initiation or onset in the subject of any deleterious effect of GCase deficiency; is effective to prevent initiation or onset in the subject of a reduction in the amount of GCase activity; is effective to prevent initiation or onset in the subject of at least one symptom or sign associated with Gaucher disease or GCase deficiency; is effective to prevent initiation or onset in the subject of at least one disorder or dysfunction of the body, organ, tissue, or cell, caused by Gaucher disease or GCase deficiency; is effective to prevent initiation or onset in the subject of any reduction in the quality of life caused by Gaucher disease or GCase deficiency which, in each case, would otherwise have occurred in the absence of prophylaxis.

In some embodiments of the methods of prophylaxis described herein, the subject is a human subject with a homozygous or heterozygous deleterious mutation in the GBA gene, the existence of which is determined by genotyping before the onset of any detectable symptom or sign of Gaucher disease, or other symptom or sign associated with GCase deficiency. Methods for genotyping, such as by RFLP analysis or gene or genomic sequencing, are familiar to those of ordinary skill in the art.

### AAV Vector Compositions, Formulations, Methods of Administration, Dosages

In addition to AAV vectors, the present disclosure provides compositions comprising such vectors and as at least one pharmaceutically acceptable excipient, diluent, or carrier. Such vectors may be used, among other things, in the methods of prevention and treatment of GD1 also described herein.

Compositions comprising AAV vectors of the disclosure can be provided as aqueous solutions or suspensions, emulsions, and in other forms, such as lyophilized cakes. Vector compositions can be formulated using any suitable diluent and excipients that may be necessary to achieve desired properties, such as pH, ionic strength, tonicity, stability, shelf-life, resistance to freeze-thaw cycles, and ability to be freeze dried, as well as considering the mode of administration. Exemplary diluents and carriers include, without limitation, sterile water for injection, ethanol, and glycerol. Exemplary excipients include, without limitation, salts, buffers, acids, bases, surfactants, saccharides, sugar alcohols, and many others known in the art. Compositions comprising AAV vectors of the disclosure for use in preventing or treating a disease or disorder in a subject, such as GD1, can be packaged in any suitable form, such as vials or pre-filled syringes. In some embodiments, kits are provided with a plurality of vials containing sufficient total amount of vector to achieve a desired total dose to be delivered to a particular subject based on relevant variables, such as such subject's disease severity, body mass, sex, or others.

Compositions comprising AAV vectors of the disclosure can be administered by any suitable route of administration, non-limiting examples of which include systemic administration, administration directly into a tissue or organ, intravenous administration, intraarterial administration, intralymphatic administration, intraperitoneal administration, intramuscular administration, intraparenchymal administration, intrathecal administration, intracerebroventricular administration, or intracisternal magna administration, with others being possible. In some embodiments, for example, where an AAV vector is desired to be targeted preferentially to the liver, a composition comprising such vector may be administered into the portal vein.

Compositions comprising AAV vectors of the disclosure can be administered alone, without any other kinds of therapy, or can be administered simultaneously, contemporaneously, or at any suitable dosing interval with a standard of care treatment, or some other agent, compound, drug, treatment or therapeutic regimen. In some embodiments, compositions comprising AAV vectors of the disclosure can be administered after prophylaxis with immunosuppressive agent, such as a steroid or tacrolimus, or other immunosuppresent drug, or immunosuppresent drugs can be administered afterward to control any humoral and/or cellular immune reaction to the gene therapy.

Vector compositions can contain any suitable amount of an AAV vector calculated to deliver a prophylactically or therapeutically effective amount of such vector to a subject in a volume that is easily handled or administered to the subject, and/or would not be expected to cause any discomfort or undesirable side effects to the subject.

In connection with the methods of prevention and treatment provided by the disclosure, AAV vectors and compositions comprising such vectors can be administered in any suitable dose predicted or determined to be effective to achieve the desired degree of prevention or treatment. In some embodiments, doses of an AAV vector of the disclosure for preventing or treating Gaucher disease Type 1 can be quantified and expressed as vector genomes (vg) per kilogram of subject body weight, abbreviated “vg/kg.” In some embodiments, exemplary efficacious doses of an AAV vector of the disclosure, including, for example, an AAV vector comprising an AAV3B capsid and a genome comprising the nucleotide sequence of SEQ ID NO:17, include, without limitation, at least or about 1×10⁹ vg/kg, 1×10¹⁰ vg/kg, 1×10¹¹ vg/kg, 1×10¹² vg/kg, 1×10¹³ vg/kg, 1×10¹⁴ vg/kg, or 1×10¹⁵ vg/kg, or a range of doses between and including any of the foregoing specifically enumerated doses, other doses being possible.

Other objects, features and advantages of the present invention will be apparent from the foregoing detailed description. It should be understood, however, that the detailed description and the specific examples that follow, while indicating specific embodiments of the invention, are given by way of illustration only, since various changes, modifications and equivalents within the spirit and scope of the invention will be apparent from the detailed description and examples to those of ordinary skill in the art, and fall within the scope of the appended claims.

Unless otherwise indicated, use of the term “or” in reference to one or more members of a set of embodiments is equivalent in meaning to “and/or,” and does not require that they be mutually exclusive of each other. Unless otherwise indicated, a plurality of expressly recited numeric ranges also describes a range the lower bound of which is derived from the lower or upper bound of any one of the expressly recited ranges, and the upper bound of which is derived from the lower or upper bound of any other of the expressly recited ranges. Thus, for example, the series of expressly recited ranges “10-20, 20-30, 30-40, 40-50, 100-150, 200-250, 275-300,” also describes the ranges 10-50, 50-100, 100-200, and 150-250, among many others. Unless otherwise indicated, use of the term “about” before a series of numerical values or ranges is intended to modify not only the value or range appearing immediately after it but also each and every value or range appearing thereafter in the same series. Thus, for example, the phrase “about 1, 2, or 3,” is equivalent to “about 1, about 2, or about 3.”

All publications and references, including but not limited to articles, abstracts, patents, patent applications (whether published or unpublished), and biological sequences (including, but not limited to those identified by specific database reference numbers) cited herein are hereby incorporated herein by reference in their entirety for all purposes to the same extent as if each individual publication or reference were specifically and individually indicated to be so incorporated by reference. Any patent application to which this application claims priority directly or indirectly is also incorporated herein by reference in its entirety.

Unless otherwise indicated, the examples below describe experiments that were or are performed using standard techniques well known and routine to those of ordinary skill in the art. The examples are illustrative, but do not limit the invention.

### EXAMPLES

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Design and Production of AAV Vectors to Express GCase

AAV vectors comprising a transgene for expressing human beta-glucocerebrosidase (GCase) were designed, and produced using standard techniques. The protein expressed by the vectors is full length wild type human GCase, except that the native secretion signal peptide sequence was replaced with an immunuglobulin secretion signal peptide sequence. One vector genome (clone 118) included the native human nucleotide sequence encoding hGCase protein (other than for the signal peptide), whereas the transgene in the other vector genome (clone 128) encoded the same protein, but was modified to reduce the number of CpG motifs (66 CpG motifs in clone 128 compared to 96 CpG motifs in clone 118). Both genomes comprised in 5′ to 3′ order, a 5′ ITR from AAV2, two copies of an enhancer region from the alpha1-microglobulin/bikunin precursor (A1 MB enh), one copy of the proximal enhancer region from the human albumin (hALB) gene, one copy of the core promoter region from the hALB gene (hALB pro), one copy of a human beta globin gene intron (which does not interupt coding sequence), coding sequence for the IgVH signal peptide in frame with the coding sequence for the GCase enzyme (the IgVH signal peptide replacing the native signal peptide), a stop codon, a bovine growth hormone (bGH) gene polyadenylation (polyA) signal sequence (i.e., terminator), a modified TBP intron 2 sequence, and a 3′ ITR from AAV2.

With respect to the vector genome of clone 128, the complete sequence of which is reported as SEQ ID NO:17 (inclusive of both ITRs), the nominal starting and ending nucleotide numbers for each genome component is set forth in the following Table 1.

TABLE 1

| | Starting Nucleotide | Ending Nucleotide |
|---|---|---|
| Genome Component | in SEQ ID NO: 17 | in SEQ ID NO: 17 |
| 5′ ITR | 1 | 145 |
| A1MB Enhancer | 320 | 464 |
| hALB Proximal Enhancer | 479 | 792 |
| hALB Core Promoter | 793 | 1,002 |
| hBetaGlobin Intron | 1,010 | 1,859 |
| IgVH Signal Peptide | 1,896 | 1,952 |
| hGCase Coding Sequence | 1,953 | 3,446 |
| bGH polyA | 3,454 | 3,681 |
| 3′ ITR | 4,057 | 4,202 |

Additional polypeptide and nucleotide sequences relating to the vectors described herein are set forth in the Table of Sequences, below.

To produce vector particles, HEK 293 cells in suspension culture were transfected using the classical triple transfection method. HEK 293 cells were expanded from a working cell bank aliquot through multiple passages, starting from shake flask, through wave bag, to single use bioreactor (SUB) at 250 L scale. Cells were transfected by addition of transfection cocktail containing PEI and three different plasmids: pHelper, to express AdV helper factors; pRepCap, to express the AAV3B capsid proteins and AAV3B Rep proteins; and the transgene plasmid. After transfection for 3 hours, transfection was quenched by adding CDM4 media, followed by a 72 hour incubation period to allow AAV vector production by the cells. Vector was harvested by lysing the cells with Triton X-100, adding domiphen bromide to flocculate host cell DNA, filtering the supernatant, and then purifying vector in three stages, including affinity chromatography, anion exchange chromatography, and tangential flow filtration. After harvest and purification, vectors were titered by quantitative PCR, and then tested in vitro and in vivo for potency and toxicity.

### Example 2

### Testing of AAV GCase Vectors In Vitro

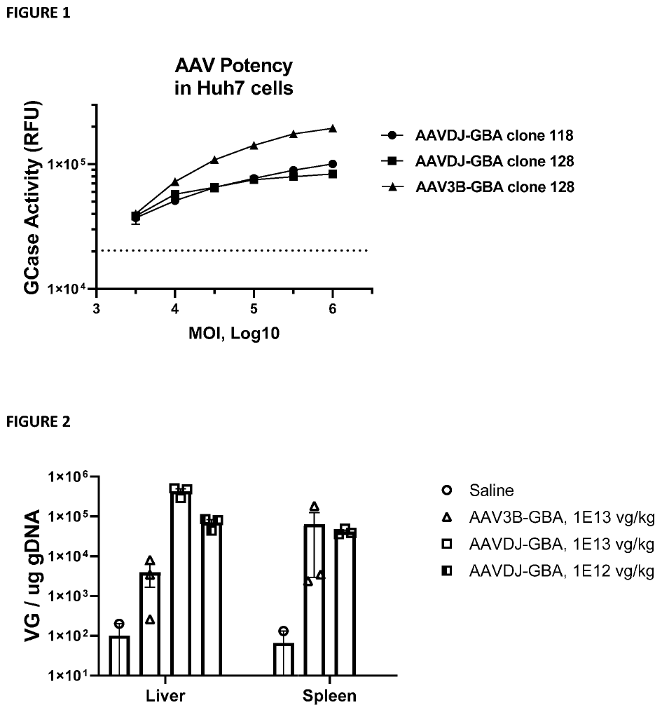

Potency of two vectors containing the clone 128 genome, one using the AAV3B capsid (AAV3B-GBA clone 128) and the other using the AAVDJ capsid (AAVDJ-GBA clone 128), was tested in vitro by transducing Huh7 cells, a human hepatoma cell line, and measuring GCase enzymatic activity in an assay with fluorogenic substrates. A vector using the AAVDJ capsid was tested because it is known that AAV3B capsid transduces mouse liver tissue with low efficiency, potentially confounding the results of the in vivo experiments described below. In the Huh7 cells, both vectors showed dose-dependent increases of GCase enzymatic activity in Huh7 cells (FIG. 1).

### Example 3

### Testing of AAV GCase Vectors in a Mouse Model of Gaucher Disease Type 1

The efficacy of the clone 128 vector genome delivered by both AAV3B and AAVDJ capsids was tested in the D409V transgenic mouse animal model of Gaucher Disease Type 1. D409V knock-in mice express a mutant mouse GBA gene that corresponds to the D409V mutation found in human GCase. Similar to the residual GCase level found in Type 1 GD patients, the GCase activity in D409V mice is ˜10% that of wild type (WT) animals. Sphingolipid accumulation can be detected in serum and various organs, including liver and spleen. The D409V mouse model is described futher in, e.g., Xu, Y-H, et al., Viable Mouse Models of Acid B-Glucosidase Deficiency, Am J Pathol. 2003 November; 163 (5): 2093-2101 (DOI: 10.1016/s0002-9440 (10) 63566-3); Sun, Y, et al., Gaucher disease mouse models: point mutations at the acid beta-glucosidase locus combined with low-level prosaposin expression lead to disease variants, J Lipid Res. 2005 October; 46 (10): 2102-13 (DOI: 10.1194/jlr.M500202-JLR200).

The vectors in which the AAV3B and AAVDJ capsids encapsidate the clone 128 vector genome may be referred to herein as “AAV3B-GBA” and “AAVDJ-GBA”, respectively. Vector doses may alternatively be expressed using exponential notation or E notation. Thus, for example, a dose of “3×10¹² vg/kg” has the same meaning as “3E12 vg/kg”.

### Materials and Methods

### Vector Administration and Sample Collection

AAV3B-GBA clone 128 vector (1E13 vg/kg) and AAVDJ-GBA clone 128 (1E12 vg/kg and 1E13 vg/kg) were administered in single doses by intravenous injection via retro-orbital route to 5 month old male D409V homozygous knock-in transgenic mice (Jackson Lab #019106). Saline was administered to 4 test animals of the same type as a negative control. Five test animals were used for each vector dose. Twenty-eight days later, test animals were euthanized and serum and tissue samples collected and analyzed. Tissue samples were snap frozen and stored at −80° C. until use. For IHC, tissues were fixed in 10% neutral buffered formalin and processed to paraffin sections.

### DNA Isolation

Frozen tissue samples were used for DNA isolation using phenol/chloroform DNA extraction methods. Frozen tissue samples were pulverized into a frozen dry powder using a Covaris CP02 Cyroprep Pulverizer following manufacturer instructions. Approximately 30 mg of each frozen tissue powder was transferred into a new tube and resuspended in 292.5 mL of TENS lysis buffer containing 1% SDS. Next, 7.5 mL Proteinase K was added to each tube and incubate at 56° C. in a thermomixer shaking at 1000 rpm for 3 hours, and vortexed for 15 seconds every 30 minutes. Samples were then cooled to room temperature and treated with RNase A for 5 minutes. Each sample was treated with 300 ml of phenol: chloroform: isoamyl alcohol (25:24:1) and vortexed for 20 seconds. The upper phase liquid was collected for DNA precipitation by the addition of 7.5 M NH₄OAc at approximately 0.5 times of the upper phase volume and the addition of pure ethanol at 3 times the total volume. Samples were chilled at −20° C. for at least 2 hours followed by centrifugation at 4° C. for 30 minutes at 16,000 g. DNA pellet was washed with ice cold 70% ethanol and resuspended in nuclease free water. DNA concentration was measured with Nanodrop spectrophotometer

### RNA Isolation

Frozen tissue powder was prepared as described in the above section. Approximately 30 mg of tissue sample was used for homogenization in 1 mL Trizol with the presence of a 5 mm stainless steel bead using a Tissuelyzer II set at frequency 25 1/s for 5 minutes. Lysates were transferred to phasemaker tubes and 200 mL chloroform was added. Samples were vortexed for 15 seconds, incubated at room temperature for 5 minutes, and centrifuged at 14,000 g for 5 min at 4° C. The clear supernatant was transfer to a new tube and processed with the Purelink RNA mini kit per manufacturer instructions. DNAse treatment was performed on column for 20 min before elution with nuclease free water. RNA concentration was measured using Nanodrop spectrophotometer.

### Vector Genome Copy Number

VGC quantification was performed using ddPCR using the following primer and probe sequences to amplify GBA transgene in all samples. In the ddPCR assay, annealing temperature was 60° C., probe primer sequence was TTAGGAAAGGACAGTGGGAGTGGC (SEQ ID NO:43), forward primer sequence was AATGCGATGCAATTTCCTCATT (SEQ ID NO:44), reverse primer sequence was TGCCAGCCATCTGTTGTT (SEQ ID NO:45), probe flurophore was FAM, and the quencher was NFQ. DNA was diluted to 10 ng/ml. 20 μl PCR reaction mixture containing ddPCR Supermix, isolated gDNA, GBA primers and FAM probes (see above), housekeeping gene (ms) Tfrc primers and VIC probes were prepared in a Airclean 600 PCR workstation cleaned with DNAZap and added to a ddPCR 96-well plate. Droplets were generated using QX200 droplet generator. Plates were sealed with PX1 PCR plate sealer. PCR program: 95° C. for 10 min→[94° C. for 30 sec→60° C. for 1 min] (×40 cycles)→98° C. for 10 min→4° C. hold was completed. Droplet counts and amplitude was acquired by a QX200 reader and analyzed with QuantaSoft software to calculate the number of DNA copies. VGC obtained from the FAM channel are normalized to ug of gDNA input or reported as VGC per haploid genome by calculating the ratio of FAM to VIC channel

### GBA Transgene Expression

Isolated RNA samples were reverse transcribed to generate cDNA library using iScript cDNA Synthesis kit (Bio-Rad, cat #1708891) following manufacturer's protocol. 500 ng of total RNA was used in 20 μL reaction. Reverse transcription was performed at 37° C. for 60 minutes followed by 95° C. for 5 minutes. cDNA samples were stored at −20° C. until RT-PCR analysis. RNA expression quantification of GBA transgenes was determined using ddPCR with the same GBA primers and probes described above and housekeeping gene MmHprt primer probes. PCR reaction mixture containing ddPCR Supermix (no dUTP) and above primer probes was prepared in a Airclean 600 PCR workstation cleaned with DNAZap and added to a ddPCR 96-well plate. Droplet generation, PCR programs, and data acquisition were performed as described above VGC was normalized to ug of gDNA or the count of MmHprt.

### GCase Protein Expression by LC-MS/MS

Human recombinant GCase protein produced in vector treated test animals was enriched from samples of serum, PBMCs, and lysates of liver and spleen tissue with an antibody that specifically binds GCase, and then quantified by liquid chromatography tandem mass spectrometry (LC-MS/MS). Spleen and liver sections were prepared by adding a volume of M-PER+protease inhibitor to prepare 100 mg/mL tissue to buffer, samples were processed using a Next Advance bullet blender for 7 minutes. Samples were assayed at various volumes, 20 μL serum and 200 μL tissue lysate against a standard curve surrogate matrix of 1% BSA in PBS spiked with taliglucerase protein. The assay linear range was 0.25 to 1,000 ng/ml for tissue samples and 0.5 to 1,000 ng/ml for serum samples. Dual IP was performed using 2 μg of anti-GCase antibody+biotin and streptavidin magnetic beads. Samples were processed on a Thermo KingFisher Flex and eluted with 30 mM HCl+5% MeCl₂. Samples were adjusted to pH 8.3 using 1M Tris-HCl, reduced, alkylated, and digested overnight using Promega LysC-Trypsin. Samples were injected on either a Thermo Altis MS or Sciex 6500E using OptiFlow system at a volume of 85 μL using trap and elute nanoflow LC conditions. LC-MS/MS assay data was acquired using either a Sciex 6500 (SciexE) using Analyst 1.7.1 with HotFix 1 acquisition software or a Thermo Altis (Altis2) using Xcalibur 4.2.47 acquisition software. Specifically, serum samples were run on Sciex 6500E with data processing completed using Sciex MultiQuant 3.0.3 software. Tissue samples were run on Altis2 with sample data processing completed using Skyline v. 21.1.0.146. A calibration curve was generated from peak area ratios of analyte peptide over SIL peptide and the concentration of study samples were determined by back-calculating the peak-area-ratio against the standard curve in both software packages. LC-MS/MS data for tissue lysate was normalized to total protein weight.

### GCase Protein Expression by ICH

Paraffin sections were stained with anti-GCase antibody using a fully automated stainer (Leica Biosystems Bond Rx). Briefly, the slides were dewaxed and treated with peroxide for 10 minutes. Then, the slides were rinsed with deionized water and permeabilized with 0.3% Triton solution for 1 hour before incubation with a primary anti-GCase antibody (Sigma-Aldrich) for 1 hour. The primary antibody was diluted in Bond primary antibody diluent at 1:250 dilution. Next, the slides were rinsed and incubated in a secondary antibody (Leica #DS9800) for 8 minutes. The presence of antibodies is visualized by an incubation with DAB (Leica #DS9800) for 10 minutes. Finally, the slides were rinsed in water and dehydrated from graded ethanol to xylene before cover slipping and imaged on a slide scanner (Axio Scan Z1, Zeiss). Images were rendered with ZEN Blue software

### GCase Activity Assay

Frozen tissue powder was prepared as described above. Approximately 20 mg of tissue samples were used to generate lysates with the presence of stainless steel beads in lysis buffer (citrate phosphate buffer pH5.4 containing 0.25% (w/w) sodium taurocholate, 0.25% Triton X-100 and protease inhibitor) using a Tissuelyzer II set at frequency 25 1/s for ˜8 minutes. Protein concentration in the tissue lysate was measured by a BCA protein assay kit. In a 96-well black non-binding plate, 25 μg of tissue lysate, 10 μL of serum, or Taliglucerase standard was mixed with 3 mM synthetic GCase substrates (4-methylumbelliferyl β-D-glucopyranoside) in 0.2M sodium acetate solution (pH 4) at 37° C. for 30 minutes. Reactions were attenuated by adding 0.7× volume of 1M glycine pH10. Envision plate reader was used to acquire fluorescent signals (excitation/emission wavelength: 355/450 nm). Values were calculated by subtracting background signal and then fitting the Taliglucerase standard curve

### GluSph Assay

Concentration of D-glucosyl-β1-1′-D-erythro-sphingosine (GluSph) in serum and tissue samples from test animals was analyzed by LC-MS/MS. Protein in samples was precipated with a mixture of 97.5 parts Mobile Phase A (100:0.5 Acetonitrile: Formic acid) 2.5 parts Mobile Phase B (50 mM Ammonium Formate in 60:40:0.5 Methanol: Deionized Water: Formic Acid) (MPAB), GluSph extracted, dried down and reconstituted. MPAB was chosen as a surrogate matrix for the standards. Quality controls were run in both surrogate and actual matrix (serum, liver and spleen homogenates). For tissue homogenization, tissue was weighed out, homogenization buffer (MPAB) added in a 4:1 μL/mg ratio, homogenized, supernatant transferred to new tube, and standard curves run (0.005 ng/mL-81.92 ng/mL). For sample preparation, 20 μL aliquots of standards, quality controls, blank matrix and samples were dispensed into a 96-well round bottom plate, 20 μL matrix or surrogate matrix added, 120 μL working internal standard (ISTD) solution in MPAB added, followed by vortexing, centrifugation, and transferring the sample mixtures to wells of a new plate. Liquid was removed by evaporation by incubation at 45° C., residue reconstituted in 100 μL of MPAB, followed by vortexing and injecting into a MDS-SCIEX/Applied Biosystems 5500 QTrap with Turbo-Ion Spray Interface. Surrogate standards were prepared with MPAB in triplicate at 0.005, 0.01, 0.02, 0.04, 0.08, 0.16, 0.32, 0.64, 1.28, 2.56, 5.12, 10.24, 20.48, 40.96 and 81.92 ng/ml concentrations. Surrogate matrix quality controls were prepared with MPAB in triplicate at 0.5, 1.25, 5 and 25 ng/ml concentrations. Surrogate matrix and mouse tissue matrix was then spiked into the prepared quality control. MPAB was used for surrogate matrix quality controls while serum and homogenized tissues were used for matrix quality controls. Quality controls met the acceptance criteria of +20% and replicates passed within +20% of the nominal concentration. To ensure stability across each assay, standard curves were run before the assay, after the quality controls, and again at the end of the assay. Calibration curve regression was performed using Sciex OS software curve fitting setting of quadratic with 1/x² weighting. GluSph-d5 was used as an internal standard.

### Results

### Vector Genome Copy Number

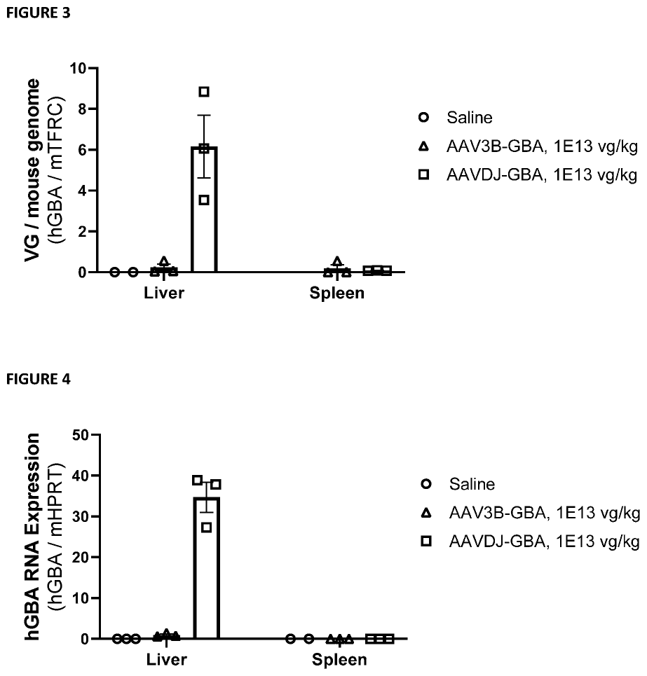

AAVDJ-GBA vector transduced liver and spleen as determined by quantifying vector genome copy numbers in tissue samples taken from test animals, with a trend toward dose responsiveness in liver (FIG. 2). AAV3B-GBA vector also transduced liver, but less efficiently compared to AAVDJ-GBA (FIG. 2). Normalizing vector genome copy numbers to copies of a mouse housekeeping gene, Tfrc, demonstrated that AAVDJ-GBA was 27× more efficient at transducing liver compared to the same dose of the transgene delivered by the AAV3B capsid (1E13 vg/kg), confirming that AAVDJ capsid transduces mouse liver more efficiently than AAV3B (FIG. 3). Transduction of spleen by both vectors was evident when vector genome copy numbers were normalized to amount of input gDNA, but minimal transduction was evident when vector genome copy numbers were normalized to copies of the housekeeping gene (serving as proxy for a host cell haploid genome) (FIG. 3). Transduction data for individual test animals is provided in Table 2, below.

### GBA Transgene Expression

GBA transgene mRNA expression normalized to expression of a host cell housekeeping gene, Hprt, was consistent with the pattern of vector transduction. Transgene expression in liver in test animals administered the AAVDJ-GBA vector was substantially greater than that produced from the same dose of AAV3B-GBA vector (FIG. 4). Transgene expression was not detected from either vector in spleen (FIG. 4). GBA transgene mRNA expression levels for individual test animals is presented in Table 2, below.

TABLE 2

| Animal | | | Dose | VG/μg | VG/mouse genome | hGBA RNA |
|---|---|---|---|---|---|---|
| ID | Tissue | Test Article | (vg/kg) | gDNA | (hGBA/mTFRC) | (hGBA/mHPRT) |
| 26 | Liver | Vehicle | 0 | 0 | 0.000 | 0.001 |
| 27 | Liver | Vehicle | 0 | 200 | 0.002 | 0.000 |
| 28 | Liver | Vehicle | 0 | N/A | N/A | 0.001 |
| 11 | Liver | AAV3B-GBA | 1E13 | 3420 | 0.047 | 0.651 |
| 12 | Liver | AAV3B-GBA | 1E13 | 260 | 0.565 | 1.422 |
| 13 | Liver | AAV3B-GBA | 1E13 | 8060 | 0.065 | 0.761 |
| 6 | Liver | AAVDJ-GBA | 1E13 | 480400 | 6.063 | 38.871 |
| 7 | Liver | AAVDJ-GBA | 1E13 | 290000 | 3.539 | 27.325 |
| 8 | Liver | AAVDJ-GBA | 1E13 | 507800 | 8.849 | 37.834 |
| 21 | Liver | AAVDJ-GBA | 1E12 | 85600 | N/A | N/A |
| 22 | Liver | AAVDJ-GBA | 1E12 | 80800 | N/A | N/A |
| 23 | Liver | AAVDJ-GBA | 1E12 | 44200 | N/A | N/A |
| 26 | Spleen | Vehicle | 0 | 132 | N/A | 0.000 |
| 27 | Spleen | Vehicle | 0 | 0 | N/A | 0.001 |
| 11 | Spleen | AAV3B-GBA | 1E13 | 3460 | 0.009 | 0.000 |
| 12 | Spleen | AAV3B-GBA | 1E13 | 184000 | 0.552 | 0.004 |
| 13 | Spleen | AAV3B-GBA | 1E13 | 2380 | 0.007 | 0.000 |
| 6 | Spleen | AAVDJ-GBA | 1E13 | 49200 | 0.099 | 0.001 |
| 7 | Spleen | AAVDJ-GBA | 1E13 | 35580 | 0.076 | 0.003 |
| 8 | Spleen | AAVDJ-GBA | 1E13 | 38400 | 0.074 | 0.002 |

### GCase Protein Expression by LC-MS/MS

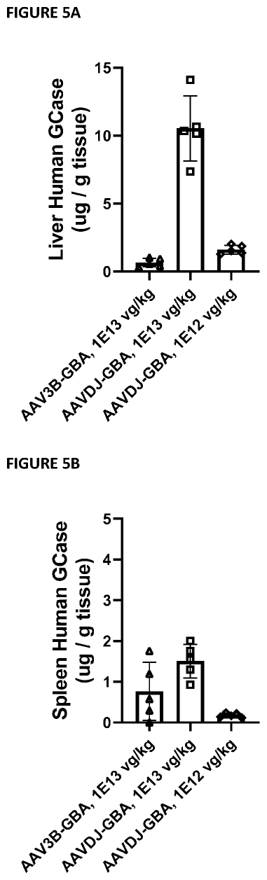

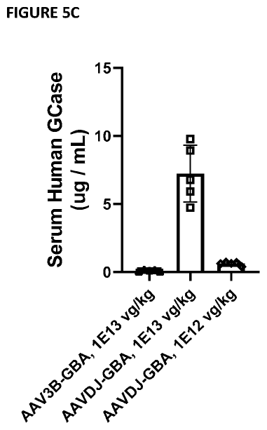

Human GCase protein in samples from test animals administered GBA vectors was quantified using an immunoaffinity LC-MS/MS assay. Liver GCase protein levels resulting from treatment with the AAVDJ-GBA vector was dose responsive and, consistent with transgene mRNA expression, produced higher GCase levels in liver compared to the same dose of AAV3B-GBA vector (FIG. 5A). This pattern was also reflected in GCase protein levels measured in serum samples from test animals (FIG. 5C), suggesting that AAV-mediated delivery of a GBA expression cassette can produce GCase in the liver which is then released into the blood stream. Despite the absence of GBA mRNA expression, GCase protein was detectable in spleen samples from test animals treated with both vectors (FIG. 5B), suggesting that GCase was taken up by the spleen from protein in the blood stream originating from the liver. As in liver, the amount of GCase produced by the AAVDJ-GBA vector in spleen samples was dose dependent. GCase protein concentration in serum and tissue samples from individual test animals is provided in Table 3, below.

### GCase Protein Expression by ICH

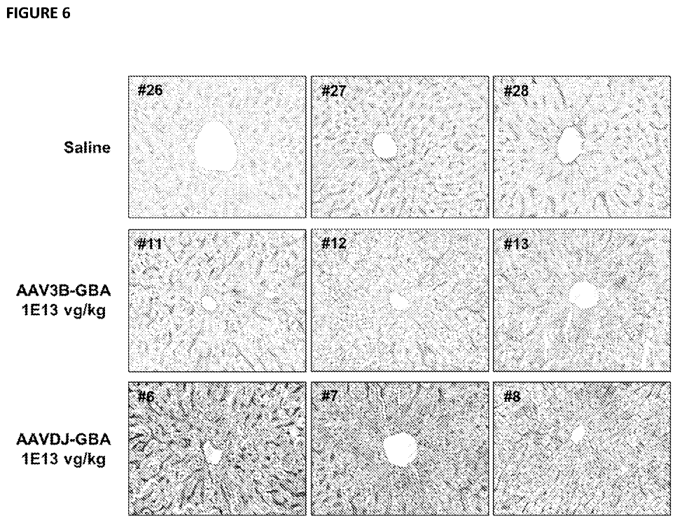

Human GCase protein in test animals treated with both GBA vectors was also studied by immunohistochemistry. Low intensity positive staining was detected in hepatocytes while high intensity staining was detected in Kupffer cells located along the sinusoid (FIG. 6). GCase protein staining intensity by ICH was greater for AAVDJ-GBA vector compared to the same dose of the AAV3B-GBA vector, consistent with liver GCase protein concentrations determined by LC-MS/MS.

### GCase Activity in Serum and Tissues

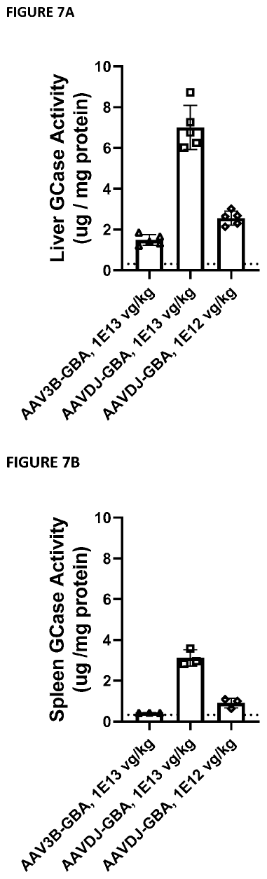

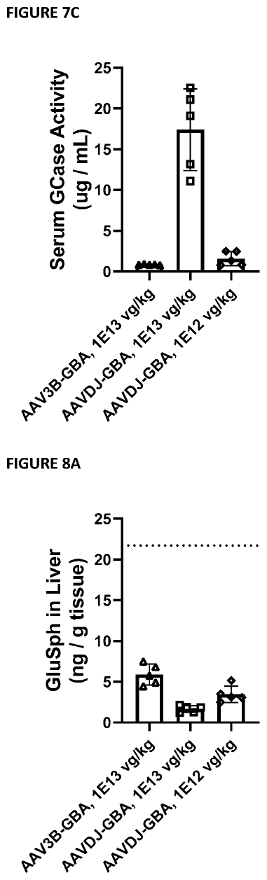

As measured using a fluorogenic substrate assay GCase activity in serum and tissue samples from test animals increased after treatment with AAVDJ-GBA and AAV3B-GBA vectors relative to vehicle control (FIGS. 7A, 7B, 7C). In liver, both vectors produced GCase activity above that in the test animals which received only vehicle (FIG. 7A), whereas only AAVDJ-GBA vector produced GCase activity levels in spleen (FIG. 7B) and serum (FIG. 7C) which exceeded negative controls, including at the lower dose tested (1E12 vg/kg). In serum, liver and spleen, GCase activity produced by AAVDJ-GBA vector was dose responsive. Collectively, these results demonstrate that the human GCase enzyme produced from the vectors in liver was functional.

### GluSph Levels in Serum and Tissues

Glucosylsphingosine (GluSph) is biomarker for Gaucher disease. GCase deficiency in the disorder results in intra-lysosomal accumulation of glucosylceramide, which is then deacylated by acid ceramidase to form glucosylsphingosine. Recovery of GCase activity, such as by factor replacement, or gene therapy, would be expected to reduce GluSph levels.

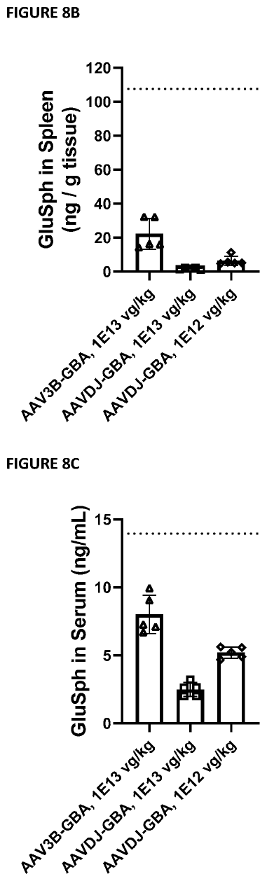

GluSph concentrations in serum and tissue samples from vector and vehicle treated test animals were determined by LC-MS/MS. D409V transgenic mice are deficient in GCase activity and therefore have elevated GluSph levels in liver, spleen and serum. When treated with the AAVDJ-GBA or AAV3B-GBA vectors, GluSph levels were substantially reduced in the same tissues and serum compared to the levels prevailing in vehicle treated test animals (FIGS. 8A, 8B, 8C). Consistent with other results, treatment with AAVDJ-GBA produced a greater decline in GluSph levels compared to the same dose of AAV3B-GBA vector. The reduction in GluSph levels in response to AAVDJ-GBA vector was dose responsive, and greater than 84% reduction in liver and spleen GluSph levels was achieved at a dose of 1E12 vg/kg. Notably, although the amount of GCase protein detected in liver from test animals treated with 1E13 vg/kg AAV3B-GBA vector was only 6% of that in animals treated with the same dose of AAVDJ-GBA, liver GluSph levels were reduced more than 70% (FIG. 8A). Similar declines in GluSph concentration occurred in spleen (FIG. 8B) and serum (FIG. 8C). These results demonstrated that one of the most significant markers of Type 1 Gaucher disease, hepatic and splenic accumulation of sphingolipids could be reduced by treating an animal model of the disease with vectors designed to transduce liver and produce enzymatically active hGCase protein which is secreted into the blood circulation.

TABLE 3

| | | | hGCase | hGCase | hGCase | hGCase | | hGCase | hGCase | | GluSph | GluSph |
|---|---|---|---|---|---|---|---|---|---|---|---|---|
| | | | conc | conc | conc | conc | hGCase | activity | activity | GluSph | conc | conc |
| Test | | | ng/mL | ng/mL | ng/g | ng/g | activity | μg/mg | μg/mg | conc | ng/g | ng/g |
| Animal | | Dose | Serum | Serum | tissue | tissue | μg/mL | protein | protein | μg/mL | tissue | tissue |
| No. | Test Article | (vg/kg) | Day 14 | Day 28 | Spleen | Liver | Serum | Spleen | Liver | Serum | Spleen | Liver |
| 26 | Vehicle | NA | BLOQ | BLOQ | BLOQ | BLOQ | 0.51 | 0.28 | 0.28 | 13.50 | 80.90 | 18.80 |
| 27 | Vehicle | NA | BLOQ | BLOQ | BLOQ | BLOQ | 0.59 | 0.37 | 0.33 | 13.50 | 139.00 | 21.20 |
| 28 | Vehicle | NA | BLOQ | BLOQ | BLOQ | BLOQ | 0.60 | 0.35 | 0.31 | 14.10 | 114.00 | 23.50 |
| 29 | Vehicle | NA | BLOQ | BLOQ | BLOQ | BLOQ | 0.59 | N/A | 0.33 | 14.70 | 96.40 | 23.40 |
| 21 | AAVDJ-GBA | 1E12 | 543 | 724 | 141 | 2030 | 2.48 | 1.00 | 2.68 | 5.58 | 5.00 | 3.52 |
| 22 | AAVDJ-GBA | 1E12 | 250 | 396 | 118 | 1380 | 0.77 | 0.64 | 2.14 | 5.24 | 11.30 | 5.15 |
| 23 | AAVDJ-GBA | 1E12 | 677 | 729 | 221 | 1860 | 2.49 | 1.09 | 2.64 | 4.89 | 5.11 | 3.11 |
| 24 | AAVDJ-GBA | 1E12 | 407 | 621 | 164 | 1280 | 0.82 | N/A | 2.30 | 5.62 | 5.67 | 3.08 |
| 25 | AAVDJ-GBA | 1E12 | 630 | 639 | 244 | 1530 | 1.34 | N/A | 3.02 | 4.68 | 4.76 | 2.51 |
| 6 | AAVDJ-GBA | 1E13 | 5230 | 6780 | 1550 | 14100 | 13.18 | 2.83 | 7.27 | 2.62 | 2.10 | 1.86 |
| 7 | AAVDJ-GBA | 1E13 | 5440 | 5920 | 1300 | 10200 | 19.11 | 2.96 | 6.76 | 2.64 | 1.85 | 1.88 |
| 8 | AAVDJ-GBA | 1E13 | 7770 | 9770 | 2000 | 10700 | 21.09 | 3.58 | 8.73 | 2.01 | 1.23 | 1.21 |
| 9 | AAVDJ-GBA | 1E13 | 4110 | 4750 | 927 | 7370 | 11.09 | N/A | 6.02 | 3.22 | 2.12 | 2.16 |
| 10 | AAVDJ-GBA | 1E13 | 223 | 8930 | 1750 | 10300 | 22.54 | N/A | 6.25 | 2.00 | 1.39 | 1.25 |
| 11 | AAV3B-GBA | 1E13 | 49.8 | 44.4 | BLOQ | 334 | 0.76 | 0.44 | 1.32 | 9.94 | 32.10 | 6.83 |
| 12 | AAV3B-GBA | 1E13 | 154 | 118 | 297 | 561 | 0.83 | 0.43 | 1.41 | 7.27 | 16.40 | 4.92 |
| 13 | AAV3B-GBA | 1E13 | 53.2 | 57.0 | 1190 | 424 | 0.83 | 0.43 | 1.21 | 9.06 | 32.20 | 7.50 |
| 14 | AAV3B-GBA | 1E13 | 169 | 162 | 1760 | 1030 | 0.89 | N/A | 1.65 | 7.11 | 16.10 | 5.76 |
| 15 | AAV3B-GBA | 1E13 | 174 | 179 | 581 | 939 | 0.92 | N/A | 1.85 | 6.70 | 14.30 | 4.44 |

BLOQ = below limit of quantitation

### Example 4

### Testing of AAV GCase Vector in Healthy Non-Human Primates

Extending the mouse studies, AAV3B-GBA clone 128 vector and AAVDJ-GBA clone 128 vector (referred to in this Example and related figures as AAV3B-GBA and AAVDJ-GBA, respectively) were administered by intravenous injection to healthy cynomolgus monkeys and samples taken after necropsy to analyze vector genome copy number in transduced cells, and RNA and protein expression resulting from vector transduction.

### Materials and Methods

### Vector Administration and Sample Collection

AAV3B-GBA clone 128 vector was administered to cynomolgus monkeys at four escalating doses by intravenous injection. Two males and one female received a dose of 3E12 vector genome per kg of body weight (vg/kg); one male and two females received a dose of 1E13 vg/kg; one male and one female received a dose of 3E13 vg/kg; and one male and one female received a dose of 5.3E13 vg/kg and 5.2E13 vg/kg, respectively. AAVDJ-GBA clone 128 vector was administered IV to one male and one female monkey at a dose of 3E13 vg/kg. A total of four animals, two males and two females, received vehicle as a negative control. Animals were maintained on methylprednisolone throughout the study to suppress possible immune response. Necropsy was conducted approximately 30 days after dosing. Serum samples and 100-200 mg tissue samples from liver, spleen and dorsal root ganglion were collected and stored at −80° C. until use. For immunohistochemistry (IHC), tissues were fixed in 10% neutral buffered formalin, processed to paraffin blocks and sections.

### DNA Isolation

Frozen tissue samples were used for DNA isolation using phenol/chloroform DNA extraction methods. Frozen tissue samples were pulverized into a frozen dry powder using a Covaris CP02 Cyroprep Pulverizer following manufacturer instructions. Approximately 30 mg of each frozen tissue powder was transferred into a new tube and resuspended in 292.5 mL of TENS lysis buffer containing 1% SDS. Next, 7.5 mL Proteinase K was added to each tube and incubate at 56° C. in a thermomixer shaking at 1000 rpm for 3 hours, and vortexed for 15 seconds every 30 minutes. Samples were then cooled to room temperature and treated with RNase A for 5 minutes. Each sample was treated with 300 ml of phenol: chloroform: isoamyl alcohol (25:24:1) and vortexed for 20 seconds. The upper phase liquid was collected for DNA precipitation by the addition of 7.5 M NH₄OAc at approximately 0.5 times of the upper phase volume and the addition of pure ethanol at 3 times the total volume. Samples were chilled at −20° C. for at least 2 hours followed by centrifugation at 4° C. for 30 minutes at 16,000 g. DNA pellet was washed with ice cold 70% ethanol and resuspended in nuclease free water. DNA concentration was measured with Nanodrop spectrophotometer.

### RNA Isolation

Frozen tissue powder was prepared as described in the above section. Approximately 30 mg of tissue sample was used for homogenization in 1 mL Trizol with the presence of a 5 mm stainless steel bead using a Tissuelyzer II set at frequency 25 1/s for 5 minutes. Lysates were transferred to phasemaker tubes and 200 mL chloroform was added. Samples were vortexed for 15 seconds, incubated at room temperature for 5 minutes, and centrifuged at 14,000 g for 5 min at 4° C. The clear supernatant was transfer to a new tube and processed with the Purelink RNA mini kit per manufacturer instructions. DNAse treatment was performed on column for 20 min before elution with nuclease free water. RNA concentration was measured using Nanodrop spectrophotometer.

### Vector Genome Copy Number

Genomic DNA (gDNA) was extracted from liver, spleen, and DRG, and vector genome copies (VGC) determined by ddPCR of the GBA transgene to quantify vector transduction. VGC was normalized either to gDNA input or to the expression of housekeeping gene Tfrc. In the ddPCR assay, annealing temperature was 60° C., probe primer sequence was TTAGGAAAGGACAGTGGGAGTGGC (SEQ ID NO:43), forward primer sequence was AATGCGATGCAATTTCCTCATT (SEQ ID NO:44), reverse primer sequence was TGCCAGCCATCTGTTGTT (SEQ ID NO:45), probe flurophore was FAM, and the quencher was NFQ. DNA was diluted to 10 ng/ml. Twenty μL PCR reaction mixture containing ddPCR Supermix, isolated gDNA, GBA primers and FAM probes (see above), housekeeping gene (mf) Tfrc primers and VIC probes were prepared in a Airclean 600 PCR workstation cleaned with DNAZap and added to a ddPCR 96-well plate. Droplets were generated using QX200 droplet generator. Plates were sealed with PX1 PCR plate sealer. PCR program: 95° C. for 10 min→ [94° C. for 30 sec→60° C. for 1 min] (×40 cycles)→98° C. for 10 min→4° C. hold was completed. Droplet counts and amplitude was acquired by a QX200 reader and analyzed with QuantaSoft software to calculate the number of DNA copies. VGC obtained from the FAM channel are normalized to ug of gDNA input or reported as VGC per haploid genome by calculating the ratio of FAM to VIC channel.

### GBA Transgene Expression

GBA mRNA expression from the transduced transgene was quantified by reverse transcription and ddPCR of RNA samples from liver, spleen, and DRG. Transgene mRNA levels were normalized to expression of housekeeping gene Hprt. Isolated RNA samples were reverse transcribed to generate cDNA library using the iScript cDNA Synthesis kit (Bio-Rad, cat #1708891) following manufacturer's protocol. 500 ng of total RNA was used in 20 μL reaction. Reverse transcription was performed at 37° C. for 60 minutes followed by 95° C. for 5 minutes. cDNA samples were stored at −20° C. until RT-PCR analysis. RNA expression quantification of GBA transgenes was determined using ddPCR with the same GBA primers and probes as described above and primer probes for housekeeping gene mfHprt. PCR reaction mixture containing ddPCR Supermix (no dUTP) and above primer probes was prepared in a Airclean 600 PCR workstation cleaned with DNAZap and added to a ddPCR 96-well plate. Droplet generation, PCR programs, and data acquisition were performed as described above. VGC was normalized to ug of gDNA or the count of mfHPRT.

### GCase Protein Expression by LC-MS/MS

Human recombinant GCase protein produced in vector treated test animals was enriched from samples of serum, PBMCs, and lysates of liver and spleen tissue with an antibody that specifically binds GCase, and then quantified by liquid chromatography tandem mass spectrometry (LC-MS/MS). PBMC pellets were combined by vial in M-PER buffer+protease inhibitor and lysed by bead milling for 6 minutes in a Bullet Blender STORM 5. Spleen and liver sections were prepared by adding a volume of M-PER+protease inhibitor to prepare 50 mg/mL tissue to buffer, samples were processed using an Omni Bead Ruptor Elite for 1.5 minutes. Samples were assayed at various volumes, 50 μL serum, 200 μL tissue lysate and 200-800 μL PBMC lysate against a standard curve surrogate matrix of 1% BSA in PBS spiked with taliglucerase protein. The assay linear range was 0.25 to 1,000 ng/ml. Dual IP was performed using 2 μg of anti-GCase antibody+biotin and streptavidin magnetic beads. Samples were processed on a Thermo KingFisher Flex and eluted with 30 mM HCl+5% ACN. Samples were adjusted to pH 8.3 using 1M Tris-HCl, reduced, alkylated, and digested overnight using Promega LysC-Trypsin. Samples were injected on Thermo Altis MS system at a volume of 85 μL using trap and elute nanoflow LC conditions. All data for the LC-MS/MS assay was acquired by Xcalibur v. 4.1 on a Thermo Altis. Data processing occurred using Skyline v. 21.2.0.369 and exported to Excel. A calibration curve was generated from peak area ratios of analyte peptide over SIL peptide and the concentration of study samples were determined by back-calculating the peak-area-ratio against the standard curve. LC-MS/MS data for PBMC and tissue lysate was normalized to total protein weight for tissue and total number of million cells collected for PBMC pellets.

### GCase Protein Expression by ICH

Paraffin sections of liver, spleen, and DRG were stained with anti-GCase antibody using a fully automated stainer (Leica Biosystems Bond Rx). Briefly, the slides were dewaxed and treated with peroxide for 10 minutes. Then, the slides were rinsed with deionized water and permeabilized with 0.3% Triton solution for 1 hour before incubation with a primary anti-GCase antibody (Sigma-Aldrich) for 1 hour. The primary antibody was diluted in Bond primary antibody diluent at 1:250 dilution. Next, the slides were rinsed and incubated in a secondary antibody (Leica #DS9800) for 8 minutes. The presence of antibodies is visualized by an incubation with DAB (Leica #DS9800) for 10 minutes. Finally, the slides were rinsed in water and dehydrated from graded ethanol to xylene before cover slipping and imaged on a slide scanner (Axio Scan Z1, Zeiss). Images were rendered with ZEN Blue software.

### Capsid Neutralizing Antibody Titer

To control for the possiblity that presence of neutralizing antibodies to the vector capsid could affect transduction efficiency, nAb titers against AAV3B in the test animals was determined on the day of vector administration. Cell-based transduction inhibition assay was conducted to assess NAb titer. Briefly, HEK293T cells were seeded on Poly D-lysine 96 well plates at 60K cells per 100 μL per well and incubated at 37° C., 5% CO2 overnight. Next day, add 10 μL of WT Adenovirus to cell plate at MOI 100 and incubate the plate for 2 hours. Make serial dilutions of serum sample and heat-inactivated serum (used as a control) and mix the AAV vector (at MOI 10000), incubate the plate for 1 hour at 37° C., 5% CO2 to allow vector-serum complex formation. The vector serum complex was then transferred (120 μL) to the cells and incubate the plate at 37° C., 5% CO2 for 18-24 hours. Then add 100 μL of Bight Glo Reagent to the plate as per user manual. Read the plate on Envision. The NAb titers were calculated using the heat inactivated serum cut off. The titers were reported as the lowest reciprocal serum dilution (5, 10, 20, 40 up to 5120) that had ≥50% transduction inhibition.

### Results

### Vector Genome Copy Number

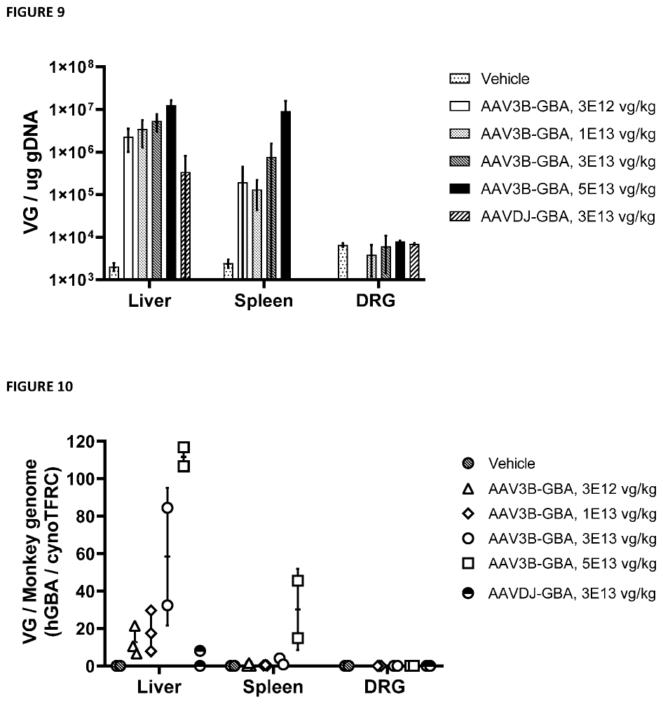

AAV3B-GBA vector transduced liver and spleen as determined by quantifying vector genome copy numbers in tissue samples taken from test animals, with a trend toward dose responsiveness (FIG. 9). AAVDJ-GBA vector also transduced liver, but about 29-fold less efficiently compared to AAV3B-GBA vector at the same dose (3E13 vg/kg), confirming that AAV3B capsid transduces NHP liver more efficiently than AAVDJ. Vector genome copy numbers detected in dorsal root ganglia (DRG) were at background levels, indicating no transduction of that tissue by either vector (FIG. 9). Similar results were observed when vector genome copy numbers were normalized to that of a cynomolgus housekeeping gene, Tfrc. As dose of the AAV3B-GBA vector increased, the relative number of vector genomes per copy of the housekeeping gene (serving as proxy for a host cell haploid genome) also increased, from 7× at the lowest dose of 3E12 vg/kg to 117× at the highest dose of 5E13 vg/kg (FIG. 10). Based on the data normalized to the housekeeping gene, the AAV3B vector transduced spleen, but only at the two highest doses tested, and therefore less efficiently than liver (FIG. 10). The normalized data confirmed no transduction of DRG. Transduction data for individual test animals is provided in Table 4, below.

### GBA Transgene Expression

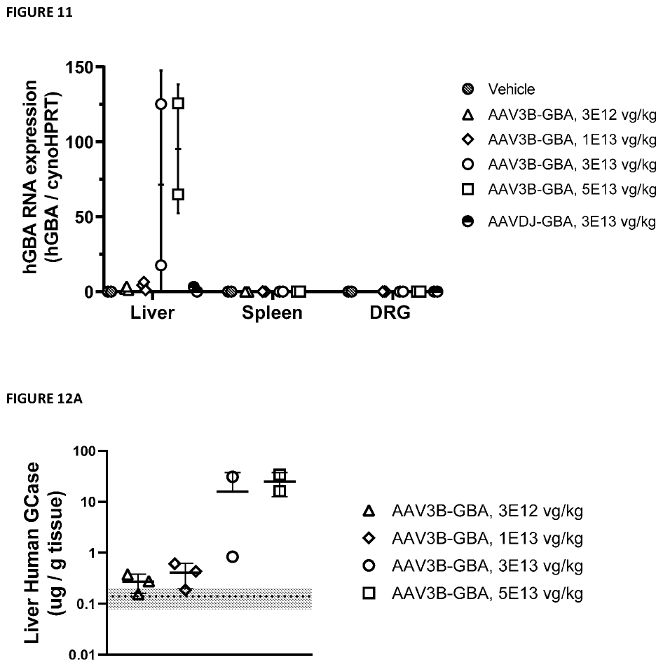

Although vector genomes after transduction with AAV3B-GBA vector were detectible in both liver and spleen (FIG. 9 and FIG. 10), hGCase mRNA produced by that vector was detected only in liver and not spleen (FIG. 11), suggesting that the liver specific enhancer and promoter used in the expression cassette were effective at restricting expression of the GBA transgene to transduced liver cells. Consistent with the transduction efficiency determined by detecting vector genome DNA, very low levels of expression resulted from liver transduction with the AAVDJ-GBA vector, and no RNA expression was seen in spleen or DRG from either vector. Liver expression from the GBA transgene delivered by the AAV3B vector was also dose responsive. When normalized to mRNA levels from a host cell housekeeping gene, Hprt, GBA mRNA levels increased from 1× at a dose of 3E12 vg/kg, 7× at a dose of 1E13 vg/kg, 17× at a dose of 3E13 vg/kg, and 125× at a dose of 5E13 vg/kg. GBA transgene mRNA expression data for individual test animals is provided in Table 4, below.

TABLE 4

| Animal | | | Dose | VG/μg | VG/Monkey genome | hGBA RNA |
|---|---|---|---|---|---|---|
| ID | Tissue | Test Article | (vg/kg) | gDNA | (hGBA/cynoTFRC) | (hGBA/cynoHPRT) |
| 1A | Liver | Vehicle | 0 | 2340 | 0.025 | 0.007 |
| 6A | Liver | Vehicle | 0 | 1720 | 0.013 | 0.000 |
| 2A | Liver | AAV3B-GBA | 3E13 | 6974000 | 84.404 | 125.262 |
| 7A | Liver | AAV3B-GBA | 3E13 | 3714000 | 32.333 | 17.578 |
| 3A | Liver | AAV3B-GBA | 5E13 | 10076000 | 106.604 | 64.914 |
| 8A | Liver | AAV3B-GBA | 5E13 | 15200000 | 116.741 | 125.659 |
| 4A | Liver | AAVDJ-GBA | 3E13 | 676000 | 8.063 | 3.131 |
| 9A | Liver | AAVDJ-GBA | 3E13 | 3880 | 0.032 | 0.000 |
| 1A | Spleen | Vehicle | 0 | 2060 | 0.007 | 0.001 |
| 6A | Spleen | Vehicle | 0 | 2840 | 0.007 | 0.001 |
| 2A | Spleen | AAV3B-GBA | 3E13 | 1342000 | 4.056 | 0.032 |
| 7A | Spleen | AAV3B-GBA | 3E13 | 197000 | 0.786 | 0.004 |
| 3A | Spleen | AAV3B-GBA | 5E13 | 13800000 | 45.590 | 0.052 |
| 8A | Spleen | AAV3B-GBA | 5E13 | 4386000 | 14.860 | 0.029 |
| 1A | DRG | Vehicle | 0 | 6160 | 0.034 | 0.046 |
| 6A | DRG | Vehicle | 0 | 7000 | 0.055 | 0.000 |
| 2A | DRG | AAV3B-GBA | 3E13 | 9400 | 0.065 | 0.016 |
| 7A | DRG | AAV3B-GBA | 3E13 | 2800 | 0.031 | 0.000 |
| 3A | DRG | AAV3B-GBA | 5E13 | 8200 | 0.058 | 0.000 |
| 8A | DRG | AAV3B-GBA | 5E13 | 7600 | 0.063 | 0.002 |
| 4A | DRG | AAVDJ-GBA | 3E13 | 7200 | 0.050 | 0.001 |
| 9A | DRG | AAVDJ-GBA | 3E13 | 6600 | 0.053 | 0.000 |
| 1B | Liver | Vehicle | 0 | 100 | 0.001 | 0.000 |
| 5B | Liver | Vehicle | 0 | 680 | 0.006 | 0.000 |
| 2B | Liver | AAV3B-GBA | 3E12 | 1766000 | 10.600 | 1.770 |
| 3B | Liver | AAV3B-GBA | 3E12 | 1330000 | 6.604 | 1.160 |
| 6B | Liver | AAV3B-GBA | 3E12 | 3714000 | 21.419 | 3.550 |
| 4B | Liver | AAV3B-GBA | 1E13 | 3660000 | 17.395 | 4.420 |
| 7B | Liver | AAV3B-GBA | 1E13 | 5518000 | 29.667 | 6.560 |
| 8B | Liver | AAV3B-GBA | 1E13 | 1190000 | 7.881 | 1.020 |
| 1B | Spleen | Vehicle | 0 | 122 | 0.000 | 0.000 |
| 5B | Spleen | Vehicle | 0 | 720 | 0.002 | 0.000 |
| 2B | Spleen | AAV3B-GBA | 3E12 | 41760 | 0.059 | 0.010 |
| 3B | Spleen | AAV3B-GBA | 3E12 | 59380 | 0.143 | 0.000 |
| 6B | Spleen | AAV3B-GBA | 3E12 | 486000 | 1.559 | 0.000 |
| 4B | Spleen | AAV3B-GBA | 1E13 | 70000 | 0.227 | 0.000 |
| 7B | Spleen | AAV3B-GBA | 1E13 | 90800 | 0.245 | 0.000 |
| 8B | Spleen | AAV3B-GBA | 1E13 | 232400 | 0.521 | 0.000 |
| 1B | DRG | Vehicle | 0 | 198 | 0.002 | 0.000 |
| 5B | DRG | Vehicle | 0 | 360 | 0.004 | 0.000 |
| 4B | DRG | AAV3B-GBA | 1E13 | 1140 | 0.008 | 0.000 |
| 7B | DRG | AAV3B-GBA | 1E13 | 3960 | 0.022 | 0.000 |
| 8B | DRG | AAV3B-GBA | 1E13 | 6520 | 0.038 | 0.000 |

### GCase Protein Expression by LC-MS/MS

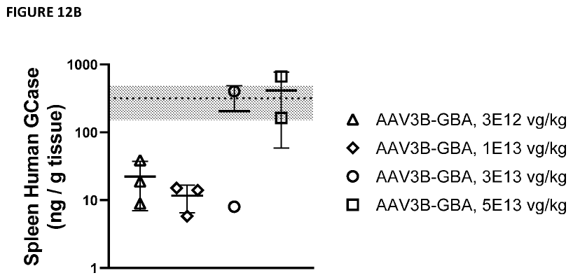

Human GCase protein in samples from test animals administered GBA vectors was quantified using an immunoaffinity LC-MS/MS assay. GCase protein levels from the AAV3B-GBA vector were on average higher in liver than spleen, and correlated with vector dose in both tissues (FIG. 12A and FIG. 12B). Because GBA mRNA was detected in liver, but not spleen, of vector treated test animals, presence of GCase protein in spleen likely resulted from its uptake from circulating protein secreted into the blood by transduced hepatocytes. Levels of hGCase protein observed in liver of test animals treated with AAV3B-GBA exceeded the average level in liver samples from healthy humans at all doses tested (FIG. 12A), while protein levels in spleen of test animals treated with vector at the two highest doses was consistent with levels of GCase in spleen of healthy humans (FIG. 12B). Blood samples were also collected at multiple time points after vector administration and amounts of hGCase protein quantified in serum and peripheral blood mononuclear cells (PBMC) prepared from the blood. The results were variable, but demonstrate that hGCase produced by liver cells transduced with both GBA vectors, including at the lowest doses tested, is detectible in serum and can be taken up by PBMCs in the blood. GCase protein levels in serum and tissue samples from individual test animals is provided in Table 5 and Table 6.

TABLE 5

| | | | | hGCase | hGCase | hGCase | hGCase | hGCase | hGCase | hGCase | hGCase | hGCase |
|---|---|---|---|---|---|---|---|---|---|---|---|---|
| | | | | conc | conc | conc | conc | conc | conc | conc | conc | conc |
| Test | | | | ng/mL | ng/mL | ng/mL | ng/mL | ng/mL | ng/mL | ng/mL | ng/mL | ng/mL |
| Animal | | Dose | | Serum | Serum | Serum | Serum | Serum | Serum | Serum | Serum | Serum |
| No. | Test Article | (vg/kg) | Sex | Day 3 | Day 8 | Day 15 | Day 22 | Day 29 | Day 30 | Day 37 | Day 50 | Day 58 |
| 1A | Vehicle | NA | M | 13 | BLOQ | BLOQ | BLOQ | BLOQ | — | — | — | — |
| 6A | Vehicle | NA | F | BLOQ | BLOQ | BLOQ | BLOQ | BLOQ | — | — | — | — |
| 1B | Vehicle | NA | M | BLOQ | BLOQ | BLOQ | BLOQ | BLOQ | BLOQ | BLOQ | BLOQ | BLOQ |
| 5B | Vehicle | NA | F | BLOQ | BLOQ | BLOQ | BLOQ | BLOQ | BLOQ | BLOQ | BLOQ | BLOQ |
| 2B | AAV3B-GBA | 3.0E12 | M | 129 | 236 | 200 | 176 | — | 119 | 127 | 87 | 93 |
| 3B | AAV3B-GBA | 3.0E12 | M | 117 | 264 | 141 | 142 | — | 76 | 95 | 72 | 45 |
| 6B | AAV3B-GBA | 3.0E12 | F | 80 | 324 | 259 | 217 | — | 242 | 176 | 114 | 99 |
| 4B | AAV3B-GBA | 1.0E13 | M | 94 | 300 | 344 | 380 | — | 267 | 237 | 182 | 193 |
| 7B | AAV3B-GBA | 1.0E13 | F | 136 | 364 | 282 | 371 | — | 330 | 300 | 188 | 144 |
| 8B | AAV3B-GBA | 1.0E13 | F | 100 | 220 | 79 | 86 | — | 63 | 59 | 44 | 35 |
| 2A | AAV3B-GBA | 3.0E13 | M | 200 | 623 | 6067 | 8240 | 8226 | — | — | — | — |
| 7A | AAV3B-GBA | 3.0E13 | F | 140 | 187 | 228 | 231 | 161 | — | — | — | — |
| 3A | AAV3B-GBA | 5.3E13 | M | 278 | 496 | 904 | 2871 | 3930 | — | — | — | — |
| 8A | AAV3B-GBA | 5.2E13 | F | 373 | 736 | 2581 | 3550 | 7366 | — | — | — | — |
| 4A | AAVDJ-GBA | 3.0E13 | M | 23 | 86 | 99 | 51 | 40 | — | — | — | — |
| 9A | AAVDJ-GBA | 3.0E13 | F | 0.4 | 0.9 | 2.7 | BLOQ | 0.5 | — | — | — | — |

BLOQ = below limit of quantitation

TABLE 6

| | | | | hGCase | hGCase | hGCase | hGCase | hGCase | hGCase | hGCase | hGCase | hGCase |
|---|---|---|---|---|---|---|---|---|---|---|---|---|
| | | | | conc | conc | conc | conc | conc | conc | conc | conc | conc |
| | | | | pg/1E6 | pg/1E6 | pg/1E6 | pg/1E6 | pg/1E6 | ng/g | ng/g | ng/g | ng/g |
| Test | | | | cells | cells | cells | cells | cells | tissue | tissue | tissue | tissue |
| Animal | | Dose | | PBMC | PBMC | PBMC | PBMC | PBMC | Spleen | Spleen | Liver | Liver |
| No. | Test Article | (vg/kg) | Sex | Day 15 | Day 29 | Day 30 | Day 50 | Day 58 | Day 29 | Day 58 | Day 29 | Day 58 |
| 1A | Vehicle | NA | M | BLOQ | BLOQ | — | — | — | BLOQ | — | BLOQ | — |
| 6A | Vehicle | NA | F | BLOQ | BLOQ | — | — | — | BLOQ | — | BLOQ | — |
| 1B | Vehicle | NA | M | BLOQ | — | BLOQ | BLOQ | BLOQ | — | BLOQ | — | BLOQ |
| 5B | Vehicle | NA | F | BLOQ | — | BLOQ | BLOQ | BLOQ | — | BLOQ | — | BLOQ |
| 2B | AAV3B-GBA | 3.0E12 | M | BLOQ | — | BLOQ | BLOQ | BLOQ | — | 19 | — | 276 |
| 3B | AAV3B-GBA | 3.0E12 | M | 5 | — | 4 | BLOQ | BLOQ | — | 9 | — | 156 |
| 6B | AAV3B-GBA | 3.0E12 | F | BLOQ | — | 9 | BLOQ | BLOQ | — | 39 | — | 377 |
| 4B | AAV3B-GBA | 1.0E13 | M | BLOQ | — | BLOQ | BLOQ | BLOQ | — | 15 | — | 430 |
| 7B | AAV3B-GBA | 1.0E13 | F | 4 | — | 25 | 3 | BLOQ | — | 14 | — | 608 |
| 8B | AAV3B-GBA | 1.0E13 | F | BLOQ | — | BLOQ | BLOQ | BLOQ | — | 6 | — | 187 |
| 2A | AAV3B-GBA | 3.0E13 | M | 52 | 63 | — | — | — | 403 | — | 31123 | — |
| 7A | AAV3B-GBA | 3.0E13 | F | 249 | 12 | — | — | — | 8 | — | 837 | — |
| 3A | AAV3B-GBA | 5.3E13 | M | 9 | 40 | — | — | — | 163 | — | 16275 | — |
| 8A | AAV3B-GBA | 5.2E13 | F | 457 | 315 | — | — | — | 668 | — | 33891 | — |
| 4A | AAVDJ-GBA | 3.0E13 | M | BLOQ | BLOQ | — | — | — | 7 | — | 354 | — |
| 9A | AAVDJ-GBA | 3.0E13 | F | BLOQ | BLOQ | — | — | — | BLOQ | — | BLOQ | — |

BLOQ = below limit of quantitation

### GCase Protein Expression by ICH

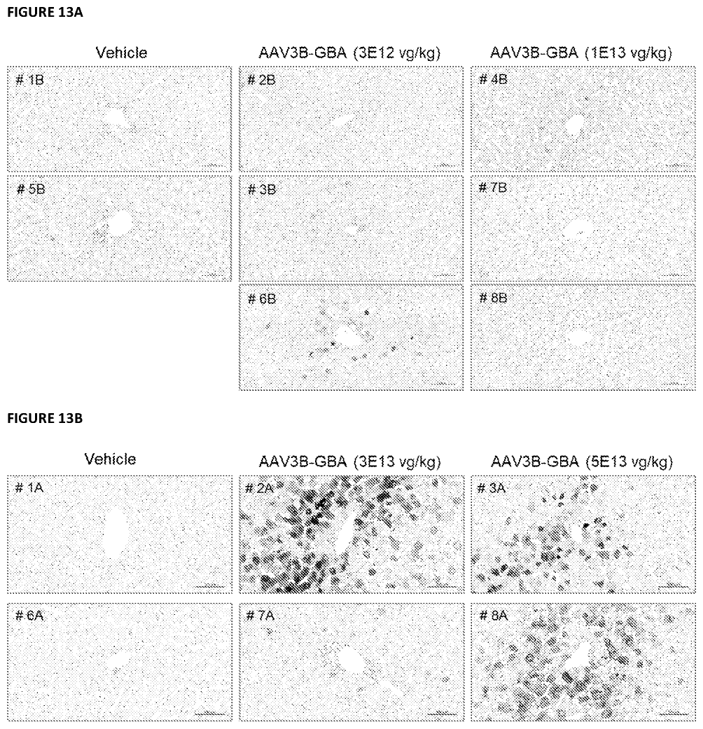

Human GCase protein in test animals treated with AAV3B-GBA vector was also studied by immunohistochemistry. Positive staining above background was observed in hepatocytes surrounding periportal veins in liver samples from animals treated at all vector doses, although the number of positive cells was much greater at the two highest doses tested (FIG. 13B) versus the two lower doses (FIG. 13A). Further, at the two highest doses, there was correspondence in individual test animals between the number of liver cells staining positive for GCase and the amount of GBA mRNA detectible in liver samples. In spleen and DRG samples from vector treated test animals, hGCase staining was at background levels (data not shown).

### Capsid Neutralizing Antibody Titer

Neutralizing antibody titer to AAV3B capsid was measured in serum from test animals collected before administering AAV3B-GBA vector and 22 days after. Before treatment, nAb titers were negative or low (20-40 dilution factor for ≥50% transduction inhibition in the assay), suggesting that high nAb titer was not the main cause of low transduction efficiency observed in some test animals. At the two highest vector doses, test animals had high AAV3B capsid nAb titers (up to 5120 dilution factor) 22 days after treatment. By contrast, nAb titers remained low in the test animals receiving the lower vector doses, indicating that at least 3E13 vg/kg vector was required for the animals to seroconvert. Anti-capsid neutralizing antibody titers in individual test animals is provided in Table 7.

TABLE 7
Anti-AAV3B nAb Titer

| Animal | | | | |
|---|---|---|---|---|
| number | Gender | Dose (vg/kg) | Day 1 NAb Titer | Day 22 NAb Titer |
| 1B | M | 0 | <5 | <5 |
| 5B | F | 0 | <5 | <5 |
| 2B | M | 3E+12 | <5 | >40 |
| 3B | M | 3E+12 | <5 | >40 |
| 6B | F | 3E+12 | <5 | >40 |
| 4B | M | 1E+13 | <5 | >40 |
| 7B | F | 1E+13 | <5 | >40 |
| 8B | F | 1E+13 | <5 | >40 |
| 1A | M | 0 | 80 | 40 |
| 6A | F | 0 | 40 | 10 |
| 2A | M | 3E+13 | 40 | 2560 |
| 7A | F | 3E+13 | 20 | 5120 |
| 3A | M | 5E+13 | 20 | 2560 |
| 8A | F | 5E+13 | 20 | 2560 |

### Other Observations

Over the course of the experiment, test animal health and markers associated with immune reactivity to the vector were assessed at multiple time points. No test article related clinical changes or changes in body weight or food consumption were observed. Test article-related increases in cytokines IL-10 (peak changes up to 3.83× baseline) and IP-10 (peak increases up to 3.99× baseline) were observed in animals administered AAV3B-GBA vector ≥3E13 vg/kg, but not at lower doses. No increases in IFNalpha-2a, MCP-1, IL-6, IFN-gamma, TNF-alpha, or complement activation products C3a and C5b-9 were observed in any test animal. Increases in serum levels of the liver enzyme alanine aminotransferase (ALT), but not aspartate aminotransferase (AST), considered test article related were observed in four animals, ranging from 2.1-11.5 fold over the levels observed before vector administration, but did not correlate with the AAV3B nAb titer or extent of liver transduction or GBA expression in the corresponding test animal. Transient test article related increases in C-reactive protein (CRPHS) were observed in two test animals. Increased numbers of reticulocytes, white blood cells, neutrophils, lymphocytes, or monocytes, or serum triglyceride levels, were also observed in certain test animals on certain days.

TABLE OF SEQUENCES

| SEQ ID | | |
|---|---|---|
| NO. | Description | Sequence |
| 1 | AAV3B VP1 capsid | MAADGYLPDWLEDNLSEGIREWWALKPGVPQPKANQQHQDNRRGLVLPGYKYLGPGNGLDKGEPVNEADAAALEHDKAYDQQLKAGDNPY |
| | protein amino | LKYNHADAEFQERLQEDTSFGGNLGRAVFQAKKRILEPLGLVEEAAKTAPGKKRPVDQSPQEPDSSSGVGKSGKQPARKRLNFGQTGDSE |
| | acid sequence | SVPDPQPLGEPPAAPTSLGSNTMASGGGAPMADNNEGADGVGNSSGNWHCDSQWLGDRVITTSTRTWALPTYNNHLYKQISSQSGASNDN |
| | | HYFGYSTPWGYFDFNRFHCHFSPRDWQRLINNNWGFRPKKLSFKLFNIQVKEVTQNDGTTTIANNLTSTVQVFTDSEYQLPYVLGSAHQG |
| | | CLPPFPADVFMVPQYGYLTLNNGSQAVGRSSFYCLEYFPSQMLRTGNNFQFSYTFEDVPFHSSYAHSQSLDRLMNPLIDQYLYYLNRTQG |
| | | TTSGTTNQSRLLFSQAGPQSMSLQARNWLPGPCYRQQRLSKTANDNNNSNFPWTAASKYHLNGRDSLVNPGPAMASHKDDEEKFFPMHGN |
| | | LIFGKEGTTASNAELDNVMITDEEEIRTTNPVATEQYGTVANNLQSSNTAPTTRTVNDQGALPGMVWQDRDVYLQGPIWAKIPHTDGHFH |
| | | PSPLMGGFGLKHPPPQIMIKNTPVPANPPTTFSPAKFASFITQYSTGQVSVEIEWELQKENSKRWNPEIQYTSNYNKSVNVDFTVDTNGV |
| | | YSEPRPIGTRYLTRNL |
| 2 | AAV3B VP2 capsid | MAPGKKRPVDQSPQEPDSSSGVGKSGKQPARKRLNFGQTGDSESVPDPQPLGEPPAAPTSLGSNTMASGGGAPMADNNEGADGVGNSSGN |
| | protein amino | WHCDSQWLGDRVITTSTRTWALPTYNNHLYKQISSQSGASNDNHYFGYSTPWGYFDFNRFHCHFSPRDWQRLINNNWGFRPKKLSFKLFN |
| | acid sequence | IQVKEVTQNDGTTTIANNLTSTVQVFTDSEYQLPYVLGSAHQGCLPPFPADVFMVPQYGYLTLNNGSQAVGRSSFYCLEYFPSQMLRTGN |
| | | NFQFSYTFEDVPFHSSYAHSQSLDRLMNPLIDQYLYYLNRTQGTTSGTTNQSRLLFSQAGPQSMSLQARNWLPGPCYRQQRLSKTANDNN |
| | | NSNFPWTAASKYHLNGRDSLVNPGPAMASHKDDEEKFFPMHGNLIFGKEGTTASNAELDNVMITDEEEIRTTNPVATEQYGTVANNLQSS |
| | | NTAPTTRTVNDQGALPGMVWQDRDVYLQGPIWAKIPHTDGHFHPSPLMGGFGLKHPPPQIMIKNTPVPANPPTTFSPAKFASFITQYSTG |
| | | QVSVEIEWELQKENSKRWNPEIQYTSNYNKSVNVDFTVDINGVYSEPRPIGTRYLTRNL |
| 3 | AAV3B VP3 capsid | MASGGGAPMADNNEGADGVGNSSGNWHCDSQWLGDRVITTSTRTWALPTYNNHLYKQISSQSGASNDNHYFGYSTPWGYFDFNRFHCHFS |
| | protein amino | PRDWQRLINNNWGFRPKKLSFKLFNIQVKEVTQNDGITTIANNLTSTVQVFTDSEYQLPYVLGSAHQGCLPPFPADVEMVPQYGYLTLNN |
| | acid sequence | GSQAVGRSSFYCLEYFPSQMLRTGNNFQFSYTFEDVPFHSSYAHSQSLDRLMNPLIDQYLYYLNRTQGTTSGTTNQSRLLESQAGPQSMS |
| | | LQARNWLPGPCYRQQRLSKTANDNNNSNFPWTAASKYHLNGRDSLVNPGPAMASHKDDEEKFFPMHGNLIFGKEGTTASNAELDNVMITD |
| | | EEEIRTTNPVATEQYGTVANNLQSSNTAPTTRTVNDQGALPGMVWQDRDVYLQGPIWAKIPHTDGHFHPSPLMGGFGLKHPPPQIMIKNT |
| | | PVPANPPTTESPAKFASFITQYSTGQVSVEIEWELQKENSKRWNPEIQYTSNYNKSVNVDFTVDINGVYSEPRPIGTRYLTRNL |
| 4 | AAV-DJ VP1 | MAADGYLPDWLEDTLSEGIRQWWKLKPGPPPPKPAERHKDDSRGLVLPGYKYLGPFNGLDKGEPVNEADAAALEHDKAYDRQLDSGDNPY |
| | capsid protein | LKYNHADAEFQERLKEDTSFGGNLGRAVFQAKKRLLEPLGLVEEAAKTAPGKKRPVEHSPVEPDSSSGTGKAGQQPARKRLNFGQTGDAD |
| | amino acid | SVPDPQPIGEPPAAPSGVGSLTMAAGGGAPMADNNEGADGVGNSSGNWHCDSTWMGDRVITTSTRTWALPTYNNHLYKQISNSTSGGSSN |
| | sequence | DNAYFGYSTPWGYFDFNRFHCHFSPRDWQRLINNNWGFRPKRLSFKLFNIQVKEVTQNEGTKTIANNLTSTIQVFTDSEYQLPYVLGSAH |
| | | QGCLPPFPADVFMIPQYGYLTLNNGSQAVGRSSFYCLEYFPSQMLRTGNNFQFTYTFEDVPFHSSYAHSQSLDRLMNPLIDQYLYYLSRT |
| | | QTTGGTTNTQTLGFSQGGPNTMANQAKNWLPGPCYRQQRVSKTSADNNNSEYSWTGATKYHLNGRDSLVNPGPAMASHKDDEEKFFPQSG |
| | | VLIFGKQGSEKTNVDIEKVMITDEEEIRTTNPVATEQYGSVSTNLQRGNRQAATADVNTQGVLPGMVWQDRDVYLQGPIWAKIPHTDGHF |
| | | HPSPLMGGFGLKHPPPQILIKNTPVPADPPTTENQSKLNSFITQYSTGQVSVEIEWELQKENSKRWNPEIQYTSNYYKSTSVDFAVNTEG |
| | | VYSEPRPIGTRYLTRNL |
| 5 | AAV-DJ VP2 | MAPGKKRPVEHSPVEPDSSSGTGKAGQQPARKRLNFGQTGDADSVPDPQPIGEPPAAPSGVGSLTMAAGGGAPMADNNEGADGVGNSSGN |
| | capsid protein | WHCDSTWMGDRVITTSTRTWALPTYNNHLYKQISNSTSGGSSNDNAYFGYSTPWGYFDFNRFHCHFSPRDWQRLINNNWGFRPKRLSFKL |
| | amino acid | FNIQVKEVTQNEGTKTIANNLTSTIQVFTDSEYQLPYVLGSAHQGCLPPFPADVFMIPQYGYLTLNNGSQAVGRSSFYCLEYFPSQMLRT |
| | sequence | GNNFQFTYTFEDVPFHSSYAHSQSLDRLMNPLIDQYLYYLSRTQTTGGTTNTQTLGFSQGGPNTMANQAKNWLPGPCYRQQRVSKTSADN |
| | | NNSEYSWTGATKYHLNGRDSLVNPGPAMASHKDDEEKFFPQSGVLIFGKQGSEKTNVDIEKVMITDEEEIRTTNPVATEQYGSVSTNLQR |
| | | GNRQAATADVNTQGVLPGMVWQDRDVYLQGPIWAKIPHTDGHFHPSPLMGGFGLKHPPPQILIKNTPVPADPPTTFNQSKLNSFITQYST |
| | | GQVSVEIEWELQKENSKRWNPEIQYTSNYYKSTSVDFAVNTEGVYSEPRPIGTRYLTRNL |
| 6 | AAV-DJ VP 3 | MAAGGGAPMADNNEGADGVGNSSGNWHCDSTWMGDRVITTSTRIWALPTYNNHLYKQISNSTSGGSSNDNAYFGYSTPWGYFDFNRFHCH |
| | capsid protein | FSPRDWQRLINNNWGFRPKRLSFKLFNIQVKEVTQNEGTKTIANNLTSTIQVFTDSEYQLPYVLGSAHQGCLPPFPADVFMIPQYGYLTL |
| | amino acid | NNGSQAVGRSSFYCLEYFPSQMLRTGNNFQFTYTFEDVPFHSSYAHSQSLDRLMNPLIDQYLYYLSRTQTTGGTTNTQTLGFSQGGPNTM |
| | sequence | ANQAKNWLPGPCYRQQRVSKTSADNNNSEYSWTGATKYHLNGRDSLVNPGPAMASHKDDEEKFFPQSGVLIFGKQGSEKTNVDIEKVMIT |
| | | DEEEIRTTNPVATEQYGSVSTNLQRGNRQAATADVNTQGVLPGMVWQDRDVYLQGPIWAKIPHTDGHFHPSPLMGGFGLKHPPPQILIKN |
| | | TPVPADPPTTFNQSKLNSFITQYSTGQVSVEIEWELQKENSKRWNPEIQYTSNYYKSTSVDFAVNTEGVYSEPRPIGTRYLTRNL |
| 7 | 5′ Inverted | TTGGCCACTCCCTCTCTGCGCGCTCGCTCGCTCACTGAGGCCGCCCGGGCAAAGCCCGGGCGTCGGGCGACCTTTGGTCGCCCGGCCTCA |
| | Terminal Repeat | GTGAGCGAGCGAGCGCGCAGAGAGGGAGTGGCCAACTCCATCACTAGGGGTTCCT |
| | (5′-ITR) | |
| | nucleotide | |
| | sequence (flop | |
| | configuration) | |
| 8 | AMBP enhancer | GTTAATTTTTAAAAAGCAGTCAAAAGTCCAAGTGGCCCTTGGCAGCATTTACTCTCTCTGTTTGCTCTGGTTAATAATCTCAGGAGCACA |
| | nucleotide | AACATTCCTGGAGGCAGGAGAAGAAATCAACATCCTGGACTTATCCTCTGGGCCT |
| | sequence | |
| 9 | hALB proximal | GCATGCGTGAGTACTTGTGTGTAAATTTTTCATTATCTATAGGTAAAAGCACACTTGGAATTAGCAATAGATGCAATTTGGGACTTAACT |
| | enhancer | CTTTCAGTATGTCTTATTTCTAAGCAAAGTATTTAGTTTGGTTAGTAATTACTAAACACTGAGAACTAAATTGCAAACACCAAGAACTAA |
| | nucleotide | AATGTTCAAGTGGGAAATTACAGTTAAATACCATGGTAATGAATAAAAGGTACAAATCGTTTAAACTCTTATGTAAAATTTGATAAGATG |
| | sequence | TTTTACACAACTTTAATACATTGACAAGGTCTTGTGGAGAAAAC |
| 10 | hALB core | AGTTCCAGATGGTAAATATACACAAGGGATTTAGTCAAACAATTTTTTGGCAAGAATATTATGAATTTTGTAATCGGTTGGCAGCCAATG |
| | promoter | AAATACAAAGATGAGTCTAGTTAATAATCTACAATTATTGGTTAAAGAAGTATATTAGTGCTAATTTCCCTCCGTTTGTCCTAGCTTTTC |
| | nucleotide | TCTTCTGTCAACCCCACACGCCTTTGGCAC |
| | sequence | |
| 11 | hBetaGlobin | GTGAGTCTATGGGACCCTTGATGTTTTCTTTCCCCTTCTTTTCTATGGTTAAGTTCATGTCATAGGAAGGGGAGAAGTAACAGGGTACAG |
| | intron nucleotide | TTTAGAATGGGAAACAGACGAATGATTGCATCAGTGTGGAAGTCTCAGGATCGTTTTAGTTTCTTTTATTTGCTGTTCATAACAATTGTT |
| | sequence | TTCTTTTGTTTAATTCTTGCTTTCTTTTTTTTTCTTCTCCGCAATTTTTACTATTATACTTAATGCCTTAACATTGTGTATAACAAAAGG |
| | | AAATATCTCTGAGATACATTAAGTAACTTAAAAAAAAACTTTACACAGTCTGCCTAGTACATTACTATTTGGAATATATGTGTGCTTATT |
| | | TGCATATTCATAATCTCCCTACTTTATTTTCTTTTATTTTTAATTGATACATAATCATTATACATATTTATGGGTTAAAGTGTAATGTTT |
| | | TAATATGTGTACACATATTGACCAAATCAGGGTAATTTTGCATTTGTAATTTTAAAAAATGCTTTCTTCTTTTAATATACTTTTTTGTTT |
| | | ATCTTATTTCTAATACTTTCCCTAATCTCTTTCTTTCAGGGCAATAATGATACAATGTATCATGCCTCTTTGCACCATTCTAAAGAATAA |
| | | CAGTGATAATTTCTGGGTTAAGGCAATAGCAATATTTCTGCATATAAATATTTCTGCATATAAATTGTAACTGATGTAAGAGGITTCATA |
| | | TTGCTAATAGCAGCTACAATCCAGCTACCATTCTGCTTTTATTTTATGGTTGGGATAAGGCTGGATTATTCTGAGTCCAAGCTAGGCCCT |
| | | TTTGCTAATCATGTTCATACCTCTTATCTTCCTCCCACAG |
| 12 | IgVH protein | ATGGGATGGAGCTGTATCATCCTCTTCTTGGTGGCAACAGCTACAGGTGTGCACTCC |
| | secretion signal | |
| | peptide encoding | |
| | nucleotide | |
| | sequence | |
| 13 | Human GCase | GCCAGACCCTGCATCCCTAAGAGCTTTGGCTATAGTTCTGTGGTTTGTGTGTGCAATGCCACATACTGTGACTCATTTGACCCTCCTACA |
| | protein mature | TTTCCTGCACTGGGCACTTTCAGCAGGTATGAGTCTACCAGATCTGGCAGGAGGATGGAGCTGAGCATGGGGCCTATCCAGGCTAACCAC |
| | polypeptide | ACCGGCACAGGGCTGCTGCTGACTCTGCAGCCCGAACAGAAATTCCAGAAGGTTAAGGGTTTTGGAGGAGCCATGACAGATGCAGCAGCC |
| | encoding | CTGAACATTTTGGCTCTCAGCCCTCCAGCTCAGAACCTGCTCCTTAAATCATACTTCAGTGAAGAAGGAATTGGCTACAACATCATAAGA |
| | nucleotide | GTACCCATGGCCAGCTGTGATTTTTCCATTAGAACCTACACTTATGCTGATACTCCTGATGATTTTCAGCTGCACAACTTTTCCCTCCCT |
| | sequence from | GAAGAGGACACAAAGCTCAAGATTCCCCTCATTCACAGAGCCCTGCAACTGGCCCAGAGGCCTGTGTCCCTGCTGGCCAGCCCCTGGACA |
| | pAAV-GBA Clone | TCTCCAACATGGCTGAAAACCAATGGAGCAGTAAATGGCAAAGGTTCCCTTAAGGGCCAGCCAGGAGACATCTACCACCAAACCTGGGCC |
| | 128 vector genome | AGGTATTTTGTGAAGTTCCTGGATGCCTATGCAGAGCATAAGCTGCAGTTTTGGGCAGTGACTGCTGAAAATGAACCCTCTGCTGGACTG |
| | | CTCTCAGGGTATCCCTTCCAGTGCCTGGGATTTACTCCTGAGCACCAGAGAGATTTCATTGCCAGAGACCTGGGGCCCACCCTTGCCAAC |
| | | AGCACACACCACAATGTTAGACTGCTGATGCTGGATGACCAGAGGCTGCTCCTTCCCCACTGGGCAAAAGTGGTGCTGACTGACCCAGAG |
| | | GCCGCCAAGTATGTCCATGGCATCGCCGTTCATTGGTACCTGGACTTCCTGGCCCCTGCCAAGGCCACCTTGGGGGAGACACATAGGCTC |
| | | TTCCCCAACACCATGCTGTTTGCCTCAGAAGCCTGTGTGGGTTCCAAATTTTGGGAGCAGTCTGTTAGGCTGGGCTCCTGGGACAGGGGC |
| | | ATGCAGTACAGCCATTCCATCATCACCAATCTGCTGTACCATGTGGTAGGCTGGACCGACTGGAATCTTGCCCTCAACCCAGAAGGTGGC |
| | | CCCAACTGGGTGAGAAACTTCGTGGACAGCCCCATCATCGTGGATATTACCAAAGACACCTTCTACAAGCAGCCCATGTTTTATCACCTG |
| | | GGCCACTTCTCCAAGTTCATCCCTGAGGGAAGCCAGAGAGTGGGATTGGTTGCCTCCCAGAAGAATGATCTCGATGCTGTGGCCCTCATG |
| | | CACCCAGATGGCAGCGCAGTGGTAGTAGTACTGAACAGGTCATCTAAGGATGTGCCTCTCACTATTAAAGATCCTGCAGTGGGCTTCTTG |
| | | GAGACCATCAGCCCCGGATACAGCATCCACACCTACCTGTGGAGGAGACAGTGA |
| 14 | bGH polyA | CGACTGTGCCTTCTAGTTGCCAGCCATCTGTTGTTTGCCCCTCCCCCGTGCCTTCCTTGACCCTGGAAGGTGCCACTCCCACTGTCCTTT |
| | nucleotide | CCTAATAAAATGAGGAAATTGCATCGCATTGTCTGAGTAGGTGTCATTCTATTCTGGGGGGTGGGGTGGGGCAGGACAGCAAGGGGGAGG |
| | sequence | ATTGGGAAGACAATAGCAGGCATGCTGGGGATGCGGTGGGCTCTATGG |
| 15 | 3′ Inverted | AGGAACCCCTAGTGATGGAGTTGGCCACTCCCTCTCTGCGCGCTCGCTCGCTCACTGAGGCCGGGCGACCAAAGGTCGCCCGACGCCCGG |
| | Terminal Repeat | GCTTTGCCCGGGCGGCCTCAGTGAGCGAGCGAGCGCGCAGAGAGGGAGTGGCCAA |
| | (3′-ITR) | |
| | nucleotide | |
| | sequence (flop | |
| | configuration) | |
| 16 | Human GCase | MGWSCIILFLVATATGVHSARPCIPKSEGYSSVVCVCNATYCDSFDPPTFPALGTFSRYESTRSGRRMELSMGPIQANHTGTGLLLTLQP |
| | protein precursor | EQKFQKVKGFGGAMTDAAALNILALSPPAQNLLLKSYFSEEGIGYNIIRVPMASCDFSIRTYTYADTPDDFQLHNFSLPEEDTKLKIPLI |
| | amino acid | HRALQLAQRPVSLLASPWTSPTWLKINGAVNGKGSLKGQPGDIYHQTWARYFVKFLDAYAEHKLQFWAVTAENEPSAGLLSGYPFQCLGF |
| | sequence encoded | TPEHQRDFIARDLGPTLANSTHHNVRLLMLDDQRLLLPHWAKVVLTDPEAAKYVHGIAVHWYLDFLAPAKATLGETHRLFPNTMLFASEA |
| | by GCase | CVGSKFWEQSVRLGSWDRGMQYSHSIITNLLYHVVGWTDWNLALNPEGGPNWVRNFVDSPIIVDITKDTFYKQPMFYHLGHFSKFIPEGS |
| | transgene in | QRVGLVASQKNDLDAVALMHPDGSAVVVVLNRSSKDVPLTIKDPAVGFLETISPGYSIHTYLWRRQ |
| | pAAV-GBA Clone | |
| | 128 vector genome | |
| | (IgVH secretion | |
| | signal peptide | |
| | sequence) | |
| 17 | pAAV-GBA Clone | TTGGCCACTCCCTCTCTGCGCGCTCGCTCGCTCACTGAGGCCGCCCGGGCAAAGCCCGGGCGTCGGGCGACCTTTGGTCGCCCGGCCTCA |
| | 128 vector genome | GTGAGCGAGCGAGCGCGCAGAGAGGGAGTGGCCAACTCCATCACTAGGGGTTCCTTACGTACCTAGGCTCGAGAATTCGTTAATTTTTAA |
| | nucleotide | AAAGCAGTCAAAAGTCCAAGTGGCCCTTGGCAGCATTTACTCTCTCTGTTTGCTCTGGTTAATAATCTCAGGAGCACAAACATTCCTGGA |
| | sequence (5′-ITR | GGCAGGAGAAGAAATCAACATCCTGGACTTATCCTCTGGGCCTGGATCCGTTAATTTTTAAAAAGCAGTCAAAAGTCCAAGTGGCCCTTG |
| | to 3′-ITR) | GCAGCATTTACTCTCTCTGTTTGCTCTGGTTAATAATCTCAGGAGCACAAACATTCCTGGAGGCAGGAGAAGAAATCAACATCCTGGACT |
| | | TATCCTCTGGGCCTCAGATCTGTTAACAGCATGCGTGAGTACTTGTGTGTAAATTTTTCATTATCTATAGGTAAAAGCACACTTGGAATT |
| | | AGCAATAGATGCAATTTGGGACTTAACTCTTTCAGTATGTCTTATTTCTAAGCAAAGTATTTAGTTTGGTTAGTAATTACTAAACACTGA |
| | | GAACTAAATTGCAAACACCAAGAACTAAAATGTTCAAGTGGGAAATTACAGTTAAATACCATGGTAATGAATAAAAGGTACAAATCGTTT |
| | | AAACTCTTATGTAAAATTTGATAAGATGTTTTACACAACTTTAATACATTGACAAGGTCTTGTGGAGAAAACAGTTCCAGATGGTAAATA |
| | | TACACAAGGGATTTAGTCAAACAATTTTTTGGCAAGAATATTATGAATTTTGTAATCGGTTGGCAGCCAATGAAATACAAAGATGAGTCT |
| | | AGTTAATAATCTACAATTATTGGTTAAAGAAGTATATTAGTGCTAATTTCCCTCCGTTTGTCCTAGCTTTTCTCTTCTGTCAACCCCACA |
| | | CGCCTTTGGCACTAGTCAGGTGAGTCTATGGGACCCTTGATGTTTTCTTTCCCCTTCTTTTCTATGGTTAAGTTCATGTCATAGGAAGGG |
| | | GAGAAGTAACAGGGTACAGTTTAGAATGGGAAACAGACGAATGATTGCATCAGTGTGGAAGTCTCAGGATCGTTTTAGTTTCTTTTATTT |
| | | GCTGTTCATAACAATTGTTTTCTTTTGTTTAATTCTTGCTTTCTTTTTTTTTCTTCTCCGCAATTTTTACTATTATACTTAATGCCTTAA |
| | | CATTGTGTATAACAAAAGGAAATATCTCTGAGATACATTAAGTAACTTAAAAAAAAACTTTACACAGTCTGCCTAGTACATTACTATTTG |
| | | GAATATATGTGTGCTTATTTGCATATTCATAATCTCCCTACTTTATTTTCTTTTATTTTTAATTGATACATAATCATTATACATATTTAT |
| | | GGGTTAAAGTGTAATGTTTTAATATGTGTACACATATTGACCAAATCAGGGTAATTTTGCATTTGTAATTTTAAAAAATGCTTTCTTCTT |
| | | TTAATATACTTTTTTGTTTATCTTATTTCTAATACTTTCCCTAATCTCTTTCTTTCAGGGCAATAATGATACAATGTATCATGCCTCTTT |
| | | GCACCATTCTAAAGAATAACAGTGATAATTTCTGGGTTAAGGCAATAGCAATATTTCTGCATATAAATATTTCTGCATATAAATTGTAAC |
| | | TGATGTAAGAGGITTCATATTGCTAATAGCAGCTACAATCCAGCTACCATTCTGCTTTTATTTTATGGTTGGGATAAGGCTGGATTATTC |
| | | TGAGTCCAAGCTAGGCCCTTTTGCTAATCATGTTCATACCTCTTATCTTCCTCCCACAGGTGTCGTTCTAGCACTATCAAGCTTCCCTGG |
| | | CCACCATGGGATGGAGCTGTATCATCCTCTTCTTGGTGGCAACAGCTACAGGTGTGCACTCCGCCAGACCCTGCATCCCTAAGAGCTTTG |
| | | GCTATAGTTCTGTGGTTTGTGTGTGCAATGCCACATACTGTGACTCATTTGACCCTCCTACATTTCCTGCACTGGGCACTTTCAGCAGGT |
| | | ATGAGTCTACCAGATCTGGCAGGAGGATGGAGCTGAGCATGGGGCCTATCCAGGCTAACCACACCGGCACAGGGCTGCTGCTGACTCTGC |
| | | AGCCCGAACAGAAATTCCAGAAGGTTAAGGGTTTTGGAGGAGCCATGACAGATGCAGCAGCCCTGAACATTTTGGCTCTCAGCCCTCCAG |
| | | CTCAGAACCTGCTCCTTAAATCATACTTCAGTGAAGAAGGAATTGGCTACAACATCATAAGAGTACCCATGGCCAGCTGTGATTTTTCCA |
| | | TTAGAACCTACACTTATGCTGATACTCCTGATGATTTTCAGCTGCACAACTTTTCCCTCCCTGAAGAGGACACAAAGCTCAAGATTCCCC |
| | | TCATTCACAGAGCCCTGCAACTGGCCCAGAGGCCTGTGTCCCTGCTGGCCAGCCCCTGGACATCTCCAACATGGCTGAAAACCAATGGAG |
| | | CAGTAAATGGCAAAGGTTCCCTTAAGGGCCAGCCAGGAGACATCTACCACCAAACCTGGGCCAGGTATTTTGTGAAGTTCCTGGATGCCT |
| | | ATGCAGAGCATAAGCTGCAGTTTTGGGCAGTGACTGCTGAAAATGAACCCTCTGCTGGACTGCTCTCAGGGTATCCCTTCCAGTGCCTGG |
| | | GATTTACTCCTGAGCACCAGAGAGATTTCATTGCCAGAGACCTGGGGCCCACCCTTGCCAACAGCACACACCACAATGTTAGACTGCTGA |
| | | TGCTGGATGACCAGAGGCTGCTCCTTCCCCACTGGGCAAAAGTGGTGCTGACTGACCCAGAGGCCGCCAAGTATGTCCATGGCATCGCCG |
| | | TTCATTGGTACCTGGACTTCCTGGCCCCTGCCAAGGCCACCTTGGGGGAGACACATAGGCTCTTCCCCAACACCATGCTGTTTGCCTCAG |
| | | AAGCCTGTGTGGGTTCCAAATTTTGGGAGCAGTCTGTTAGGCTGGGCTCCTGGGACAGGGGCATGCAGTACAGCCATTCCATCATCACCA |
| | | ATCTGCTGTACCATGTGGTAGGCTGGACCGACTGGAATCTTGCCCTCAACCCAGAAGGTGGCCCCAACTGGGTGAGAAACTTCGTGGACA |
| | | GCCCCATCATCGTGGATATTACCAAAGACACCTTCTACAAGCAGCCCATGTTTTATCACCTGGGCCACTTCTCCAAGTTCATCCCTGAGG |
| | | GAAGCCAGAGAGTGGGATTGGTTGCCTCCCAGAAGAATGATCTCGATGCTGTGGCCCTCATGCACCCAGATGGCAGCGCAGTGGTAGTAG |
| | | TACTGAACAGGTCATCTAAGGATGTGCCTCTCACTATTAAAGATCCTGCAGTGGGCTTCTTGGAGACCATCAGCCCCGGATACAGCATCC |
| | | ACACCTACCTGTGGAGGAGACAGTGACATATGGCGACTGTGCCTTCTAGTTGCCAGCCATCTGTTGTTTGCCCCTCCCCCGTGCCTTCCT |
| | | TGACCCTGGAAGGTGCCACTCCCACTGTCCTTTCCTAATAAAATGAGGAAATTGCATCGCATTGTCTGAGTAGGTGTCATTCTATTCTGG |
| | | GGGGTGGGGTGGGGCAGGACAGCAAGGGGGAGGATTGGGAAGACAATAGCAGGCATGCTGGGGATGCGGTGGGCTCTATGGCTAGCGTAC |
| | | GCTTGTGGCAAACTTCGTCAGTCTTATTTTAAGAAATTGCCACAGCTACCACCCTGATCAGTCAGCAGCCAACAACAACGAGGCAAGATC |
| | | CTCTGTCAGCAAAAAGATTTTGATTTGCTGCAGGCTCACAAGATTGTTAGCAATTTTAGCAATAAAGCAATTTTAAATTAAGTTATATAC |
| | | AAATTATTAGACAAATTGCTATTGCACACTTAATAGACTTTAGTGCTAACAAAACTTTTGTAGGCACTGGGAAACCAAAAAATTGTTGCC |
| | | GCTTGCTTTATTGAGTTGGTCTGGAACCTAACCTGTAGTATCTCCGAGGTTTGCCTGTATCTTTAATTGTAATTTGTCCTTCACAGCTAG |
| | | CAGTACTAGGAACCCCTAGTGATGGAGTTGGCCACTCCCTCTCTGCGCGCTCGCTCGCTCACTGAGGCCGGGCGACCAAAGGTCGCCCGA |
| | | CGCCCGGGCTTTGCCCGGGCGGCCTCAGTGAGCGAGCGAGCGCGCAGAGAGGGAGTGGCCAA |
| 18 | AAV2 ITR (130 bp) | aggaacccctagtgatggagttggccactccctctctgcgcgctcgctcgctcactgaggccgggcgaccaaaggtcgcccgacgcccgg |
| | | gctttgcccgggcggcctcagtgagcgagcgagcgcgcag |
| 19 | AAV2 ITR (130 bp) | ctgcgcgctcgctcgctcactgaggccgcccgggcaaagcccgggcgtcgggcgacctttggtcgcccggcctcagtgagcgagcgagcg |
| | | cgcagagagggagtggccaactccatcactaggggttcct |
| 20 | CMV Enhancer and | gacattgattattgactagttattaatagtaatcaattacggggtcattagttcatagcccatatatggagttccgcgttacataactta |
| | Chicken Beta | cggtaaatggcccgcctggctgaccgcccaacgacccccgcccattgacgtcaataatgacgtatgttcccatagtaacgccaataggga |
| | Actin Promoter | ctttccattgacgtcaatgggtggactatttacggtaaactgcccacttggcagtacatcaagtgtatcatatgccaagtacgcccccta |
| | | ttgacgtcaatgacggtaaatggcccgcctggcattatgcccagtacatgaccttatgggactttcctacttggcagtacatctacgtat |
| | | tagtcatcgctattaccatgggtcgaggtgagccccacgttctgcttcactctccccatctcccccccctccccacccccaattttgtat |
| | | ttatttattttttaattattttgtgcagcgatgggggcggggggggggggggcgcgcgccaggcggggcggggcggggcgaggggcgggg |
| | | cggggcgaggcggagaggtgcggcggcagccaatcagagcggcgcgctccgaaagtttccttttatggcgaggcggcggcggcggcggcc |
| | | ctataaaaagcgaagcgcgcggcgggcg |
| 21 | CMV Enhancer | gacattgattattgactagttattaatagtaatcaattacggggtcattagttcatagcccatatatggagttccgcgttacataactta |
| | | cggtaaatggcccgcctggctgaccgcccaacgacccccgcccattgacgtcaataatgacgtatgttcccatagtaacgccaataggga |
| | | ctttccattgacgtcaatgggtggactatttacggtaaactgcccacttggcagtacatcaagtgtatcatatgccaagtacgcccccta |
| | | ttgacgtcaatgacggtaaatggcccgcctggcattatgcccagtacatgaccttatgggactttcctacttggcagtacatctacgtat |
| | | tagtcatcgctattaccatg |
| 22 | Chicken Beta | tcgaggtgagccccacgttctgcttcactctccccatctcccccccctccccacccccaattttgtatttatttattttttaattatttt |
| | Actin Promoter | gtgcagcgatgggggcggggggggggggggggsgcgcgccggggggggggggggggggggggggggggggsggggsgrggcggagaggtg |
| | | cggcggcagccaatcagagcggcgcgctccgaaagtttccttttatggcgaggcggcggcggcggcggccctataaaaagcgaagcgcgc |
| | | ggcgggcg |
| 23 | SV40 Promoter | gcatctcaattagtcagcaaccatagtcccgcccctaactccgcccatcccgcccctaactccgcccagttccgcccattctccgcccca |
| | | tggctgactaattttttttatttatgcagaggccgaggccgcctcggcctctgagctattccagaagtagtgaggaggcttttttggagg |
| | | cctaggcttttgcaaa |
| 24 | SV40 poly (A) | taagatacattgatgagtttggacaaaccacaactagaatgcagtgaaaaaaatgctttatttgtgaaatttgtgatgctattgctttat |
| | Signal | ttgtaaccattataagctgcaataaacaagtt |
| 25 | SV40 poly (A) | aacttgtttattgcagcttataatggttacaaataaagcaatagcatcacaaatttcacaaataaagcatttttttcactgcattctagt |
| | Signal | tgtggtttgtccaaactcatcaatgtatcttatcatgtctggatc |
| 26 | Rabbit Beta- | aataaaggaaatttattttcattgcaatagtgtgttggaattttttgtgtctctca |
| | Globin poly (A) | |
| | Signal | |
| 27 | WPRE | aatcaacctctggattacaaaatttgtgaaagattgactggtattcttaactatgttgctccttttacgctatgtggatacgctgcttta |
| | | atgcctttgtatcatgctattgcttcccgtatggctttcattttctcctccttgtataaatcctggttgctgtctctttatgaggagttg |
| | | tggcccgttgtcaggcaacgtggcgtggtgtgcactgtgtttgctgacgcaacccccactggttggggcattgccaccacctgtcagctc |
| | | ctttccgggactttcgctttccccctccctattgccacggcggaactcatcgccgcctgccttgcccgctgctggacaggggctcggctg |
| | | ttgggcactgacaattccgtggtgttgtcggggaaatcatcgtcctttccttggctgctcgcctgtgttgccacctggattctgcgcggg |
| | | acgtccttctgctacgtcccttcggccctcaatccagcggaccttccttcccgcggcctgctgccggctctgcggcctcttccgcgtctt |
| | | cgccttcgccctcagacgagtcggatctccctttgggccgcctccccgc |
| 28 | Homo sapiens | ATGGAGTTTTCAAGTCCTTCCAGAGAGGAATGTCCCAAGCCTTTGAGTAGGGTAAGCATCATGGCTGGCAGCCTCACAGGATTGCTTCTA |
| | glucosylceramidase | CTTCAGGCAGTGTCGTGGGCATCAGGTGCCCGCCCCTGCATCCCTAAAAGCTTCGGCTACAGCTCGGTGGTGTGTGTCTGCAATGCCACA |
| | beta (GBA), | TACTGTGACTCCTTTGACCCCCCGACCTTTCCTGCCCTTGGTACCTTCAGCCGCTATGAGAGTACACGCAGTGGGCGACGGATGGAGCTG |
| | transcript | AGTATGGGGCCCATCCAGGCTAATCACACGGGCACAGGCCTGCTACTGACCCTGCAGCCAGAACAGAAGTTCCAGAAAGTGAAGGGATTT |
| | variant 1; NCBI | GGAGGGGCCATGACAGATGCTGCTGCTCTCAACATCCTTGCCCTGTCACCCCCTGCCCAAAATTTGCTACTTAAATCGTACTTCTCTGAA |
| | Reference | GAAGGAATCGGATATAACATCATCCGGGTACCCATGGCCAGCTGTGACTTCTCCATCCGCACCTACACCTATGCAGACACCCCTGATGAT |
| | Sequence: | TTCCAGTTGCACAACTTCAGCCTCCCAGAGGAAGATACCAAGCTCAAGATACCCCTGATTCACCGAGCCCTGCAGTTGGCCCAGCGTCCC |
| | NM_000157.4 | GTTTCACTCCTTGCCAGCCCCTGGACATCACCCACTTGGCTCAAGACCAATGGAGCGGTGAATGGGAAGGGGTCACTCAAGGGACAGCCC |
| | | GGAGACATCTACCACCAGACCTGGGCCAGATACTTTGTGAAGTTCCTGGATGCCTATGCTGAGCACAAGITACAGTTCTGGGCAGTGACA |
| | | GCTGAAAATGAGCCTTCTGCTGGGCTGTTGAGTGGATACCCCTTCCAGTGCCTGGGCTTCACCCCTGAACATCAGCGAGACTTCATTGCC |
| | | CGTGACCTAGGTCCTACCCTCGCCAACAGTACTCACCACAATGTCCGCCTACTCATGCTGGATGACCAACGCTTGCTGCTGCCCCACTGG |
| | | GCAAAGGTGGTACTGACAGACCCAGAAGCAGCTAAATATGTTCATGGCATTGCTGTACATTGGTACCTGGACTTTCTGGCTCCAGCCAAA |
| | | GCCACCCTAGGGGAGACACACCGCCTGTTCCCCAACACCATGCTCTTTGCCTCAGAGGCCTGTGTGGGCTCCAAGTTCTGGGAGCAGAGT |
| | | GTGCGGCTAGGCTCCTGGGATCGAGGGATGCAGTACAGCCACAGCATCATCACGAACCTCCTGTACCATGTGGTCGGCTGGACCGACTGG |
| | | AACCTTGCCCTGAACCCCGAAGGAGGACCCAATTGGGTGCGTAACTTTGTCGACAGTCCCATCATTGTAGACATCACCAAGGACACGTTT |
| | | TACAAACAGCCCATGTTCTACCACCTTGGCCACTTCAGCAAGTTCATTCCTGAGGGCTCCCAGAGAGTGGGGCTGGTTGCCAGTCAGAAG |
| | | AACGACCTGGACGCAGTGGCACTGATGCATCCCGATGGCTCTGCTGTTGTGGTCGTGCTAAACCGCTCCTCTAAGGATGTGCCTCTTACC |
| | | ATCAAGGATCCTGCTGTGGGCTTCCTGGAGACAATCTCACCTGGCTACTCCATTCACACCTACCTGTGGCGTCGCCAGTGA |
| 29 | Homo sapiens | ATGGAGTTTTCAAGTCCTTCCAGAGAGGAATGTCCCAAGCCTTTGAGTAGGGTAAGCATCATGGCTGGCAGCCTCACAGGATTGCTTCTA |
| | glucosylceramidase | CTTCAGGCAGTGTCGTGGGCATCAGGTGCCCGCCCCTGCATCCCTAAAAGCTTCGGCTACAGCTCGGTGGTGTGTGTCTGCAATGCCACA |
| | beta (GBA), | TACTGTGACTCCTTTGACCCCCCGACCTTTCCTGCCCTTGGTACCTTCAGCCGCTATGAGAGTACACGCAGTGGGCGACGGATGGAGCTG |
| | transcript | AGTATGGGGCCCATCCAGGCTAATCACACGGGCACAGGCCTGCTACTGACCCTGCAGCCAGAACAGAAGTTCCAGAAAGTGAAGGGATTT |
| | variant 2; NCBI | GGAGGGGCCATGACAGATGCTGCTGCTCTCAACATCCTTGCCCTGTCACCCCCTGCCCAAAATTTGCTACTTAAATCGTACTTCTCTGAA |
| | Reference | GAAGGAATCGGATATAACATCATCCGGGTACCCATGGCCAGCTGTGACTTCTCCATCCGCACCTACACCTATGCAGACACCCCTGATGAT |
| | Sequence: | TTCCAGTTGCACAACTTCAGCCTCCCAGAGGAAGATACCAAGCTCAAGATACCCCTGATTCACCGAGCCCTGCAGTTGGCCCAGCGTCCC |
| | NM_001005741.3 | GTTTCACTCCTTGCCAGCCCCTGGACATCACCCACTTGGCTCAAGACCAATGGAGCGGTGAATGGGAAGGGGTCACTCAAGGGACAGCCC |
| | | GGAGACATCTACCACCAGACCTGGGCCAGATACTTTGTGAAGTTCCTGGATGCCTATGCTGAGCACAAGTTACAGTTCTGGGCAGTGACA |
| | | GCTGAAAATGAGCCTTCTGCTGGGCTGTTGAGTGGATACCCCTTCCAGTGCCTGGGCTTCACCCCTGAACATCAGCGAGACTTCATTGCC |
| | | CGTGACCTAGGTCCTACCCTCGCCAACAGTACTCACCACAATGTCCGCCTACTCATGCTGGATGACCAACGCTTGCTGCTGCCCCACTGG |
| | | GCAAAGGTGGTACTGACAGACCCAGAAGCAGCTAAATATGTTCATGGCATTGCTGTACATTGGTACCTGGACTTTCTGGCTCCAGCCAAA |
| | | GCCACCCTAGGGGAGACACACCGCCTGTTCCCCAACACCATGCTCTTTGCCTCAGAGGCCTGTGTGGGCTCCAAGTTCTGGGAGCAGAGT |
| | | GTGCGGCTAGGCTCCTGGGATCGAGGGATGCAGTACAGCCACAGCATCATCACGAACCTCCTGTACCATGTGGTCGGCTGGACCGACTGG |
| | | AACCTTGCCCTGAACCCCGAAGGAGGACCCAATTGGGTGCGTAACTTTGTCGACAGTCCCATCATTGTAGACATCACCAAGGACACGTTT |
| | | TACAAACAGCCCATGTTCTACCACCTTGGCCACTTCAGCAAGTTCATTCCTGAGGGCTCCCAGAGAGTGGGGCTGGTTGCCAGTCAGAAG |
| | | AACGACCTGGACGCAGTGGCACTGATGCATCCCGATGGCTCTGCTGTTGTGGTCGTGCTAAACCGCTCCTCTAAGGATGTGCCTCTTACC |
| | | ATCAAGGATCCTGCTGTGGGCTTCCTGGAGACAATCTCACCTGGCTACTCCATTCACACCTACCTGTGGCGTCGCCAGTGA |
| 30 | Homo sapiens | ATGGAGTTTTCAAGTCCTTCCAGAGAGGAATGTCCCAAGCCTTTGAGTAGGGTAAGCATCATGGCTGGCAGCCTCACAGGATTGCTTCTA |
| | glucosylceramidase | CTTCAGGCAGTGTCGTGGGCATCAGGTGCCCGCCCCTGCATCCCTAAAAGCTTCGGCTACAGCTCGGTGGTGTGTGTCTGCAATGCCACA |
| | beta (GBA), | TACTGTGACTCCTTTGACCCCCCGACCTTTCCTGCCCTTGGTACCTTCAGCCGCTATGAGAGTACACGCAGTGGGCGACGGATGGAGCTG |
| | transcript | AGTATGGGGCCCATCCAGGCTAATCACACGGGCACAGGCCTGCTACTGACCCTGCAGCCAGAACAGAAGTTCCAGAAAGTGAAGGGATTT |
| | variant 3; NCBI | GGAGGGGCCATGACAGATGCTGCTGCTCTCAACATCCTTGCCCTGTCACCCCCTGCCCAAAATTTGCTACTTAAATCGTACTTCTCTGAA |
| | Reference | GAAGGAATCGGATATAACATCATCCGGGTACCCATGGCCAGCTGTGACTTCTCCATCCGCACCTACACCTATGCAGACACCCCTGATGAT |
| | Sequence: | TTCCAGTTGCACAACTTCAGCCTCCCAGAGGAAGATACCAAGCTCAAGATACCCCTGATTCACCGAGCCCTGCAGTTGGCCCAGCGTCCC |
| | NM_001005742.3 | GTTTCACTCCTTGCCAGCCCCTGGACATCACCCACTTGGCTCAAGACCAATGGAGCGGTGAATGGGAAGGGGTCACTCAAGGGACAGCCC |
| | | GGAGACATCTACCACCAGACCTGGGCCAGATACTTTGTGAAGTTCCTGGATGCCTATGCTGAGCACAAGTTACAGTTCTGGGCAGTGACA |
| | | GCTGAAAATGAGCCTTCTGCTGGGCTGTTGAGTGGATACCCCTTCCAGTGCCTGGGCTTCACCCCTGAACATCAGCGAGACTTCATTGCC |
| | | CGTGACCTAGGTCCTACCCTCGCCAACAGTACTCACCACAATGTCCGCCTACTCATGCTGGATGACCAACGCTTGCTGCTGCCCCACTGG |
| | | GCAAAGGTGGTACTGACAGACCCAGAAGCAGCTAAATATGTTCATGGCATTGCTGTACATTGGTACCTGGACTTTCTGGCTCCAGCCAAA |
| | | GCCACCCTAGGGGAGACACACCGCCTGTTCCCCAACACCATGCTCTTTGCCTCAGAGGCCTGTGTGGGCTCCAAGTTCTGGGAGCAGAGT |
| | | GTGCGGCTAGGCTCCTGGGATCGAGGGATGCAGTACAGCCACAGCATCATCACGAACCTCCTGTACCATGTGGTCGGCTGGACCGACTGG |
| | | AACCTTGCCCTGAACCCCGAAGGAGGACCCAATTGGGTGCGTAACTTTGTCGACAGTCCCATCATTGTAGACATCACCAAGGACACGTTT |
| | | TACAAACAGCCCATGTTCTACCACCTTGGCCACTTCAGCAAGTTCATTCCTGAGGGCTCCCAGAGAGTGGGGCTGGTTGCCAGTCAGAAG |
| | | AACGACCTGGACGCAGTGGCACTGATGCATCCCGATGGCTCTGCTGTTGTGGTCGTGCTAAACCGCTCCTCTAAGGATGTGCCTCTTACC |
| | | ATCAAGGATCCTGCTGTGGGCTTCCTGGAGACAATCTCACCTGGCTACTCCATTCACACCTACCTGTGGCGTCGCCAGTGA |
| 31 | Homo sapiens | ATGGAGCTGAGTATGGGGCCCATCCAGGCTAATCACACGGGCACAGGCCTGCTACTGACCCTGCAGCCAGAACAGAAGTTCCAGAAAGTG |
| | glucosylceramidase | AAGGGATTTGGAGGGGCCATGACAGATGCTGCTGCTCTCAACATCCTTGCCCTGTCACCCCCTGCCCAAAATTTGCTACTTAAATCGTAC |
| | beta (GBA), | TTCTCTGAAGAAGGAATCGGATATAACATCATCCGGGTACCCATGGCCAGCTGTGACTTCTCCATCCGCACCTACACCTATGCAGACACC |
| | transcript | CCTGATGATTTCCAGTTGCACAACTTCAGCCTCCCAGAGGAAGATACCAAGCTCAAGATACCCCTGATTCACCGAGCCCTGCAGTTGGCC |
| | variant 4; NCBI | CAGCGTCCCGTTTCACTCCTTGCCAGCCCCTGGACATCACCCACTTGGCTCAAGACCAATGGAGCGGTGAATGGGAAGGGGTCACTCAAG |
| | Reference | GGACAGCCCGGAGACATCTACCACCAGACCTGGGCCAGATACTTTGTGAAGTTCCTGGATGCCTATGCTGAGCACAAGTTACAGTTCTGG |
| | Sequence: | GCAGTGACAGCTGAAAATGAGCCTTCTGCTGGGCTGTTGAGTGGATACCCCTTCCAGTGCCTGGGCTTCACCCCTGAACATCAGCGAGAC |
| | NM_001171811.2 | TTCATTGCCCGTGACCTAGGTCCTACCCTCGCCAACAGTACTCACCACAATGTCCGCCTACTCATGCTGGATGACCAACGCTTGCTGCTG |
| | | CCCCACTGGGCAAAGGTGGTACTGACAGACCCAGAAGCAGCTAAATATGTTCATGGCATTGCTGTACATTGGTACCTGGACTTTCTGGCT |
| | | CCAGCCAAAGCCACCCTAGGGGAGACACACCGCCTGTTCCCCAACACCATGCTCTTTGCCTCAGAGGCCTGTGTGGGCTCCAAGTTCTGG |
| | | GAGCAGAGTGTGCGGCTAGGCTCCTGGGATCGAGGGATGCAGTACAGCCACAGCATCATCACGAACCTCCTGTACCATGTGGTCGGCTGG |
| | | ACCGACTGGAACCTTGCCCTGAACCCCGAAGGAGGACCCAATTGGGTGCGTAACTTTGTCGACAGTCCCATCATTGTAGACATCACCAAG |
| | | GACACGITTTACAAACAGCCCATGTTCTACCACCTTGGCCACTTCAGCAAGTTCATTCCTGAGGGCTCCCAGAGAGTGGGGCTGGTTGCC |
| | | AGTCAGAAGAACGACCTGGACGCAGTGGCACTGATGCATCCCGATGGCTCTGCTGTTGTGGTCGTGCTAAACCGCTCCTCTAAGGATGTG |
| | | CCTCTTACCATCAAGGATCCTGCTGTGGGCTTCCTGGAGACAATCTCACCTGGCTACTCCATTCACACCTACCTGTGGCGTCGCCAGTGA |
| 32 | Homo sapiens | ATGGAGTTTTCAAGTCCTTCCAGAGAGGAATGTCCCAAGCCTTTGAGTAGGGTAAGCATCATGGCTGGCAGCCTCACAGGATTGCTTCTA |
| | glucosylceramidase | CTTCAGGCAGTGTCGTGGGCATCAGGTGCCCGCCCCTGCATCCCTAAAAGCTTCGGCTACAGCTCGGTGGTGTGTGTCTGCAATGCCACA |
| | beta (GBA), | TACTGTGACTCCTTTGACCCCCCGACCTTTCCTGCCCTTGGTACCTTCAGCCGCTATGAGAGTACACGCAGTGGGCGACGGATGGAGCTG |
| | transcript | AGTATGGGGCCCATCCAGGCTAATCACACGGGCACAGGAATCGGATATAACATCATCCGGGTACCCATGGCCAGCTGTGACTTCTCCATC |
| | variant 5; NCBI | CGCACCTACACCTATGCAGACACCCCTGATGATTTCCAGTTGCACAACTTCAGCCTCCCAGAGGAAGATACCAAGCTCAAGATACCCCTG |
| | Reference | ATTCACCGAGCCCTGCAGTTGGCCCAGCGTCCCGTTTCACTCCTTGCCAGCCCCTGGACATCACCCACTTGGCTCAAGACCAATGGAGCG |
| | Sequence: | GTGAATGGGAAGGGGTCACTCAAGGGACAGCCCGGAGACATCTACCACCAGACCTGGGCCAGATACTTTGTGAAGTTCCTGGATGCCTAT |
| | NM_001171812.2 | GCTGAGCACAAGTTACAGTTCTGGGCAGTGACAGCTGAAAATGAGCCTTCTGCTGGGCTGTTGAGTGGATACCCCTTCCAGTGCCTGGGC |
| | | TTCACCCCTGAACATCAGCGAGACTTCATTGCCCGTGACCTAGGTCCTACCCTCGCCAACAGTACTCACCACAATGTCCGCCTACTCATG |
| | | CTGGATGACCAACGCTTGCTGCTGCCCCACTGGGCAAAGGTGGTACTGACAGACCCAGAAGCAGCTAAATATGTTCATGGCATTGCTGTA |
| | | CATTGGTACCTGGACTTTCTGGCTCCAGCCAAAGCCACCCTAGGGGAGACACACCGCCTGTTCCCCAACACCATGCTCTTTGCCTCAGAG |
| | | GCCTGTGTGGGCTCCAAGTTCTGGGAGCAGAGTGTGCGGCTAGGCTCCTGGGATCGAGGGATGCAGTACAGCCACAGCATCATCACGAAC |
| | | CTCCTGTACCATGTGGTCGGCTGGACCGACTGGAACCTTGCCCTGAACCCCGAAGGAGGACCCAATTGGGTGCGTAACTTTGTCGACAGT |
| | | CCCATCATTGTAGACATCACCAAGGACACGTTTTACAAACAGCCCATGTTCTACCACCTTGGCCACTTCAGCAAGTTCATTCCTGAGGGC |
| | | TCCCAGAGAGTGGGGCTGGTTGCCAGTCAGAAGAACGACCTGGACGCAGTGGCACTGATGCATCCCGATGGCTCTGCTGTTGTGGTCGTG |
| | | CTAAACCGCTCCTCTAAGGATGTGCCTCTTACCATCAAGGATCCTGCTGTGGGCTTCCTGGAGACAATCTCACCTGGCTACTCCATTCAC |
| | | ACCTACCTGTGGCGTCGCCAGTGA |
| 33 | WO2019070894_15 | ATGGAATTCAGCAGCCCCAGCAGAGAGGAATGCCCCAAGCCTCTGAGCCGGGTGTCAATCATGGCCGGATCTCTGACAGGACTGCTGCTG |
| | | CTTCAGGCCGTGTCTTGGGCTTCTGGCGCTAGACCTTGCATCCCCAAGAGCTTCGGCTACAGCAGCGTCGTGTGCGTGTGCAATGCCACC |
| | | TACTGCGACAGCTTCGACCCTCCTACCTTTCCTGCTCTGGGCACCTTCAGCAGATACGAGAGCACCAGATCCGGCAGACGGATGGAACTG |
| | | AGCATGGGACCCATCCAGGCCAATCACACAGGCACTGGCCTGCTGCTGACACTGCAGCCTGAGCAGAAATTCCAGAAAGTGAAAGGCTTC |
| | | GGCGGAGCCATGACAGATGCCGCCGCTCTGAATATCCTGGCTCTGTCTCCACCAGCTCAGAACCTGCTGCTCAAGAGCTACTTCAGCGAG |
| | | GAAGGCATCGGCTACAACATCATCAGAGTGCCCATGGCCAGCTGCGACTTCAGCATCAGGACCTACACCTACGCCGACACACCCGACGAT |
| | | TTCCAGCTGCACAACTTCAGCCTGCCTGAAGAGGACACCAAGCTGAAGATCCCTCTGATCCACAGAGCCCTGCAGCIGGCACAAAGACCC |
| | | GTGTCACTGCTGGCCTCTCCATGGACATCTCCCACCTGGCTGAAAACAAATGGCGCCGTGAATGGCAAGGGCAGCCTGAAAGGCCAACCT |
| | | GGCGACATCTACCACCAGACCTGGGCCAGATACTTCGTGAAGTTCCTGGACGCCTATGCCGAGCACAAGCTGCAGTTTTGGGCCGTGACA |
| | | GCCGAGAACGAACCTTCTGCTGGACTGCTGAGCGGCTACCCCTTTCAGTGCCTGGGCTTTACACCCGAGCACCAGCGGGACTTTATCGCC |
| | | CGTGATCTGGGACCCACACTGGCCAATAGCACCCACCATAATGTGCGGCTGCTGATGCTGGACGACCAGAGACTGCTTCTGCCCCACTGG |
| | | GCTAAAGTGGTGCTGACAGATCCTGAGGCCGCCAAATACGTGCACGGAATCGCCGTGCACTGGTATCTGGACTTTCTGGCCCCTGCCAAG |
| | | GCCACACTGGGAGAGACACACAGACTGTTCCCCAACACCATGCTGTTCGCCAGCGAAGCCTGTGTGGGCAGCAAGTTTTGGGAACAGAGC |
| | | GTGCGGCTCGGCAGCTGGGATAGAGGCATGCAGTACAGCCACAGCATCATCACCAACCTGCTGTACCACGTCGTCGGCTGGACCGACTGG |
| | | AATCTGGCCCTGAATCCTGAAGGCGGCCCTAACTGGGTCCGAAACTTCGTGGACAGCCCCATCATCGTGGACATCACCAAGGACACCTTC |
| | | TACAAGCAGCCCATGTTCTACCACCTGGGACACTTCAGCAAGTTCATCCCCGAGGGCTCTCAGCGCGTTGGACTGGTGGCTTCCCAGAAG |
| | | AACGATCTGGACGCCGTGGCTCTGATGCACCCTGATGGATCTGCTGTGGTGGTGGTCCTGAACCGCAGCAGCAAAGATGTGCCCCTGACC |
| | | ATCAAGGATCCCGCCGTGGGATTCCTGGAAACAATCAGCCCTGGCTACTCCATCCACACCTACCTGTGGCGTAGACAG |
| 34 | WO2020012164_13 | ATGGAGTTCAGCTCCCCTTCTAGGGAGGAGTGCCCTAAGCCACTGTCTCGCGTGAGCATCATGGCAGGAAGCCTGACCGGACTGCTGCTG |
| | | CTGCAGGCCGTGTCCTGGGCATCTGGAGCAAGGCCCTGCATCCCTAAGTCCTTCGGCTACTCTAGCGTGGTGTGCGTGTGCAACGCCACC |
| | | TATTGTGACTCCTTCGATCCCCCTACCTTTCCCGCCCTGGGCACATTTTCTCGGTACGAGAGCACAAGATCCGGCCGGAGAATGGAGCTG |
| | | TCTATGGGCCCCATCCAGGCCAATCACACCGGAACAGGACTGCTGCTGACCCTGCAGCCTGAGCAGAAGTTCCAGAAGGTGAAGGGCTTT |
| | | GGAGGAGCAATGACAGACGCAGCCGCCCTGAACATCCTGGCCCTGAGCCCACCCGCCCAGAATCTGCTGCTGAAGTCTTACTTCAGCGAG |
| | | GAGGGCATCGGCTATAACATCATCCGGGTGCCAATGGCCTCCTGTGACTTTTCTATCAGAACCTACACATATGCCGATACCCCCGACGAT |
| | | TTCCAGCTGCACAATTTTTCCCTGCCTGAGGAGGATACAAAGCTGAAGATCCCACTGATCCACAGGGCCCTGCAGCTGGCACAGAGGCCT |
| | | GTGAGCCTGCTGGCATCCCCATGGACCTCTCCCACATGGCTGAAGACCAACGGCGCCGTGAATGGCAAGGGAAGCCTGAAGGGACAGCCA |
| | | GGCGACATCTACCACCAGACATGGGCCCGGTATTTCGTGAAGTTTCTGGATGCCTACGCCGAGCACAAGCTGCAGTTCTGGGCAGTGACC |
| | | GCAGAGAACGAGCCATCTGCCGGACTGCTGAGCGGATATCCATTCCAGTGCCTGGGCTTTACACCCGAGCACCAGAGGGACTTTATCGCA |
| | | AGGGATCTGGGACCTACCCTGGCCAACTCCACACACCACAATGTGCGGCTGCTGATGCTGGACGATCAGAGACTGCTGCTGCCCCACTGG |
| | | GCCAAGGTGGTGCTGACCGACCCTGAGGCCGCCAAGTACGTGCACGGCATCGCCGTGCACTGGTATCTGGATTTCCTGGCACCAGCAAAG |
| | | GCCACCCTGGGAGAGACACACAGGCTGTTCCCCAACACCATGCTGTTTGCCAGCGAGGCCTGCGTGGGCTCCAAGTTTTGGGAGCAGTCC |
| | | GTGCGGCTGGGATCTTGGGACAGAGGCATGCAGTACAGCCACTCCATCATCACCAATCTGCTGTATCACGTGGTGGGCTGGACAGACTGG |
| | | AACCTGGCCCTGAATCCTGAGGGCGGCCCAAACTGGGTGAGAAATTTCGTGGATAGCCCCATCATCGTGGACATCACCAAGGATACATTC |
| | | TACAAGCAGCCTATGTTTTATCACCTGGGCCACTTCTCTAAGTTTATCCCAGAGGGAAGCCAGAGGGTGGGACTGGTGGCAAGCCAGAAG |
| | | AACGACCTGGATGCCGTGGCCCTGATGCACCCAGATGGCTCCGCCGTGGTGGTGGTGCTGAATCGCTCCTCTAAGGACGTGCCCCTGACC |
| | | ATCAAGGATCCTGCCGTGGGCTTTCTGGAGACCATCAGCCCCGGCTACTCCATCCACACATATCTGTGGAGGCGCCAGTGA |
| 35 | WO2020012164_14 | ATGGAGTTCTCTTCTCCATCTCGCGAGGAGTGCCCTAAGCCACTGAGTCGAGTGTCTATCATGGCCGGCAGTCTGACAGGACTCCTCCTC |
| | | CTACAAGCTGTGTCTTGGGCCAGTGGCGCCCGGCCATGCATCCCAAAGTCTTTCGGGTACTCTAGCGTGGTGTGCGTGTGCAACGCTACA |
| | | TACTGCGACTCTTTCGACCCACCTACATTCCCCGCCCTGGGAACCTTCAGCCGATACGAGTCTACACGGTCCGGGCGGCGCATGGAGCTG |
| | | TCAATGGGGCCAATTCAGGCTAACCACACCGGAACAGGACTCCTGCTGACATTACAACCCGAGCAGAAGTTCCAGAAGGTGAAGGGGTTC |
| | | GGCGGAGCTATGACCGACGCTGCCGCTCTGAACATTCTGGCCCTGTCCCCACCCGCTCAGAACCTGCTCCTCAAGTCTTACTTCAGCGAG |
| | | GAGGGAATCGGGTACAACATTATTAGAGTGCCTATGGCTTCTTGCGACTTCAGCATCCGCACATACACATACGCCGACACCCCTGACGAC |
| | | TTCCAGCTGCACAACTTCAGCCTCCCAGAGGAGGACACAAAGCTCAAGATCCCACTGATTCACCGCGCTTTACAGCTCGCTCAGAGGCCA |
| | | GTGTCTCTGCTCGCTTCCCCTTGGACATCTCCAACATGGTTAAAAACAAACGGCGCCGTGAACGGAAAGGGGTCCCTGAAGGGGCAGCCC |
| | | GGAGACATTTACCACCAGACATGGGCCAGGTACTTCGTGAAGTTCCTCGACGCTTACGCTGAGCACAAGCTCCAGTTCTGGGCCGTGACC |
| | | GCCGAGAACGAGCCTAGCGCCGGCCTGCTGTCCGGATACCCTTTCCAGTGCCTCGGATTCACACCAGAGCACCAGCGGGACTTCATTGCT |
| | | AGAGACCTCGGGCCAACACTCGCTAACTCTACCCACCACAACGTGAGACTGCTGATGCTCGACGACCAGCGCCTCCTGCTGCCTCACTGG |
| | | GCTAAGGTGGTGCTCACAGACCCCGAGGCCGCCAAGTACGTGCACGGAATTGCCGTGCACTGGTATCTCGACTTCCTCGCCCCAGCTAAG |
| | | GCTACACTCGGCGAGACCCACAGGCTGTTCCCTAACACAATGCTGTTCGCCAGCGAGGCTTGCGTGGGAAGCAAGTTCTGGGAGCAGTCC |
| | | GTGAGACTCGGATCTTGGGACCGCGGAATGCAGTACTCTCACTCTATCATTACAAACCTCCTGTACCACGTGGTGGGATGGACCGACTGG |
| | | AACCTGGCTCTCAACCCCGAGGGCGGCCCAAACTGGGTGAGAAACTTCGTGGACAGCCCTATTATTGTGGACATTACAAAGGACACCTTC |
| | | TACAAGCAGCCTATGTTCTACCACCTCGGGCACTTCAGCAAGTTCATCCCCGAGGGATCTCAGAGAGTGGGACTGGTGGCCTCTCAGAAG |
| | | AACGACCTCGACGCTGTGGCTCTGATGCACCCTGACGGGTCGGCCGTGGTGGTGGTGCTCAACCGATCTAGCAAGGACGTGCCACTGACA |
| | | ATTAAGGACCCCGCCGTGGGGTTCCTGGAGACAATTAGCCCCGGATACTCCATTCACACATACCTGTGGCGCAGGCAGTGA |
| 36 | WO2020012164_15 | ATGGAGTTTAGCAGCCCTAGCAGAGAAGAGTGCCCTAAGCCTCTGTCTCGTGTGAGCATCATGGCCGGCTCCTTAACCGGATTATTACTG |
| | | CTGCAAGCTGTGTCTTGGGCTTCCGGCGCTAGGCCTTGTATTCCCAAGTCCTTCGGCTACTCCTCCGTGGTGTGCGTCTGTAATGCCACC |
| | | TACTGTGACAGCTTCGATCCCCCCACATTCCCCGCTCTGGGCACATTTTCTCGTTACGAAAGCACTCGTAGCGGAAGGAGGATGGAGCTG |
| | | AGCATGGGCCCTATTCAAGCTAACCACACCGGCACCGGTTTATTACTGACTTTACAACCCGAGCAGAAGTTCCAGAAGGTGAAGGGCTTT |
| | | GGAGGCGCCATGACAGACGCCGCCGCTTTAAATATTCTGGCTTTAAGCCCTCCCGCCCAAAATCTGCTGCTGAAGTCCTACTTCTCCGAG |
| | | GAAGGCATCGGCTACAACATCATTAGGGTCCCTATGGCCTCTTGTGACTTCAGCATTAGGACCTACACCTACGCCGACACCCCCGACGAC |
| | | TTCCAGCTGCACAACTTCTCTTTACCCGAGGAGGATACAAAGCTGAAGATCCCTTTAATCCATCGTGCCCTCCAGCTGGCTCAGAGGCCC |
| | | GTTAGCTTATTAGCCTCCCCTTGGACATCCCCTACTTGGCTGAAAACCAACGGCGCTGTCAACGGAAAGGGCTCTTTAAAGGGACAGCCC |
| | | GGCGACATTTACCACCAGACATGGGCTAGGTACTTCGTGAAGTTTTTAGACGCTTATGCCGAACACAAGCTGCAGTTCTGGGCCGTGACC |
| | | GCTGAGAACGAGCCTTCCGCCGGTTTACTGTCCGGATACCCCTTCCAATGTCTGGGATTCACCCCCGAACACCAGAGGGACTTCATCGCC |
| | | AGAGATCTGGGCCCCACACTGGCCAATTCCACACACCACAACGTGAGACTGCTCATGCTGGACGACCAGAGGTTATTACTCCCTCACTGG |
| | | GCCAAAGTGGTGCTGACCGACCCCGAAGCTGCCAAGTATGTGCATGGAATCGCCGTGCACTGGTATCTGGACTTTCTGGCCCCCGCCAAG |
| | | GCTACACTGGGCGAGACCCACAGACTGTTCCCTAACACCATGCTCTTCGCCAGCGAAGCTTGCGTGGGAAGCAAGTTCTGGGAGCAATCC |
| | | GTGAGGCTGGGCAGCTGGGATCGTGGCATGCAGTACTCCCACTCCATTATCACAAATCTGCTCTACCACGTCGTGGGATGGACCGATTGG |
| | | AATTTAGCTCTGAACCCCGAGGGAGGCCCCAATTGGGTGAGGAACTTCGTGGATTCCCCCATCATCGTCGATATCACCAAGGACACATTC |
| | | TACAAGCAGCCCATGTTCTACCATTTAGGCCACTTTAGCAAGTTCATCCCCGAGGGAAGCCAGAGAGTGGGTTTAGTGGCCTCCCAGAAG |
| | | AACGATCTGGACGCCGTCGCCCTCATGCATCCCGATGGCAGCGCTGTGGTCGTCGTGCTGAATAGAAGCTCCAAAGACGTCCCTTTAACC |
| | | ATCAAGGACCCCGCCGTGGGCTTTCTGGAAACCATCTCCCCCGGTTATAGCATCCACACCTATTTATGGAGAAGACAGTAA |
| 37 | WO2020012164_16 | ATGGAATTTTCTAGCCCCTCCAGAGAGGAATGCCCCAAGCCTCTGAGCCGGGTGTCAATCATGGCCGGATCTCTGACAGGACTGCTGCTG |
| | | CTTCAGGCCGTGTCTTGGGCTTCTGGCGCTAGACCTTGCATCCCCAAGAGCTTCGGCTACAGCAGCGTCGTGTGCGTGTGCAATGCCACC |
| | | TACTGCGACAGCTTCGACCCTCCTACCTTTCCTGCTCTGGGCACCTTCAGCAGATACGAGAGCACCAGATCCGGCAGACGGATGGAACTG |
| | | AGCATGGGACCCATCCAGGCCAATCACACAGGCACTGGCCTGCTGCTGACACTGCAGCCCGAGCAGAAATTCCAGAAAGTGAAAGGCTTC |
| | | GGCGGAGCCATGACAGATGCCGCCGCTCTGAATATCCTGGCTCTGTCTCCACCAGCTCAGAACCTGCTGCTCAAGAGCTACTTCAGCGAG |
| | | GAAGGCATCGGCTACAACATCATCAGAGTGCCCATGGCCAGCTGCGACTTCAGCATCAGGACCTACACCTACGCCGACACACCCGACGAT |
| | | TTCCAGCTGCACAACTTCAGCCTGCCTGAAGAGGACACCAAGCTGAAGATCCCTCTGATCCACAGAGCCCTGCAGCTGGCACAAAGACCC |
| | | GTTTCTCTGCTGGCTAGCCCCTGGACATCTCCCACCTGGCTGAAAACAAATGGCGCCGTGAATGGCAAGGGCAGCCTGAAAGGCCAACCT |
| | | GGCGATATCTACCACCAGACCTGGGCCAGATACTTCGTGAAGTTCCTGGACGCCTATGCCGAGCACAAGCTGCAGTTTTGGGCCGTGACA |
| | | GCCGAGAACGAACCTTCTGCTGGACTGCTGAGCGGCTACCCCTTTCAGTGCCTGGGCTTTACACCCGAGCACCAGCGGGACTTTATCGCC |
| | | AGAGATCTGGGACCCACACTGGCCAATAGCACCCACCATAATGTGCGGCTGCTGATGCTGGACGACCAGAGACTGCTTCTGCCCCACTGG |
| | | GCTAAAGTGGTGCTGACAGATCCTGAGGCCGCCAAATACGTGCACGGAATCGCCGTGCACTGGTATCTGGACTTTCTGGCCCCTGCCAAG |
| | | GCCACACTGGGAGAGACACACAGACTGTTCCCCAACACCATGCTGTTCGCCAGCGAAGCCTGTGTGGGCAGCAAGTTTTGGGAACAGAGC |
| | | GTGCGGCTCGGCAGCTGGGATAGAGGCATGCAGTACAGCCACAGCATCATCACCAACCTGCTGTACCACGTCGTCGGCTGGACCGACTGG |
| | | AATCTGGCCCTGAATCCTGAAGGCGGCCCTAACTGGGTCCGAAACTTCGTGGACAGCCCCATCATCGTGGACATCACCAAGGACACCTTC |
| | | TACAAGCAGCCCATGTTCTACCACCTGGGACACTTCAGCAAGTTCATCCCCGAGGGCTCTCAGCGCGTTGGACTGGTGGCCAGCCAGAAG |
| | | AATGATCTGGACGCCGTGGCTCTGATGCACCCTGATGGATCTGCTGTGGTGGTGGTCCTGAACCGGTCCAGCAAAGATGTGCCCCTGACC |
| | | ATCAAGGATCCCGCCGTGGGATTCCTGGAAACAATCAGCCCTGGCTACTCCATCCACACCTACCTGTGGCGGAGACAGTAG |
| 38 | IgG heavy chain | MGWSCIILFLVATATGVHS |
| | secretion signal | |
| | peptide amino | |
| | acid sequence | |
| | encoded by SEQ ID | |
| | NO: 12 | |
| 39 | Human GCase | MEFSSPSREECPKPLSRVSIMAGSLTGLLLLQAVSWASG |
| | protein isoform 1 | |
| | secretion signal | |
| | peptide amino | |
| | acid sequence | |
| | (encoded by NCBI | |
| | Reference | |
| | Sequence: | |
| | NM_000157.4) | |
| 40 | Human GCase | ARPCIPKSFGYSSVVCVCNATYCDSFDPPTFPALGTFSRYESTRSGRRMELSMGPIQANHTGTGLLLTLQPEQKFQKVKGfGGAMTDAAA |
| | protein isoform 1 | LNILALSPPAQNLLLKSYFSEEGIGYNIIRVPMASCDFSIRTYTYADTPDDFQLHNFSLPEEDTKLKIPLIHRALQLAQRPVSLLASPWT |
| | mature | SPTWLKTNGAVNGKGSLKGQPGDIYHQTWARYFVKFLDAYAEHKLQFWAVTAENEPSAGLLSGYPFQCLGFTPEHQRDFIARDLGPTLAN |
| | polypeptide amino | STHHNVRLLMLDDQRLLLPHWAKVVLTDPEAAKYVHGIAVHWYLDFLAPAKATLGETHRLEPNTMLFASEACVGSKFWEQSVRLGSWDRG |
| | acid sequence | MQYSHSIITNLLYHVVGWTDWNLALNPEGGPNWVRNFVDSPIIVDITKDTFYKQPMFYHLGHFSKFIPEGSQRVGLVASQKNDLDAVALM |
| | (encoded by NCBI | HPDGSAVVVVLNRSSKDVPLTIKDPAVGFLETISPGYSIHTYLWRRQ |
| | Reference | |
| | Sequence: | |
| | NM_000157.4) | |
| 41 | Human GCase | ATGGGATGGAGCTGTATCATCCTCTTCTTGGTGGCAACAGCTACAGGTGTGCACTCCGCCAGACCCTGCATCCCTAAGAGCTTTGGCTAT |
| | protein precursor | AGTTCTGTGGTTTGTGTGTGCAATGCCACATACTGTGACTCATTTGACCCTCCTACATTTCCTGCACTGGGCACTTTCAGCAGGTATGAG |
| | (SEQ ID NO: 16) | TCTACCAGATCTGGCAGGAGGATGGAGCTGAGCATGGGGCCTATCCAGGCTAACCACACCGGCACAGGGCTGCTGCTGACTCTGCAGCCC |
| | encoding | GAACAGAAATTCCAGAAGGTTAAGGGTTTTGGAGGAGCCATGACAGATGCAGCAGCCCTGAACATTTTGGCTCTCAGCCCTCCAGCTCAG |
| | nucleotide | AACCTGCTCCTTAAATCATACTTCAGTGAAGAAGGAATTGGCTACAACATCATAAGAGTACCCATGGCCAGCTGTGATTTTTCCATTAGA |
| | sequence; in | ACCTACACTTATGCTGATACTCCTGATGATTTTCAGCTGCACAACTTTTCCCTCCCTGAAGAGGACACAAAGCTCAAGATTCCCCTCATT |
| | frame combination | CACAGAGCCCTGCAACTGGCCCAGAGGCCTGTGTCCCTGCTGGCCAGCCCCTGGACATCTCCAACATGGCTGAAAACCAATGGAGCAGTA |
| | of SEQ ID NO: 12 & | AATGGCAAAGGTTCCCTTAAGGGCCAGCCAGGAGACATCTACCACCAAACCTGGGCCAGGTATTTTGTGAAGTTCCTGGATGCCTATGCA |
| | SEQ ID NO: 13 | GAGCATAAGCTGCAGTTTTGGGCAGTGACTGCTGAAAATGAACCCTCTGCTGGACTGCTCTCAGGGTATCCCTTCCAGTGCCTGGGATTT |
| | | ACTCCTGAGCACCAGAGAGATTTCATTGCCAGAGACCTGGGGCCCACCCTTGCCAACAGCACACACCACAATGTTAGACTGCTGATGCTG |
| | | GATGACCAGAGGCTGCTCCTTCCCCACTGGGCAAAAGTGGTGCTGACTGACCCAGAGGCCGCCAAGTATGTCCATGGCATCGCCGTTCAT |
| | | TGGTACCTGGACTTCCTGGCCCCTGCCAAGGCCACCTTGGGGGAGACACATAGGCTCTTCCCCAACACCATGCTGTTTGCCTCAGAAGCC |
| | | TGTGTGGGTTCCAAATTTTGGGAGCAGTCTGTTAGGCTGGGCTCCTGGGACAGGGGCATGCAGTACAGCCATTCCATCATCACCAATCTG |
| | | CTGTACCATGTGGTAGGCTGGACCGACTGGAATCTTGCCCTCAACCCAGAAGGTGGCCCCAACTGGGTGAGAAACTTCGTGGACAGCCCC |
| | | ATCATCGTGGATATTACCAAAGACACCTTCTACAAGCAGCCCATGTTTTATCACCTGGGCCACTTCTCCAAGTTCATCCCTGAGGGAAGC |
| | | CAGAGAGTGGGATTGGTTGCCTCCCAGAAGAATGATCTCGATGCTGTGGCCCTCATGCACCCAGATGGCAGCGCAGTGGTAGTAGTACTG |
| | | AACAGGTCATCTAAGGATGTGCCTCTCACTATTAAAGATCCTGCAGTGGGCTTCTTGGAGACCATCAGCCCCGGATACAGCATCCACACC |
| | | TACCTGTGGAGGAGACAGTGA |
| 42 | Transcription | GTTAATTTTTAAAAAGCAGTCAAAAGTCCAAGTGGCCCTTGGCAGCATTTACTCTCTCTGTTTGCTCTGGTTAATAATCTCAGGAGCACA |
| | control region | AACATTCCTGGAGGCAGGAGAAGAAATCAACATCCTGGACTTATCCTCTGGGCCTGGATCCGTTAATTTTTAAAAAGCAGTCAAAAGTCC |
| | comprising two | AAGTGGCCCTTGGCAGCATTTACTCTCTCTGTTTGCTCTGGTTAATAATCTCAGGAGCACAAACATTCCTGGAGGCAGGAGAAGAAATCA |
| | AMBP ehn, hALB | ACATCCTGGACTTATCCTCTGGGCCTCAGATCTGTTAACAGCATGCGTGAGTACTTGTGTGTAAATTTTTCATTATCTATAGGTAAAAGC |
| | prox ehn, and | ACACTTGGAATTAGCAATAGATGCAATTTGGGACTTAACTCTTTCAGTATGTCTTATTTCTAAGCAAAGTATTTAGTTTGGTTAGTAATT |
| | hALB pro | ACTAAACACTGAGAACTAAATTGCAAACACCAAGAACTAAAATGTTCAAGTGGGAAATTACAGTTAAATACCATGGTAATGAATAAAAGG |
| | | TACAAATCGTTTAAACTCTTATGTAAAATTTGATAAGATGTTTTACACAACTTTAATACATTGACAAGGICTTGTGGAGAAAACAGTTCC |
| | | AGATGGTAAATATACACAAGGGATTTAGTCAAACAATTTTTTGGCAAGAATATTATGAATTTTGTAATCGGTTGGCAGCCAATGAAATAC |
| | | AAAGATGAGTCTAGTTAATAATCTACAATTATTGGTTAAAGAAGTATATTAGTGCTAATTTCCCTCCGTTTGTCCTAGCTTTTCTCTTCT |
| | | GTCAACCCCACACGCCTTTGGCAC |
| 43 | GBA probe primer | TTAGGAAAGGACAGTGGGAGTGGC |
| 44 | GBA forward | AATGCGATGCAATTTCCTCATT |
| | primer | |
| 45 | GBA reverse | TGCCAGCCATCTGTTGTT |
| | primer | |

1. A recombinant adeno-associated virus (AAV) vector genome comprising a nucleotide sequence encoding a beta-glucocerebrosidase (GCase) protein comprising a secretion signal peptide sequence and a mature polypeptide sequence, wherein said secretion signal peptide sequence is from wild-type human GCase protein or from a protein other than GCase.

2. The AAV vector genome of claim 1, wherein said mature GCase polypeptide sequence is identical to the mature polypeptide sequence of wild-type human GCase protein.

3. The AAV vector genome of claim 2, wherein said secretion signal peptide sequence is from an immunoglobulin protein.

4. The AAV vector genome of claim 1, wherein said mature GCase polypeptide sequence comprises the amino acid sequence of SEQ ID NO:40, and said secretion signal peptide sequence comprises the amino acid sequence of SEQ ID NO:38 or SEQ ID NO:39 and/or wherein the amino acid sequence of said GCase protein is provided by the amino acid sequence of SEQ ID NO:16.

5. (canceled)

6. The AAV vector genome of claim 1, wherein said nucleotide sequence encoding GCase protein is wild-type or codon-optimized.

7. The AAV vector genome of claim 6, wherein said codon-optimized nucleotide sequence has reduced number of CpG di-nucleotides compared to wild-type nucleotide sequence.

8. The AAV vector genome of claim 1, wherein said nucleotide sequence encoding mature GCase polypeptide is at least 70% identical to the nucleotide sequence of SEQ ID NO:13.

9. The AAV vector genome of claim 1, wherein said vector genome comprises at least one AAV inverted terminal repeat (ITR) and/or wherein said vector genome further comprises a transcription control region operably linked with said nucleotide sequence encoding GCase protein.

10. (canceled)

11. The AAV vector genome of claim 9, wherein said transcription control region is liver tissue specific.

12. The AAV vector genome of claim 11, wherein said transcription control region comprises a promoter derived from the human albumin (ALB) gene and an enhancer derived from the human albumin (ALB) gene or the alpha-1-microglobulin/bikunin precursor (AMBP) gene.

13. The AAV vector genome of claim 1, wherein said vector genome further comprises a transcription termination signal sequence and/or wherein said vector genome further comprises an intron or a post-transcriptional regulatory element (PRE).

14. (canceled)

15. The AAV vector genome of claim 1, wherein said vector genome comprises in 5′ to 3′ order:
(a) a first AAV2 ITR,
(b) a first copy of an AMBP gene enhancer sequence
(c) a second copy of an AMBP gene enhancer sequence
(d) an ALB gene enhancer sequence
(e) an ALB gene promoter sequence
(f) a human beta globin (HBB) gene intron sequence
(g) a nucleotide sequence encoding a human GCase protein operably linked with said first and second copies of an AMBP enhancer, said ALB enhancer and said ALB promoter, wherein said GCase protein comprises a secretion signal peptide sequence and a mature polypeptide sequence,
(h) a bovine growth hormone (bGH) gene transcription termination signal sequence
(i) a modified TBP intron 2 sequence; and
(j) a second AAV2 ITR.

16. The AAV vector genome of claim 15, wherein each of said first and second copies of an AMBP gene enhancer sequence comprises the nucleotide sequence of SEQ ID NO:8; or said ALB gene enhancer sequence comprises the nucleotide sequence of SEQ ID NO:9; or said ALB gene promoter sequence comprises the nucleotide sequence of SEQ ID NO:10; or said human beta globin (HBB) gene intron sequence comprises the nucleotide sequence of SEQ ID NO:11; or said nucleotide sequence encoding human GCase protein comprises the nucleotide sequence of SEQ ID NO:41; or said bGH gene transcription termination signal sequence comprises the nucleotide sequence of SEQ ID NO:14.

17. The AAV vector genome of claim 1, wherein the nucleotide sequence of said vector genome comprises the nucleotide sequence of SEQ ID NO:17, or the reverse complement thereof.

18. An AAV vector comprising the AAV vector genome of claim 1 and an AAV capsid.

19. The AAV vector of claim 18, wherein said AAV capsid is hepatotropic.

20.-25. (canceled)

26. A DNA plasmid comprising the nucleotide sequence of the AAV vector genome of claim 1.

27. A host cell for AAV vector production comprising the DNA plasmid of claim 26.

28. A method of making a AAV vector, comprising incubating the host cell of claim 27 under conditions sufficient for production of AAV vectors, and purifying the AAV vectors produced thereby.

29. A AAV vector produced by the method of claim 28.


SEQUENCE LISTING (45 sequences)

SEQ ID NO: 1 - protein, 736 aa, unidentified
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 2 - protein, 599 aa, unidentified
MAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 3 - protein, 534 aa, unidentified
MASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 4 - protein, 737 aa, unidentified
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRLLEPLGLVEEAAKTAPGKKRPVEHSPVEPDSSSGTGKAGQQPARKRLNFGQTGDADSVPDPQPIGEPPAAPSGVGSLTMAAGGGAPMADNNEGADGVGNSSGNWHCDSTWMGDRVITTSTRTWALPTYNNHLYKQISNSTSGGSSNDNAYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTTNTQTLGFSQGGPNTMANQAKNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 5 - protein, 600 aa, unidentified
MAPGKKRPVEHSPVEPDSSSGTGKAGQQPARKRLNFGQTGDADSVPDPQPIGEPPAAPSGVGSLTMAAGGGAPMADNNEGADGVGNSSGNWHCDSTWMGDRVITTSTRTWALPTYNNHLYKQISNSTSGGSSNDNAYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTTNTQTLGFSQGGPNTMANQAKNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 6 - protein, 535 aa, unidentified
MAAGGGAPMADNNEGADGVGNSSGNWHCDSTWMGDRVITTSTRTWALPTYNNHLYKQISNSTSGGSSNDNAYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTTNTQTLGFSQGGPNTMANQAKNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 7 - DNA, 145 bp, Adeno-associated dependoparvovirus A
  source 1..145: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 8 - DNA, 145 bp, unidentified
  source 1..145: mol_type = genomic DNA
gttaatttttaaaaagcagtcaaaagtccaagtggcccttggcagcatttactctctctgtttgctctggttaataatctcaggagcacaaacattcctggaggcaggagaagaaatcaacatcctggacttatcctctgggcct

SEQ ID NO: 9 - DNA, 314 bp, Homo sapiens
  source 1..314: mol_type = genomic DNA
gcatgcgtgagtacttgtgtgtaaatttttcattatctataggtaaaagcacacttggaattagcaatagatgcaatttgggacttaactctttcagtatgtcttatttctaagcaaagtatttagtttggttagtaattactaaacactgagaactaaattgcaaacaccaagaactaaaatgttcaagtgggaaattacagttaaataccatggtaatgaataaaaggtacaaatcgtttaaactcttatgtaaaatttgataagatgttttacacaactttaatacattgacaaggtcttgtggagaaaac

SEQ ID NO: 10 - DNA, 210 bp, Homo sapiens
  source 1..210: mol_type = genomic DNA
agttccagatggtaaatatacacaagggatttagtcaaacaattttttggcaagaatattatgaattttgtaatcggttggcagccaatgaaatacaaagatgagtctagttaataatctacaattattggttaaagaagtatattagtgctaatttccctccgtttgtcctagcttttctcttctgtcaaccccacacgcctttggcac

SEQ ID NO: 11 - DNA, 850 bp, Homo sapiens
  source 1..850: mol_type = other DNA
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 12 - DNA, 57 bp, unidentified
  source 1..57: mol_type = other DNA
atgggatggagctgtatcatcctcttcttggtggcaacagctacaggtgtgcactcc

SEQ ID NO: 13 - DNA, 1494 bp, unidentified
  source 1..1494: mol_type = other DNA
gccagaccctgcatccctaagagctttggctatagttctgtggtttgtgtgtgcaatgccacatactgtgactcatttgaccctcctacatttcctgcactgggcactttcagcaggtatgagtctaccagatctggcaggaggatggagctgagcatggggcctatccaggctaaccacaccggcacagggctgctgctgactctgcagcccgaacagaaattccagaaggttaagggttttggaggagccatgacagatgcagcagccctgaacattttggctctcagccctccagctcagaacctgctccttaaatcatacttcagtgaagaaggaattggctacaacatcataagagtacccatggccagctgtgatttttccattagaacctacacttatgctgatactcctgatgattttcagctgcacaacttttccctccctgaagaggacacaaagctcaagattcccctcattcacagagccctgcaactggcccagaggcctgtgtccctgctggccagcccctggacatctccaacatggctgaaaaccaatggagcagtaaatggcaaaggttcccttaagggccagccaggagacatctaccaccaaacctgggccaggtattttgtgaagttcctggatgcctatgcagagcataagctgcagttttgggcagtgactgctgaaaatgaaccctctgctggactgctctcagggtatcccttccagtgcctgggatttactcctgagcaccagagagatttcattgccagagacctggggcccacccttgccaacagcacacaccacaatgttagactgctgatgctggatgaccagaggctgctccttccccactgggcaaaagtggtgctgactgacccagaggccgccaagtatgtccatggcatcgccgttcattggtacctggacttcctggcccctgccaaggccaccttgggggagacacataggctcttccccaacaccatgctgtttgcctcagaagcctgtgtgggttccaaattttgggagcagtctgttaggctgggctcctgggacaggggcatgcagtacagccattccatcatcaccaatctgctgtaccatgtggtaggctggaccgactggaatcttgccctcaacccagaaggtggccccaactgggtgagaaacttcgtggacagccccatcatcgtggatattaccaaagacaccttctacaagcagcccatgttttatcacctgggccacttctccaagttcatccctgagggaagccagagagtgggattggttgcctcccagaagaatgatctcgatgctgtggccctcatgcacccagatggcagcgcagtggtagtagtactgaacaggtcatctaaggatgtgcctctcactattaaagatcctgcagtgggcttcttggagaccatcagccccggatacagcatccacacctacctgtggaggagacagtga

SEQ ID NO: 14 - DNA, 228 bp, Bos taurus
  source 1..228: mol_type = other DNA
cgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 15 - DNA, 145 bp, Adeno-associated dependoparvovirus A
  source 1..145: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 16 - protein, 516 aa, synthetic construct
MGWSCIILFLVATATGVHSARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 17 - DNA, 4202 bp, synthetic construct
  source 1..4202: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttccttacgtacctaggctcgagaattcgttaatttttaaaaagcagtcaaaagtccaagtggcccttggcagcatttactctctctgtttgctctggttaataatctcaggagcacaaacattcctggaggcaggagaagaaatcaacatcctggacttatcctctgggcctggatccgttaatttttaaaaagcagtcaaaagtccaagtggcccttggcagcatttactctctctgtttgctctggttaataatctcaggagcacaaacattcctggaggcaggagaagaaatcaacatcctggacttatcctctgggcctcagatctgttaacagcatgcgtgagtacttgtgtgtaaatttttcattatctataggtaaaagcacacttggaattagcaatagatgcaatttgggacttaactctttcagtatgtcttatttctaagcaaagtatttagtttggttagtaattactaaacactgagaactaaattgcaaacaccaagaactaaaatgttcaagtgggaaattacagttaaataccatggtaatgaataaaaggtacaaatcgtttaaactcttatgtaaaatttgataagatgttttacacaactttaatacattgacaaggtcttgtggagaaaacagttccagatggtaaatatacacaagggatttagtcaaacaattttttggcaagaatattatgaattttgtaatcggttggcagccaatgaaatacaaagatgagtctagttaataatctacaattattggttaaagaagtatattagtgctaatttccctccgtttgtcctagcttttctcttctgtcaaccccacacgcctttggcactagtcaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacaggtgtcgttctagcactatcaagcttccctggccaccatgggatggagctgtatcatcctcttcttggtggcaacagctacaggtgtgcactccgccagaccctgcatccctaagagctttggctatagttctgtggtttgtgtgtgcaatgccacatactgtgactcatttgaccctcctacatttcctgcactgggcactttcagcaggtatgagtctaccagatctggcaggaggatggagctgagcatggggcctatccaggctaaccacaccggcacagggctgctgctgactctgcagcccgaacagaaattccagaaggttaagggttttggaggagccatgacagatgcagcagccctgaacattttggctctcagccctccagctcagaacctgctccttaaatcatacttcagtgaagaaggaattggctacaacatcataagagtacccatggccagctgtgatttttccattagaacctacacttatgctgatactcctgatgattttcagctgcacaacttttccctccctgaagaggacacaaagctcaagattcccctcattcacagagccctgcaactggcccagaggcctgtgtccctgctggccagcccctggacatctccaacatggctgaaaaccaatggagcagtaaatggcaaaggttcccttaagggccagccaggagacatctaccaccaaacctgggccaggtattttgtgaagttcctggatgcctatgcagagcataagctgcagttttgggcagtgactgctgaaaatgaaccctctgctggactgctctcagggtatcccttccagtgcctgggatttactcctgagcaccagagagatttcattgccagagacctggggcccacccttgccaacagcacacaccacaatgttagactgctgatgctggatgaccagaggctgctccttccccactgggcaaaagtggtgctgactgacccagaggccgccaagtatgtccatggcatcgccgttcattggtacctggacttcctggcccctgccaaggccaccttgggggagacacataggctcttccccaacaccatgctgtttgcctcagaagcctgtgtgggttccaaattttgggagcagtctgttaggctgggctcctgggacaggggcatgcagtacagccattccatcatcaccaatctgctgtaccatgtggtaggctggaccgactggaatcttgccctcaacccagaaggtggccccaactgggtgagaaacttcgtggacagccccatcatcgtggatattaccaaagacaccttctacaagcagcccatgttttatcacctgggccacttctccaagttcatccctgagggaagccagagagtgggattggttgcctcccagaagaatgatctcgatgctgtggccctcatgcacccagatggcagcgcagtggtagtagtactgaacaggtcatctaaggatgtgcctctcactattaaagatcctgcagtgggcttcttggagaccatcagccccggatacagcatccacacctacctgtggaggagacagtgacatatggcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctagcgtacgcttgtggcaaacttcgtcagtcttattttaagaaattgccacagctaccaccctgatcagtcagcagccaacaacaacgaggcaagatcctctgtcagcaaaaagattttgatttgctgcaggctcacaagattgttagcaattttagcaataaagcaattttaaattaagttatatacaaattattagacaaattgctattgcacacttaatagactttagtgctaacaaaacttttgtaggcactgggaaaccaaaaaattgttgccgcttgctttattgagttggtctggaacctaacctgtagtatctccgaggtttgcctgtatctttaattgtaatttgtccttcacagctagcagtactaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 18 - DNA, 130 bp, Adeno-associated dependoparvovirus A
  source 1..130: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 19 - DNA, 130 bp, Adeno-associated dependoparvovirus A
  source 1..130: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 20 - DNA, 658 bp, synthetic construct
  source 1..658: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 21 - DNA, 380 bp, synthetic construct
  source 1..380: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 22 - DNA, 278 bp, synthetic construct
  source 1..278: mol_type = other DNA
tcgaggtgagccccacgttctgcttcactctccccatctcccccccctccccacccccaattttgtatttatttattttttaattattttgtgcagcgatgggggcggggggggggggggggsgcgcgccggggggggggggggggggggggggggggggsggggsgrggcggagaggtgcggcggcagccaatcagagcggcgcgctccgaaagtttccttttatggcgaggcggcggcggcggcggccctataaaaagcgaagcgcgcggcgggcg

SEQ ID NO: 23 - DNA, 196 bp, synthetic construct
  source 1..196: mol_type = other DNA
gcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaa

SEQ ID NO: 24 - DNA, 122 bp, synthetic construct
  source 1..122: mol_type = other DNA
taagatacattgatgagtttggacaaaccacaactagaatgcagtgaaaaaaatgctttatttgtgaaatttgtgatgctattgctttatttgtaaccattataagctgcaataaacaagtt

SEQ ID NO: 25 - DNA, 135 bp, synthetic construct
  source 1..135: mol_type = other DNA
aacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 26 - DNA, 56 bp, synthetic construct
  source 1..56: mol_type = other DNA
aataaaggaaatttattttcattgcaatagtgtgttggaattttttgtgtctctca

SEQ ID NO: 27 - DNA, 589 bp, synthetic construct
  source 1..589: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 28 - DNA, 1611 bp, Homo sapiens
  source 1..1611: mol_type = other DNA
atggagttttcaagtccttccagagaggaatgtcccaagcctttgagtagggtaagcatcatggctggcagcctcacaggattgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccagtga

SEQ ID NO: 29 - DNA, 1611 bp, Homo sapiens; identical to SEQ ID NO: 28
  source 1..1611: mol_type = other DNA

SEQ ID NO: 30 - DNA, 1611 bp, Homo sapiens; identical to SEQ ID NO: 28
  source 1..1611: mol_type = other DNA

SEQ ID NO: 31 - DNA, 1350 bp, Homo sapiens
  source 1..1350: mol_type = other DNA
atggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccagtga

SEQ ID NO: 32 - DNA, 1464 bp, Homo sapiens
  source 1..1464: mol_type = other DNA
atggagttttcaagtccttccagagaggaatgtcccaagcctttgagtagggtaagcatcatggctggcagcctcacaggattgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccagtga

SEQ ID NO: 33 - DNA, 1608 bp, synthetic construct
  source 1..1608: mol_type = other DNA
atggaattcagcagccccagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtgtcactgctggcctctccatggacatctcccacctggctgaaaacaaatggcgccgtgaatggcaagggcagcctgaaaggccaacctggcgacatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgcccgtgatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggcttcccagaagaacgatctggacgccgtggctctgatgcaccctgatggatctgctgtggtggtggtcctgaaccgcagcagcaaagatgtgcccctgaccatcaaggatcccgccgtgggattcctggaaacaatcagccctggctactccatccacacctacctgtggcgtagacag

SEQ ID NO: 34 - DNA, 1611 bp, synthetic construct
  source 1..1611: mol_type = other DNA
atggagttcagctccccttctagggaggagtgccctaagccactgtctcgcgtgagcatcatggcaggaagcctgaccggactgctgctgctgcaggccgtgtcctgggcatctggagcaaggccctgcatccctaagtccttcggctactctagcgtggtgtgcgtgtgcaacgccacctattgtgactccttcgatccccctacctttcccgccctgggcacattttctcggtacgagagcacaagatccggccggagaatggagctgtctatgggccccatccaggccaatcacaccggaacaggactgctgctgaccctgcagcctgagcagaagttccagaaggtgaagggctttggaggagcaatgacagacgcagccgccctgaacatcctggccctgagcccacccgcccagaatctgctgctgaagtcttacttcagcgaggagggcatcggctataacatcatccgggtgccaatggcctcctgtgacttttctatcagaacctacacatatgccgatacccccgacgatttccagctgcacaatttttccctgcctgaggaggatacaaagctgaagatcccactgatccacagggccctgcagctggcacagaggcctgtgagcctgctggcatccccatggacctctcccacatggctgaagaccaacggcgccgtgaatggcaagggaagcctgaagggacagccaggcgacatctaccaccagacatgggcccggtatttcgtgaagtttctggatgcctacgccgagcacaagctgcagttctgggcagtgaccgcagagaacgagccatctgccggactgctgagcggatatccattccagtgcctgggctttacacccgagcaccagagggactttatcgcaagggatctgggacctaccctggccaactccacacaccacaatgtgcggctgctgatgctggacgatcagagactgctgctgccccactgggccaaggtggtgctgaccgaccctgaggccgccaagtacgtgcacggcatcgccgtgcactggtatctggatttcctggcaccagcaaaggccaccctgggagagacacacaggctgttccccaacaccatgctgtttgccagcgaggcctgcgtgggctccaagttttgggagcagtccgtgcggctgggatcttgggacagaggcatgcagtacagccactccatcatcaccaatctgctgtatcacgtggtgggctggacagactggaacctggccctgaatcctgagggcggcccaaactgggtgagaaatttcgtggatagccccatcatcgtggacatcaccaaggatacattctacaagcagcctatgttttatcacctgggccacttctctaagtttatcccagagggaagccagagggtgggactggtggcaagccagaagaacgacctggatgccgtggccctgatgcacccagatggctccgccgtggtggtggtgctgaatcgctcctctaaggacgtgcccctgaccatcaaggatcctgccgtgggctttctggagaccatcagccccggctactccatccacacatatctgtggaggcgccagtga

SEQ ID NO: 35 - DNA, 1611 bp, synthetic construct
  source 1..1611: mol_type = other DNA
atggagttctcttctccatctcgcgaggagtgccctaagccactgagtcgagtgtctatcatggccggcagtctgacaggactcctcctcctacaagctgtgtcttgggccagtggcgcccggccatgcatcccaaagtctttcgggtactctagcgtggtgtgcgtgtgcaacgctacatactgcgactctttcgacccacctacattccccgccctgggaaccttcagccgatacgagtctacacggtccgggcggcgcatggagctgtcaatggggccaattcaggctaaccacaccggaacaggactcctgctgacattacaacccgagcagaagttccagaaggtgaaggggttcggcggagctatgaccgacgctgccgctctgaacattctggccctgtccccacccgctcagaacctgctcctcaagtcttacttcagcgaggagggaatcgggtacaacattattagagtgcctatggcttcttgcgacttcagcatccgcacatacacatacgccgacacccctgacgacttccagctgcacaacttcagcctcccagaggaggacacaaagctcaagatcccactgattcaccgcgctttacagctcgctcagaggccagtgtctctgctcgcttccccttggacatctccaacatggttaaaaacaaacggcgccgtgaacggaaaggggtccctgaaggggcagcccggagacatttaccaccagacatgggccaggtacttcgtgaagttcctcgacgcttacgctgagcacaagctccagttctgggccgtgaccgccgagaacgagcctagcgccggcctgctgtccggataccctttccagtgcctcggattcacaccagagcaccagcgggacttcattgctagagacctcgggccaacactcgctaactctacccaccacaacgtgagactgctgatgctcgacgaccagcgcctcctgctgcctcactgggctaaggtggtgctcacagaccccgaggccgccaagtacgtgcacggaattgccgtgcactggtatctcgacttcctcgccccagctaaggctacactcggcgagacccacaggctgttccctaacacaatgctgttcgccagcgaggcttgcgtgggaagcaagttctgggagcagtccgtgagactcggatcttgggaccgcggaatgcagtactctcactctatcattacaaacctcctgtaccacgtggtgggatggaccgactggaacctggctctcaaccccgagggcggcccaaactgggtgagaaacttcgtggacagccctattattgtggacattacaaaggacaccttctacaagcagcctatgttctaccacctcgggcacttcagcaagttcatccccgagggatctcagagagtgggactggtggcctctcagaagaacgacctcgacgctgtggctctgatgcaccctgacgggtcggccgtggtggtggtgctcaaccgatctagcaaggacgtgccactgacaattaaggaccccgccgtggggttcctggagacaattagccccggatactccattcacacatacctgtggcgcaggcagtga

SEQ ID NO: 36 - DNA, 1611 bp, synthetic construct
  source 1..1611: mol_type = other DNA
atggagtttagcagccctagcagagaagagtgccctaagcctctgtctcgtgtgagcatcatggccggctccttaaccggattattactgctgcaagctgtgtcttgggcttccggcgctaggccttgtattcccaagtccttcggctactcctccgtggtgtgcgtctgtaatgccacctactgtgacagcttcgatccccccacattccccgctctgggcacattttctcgttacgaaagcactcgtagcggaaggaggatggagctgagcatgggccctattcaagctaaccacaccggcaccggtttattactgactttacaacccgagcagaagttccagaaggtgaagggctttggaggcgccatgacagacgccgccgctttaaatattctggctttaagccctcccgcccaaaatctgctgctgaagtcctacttctccgaggaaggcatcggctacaacatcattagggtccctatggcctcttgtgacttcagcattaggacctacacctacgccgacacccccgacgacttccagctgcacaacttctctttacccgaggaggatacaaagctgaagatccctttaatccatcgtgccctccagctggctcagaggcccgttagcttattagcctccccttggacatcccctacttggctgaaaaccaacggcgctgtcaacggaaagggctctttaaagggacagcccggcgacatttaccaccagacatgggctaggtacttcgtgaagtttttagacgcttatgccgaacacaagctgcagttctgggccgtgaccgctgagaacgagccttccgccggtttactgtccggataccccttccaatgtctgggattcacccccgaacaccagagggacttcatcgccagagatctgggccccacactggccaattccacacaccacaacgtgagactgctcatgctggacgaccagaggttattactccctcactgggccaaagtggtgctgaccgaccccgaagctgccaagtatgtgcatggaatcgccgtgcactggtatctggactttctggcccccgccaaggctacactgggcgagacccacagactgttccctaacaccatgctcttcgccagcgaagcttgcgtgggaagcaagttctgggagcaatccgtgaggctgggcagctgggatcgtggcatgcagtactcccactccattatcacaaatctgctctaccacgtcgtgggatggaccgattggaatttagctctgaaccccgagggaggccccaattgggtgaggaacttcgtggattcccccatcatcgtcgatatcaccaaggacacattctacaagcagcccatgttctaccatttaggccactttagcaagttcatccccgagggaagccagagagtgggtttagtggcctcccagaagaacgatctggacgccgtcgccctcatgcatcccgatggcagcgctgtggtcgtcgtgctgaatagaagctccaaagacgtccctttaaccatcaaggaccccgccgtgggctttctggaaaccatctcccccggttatagcatccacacctatttatggagaagacagtaa

SEQ ID NO: 37 - DNA, 1611 bp, synthetic construct
  source 1..1611: mol_type = other DNA
atggaattttctagcccctccagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcccgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtttctctgctggctagcccctggacatctcccacctggctgaaaacaaatggcgccgtgaatggcaagggcagcctgaaaggccaacctggcgatatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgccagagatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggccagccagaagaatgatctggacgccgtggctctgatgcaccctgatggatctgctgtggtggtggtcctgaaccggtccagcaaagatgtgcccctgaccatcaaggatcccgccgtgggattcctggaaacaatcagccctggctactccatccacacctacctgtggcggagacagtag

SEQ ID NO: 38 - protein, 19 aa, synthetic construct
MGWSCIILFLVATATGVHS

SEQ ID NO: 39 - protein, 39 aa, synthetic construct
MEFSSPSREECPKPLSRVSIMAGSLTGLLLLQAVSWASG

SEQ ID NO: 40 - protein, 497 aa, synthetic construct
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 41 - DNA, 1551 bp, synthetic construct
  source 1..1551: mol_type = other DNA
atgggatggagctgtatcatcctcttcttggtggcaacagctacaggtgtgcactccgccagaccctgcatccctaagagctttggctatagttctgtggtttgtgtgtgcaatgccacatactgtgactcatttgaccctcctacatttcctgcactgggcactttcagcaggtatgagtctaccagatctggcaggaggatggagctgagcatggggcctatccaggctaaccacaccggcacagggctgctgctgactctgcagcccgaacagaaattccagaaggttaagggttttggaggagccatgacagatgcagcagccctgaacattttggctctcagccctccagctcagaacctgctccttaaatcatacttcagtgaagaaggaattggctacaacatcataagagtacccatggccagctgtgatttttccattagaacctacacttatgctgatactcctgatgattttcagctgcacaacttttccctccctgaagaggacacaaagctcaagattcccctcattcacagagccctgcaactggcccagaggcctgtgtccctgctggccagcccctggacatctccaacatggctgaaaaccaatggagcagtaaatggcaaaggttcccttaagggccagccaggagacatctaccaccaaacctgggccaggtattttgtgaagttcctggatgcctatgcagagcataagctgcagttttgggcagtgactgctgaaaatgaaccctctgctggactgctctcagggtatcccttccagtgcctgggatttactcctgagcaccagagagatttcattgccagagacctggggcccacccttgccaacagcacacaccacaatgttagactgctgatgctggatgaccagaggctgctccttccccactgggcaaaagtggtgctgactgacccagaggccgccaagtatgtccatggcatcgccgttcattggtacctggacttcctggcccctgccaaggccaccttgggggagacacataggctcttccccaacaccatgctgtttgcctcagaagcctgtgtgggttccaaattttgggagcagtctgttaggctgggctcctgggacaggggcatgcagtacagccattccatcatcaccaatctgctgtaccatgtggtaggctggaccgactggaatcttgccctcaacccagaaggtggccccaactgggtgagaaacttcgtggacagccccatcatcgtggatattaccaaagacaccttctacaagcagcccatgttttatcacctgggccacttctccaagttcatccctgagggaagccagagagtgggattggttgcctcccagaagaatgatctcgatgctgtggccctcatgcacccagatggcagcgcagtggtagtagtactgaacaggtcatctaaggatgtgcctctcactattaaagatcctgcagtgggcttcttggagaccatcagccccggatacagcatccacacctacctgtggaggagacagtga

SEQ ID NO: 42 - DNA, 834 bp, synthetic construct
  source 1..834: mol_type = other DNA
gttaatttttaaaaagcagtcaaaagtccaagtggcccttggcagcatttactctctctgtttgctctggttaataatctcaggagcacaaacattcctggaggcaggagaagaaatcaacatcctggacttatcctctgggcctggatccgttaatttttaaaaagcagtcaaaagtccaagtggcccttggcagcatttactctctctgtttgctctggttaataatctcaggagcacaaacattcctggaggcaggagaagaaatcaacatcctggacttatcctctgggcctcagatctgttaacagcatgcgtgagtacttgtgtgtaaatttttcattatctataggtaaaagcacacttggaattagcaatagatgcaatttgggacttaactctttcagtatgtcttatttctaagcaaagtatttagtttggttagtaattactaaacactgagaactaaattgcaaacaccaagaactaaaatgttcaagtgggaaattacagttaaataccatggtaatgaataaaaggtacaaatcgtttaaactcttatgtaaaatttgataagatgttttacacaactttaatacattgacaaggtcttgtggagaaaacagttccagatggtaaatatacacaagggatttagtcaaacaattttttggcaagaatattatgaattttgtaatcggttggcagccaatgaaatacaaagatgagtctagttaataatctacaattattggttaaagaagtatattagtgctaatttccctccgtttgtcctagcttttctcttctgtcaaccccacacgcctttggcac

SEQ ID NO: 43 - DNA, 24 bp, synthetic construct
  source 1..24: mol_type = other DNA
ttaggaaaggacagtgggagtggc

SEQ ID NO: 44 - DNA, 22 bp, synthetic construct
  source 1..22: mol_type = other DNA
aatgcgatgcaatttcctcatt

SEQ ID NO: 45 - DNA, 18 bp, synthetic construct
  source 1..18: mol_type = other DNA
tgccagccatctgttgtt